(19) United States
(12) Patent Application Publication
(10) Pub. No.: US 2026/0215150 A1
(43) Pub. Date: Jul. 23, 2026

(54) COMPOSITION FOR ORGANIC OPTOELECTRONIC DEVICE, ORGANIC OPTOELECTRONIC DEVICE, AND DISPLAY APPARATUS

(71) Applicant: SAMSUNG SDI CO., LTD., Yongin-si, Gyeonggi-do (KR)

(72) Inventors: Changwoo KIM, Suwon-si, Gyeonggi-do (KR); Dong Min KANG, Suwon-si, Gyeonggi-do (KR); Kipo JANG, Suwon-si, Gyeonggi-do (KR); Hyung Sun KIM, Suwon-si, Gyeonggi-do (KR); Jihee LEE, Suwon-si, Gyeonggi-do (KR); Jinseok JANG, Suwon-si, Gyeonggi-do (KR); Mijin LEE, Suwon-si, Gyeonggi-do (KR); Sung-Hyun JUNG, Suwon-si, Gyeonggi-do (KR); Hansol SEO, Suwon-si, Gyeonggi-do (KR)

(21) Appl. No.: 19/142,708

(22) PCT Filed: Dec. 26, 2023
(86) PCT No.: PCT/KR2023/021539

§ 371 Date: Jun. 24, 2025

(30) Foreign Application Priority Data
Dec. 26, 2022 (KR) 10-2022-0184759
Dec. 21, 2023 (KR) 10-2023-0188936

(51) Int. Cl. H10K 85/60; C07D 403/10; C07D 405/14; C09K 11/02; H10K 50/12

(52) U.S. Cl. CPC H10K 85/654; C07D 403/10; C07D 405/14; C09K 11/02; H10K 85/6572; H10K 85/6574; H10K 50/12

(57) ABSTRACT

Disclosed are a composition for an organic optoelectronic device including a first compound represented by Chemical Formula 1, and a second compound represented by Chemical Formula 2, and an organic optoelectronic device including the same and a display device.

The details of Chemical Formula 1 and Chemical Formula 2 are as defined in the specification.

### TECHNICAL FIELD

A composition for an organic optoelectronic device, an organic optoelectronic device, and a display device are disclosed.

### BACKGROUND ART

An organic optoelectronic device (organic optoelectronic diode) is a device capable of converting electrical energy and optical energy to each other.

Organic optoelectronic devices may be largely divided into two types according to a principle of operation. One is a photoelectric device that generates electrical energy by separating excitons formed by light energy into electrons and holes, and transferring the electrons and holes to different electrodes, respectively and the other is light emitting device that generates light energy from electrical energy by supplying voltage or current to the electrodes.

Examples of the organic optoelectronic device include an organic photoelectric device, an organic light emitting diode, an organic solar cell, and an organic photoconductor drum.

Among them, organic light emitting diodes (OLEDs) are attracting much attention in recent years due to increasing demands for flat panel display devices. The organic light emitting diode is a device that converts electrical energy into light, and the performance of the organic light emitting diode is greatly influenced by an organic material between electrodes.

### DISCLOSURE

### Technical Problem

An embodiment provides a composition for an organic optoelectronic device capable of implementing a high-efficiency and long life-span organic optoelectronic device.

Another embodiment provides an organic optoelectronic device including the composition for the organic optoelectronic device.

Another embodiment provides a display device including the organic optoelectronic device.

### Technical Solution

According to an embodiment, a composition for an organic optoelectronic device includes a first compound represented by Chemical Formula 1, and a second compound represented by Chemical Formula 2.

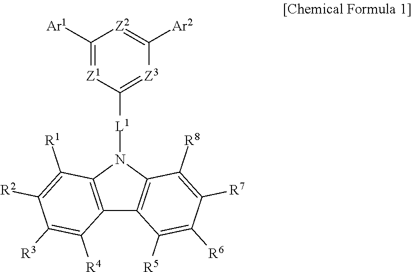

In Chemical Formula 1,
Z¹ to Z³ are each independently N or C—R^{a},
at least two of Z¹ to Z³ are N,
L¹ is a single bond or a substituted or unsubstituted C6 to C20 arylene group,
Ar¹ and Ar² are each independently a substituted or unsubstituted C6 to C30 aryl group, and
R^{a}, and R¹ to R⁸ are each independently hydrogen, deuterium, a substituted or unsubstituted C1 to C10 alkyl group, a substituted or unsubstituted C6 to C30 aryl group, a substituted or unsubstituted C2 to C30 heterocyclic group, or a combination thereof,

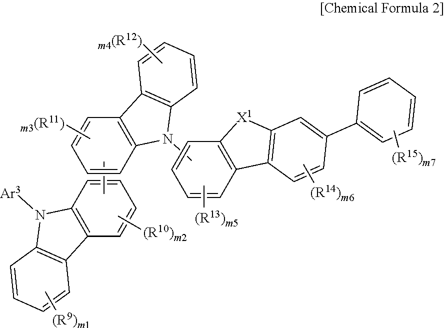

wherein, in Chemical Formula 2,
X¹ is O or S,
Ar³ is a substituted or unsubstituted C6 to C30 aryl group, a substituted or unsubstituted C2 to C30 heterocyclic group, or a combination thereof,
R⁹ to R¹² are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group,
R¹³ to R¹⁵ are each independently hydrogen, deuterium, or substituted or unsubstitutedphenyl group,
m2, m3, m5, and m6 are each independently one of integers of 1 to 3,
m1 and m4 are each independently one of integers of 1 to 4, and
m7 is one of integers of 1 to 5.

According to another embodiment, an organic optoelectronic device includes an anode and a cathode facing each other, and at least one organic layer between the anode and the cathode, wherein the organic layer includes the composition for the organic optoelectronic device.

According to another embodiment, a display device including the organic optoelectronic device is provided.

### Advantageous Effects

An organic optoelectronic device having high efficiency, long life-span, and low driving may be realized.

### DESCRIPTION OF THE DRAWINGS

FIG. 1 is a cross-sectional view illustrating an organic light emitting diode according to an embodiment.

### DESCRIPTION OF SYMBOLS

100: organic light emitting diode
105: organic layer
110: cathode
120: anode
130: light emitting layer
140: hole transport region
150: electron transport region

### BEST MODE

Hereinafter, embodiments of the present invention are described in detail. However, these embodiments are exemplary, and this disclosure is not limited thereto.

In the present specification when a definition is not otherwise provided, “substituted” refers to replacement of at least one hydrogen of a substituent or a compound by deuterium, a halogen, a hydroxyl group, an amino group, a substituted or unsubstituted C1 to C30 amine group, a nitro group, a substituted or unsubstituted C1 to C40 silyl group, a C1 to C30 alkyl group, a C1 to C10 alkylsilyl group, a C6 to C30 arylsilyl group, a C3 to C30 cycloalkyl group, a C3 to C30 heterocycloalkyl group, a C6 to C30 aryl group, a C2 to C30 heteroaryl group, a C1 to C20 alkoxy group, a C1 to C10 trifluoroalkyl group, a cyano group, or a combination thereof.

In one example of the present invention, the “substituted” refers to replacement of at least one hydrogen of a substituent or a compound by deuterium, a cyano group, a C1 to C30 alkyl group, a C1 to C10 alkylsilyl group, a C6 to C30 arylamine group, a C6 to C30 arylsilyl group, a C3 to C30 cycloalkyl group, a C3 to C30 heterocycloalkyl group, a C6 to C30 aryl group, or a C2 to C30 heteroaryl group. In specific example of the present invention, the “substituted” refers to replacement of at least one hydrogen of a substituent or a compound by deuterium, a cyano group, a C1 to C20 alkyl group, a C6 to C30 arylamine group, a C6 to C30 aryl group, or a C2 to C30 heteroaryl group. In specific example of the present invention, the “substituted” refers to replacement of at least one hydrogen of a substituent or a compound by deuterium, a cyano group, a C1 to C5 alkyl group, a C6 to C20 arylamine group, a C6 to C18 aryl group, a dibenzofuranyl group, a dibenzothiophenyl group, a carbazolyl group, or a pyridinyl group. In specific example of the present invention, the “substituted” refers to replacement of at least one hydrogen of a substituent or a compound by deuterium, a cyano group, a methyl group, an ethyl group, a propyl group, a butyl group, a C6 to C20 arylamine group, a phenyl group, a biphenyl group, a terphenyl group, a naphthyl group, a triphenyl group, a fluorenyl group, a dibenzofuranyl group, a dibenzothiophenyl group, a carbazolyl group, or a pyridinyl group.

In the present specification, “unsubstituted” refers to non-replacement of a hydrogen atom by another substituent and remaining of the hydrogen atom.

In the present specification, “hydrogen substitution (—H)” may include “deuterium substitution (-D)” or “tritium substitution (-T).”

In the present specification when a definition is not otherwise provided, “hetero” refers to one including one to three heteroatoms selected from N, O, S, P, and Si, and remaining carbons in one functional group.

In the present specification, “aryl group” refers to a group including at least one hydrocarbon aromatic moiety, and all elements of the hydrocarbon aromatic moiety have p-orbitals which form conjugation, for example a phenyl group, a naphthyl group, and the like, two or more hydrocarbon aromatic moieties may be linked by a sigma bond and may be, for example a biphenyl group, a terphenyl group, a quaterphenyl group, and the like, and two or more hydrocarbon aromatic moieties are fused directly or indirectly to provide a non-aromatic fused ring, for example a fluorenyl group.

The aryl group may include a monocyclic, polycyclic, or fused ring polycyclic (i.e., rings sharing adjacent pairs of carbon atoms) functional group.

In the present specification, “heterocyclic group” is a generic concept of a heteroaryl group, and may include at least one heteroatom selected from N, O, S, P, and Si instead of carbon (C) in a cyclic compound such as aryl group, a cycloalkyl group, a fused ring thereof, or a combination thereof. When the heterocyclic group is a fused ring, the entire ring or each ring of the heterocyclic group may include one or more heteroatoms.

For example, “heteroaryl group” may refer to aryl group including at least one heteroatom selected from N, O, S, P, and Si. Two or more heteroaryl groups are linked by a sigma bond directly, or when the heteroaryl group includes two or more rings, the two or more rings may be fused. When the heteroaryl group is a fused ring, each ring may include one to three heteroatoms.

More specifically, the substituted or unsubstituted C6 to C30 aryl group may be a substituted or unsubstituted phenyl group, a substituted or unsubstituted naphthyl group, a substituted or unsubstituted anthracenyl group, a substituted or unsubstituted phenanthrenyl group, a substituted or unsubstituted naphthacenyl group, a substituted or unsubstituted pyrenyl group, a substituted or unsubstituted biphenyl group, a substituted or unsubstituted p-terphenyl group, a substituted or unsubstituted m-terphenyl group, a substituted or unsubstituted o-terphenyl group, a substituted or unsubstituted chrysenyl group, a substituted or unsubstituted triphenylene group, a substituted or unsubstituted perylenyl group, a substituted or unsubstituted fluorenyl group, a substituted or unsubstituted indenyl group, or a combination thereof, but is not limited thereto.

More specifically, the substituted or unsubstituted C2 to C30 heterocyclic group may be a substituted or unsubstituted furanyl group, a substituted or unsubstituted thiophenyl group, a substituted or unsubstituted pyrrolyl group, a substituted or unsubstituted pyrazolyl group, a substituted or unsubstituted imidazolyl group, a substituted or unsubstituted triazolyl group, a substituted or unsubstituted oxazolyl group, a substituted or unsubstituted thiazolyl group, a substituted or unsubstituted oxadiazolyl group, a substituted or unsubstituted thiadiazolyl group, a substituted or unsubstituted pyridyl group, a substituted or unsubstituted pyrimidinyl group, a substituted or unsubstituted pyrazinyl group, a substituted or unsubstituted triazinyl group, a substituted or unsubstituted benzofuranyl group, a substituted or unsubstituted benzothiophenyl group, a substituted or unsubstituted benzimidazolyl group, a substituted or unsubstituted indolyl group, a substituted or unsubstituted quinolinyl group, a substituted or unsubstituted isoquinolinyl group, a substituted or unsubstituted quinazolinyl group, a substituted or unsubstituted quinoxalinyl group, a substituted or unsubstituted naphthyridinyl group, a substituted or unsubstituted benzoxazinyl group, a substituted or unsubstituted benzothiazinyl group, a substituted or unsubstituted acridinyl group, a substituted or unsubstituted phenazinyl group, a substituted or unsubstituted phenothiazinyl group, a substituted or unsubstituted phenoxazinyl group, a substituted or unsubstituted dibenzofuranyl group, or a substituted or unsubstituted dibenzothiophenyl group, or a combination thereof, but is not limited thereto.

In the present specification, hole characteristics refer to an ability to donate an electron to form a hole when an electric field is applied and that a hole formed in the anode may be easily injected into the light emitting layer and transported in the light emitting layer due to conductive characteristics according to a highest occupied molecular orbital (HOMO) level.

In addition, electronic characteristics refer to an ability to accept an electron when an electric field is applied and that electron formed in the cathode may be easily injected into the light emitting layer and transported in the light emitting layer due to conductive characteristics according to a lowest unoccupied molecular orbital (LUMO) level.

Hereinafter, a composition for an organic optoelectronic device according to an embodiment is described.

The composition for the organic optoelectronic device according to an embodiment includes a first compound represented by Chemical Formula 1, and a second compound represented by Chemical Formula 2.

The first compound may be represented by Chemical Formula 1.

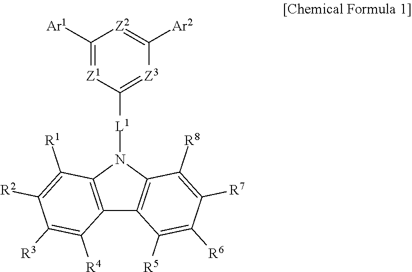

In Chemical Formula 1,
Z¹ to Z³ are each independently N or C—R^{a},
at least two of Z¹ to Z³ are N,
L¹ is a single bond or a substituted or unsubstituted C6 to C20 arylene group,
Ar¹ and Ar² are each independently a substituted or unsubstituted C6 to C30 aryl group, and
R^{a}, and R¹ to R⁸ are each independently hydrogen, deuterium, a substituted or unsubstituted C1 to C10 alkyl group, a substituted or unsubstituted C6 to C30 aryl group, a substituted or unsubstituted C2 to C30 heterocyclic group, or a combination thereof.

The first compound represented by Chemical Formula 1 has a structure in which a carbazole is used as a basic skeleton and a ring including at least one nitrogen (a nitrogen-containing 6-membered ring) is substituted at the 9th position of the carbazole.

The first compound may have a structure that is easy to receive electrons when an electric field is applied by including a ring containing at least one nitrogen, and thus, the driving voltage of an organic optoelectronic device to which the compound is applied may be lowered.

In addition, the first compound forms a bipolar structure by including carbazole, which is easy to accept holes, so that the flow of holes and electrons may be appropriately balanced, thereby improving the efficiency of an organic optoelectronic device to which the compound is applied.

In particular, by linking to the nitrogen-containing 6-membered ring in the 9-direction (N-direction) of carbazole, a π-bond through the C—N bond is broken, so that the electron cloud between the HOMO and LUMO is clearly localized into the hole transport portion and the electron transport portion, thereby widening a HOMO-LUMO bandgap, so that the efficiency of the organic light emitting diode to which it is applied may be further improved.

For example, in Chemical Formula 1, Ar¹ and Ar² may each independently be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, or a substituted or unsubstituted fluorenyl group.

As a specific example, in Chemical Formula 1, Ar¹ and Ar² may each independently be a substituted or unsubstituted phenyl group, a substituted or unsubstituted biphenyl group, or a substituted or unsubstituted triphenylene group.

For example, in Chemical Formula 1, R¹ to R⁸ may each independently be hydrogen, deuterium, a cyano group, a substituted or unsubstituted C6 to C20 aryl group, or a substituted or unsubstituted C2 to C20 heterocyclic group.

As a specific example, in Chemical Formula 1, R¹ to R⁸ may each independently be hydrogen, deuterium, a cyano group, a substituted or unsubstituted phenyl group, a substituted or unsubstituted biphenyl group, a substituted or unsubstituted phenyl group carbazolyl group, a substituted or unsubstituted phenyl group dibenzofuranyl group, or a substituted or unsubstituted phenyl group dibenzothiophenyl group.

For example, in Chemical Formula 1, L¹ may be a single bond, a substituted or unsubstituted phenylene group, a substituted or unsubstituted biphenylene group, or a substituted or unsubstituted triphenylenylene group.

For example, in Chemical Formula 1, L¹ may be a substituted or unsubstituted phenylene group.

The first compound may be, for example, one selected from the compounds listed in Group 1.

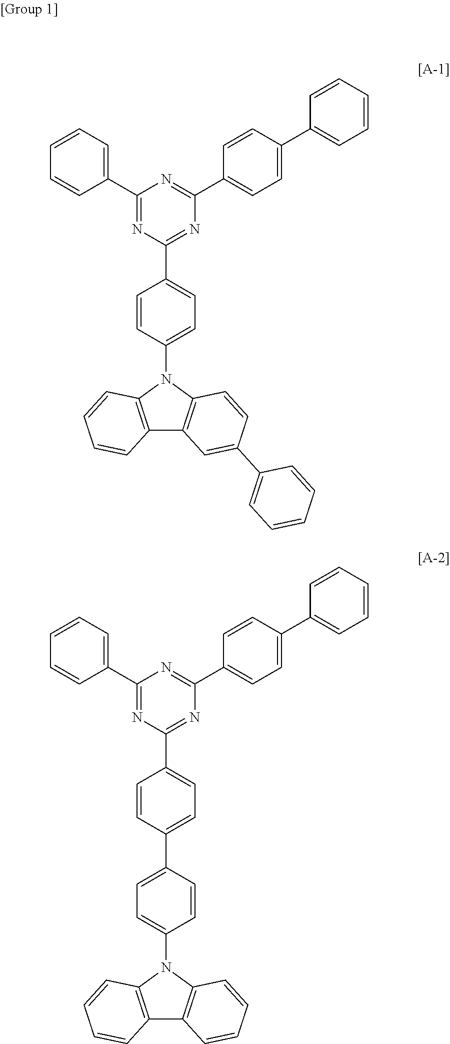 [SMILES: c1ccc(-c2ccc(-c3nc(-c4ccccc4)nc(-c4ccc(-n5c6ccccc6c6cc(-c7ccccc7)ccc65)cc4)n3)cc2)cc1 ; c1ccc(-c2ccc(-c3nc(-c4ccccc4)nc(-c4ccc(-c5ccc(-n6c7ccccc7c7ccccc76)cc5)cc4)n3)cc2)cc1]

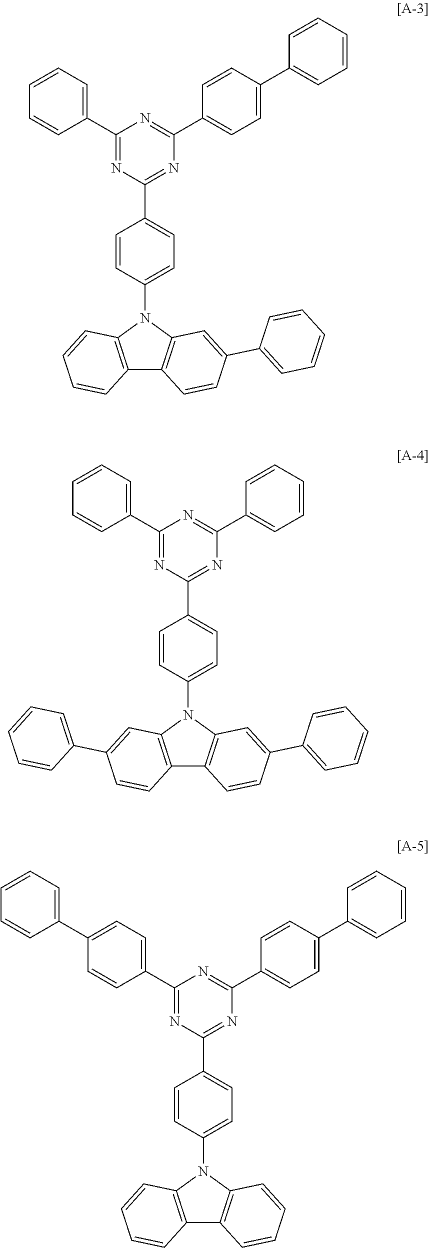 [SMILES: c1ccc(-c2ccc(-c3nc(-c4ccccc4)nc(-c4ccc(-n5c6ccccc6c6ccc(-c7ccccc7)cc65)cc4)n3)cc2)cc1 ; c1ccc(-c2ccc3c4ccc(-c5ccccc5)cc4n(-c4ccc(-c5nc(-c6ccccc6)nc(-c6ccccc6)n5)cc4)c3c2)cc1 ; c1ccc(-c2ccc(-c3nc(-c4ccc(-c5ccccc5)cc4)nc(-c4ccc(-n5c6ccccc6c6ccccc65)cc4)n3)cc2)cc1]

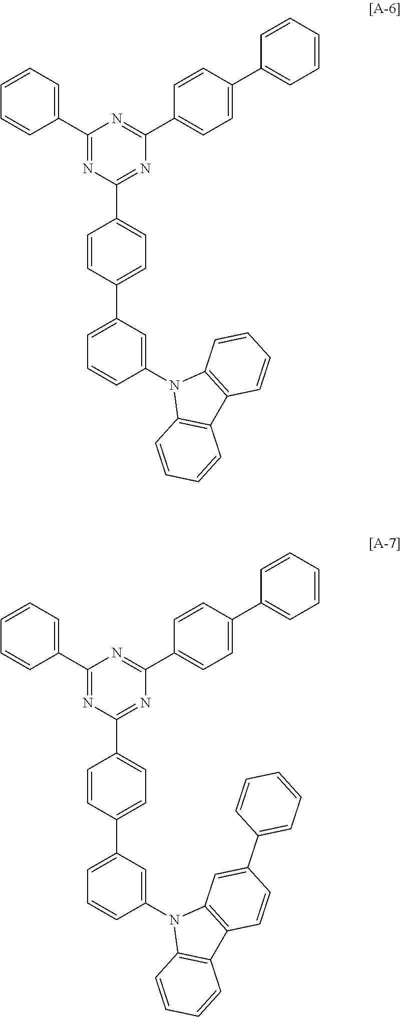 [SMILES: c1ccc(-c2ccc(-c3nc(-c4ccccc4)nc(-c4ccc(-c5cccc(-n6c7ccccc7c7ccccc76)c5)cc4)n3)cc2)cc1 ; c1ccc(-c2ccc(-c3nc(-c4ccccc4)nc(-c4ccc(-c5cccc(-n6c7ccccc7c7ccc(-c8ccccc8)cc76)c5)cc4)n3)cc2)cc1]

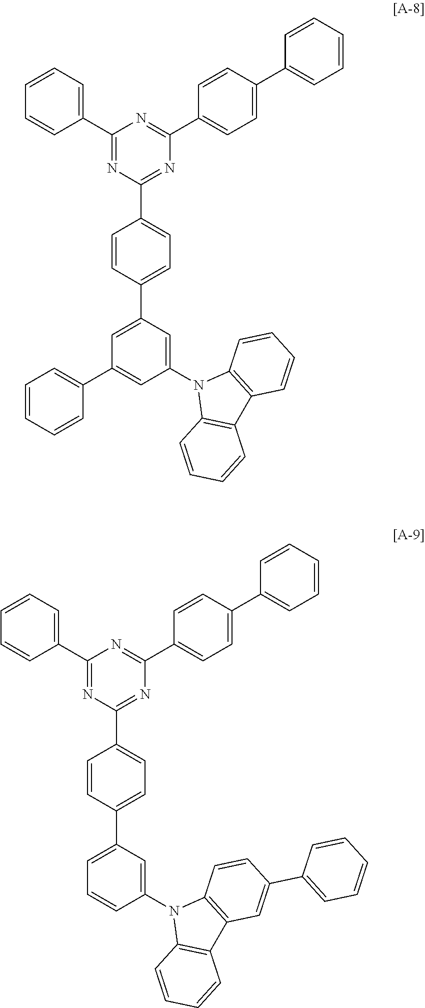 [SMILES: c1ccc(-c2ccc(-c3nc(-c4ccccc4)nc(-c4ccc(-c5cc(-c6ccccc6)cc(-n6c7ccccc7c7ccccc76)c5)cc4)n3)cc2)cc1 ; c1ccc(-c2ccc(-c3nc(-c4ccccc4)nc(-c4ccc(-c5cccc(-n6c7ccccc7c7cc(-c8ccccc8)ccc76)c5)cc4)n3)cc2)cc1]

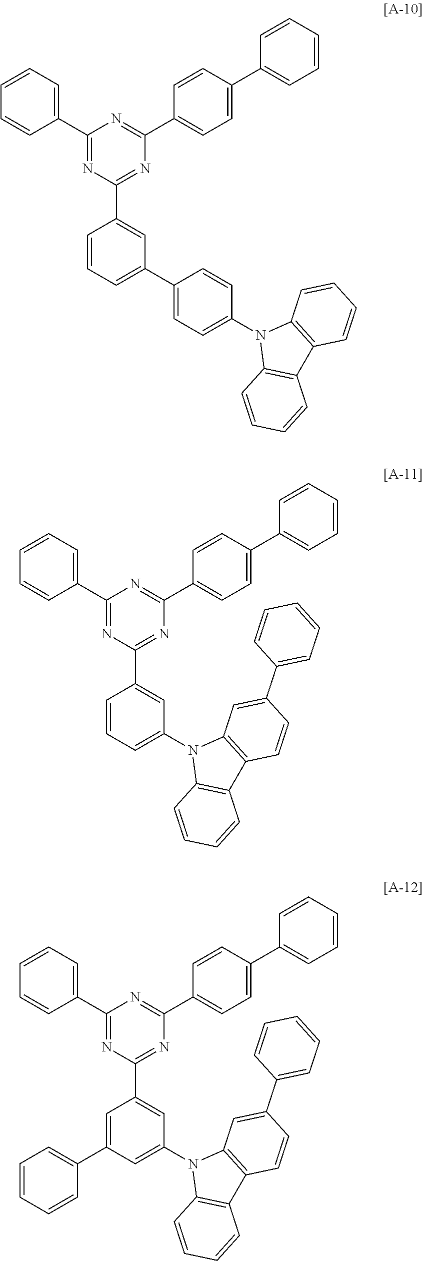 [SMILES: c1ccc(-c2ccc(-c3nc(-c4ccccc4)nc(-c4cccc(-c5ccc(-n6c7ccccc7c7ccccc76)cc5)c4)n3)cc2)cc1 ; c1ccc(-c2ccc(-c3nc(-c4ccccc4)nc(-c4cccc(-n5c6ccccc6c6ccc(-c7ccccc7)cc65)c4)n3)cc2)cc1 ; c1ccc(-c2ccc(-c3nc(-c4ccccc4)nc(-c4cc(-c5ccccc5)cc(-n5c6ccccc6c6ccc(-c7ccccc7)cc65)c4)n3)cc2)cc1]

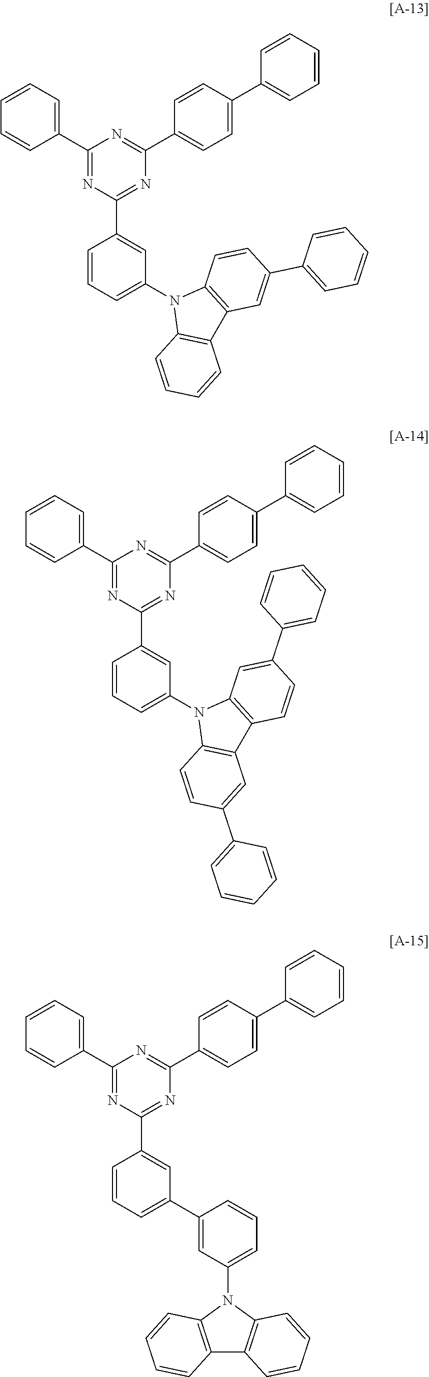 [SMILES: c1ccc(-c2ccc(-c3nc(-c4ccccc4)nc(-c4cccc(-n5c6ccccc6c6cc(-c7ccccc7)ccc65)c4)n3)cc2)cc1 ; c1ccc(-c2ccc(-c3nc(-c4ccccc4)nc(-c4cccc(-n5c6ccc(-c7ccccc7)cc6c6ccc(-c7ccccc7)cc65)c4)n3)cc2)cc1 ; c1ccc(-c2ccc(-c3nc(-c4ccccc4)nc(-c4cccc(-c5cccc(-n6c7ccccc7c7ccccc76)c5)c4)n3)cc2)cc1]

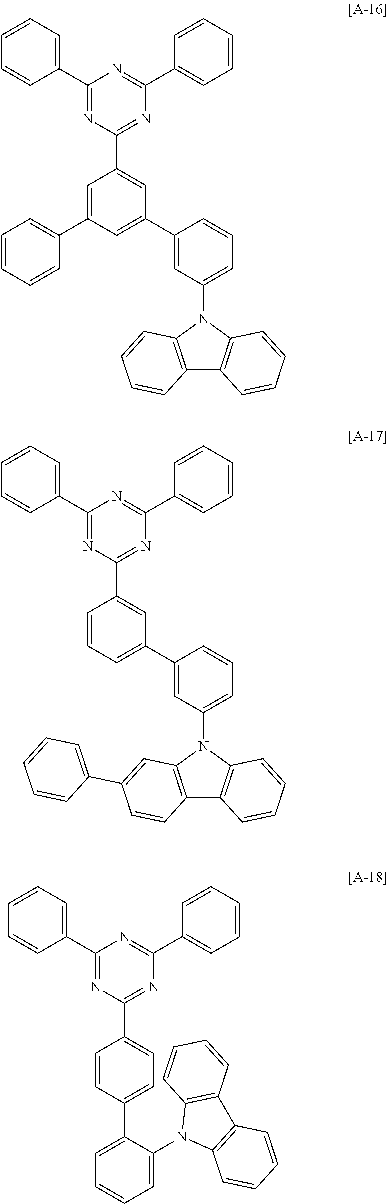 [SMILES: c1ccc(-c2cc(-c3cccc(-n4c5ccccc5c5ccccc54)c3)cc(-c3nc(-c4ccccc4)nc(-c4ccccc4)n3)c2)cc1 ; c1ccc(-c2ccc3c4ccccc4n(-c4cccc(-c5cccc(-c6nc(-c7ccccc7)nc(-c7ccccc7)n6)c5)c4)c3c2)cc1 ; c1ccc(-c2nc(-c3ccccc3)nc(-c3ccc(-c4ccccc4-n4c5ccccc5c5ccccc54)cc3)n2)cc1]

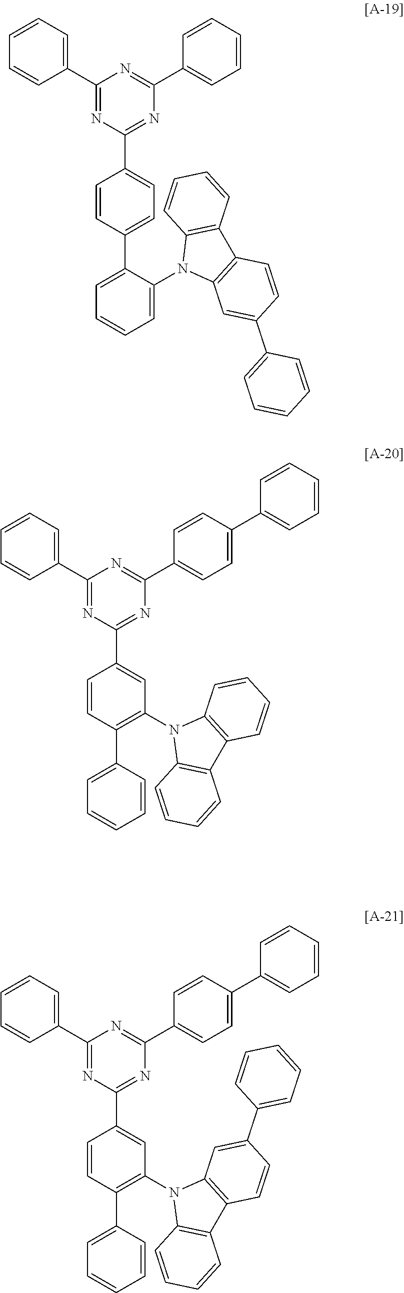 [SMILES: c1ccc(-c2ccc3c4ccccc4n(-c4ccccc4-c4ccc(-c5nc(-c6ccccc6)nc(-c6ccccc6)n5)cc4)c3c2)cc1 ; c1ccc(-c2ccc(-c3nc(-c4ccccc4)nc(-c4ccc(-c5ccccc5)c(-n5c6ccccc6c6ccccc65)c4)n3)cc2)cc1 ; c1ccc(-c2ccc(-c3nc(-c4ccccc4)nc(-c4ccc(-c5ccccc5)c(-n5c6ccccc6c6ccc(-c7ccccc7)cc65)c4)n3)cc2)cc1]

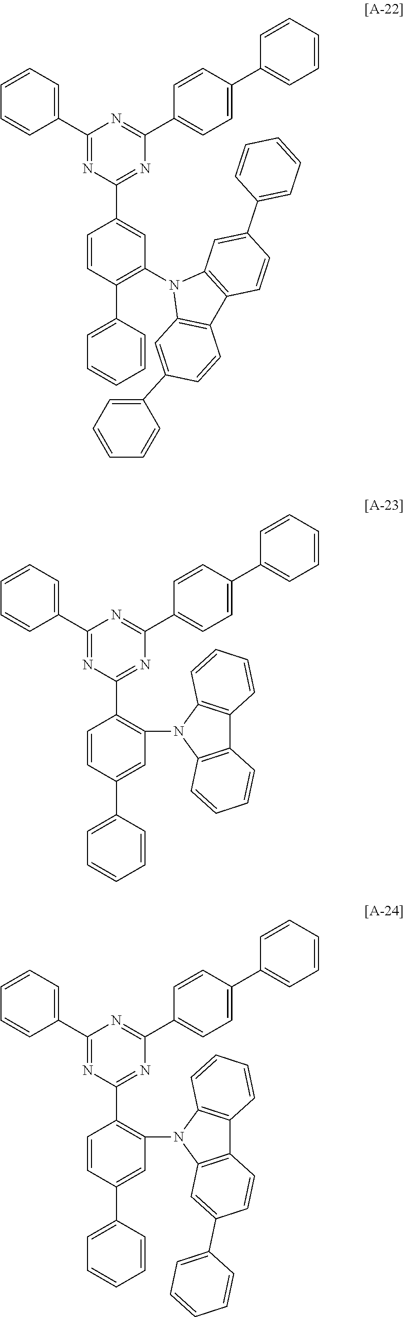 [SMILES: c1ccc(-c2ccc(-c3nc(-c4ccccc4)nc(-c4ccc(-c5ccccc5)c(-n5c6cc(-c7ccccc7)ccc6c6ccc(-c7ccccc7)cc65)c4)n3)cc2)cc1 ; c1ccc(-c2ccc(-c3nc(-c4ccccc4)nc(-c4ccc(-c5ccccc5)cc4-n4c5ccccc5c5ccccc54)n3)cc2)cc1 ; c1ccc(-c2ccc(-c3nc(-c4ccccc4)nc(-c4ccc(-c5ccccc5)cc4-n4c5ccccc5c5ccc(-c6ccccc6)cc54)n3)cc2)cc1]

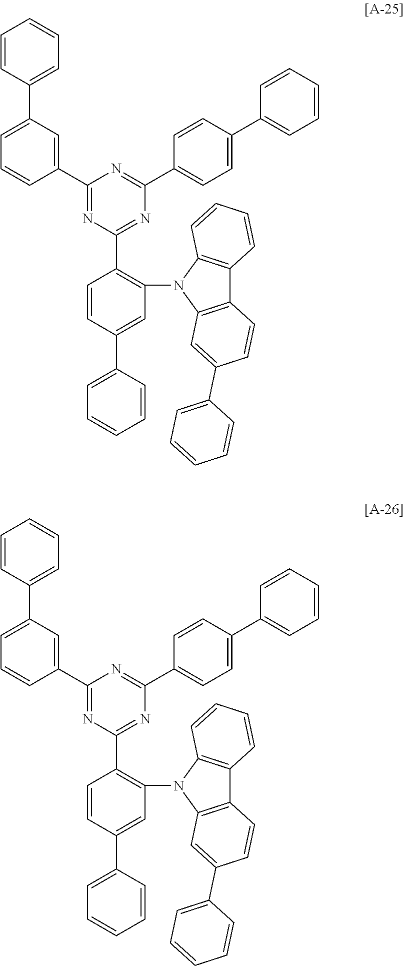 [SMILES: c1ccc(-c2ccc(-c3nc(-c4cccc(-c5ccccc5)c4)nc(-c4ccc(-c5ccccc5)cc4-n4c5ccccc5c5ccc(-c6ccccc6)cc54)n3)cc2)cc1 ; c1ccc(-c2ccc(-c3nc(-c4cccc(-c5ccccc5)c4)nc(-c4ccc(-c5ccccc5)cc4-n4c5ccccc5c5ccc(-c6ccccc6)cc54)n3)cc2)cc1]

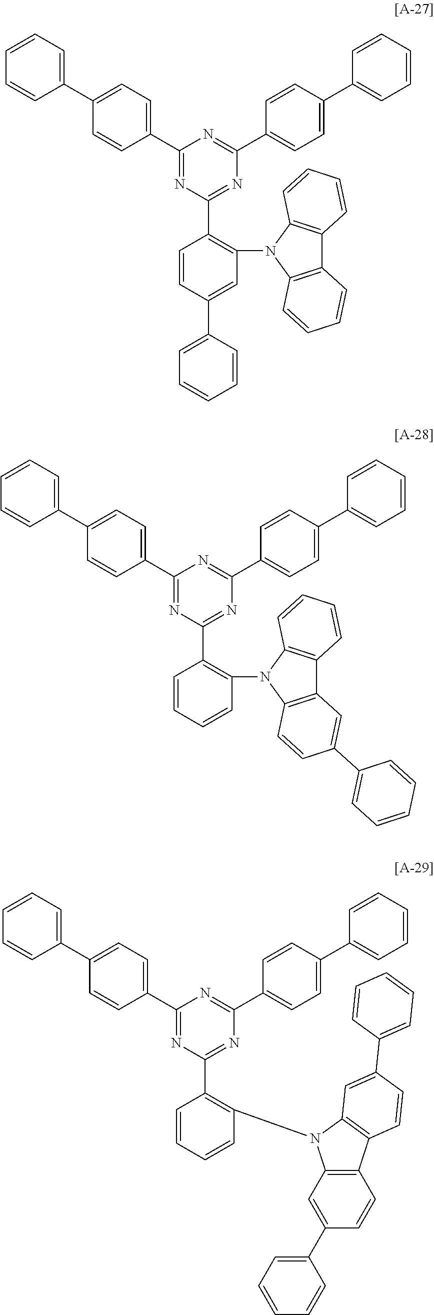 [SMILES: c1ccc(-c2ccc(-c3nc(-c4ccc(-c5ccccc5)cc4)nc(-c4ccc(-c5ccccc5)cc4-n4c5ccccc5c5ccccc54)n3)cc2)cc1 ; c1ccc(-c2ccc(-c3nc(-c4ccc(-c5ccccc5)cc4)nc(-c4ccccc4-n4c5ccccc5c5cc(-c6ccccc6)ccc54)n3)cc2)cc1 ; c1ccc(-c2ccc(-c3nc(-c4ccc(-c5ccccc5)cc4)nc(-c4ccccc4-n4c5cc(-c6ccccc6)ccc5c5ccc(-c6ccccc6)cc54)n3)cc2)cc1]

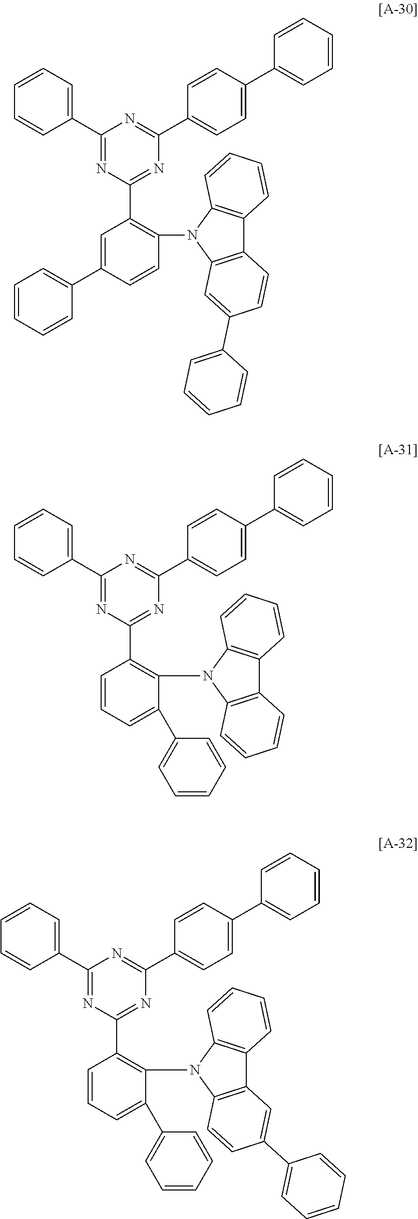 [SMILES: c1ccc(-c2ccc(-c3nc(-c4ccccc4)nc(-c4cc(-c5ccccc5)ccc4-n4c5ccccc5c5ccc(-c6ccccc6)cc54)n3)cc2)cc1 ; c1ccc(-c2ccc(-c3nc(-c4ccccc4)nc(-c4cccc(-c5ccccc5)c4-n4c5ccccc5c5ccccc54)n3)cc2)cc1 ; c1ccc(-c2ccc(-c3nc(-c4ccccc4)nc(-c4cccc(-c5ccccc5)c4-n4c5ccccc5c5cc(-c6ccccc6)ccc54)n3)cc2)cc1]

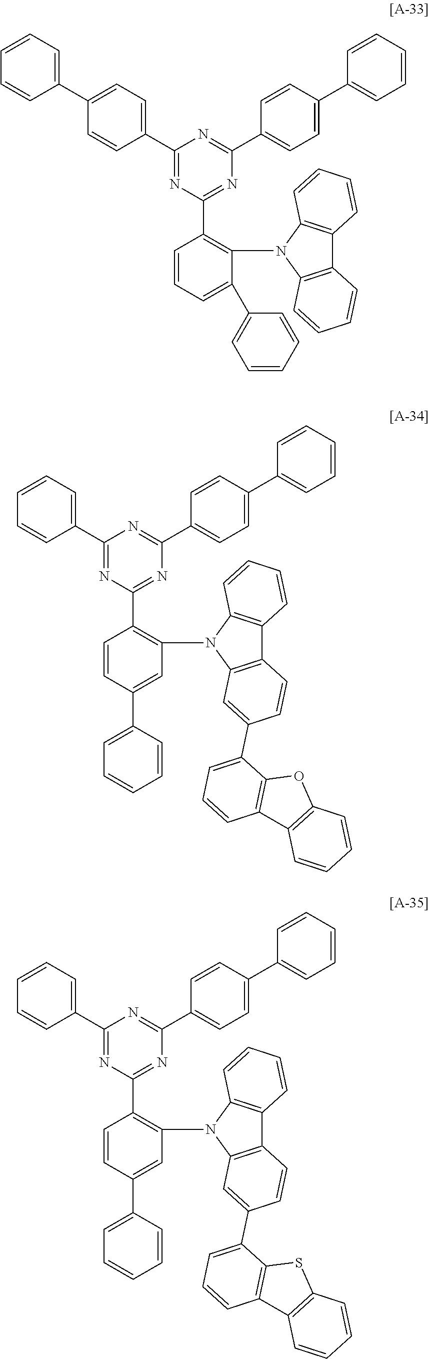

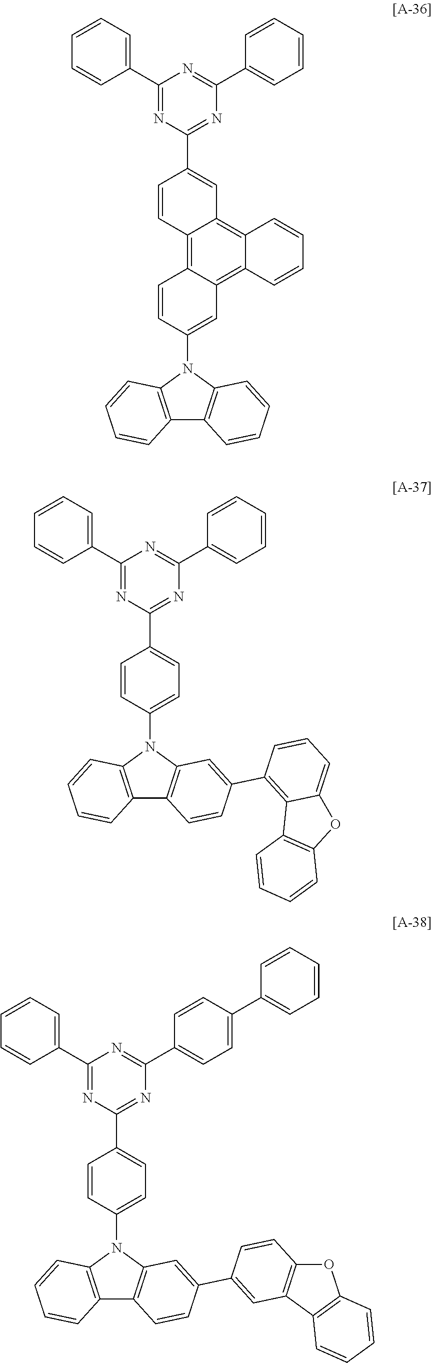

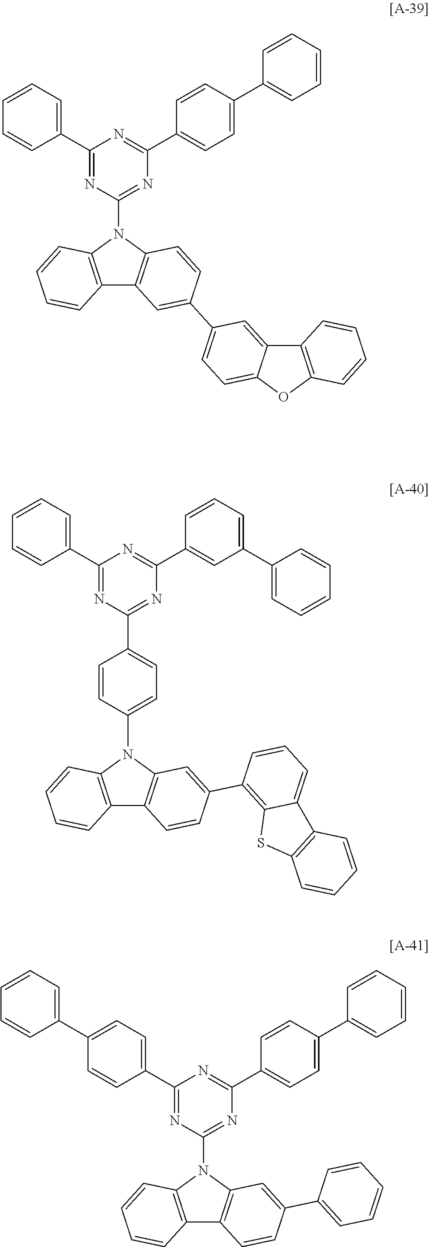 [SMILES: c1ccc(-c2ccc(-c3nc(-c4ccccc4)nc(-n4c5ccccc5c5cc(-c6ccc7oc8ccccc8c7c6)ccc54)n3)cc2)cc1 ; c1ccc(-c2cccc(-c3nc(-c4ccccc4)nc(-c4ccc(-n5c6ccccc6c6ccc(-c7cccc8c7sc7ccccc78)cc65)cc4)n3)c2)cc1 ; c1ccc(-c2ccc(-c3nc(-c4ccc(-c5ccccc5)cc4)nc(-n4c5ccccc5c5ccc(-c6ccccc6)cc54)n3)cc2)cc1]

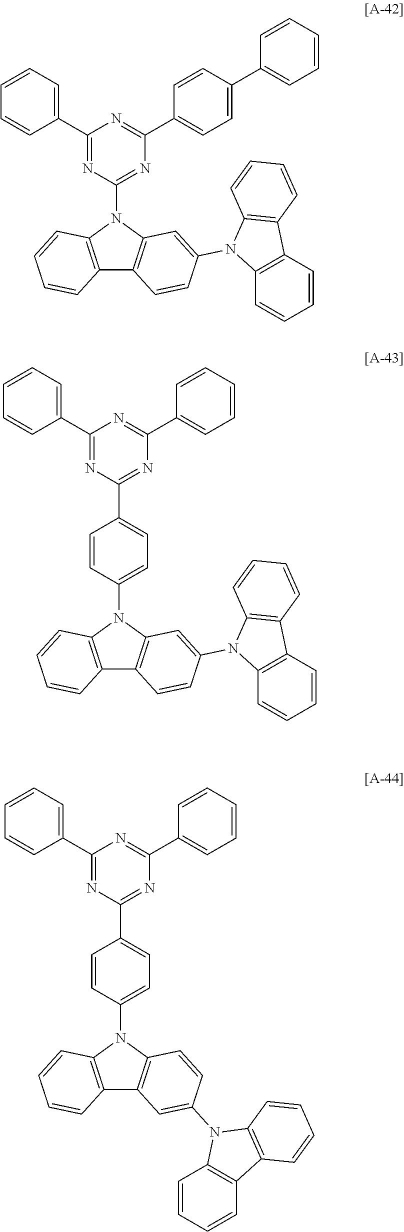 [SMILES: c1ccc(-c2ccc(-c3nc(-c4ccccc4)nc(-n4c5ccccc5c5ccc(-n6c7ccccc7c7ccccc76)cc54)n3)cc2)cc1 ; c1ccc(-c2nc(-c3ccccc3)nc(-c3ccc(-n4c5ccccc5c5ccc(-n6c7ccccc7c7ccccc76)cc54)cc3)n2)cc1 ; c1ccc(-c2nc(-c3ccccc3)nc(-c3ccc(-n4c5ccccc5c5cc(-n6c7ccccc7c7ccccc76)ccc54)cc3)n2)cc1]

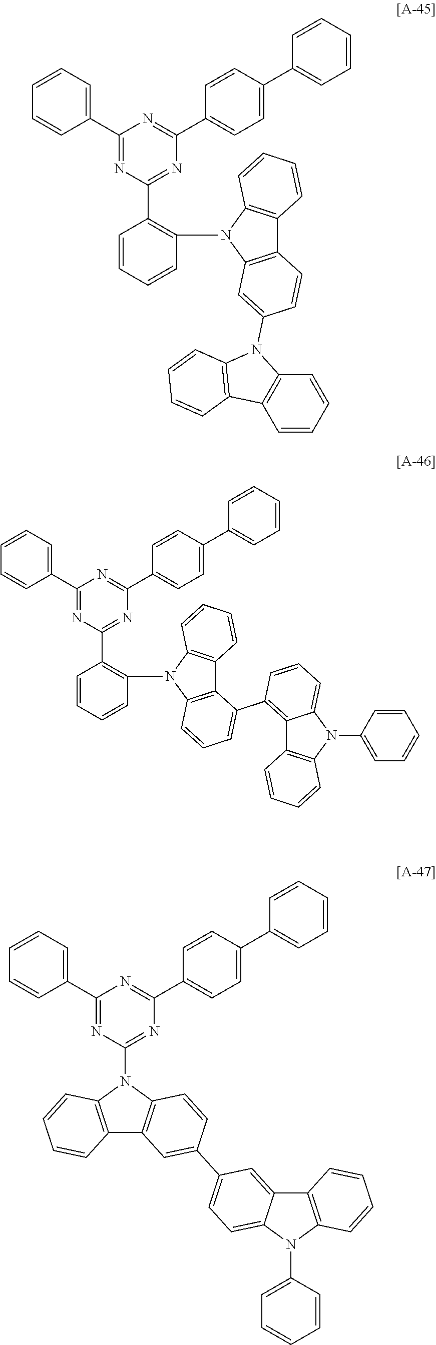 [SMILES: c1ccc(-c2ccc(-c3nc(-c4ccccc4)nc(-c4ccccc4-n4c5ccccc5c5ccc(-n6c7ccccc7c7ccccc76)cc54)n3)cc2)cc1 ; c1ccc(-c2ccc(-c3nc(-c4ccccc4)nc(-c4ccccc4-n4c5ccccc5c5c(-c6cccc7c6c6ccccc6n7-c6ccccc6)cccc54)n3)cc2)cc1 ; c1ccc(-c2ccc(-c3nc(-c4ccccc4)nc(-n4c5ccccc5c5cc(-c6ccc7c(c6)c6ccccc6n7-c6ccccc6)ccc54)n3)cc2)cc1]

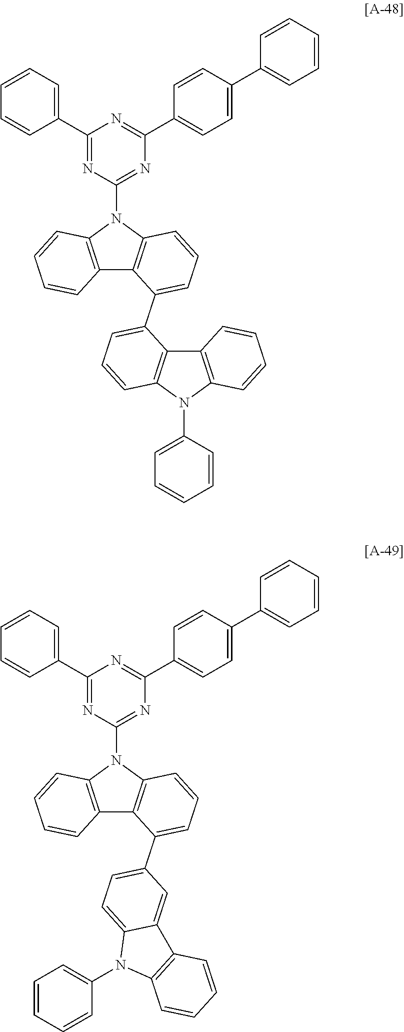 [SMILES: c1ccc(-c2ccc(-c3nc(-c4ccccc4)nc(-n4c5ccccc5c5c(-c6cccc7c6c6ccccc6n7-c6ccccc6)cccc54)n3)cc2)cc1 ; c1ccc(-c2ccc(-c3nc(-c4ccccc4)nc(-n4c5ccccc5c5c(-c6ccc7c(c6)c6ccccc6n7-c6ccccc6)cccc54)n3)cc2)cc1]

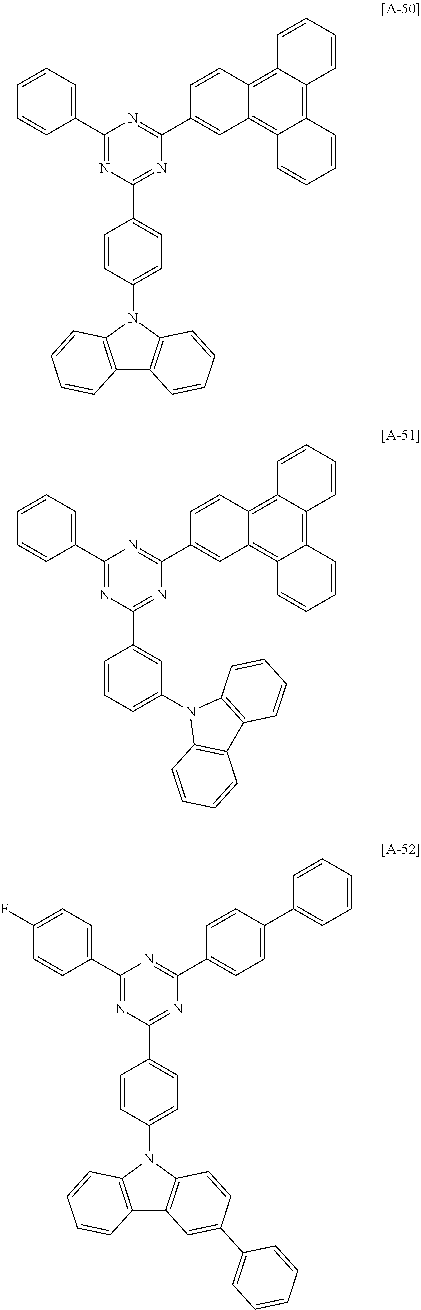 [SMILES: c1ccc(-c2nc(-c3ccc(-n4c5ccccc5c5ccccc54)cc3)nc(-c3ccc4c5ccccc5c5ccccc5c4c3)n2)cc1 ; c1ccc(-c2nc(-c3cccc(-n4c5ccccc5c5ccccc54)c3)nc(-c3ccc4c5ccccc5c5ccccc5c4c3)n2)cc1 ; Fc1ccc(-c2nc(-c3ccc(-c4ccccc4)cc3)nc(-c3ccc(-n4c5ccccc5c5cc(-c6ccccc6)ccc54)cc3)n2)cc1]

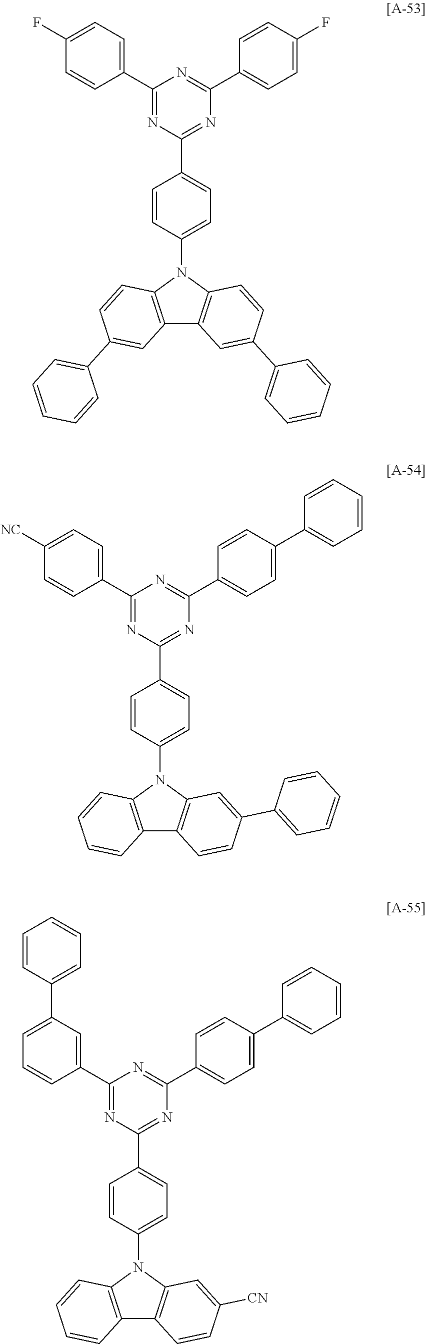 [SMILES: Fc1ccc(-c2nc(-c3ccc(F)cc3)nc(-c3ccc(-n4c5ccc(-c6ccccc6)cc5c5cc(-c6ccccc6)ccc54)cc3)n2)cc1 ; N#Cc1ccc(-c2nc(-c3ccc(-c4ccccc4)cc3)nc(-c3ccc(-n4c5ccccc5c5ccc(-c6ccccc6)cc54)cc3)n2)cc1 ; N#Cc1ccc2c3ccccc3n(-c3ccc(-c4nc(-c5ccc(-c6ccccc6)cc5)nc(-c5cccc(-c6ccccc6)c5)n4)cc3)c2c1]

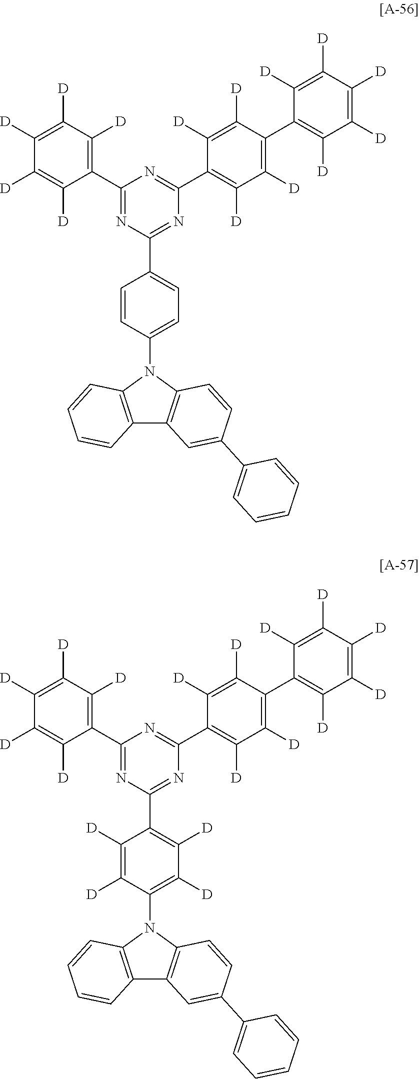 [SMILES: [2H]c1c([2H])c([2H])c(-c2nc(-c3ccc(-n4c5ccccc5c5cc(-c6ccccc6)ccc54)cc3)nc(-c3c([2H])c([2H])c(-c4c([2H])c([2H])c([2H])c([2H])c4[2H])c([2H])c3[2H])n2)c([2H])c1[2H] ; [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c(-n4c5ccccc5c5cc(-c6ccccc6)ccc54)c([2H])c3[2H])n2)c([2H])c1[2H]]

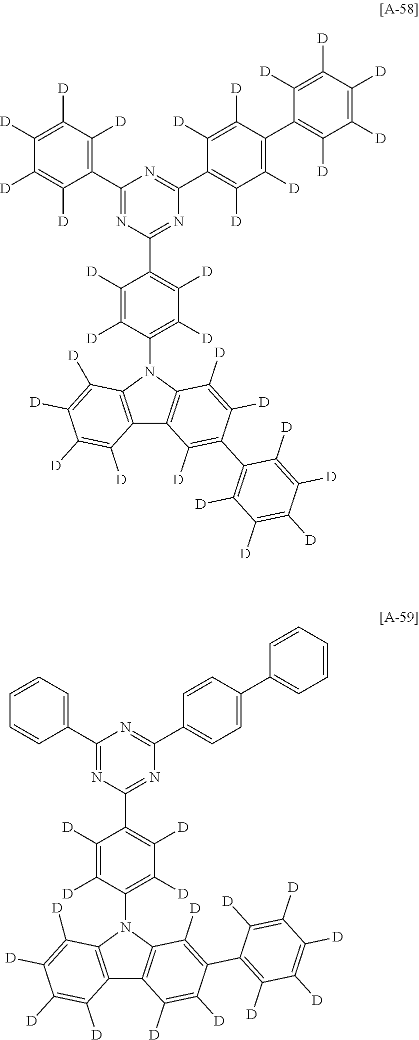 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c(-n4c5c([2H])c([2H])c([2H])c([2H])c5c5c([2H])c(-c6c([2H])c([2H])c([2H])c([2H])c6[2H])c([2H])c([2H])c54)c([2H])c3[2H])n2)c([2H])c1[2H] ; [2H]c1c([2H])c([2H])c(-c2c([2H])c([2H])c3c4c([2H])c([2H])c([2H])c([2H])c4n(-c4c([2H])c([2H])c(-c5nc(-c6ccccc6)nc(-c6ccc(-c7ccccc7)cc6)n5)c([2H])c4[2H])c3c2[2H])c([2H])c1[2H]]

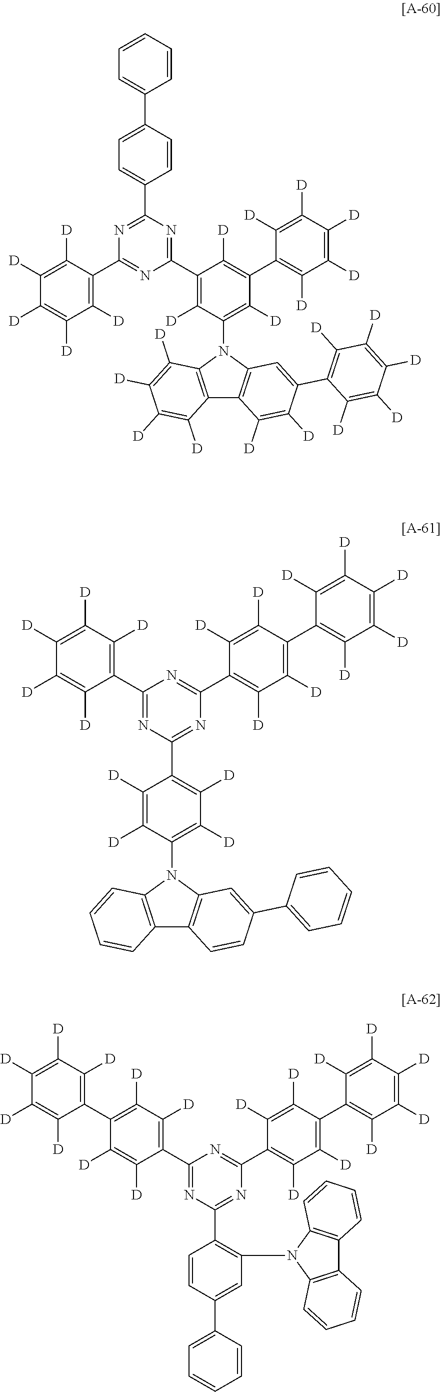

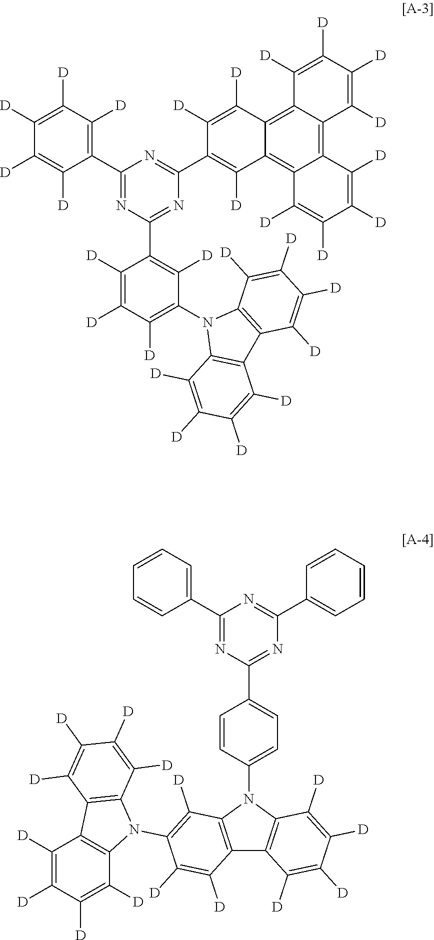 [SMILES: [2H]c1c([2H])c([2H])c(-c2nc(-c3c([2H])c([2H])c([2H])c(-n4c5c([2H])c([2H])c([2H])c([2H])c5c5c([2H])c([2H])c([2H])c([2H])c54)c3[2H])nc(-c3c([2H])c([2H])c4c5c([2H])c([2H])c([2H])c([2H])c5c5c([2H])c([2H])c([2H])c([2H])c5c4c3[2H])n2)c([2H])c1[2H] ; [2H]c1c([2H])c([2H])c2c(c1[2H])c1c([2H])c([2H])c(-n3c4c([2H])c([2H])c([2H])c([2H])c4c4c([2H])c([2H])c([2H])c([2H])c43)c([2H])c1n2-c1ccc(-c2nc(-c3ccccc3)nc(-c3ccccc3)n2)cc1]

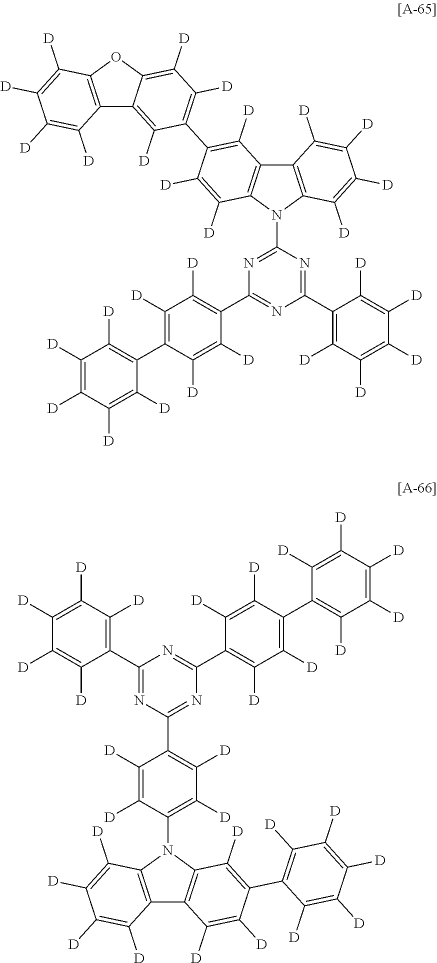 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-n3c4c([2H])c([2H])c([2H])c([2H])c4c4c([2H])c(-c5c([2H])c([2H])c6oc7c([2H])c([2H])c([2H])c([2H])c7c6c5[2H])c([2H])c([2H])c43)n2)c([2H])c1[2H] ; [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c(-n4c5c([2H])c([2H])c([2H])c([2H])c5c5c([2H])c([2H])c(-c6c([2H])c([2H])c([2H])c([2H])c6[2H])c([2H])c54)c([2H])c3[2H])n2)c([2H])c1[2H]]

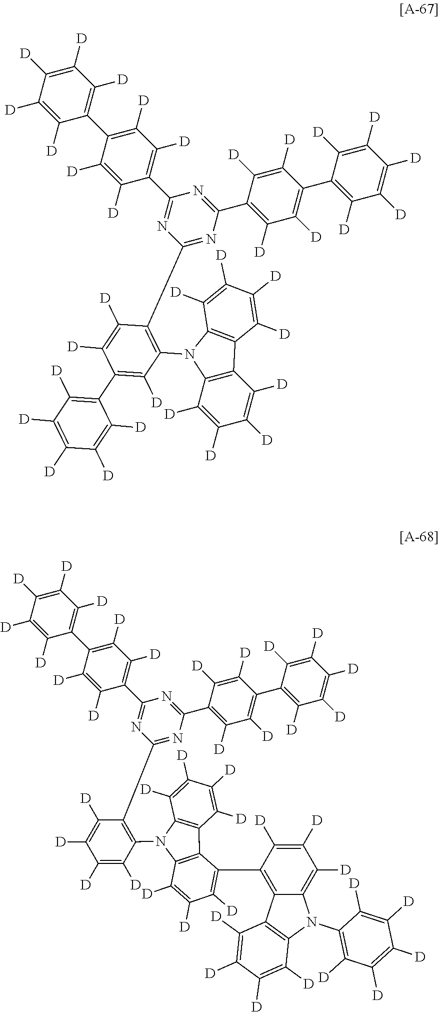

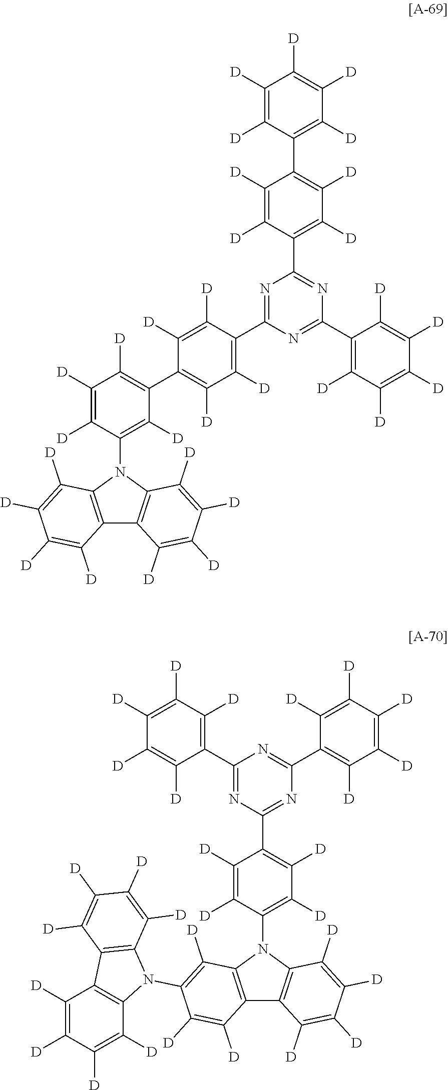

The second compound may be represented by Chemical Formula 2.

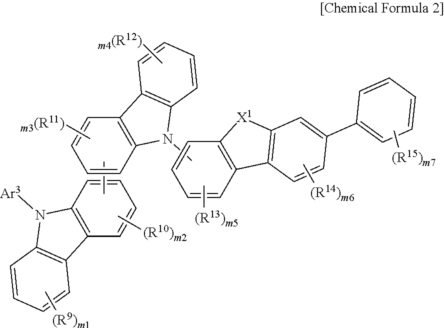

In Chemical Formula 2,
X¹ is O or S,
Ar³ is a substituted or unsubstituted C6 to C30 aryl group, a substituted or unsubstituted C2 to C30 heterocyclic group, or a combination thereof,
R⁹ to R¹² are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group,
R¹³ to R¹⁵ are each independently hydrogen, deuterium, or substituted or unsubstitutedphenyl group,
m2, m3, m5, and m6 are each independently one of integers of 1 to 3,
m1 and m4 are each independently one of integers of 1 to 4, and
m7 is one of integers of 1 to 5.

The second compound has hole characteristics, and at least one N-directed substituent of bicarbazole is dibenzofuran (or dibenzothiophene), and the dibenzofuran (or dibenzothiophene) has a structure in which phenyl is substituted at the 9th position.

The bicarbazole having a phenyl-substituted dibenzofuran (or dibenzothiophene) at the 7th position has a property of bringing the intermolecular distance between hole-characterized compounds and electron-characterized compounds closer. In particular, the LUMO of the hole characteristic compound and the LUMO of the electronic characteristic compound are closely arranged, so that the LUMO of the electronic characteristic compound extends to the LUMO of the hole characteristic compound, and may be deposited advantageously for the electron transport. Due to the structural arrangement as described above, the low-driving/high-efficiency characteristics of the organic light emitting diode applied thereto may be realized. Also, when dibenzofuran (or dibenzothiophene) is substituted with phenyl at the 7th position, it has the highest energy in terms of bond dissociation energy (BDE) of the C—N bond compared to substituting phenyl at other positions, resulting in the most stable bonding structure. Therefore, due to its high structural stability, it has high heat resistance and may exhibit long life-span characteristics.

In particular, by using it together with the aforementioned first compound, the charge balance is appropriately maintained, which favors exciton formation, and thus enables implementation of high-efficiency device characteristics.

In Chemical Formula 2, when m1 is 2 or more, each R⁹ may be the same or different from each other.

In Chemical Formula 2, when m2 is 2 or more, each R¹⁰ may be the same or different from each other.

In Chemical Formula 2, when m3 is 2 or more, each R¹¹ may be the same or different from each other.

In Chemical Formula 2, when m4 is 2 or more, each R¹² may be the same or different from each other.

In Chemical Formula 2, when m5 is 2 or more, each R¹³ may be the same or different from each other.

In Chemical Formula 2, when m6 is 2 or more, each R¹⁴ may be the same or different from each other.

In Chemical Formula 2, when m7 is 2 or more, each R¹⁵ may be the same or different from each other.

For example, the second compound may be represented by any one of Chemical Formula 2-1 to Chemical Formula 2-4.

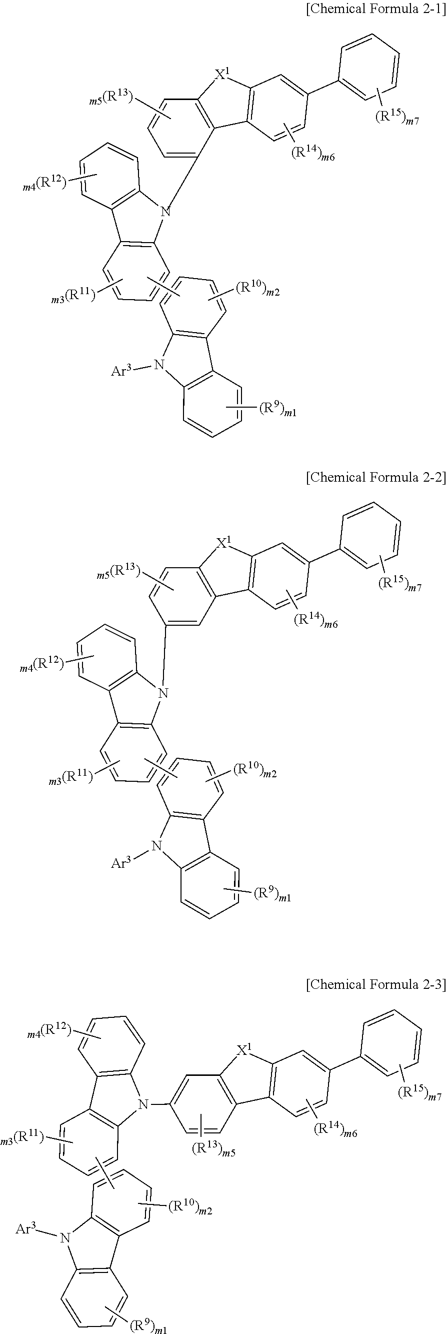

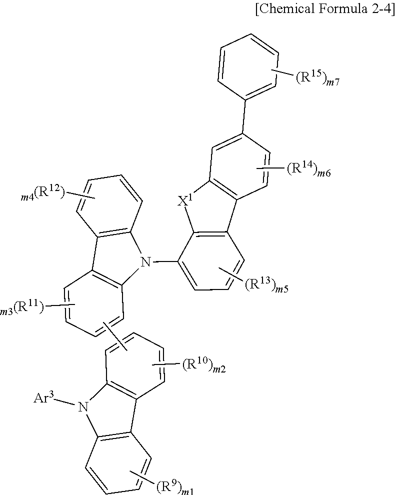

In Chemical Formula 2-1 to Chemical Formula 2-4, the definitions of X¹, Ar³, R⁹ to R¹⁵, and m1 to m7 are the same as described above.

As a specific example, Chemical Formula 2-1 may be represented by any one of Chemical Formula 2-1-1 to Chemical Formula 2-1-16.

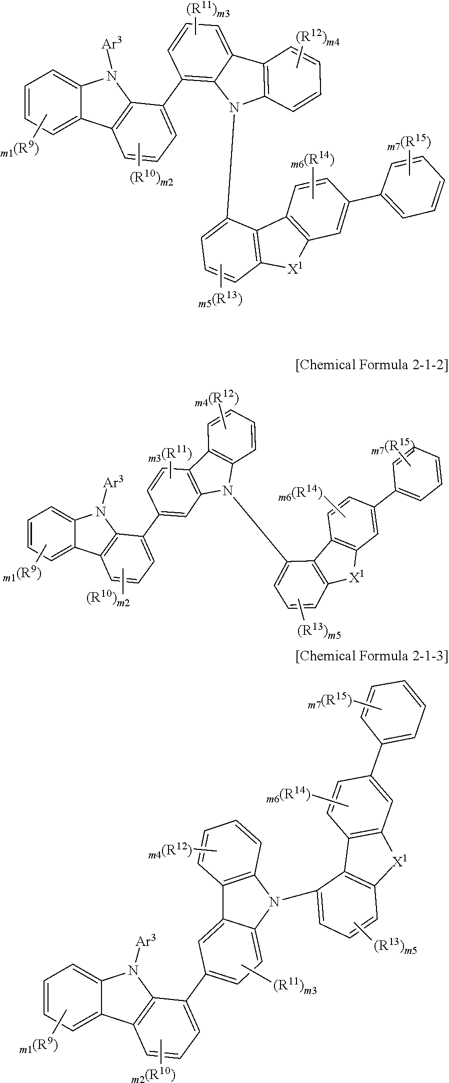

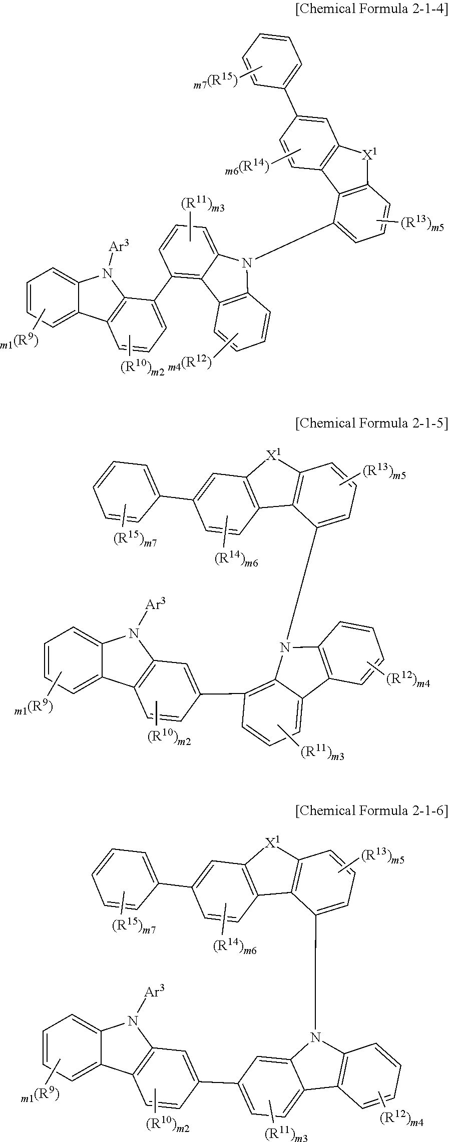

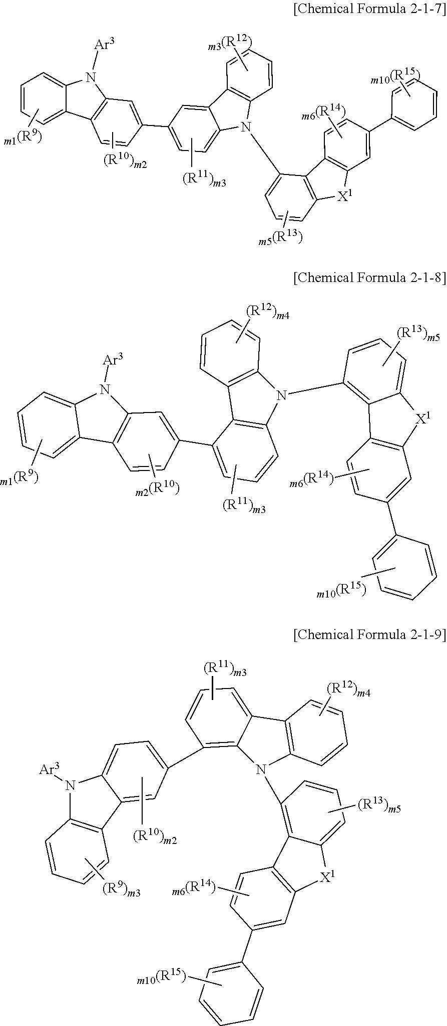

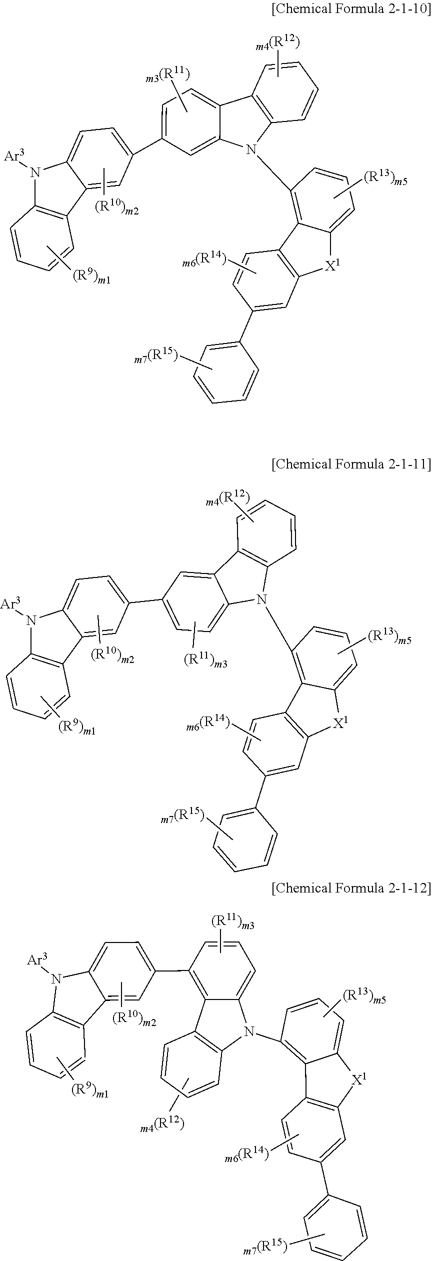

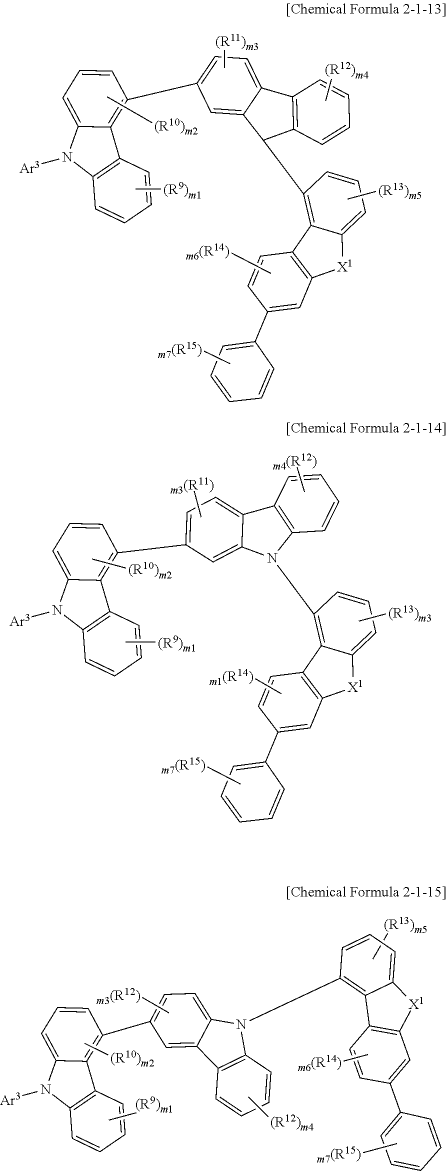

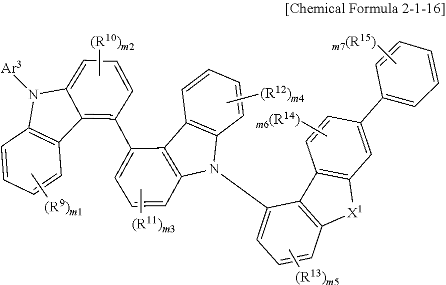

In Chemical Formula 2-1-1 to Chemical Formula 2-1-16, the definitions of X¹, Ar³, R⁹ to R¹⁵ and m1 to m7 are the same as described above.

As a specific example, Chemical Formula 2-2 may be represented by any one of Chemical Formula 2-2-1 to Chemical Formula 2-2-16.

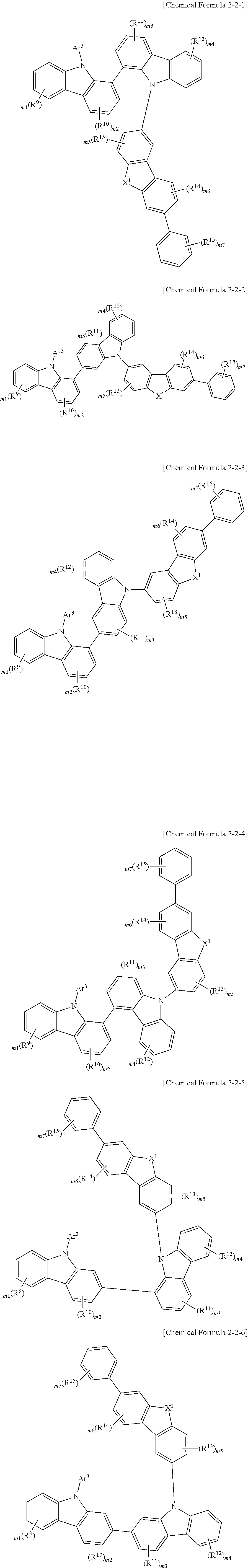

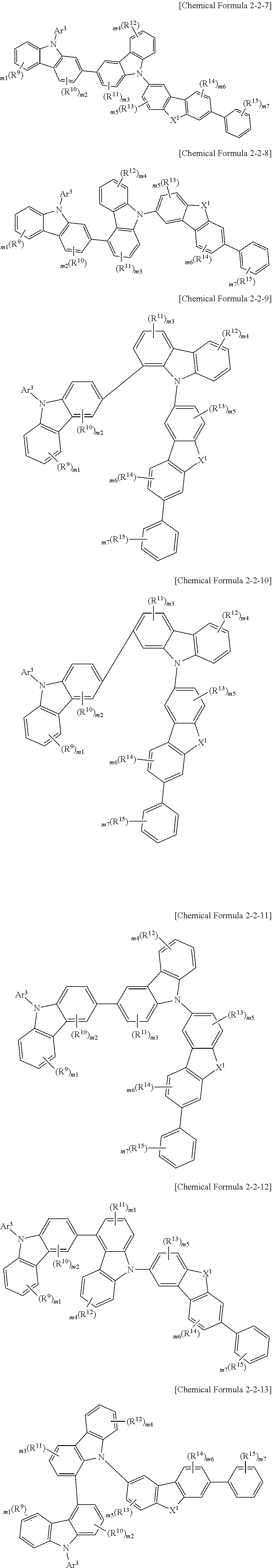

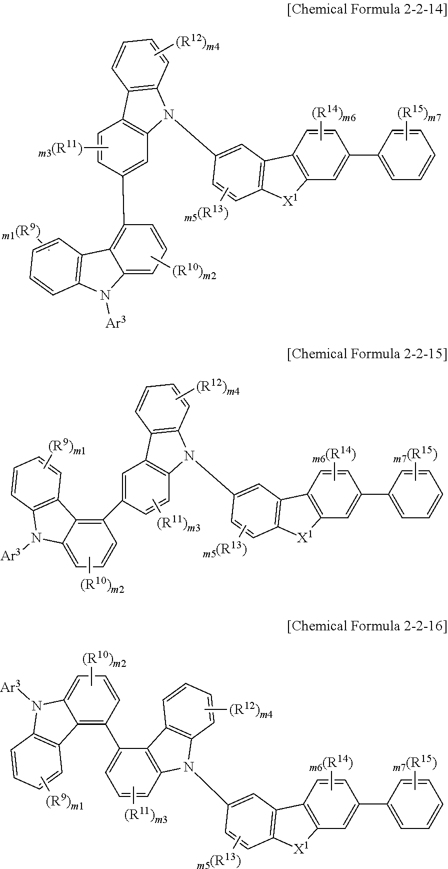

In Chemical Formula 2-2-1 to Chemical Formula 2-2-16, the definitions of X¹, Ar³, R⁹ to R¹⁵, and m1 to m7 are the same as described above.

As a specific example, above Chemical Formula 2-3 may be represented by any one of Chemical Formula 2-3-1 to Chemical Formula 2-3-16.

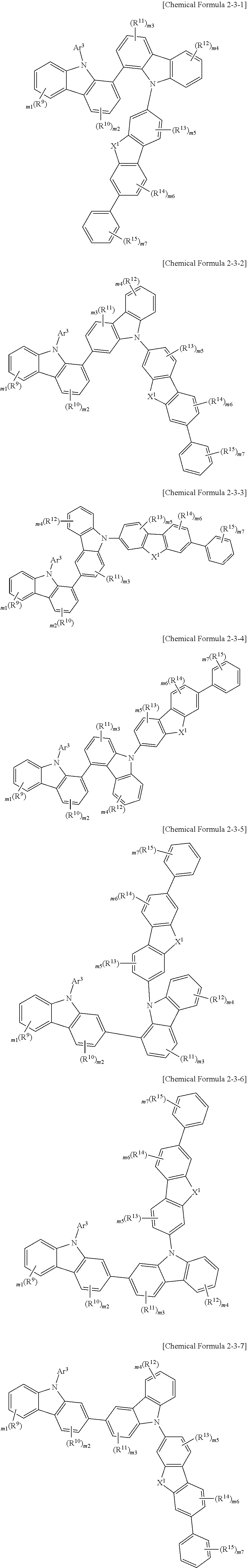

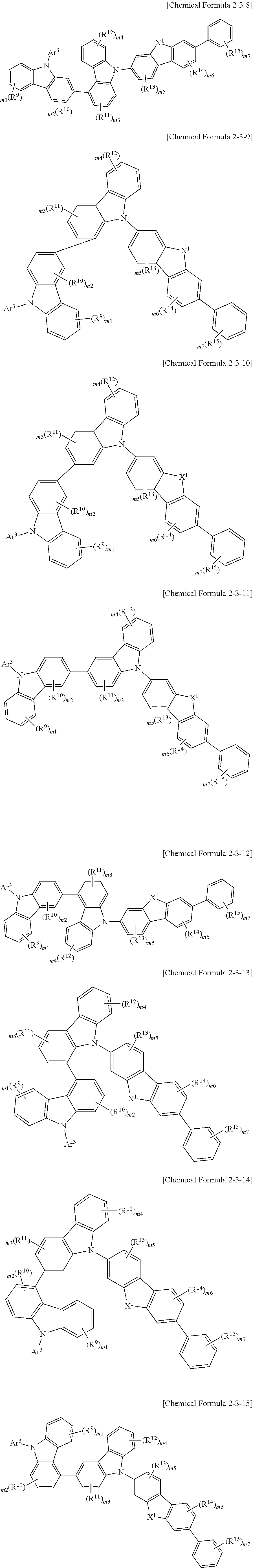

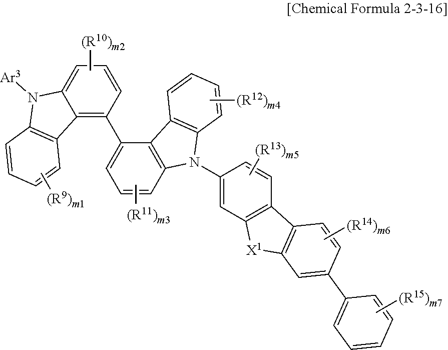

In Chemical Formula 2-3-1 to Chemical Formula 2-3-16, the definitions of X¹, Ar³, R⁹ to R¹⁵, and m1 to m7 are the same as described above.

As a specific example, Chemical Formula 2-4 may be represented by any one of Chemical Formula 2-4-1 to Chemical Formula 2-4-16.

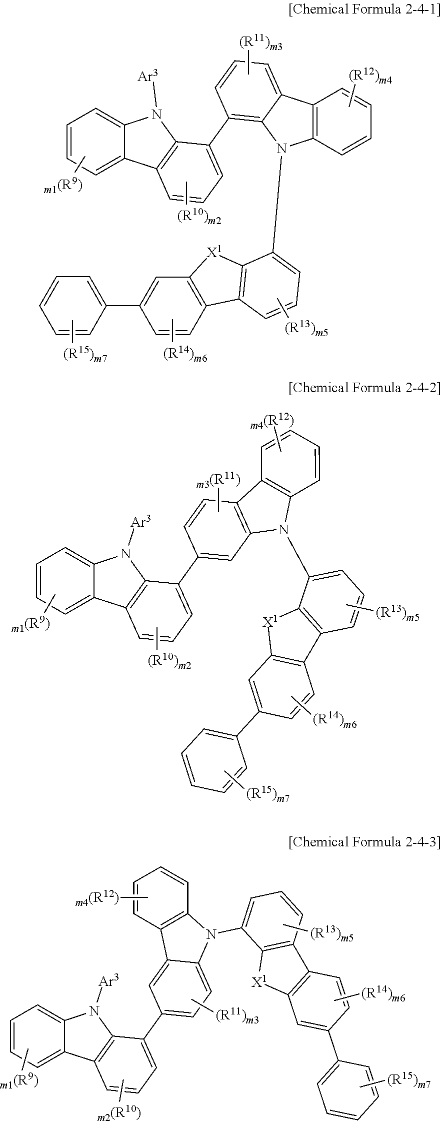

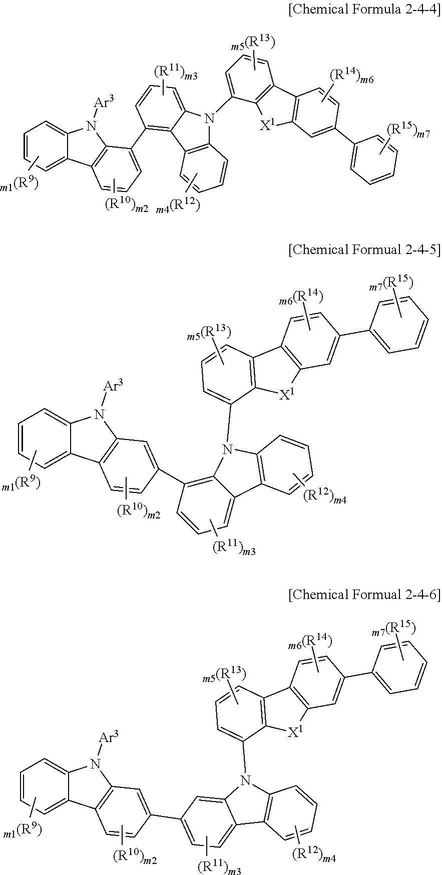

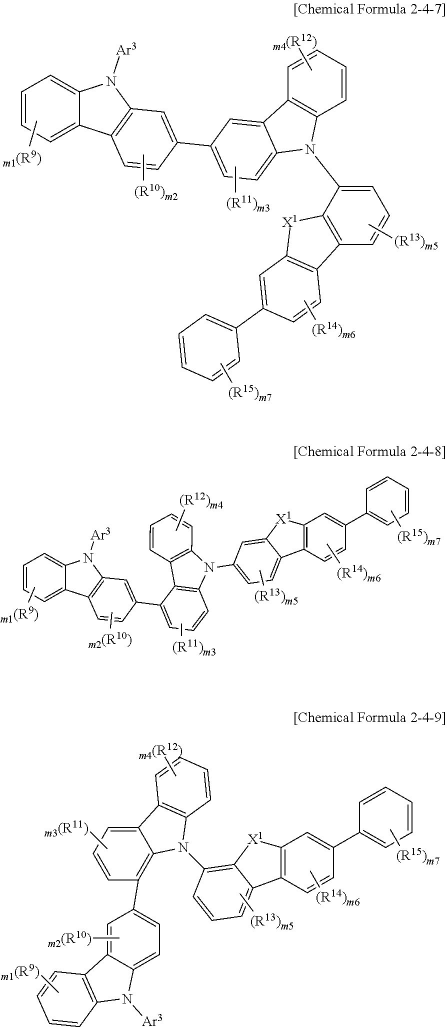

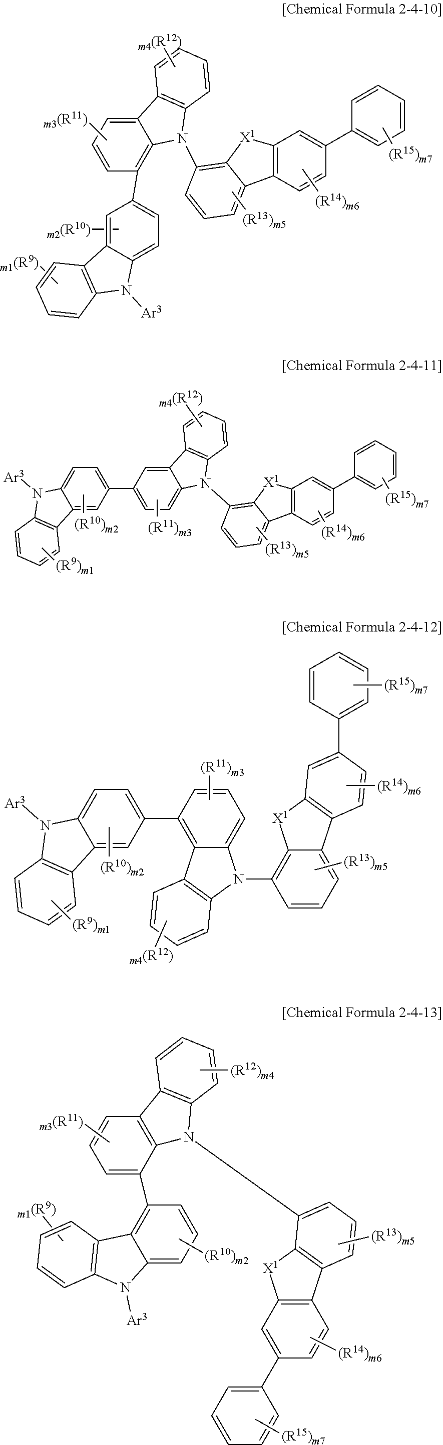

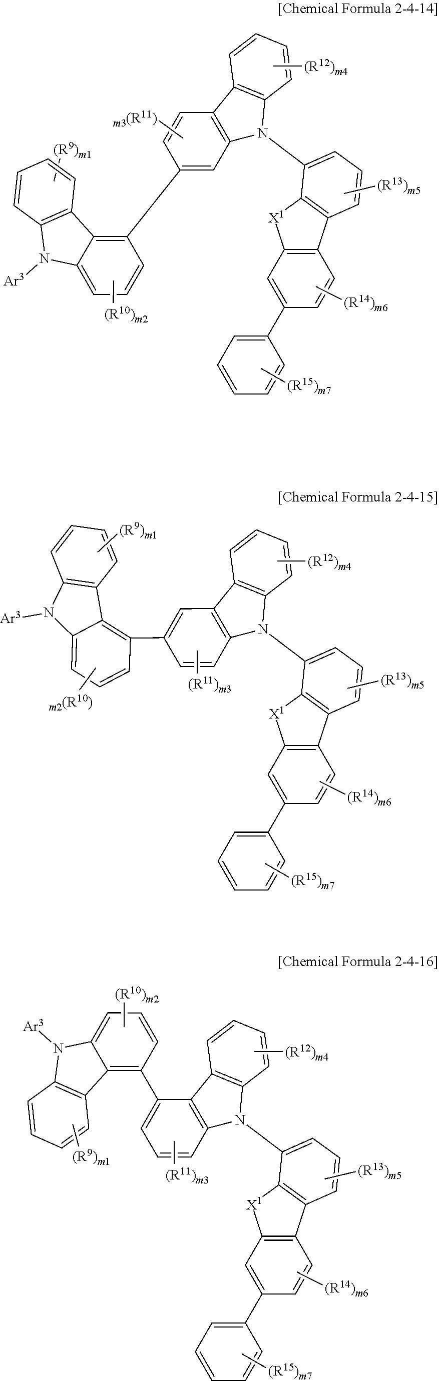

In Chemical Formula 2-4-1 to Chemical Formula 2-4-16, the definitions of X¹, Ar³, R⁹ to R¹⁵, and m1 to m7 are the same as described above.

For example, the second compound may be represented by any one of Chemical Formula 2-1-11, Chemical Formula 2-2-11, Chemical Formula 2-3-11, and Chemical Formula 2-4-11.

For example, in Chemical Formula 2, Ar³ may be a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, or a substituted or unsubstituted fluorenyl group.

As a specific example, in Chemical Formula 2, Ar³ may a substituted or unsubstituted phenyl group, or a substituted or unsubstituted biphenyl group.

For example, in Chemical Formula 2, R⁹ to R¹² may each independently be hydrogen, deuterium, a substituted or unsubstituted C1 to C10 alkyl group, a substituted or unsubstituted C6 to C20 aryl group, or a substituted or unsubstituted C2 to C20 heterocyclic group.

As a specific example, in Chemical Formula 2, R⁹ to R¹² may each independently be hydrogen, deuterium, a substituted or unsubstituted C6 to C12 aryl group, a substituted or unsubstituted carbazolyl group, a substituted or unsubstituted dibenzofuranyl group, or a substituted or unsubstituted dibenzothiophenyl group.

As a more specific example, in Chemical Formula 2, R⁹ to R¹² may each independently be hydrogen, deuterium, a substituted or unsubstituted phenyl group, a substituted or unsubstituted biphenyl group, a substituted or unsubstituted naphthyl group, a substituted or unsubstituted carbazolyl group, a substituted or unsubstituted dibenzofuranyl group, or a substituted or unsubstituted dibenzothiophenyl group.

For example, in Chemical Formula 2, R⁹ to R¹² may each independently be hydrogen or deuterium.

For example, the second compound may be one selected from the compounds listed in Group 2, but is not limited thereto.

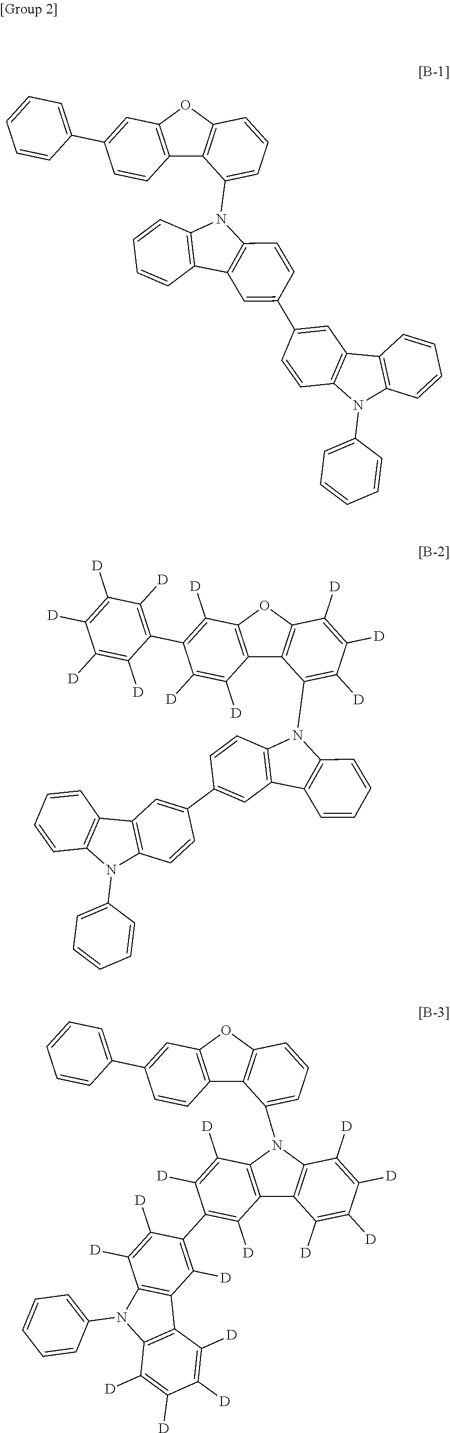 [SMILES: c1ccc(-c2ccc3c(c2)oc2cccc(-n4c5ccccc5c5cc(-c6ccc7c(c6)c6ccccc6n7-c6ccccc6)ccc54)c23)cc1 ; [2H]c1c([2H])c([2H])c(-c2c([2H])c([2H])c3c(oc4c([2H])c([2H])c([2H])c(-n5c6ccccc6c6cc(-c7ccc8c(c7)c7ccccc7n8-c7ccccc7)ccc65)c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4oc5cc(-c6ccccc6)ccc5c34)c([2H])c([2H])c1n2-c1ccccc1]

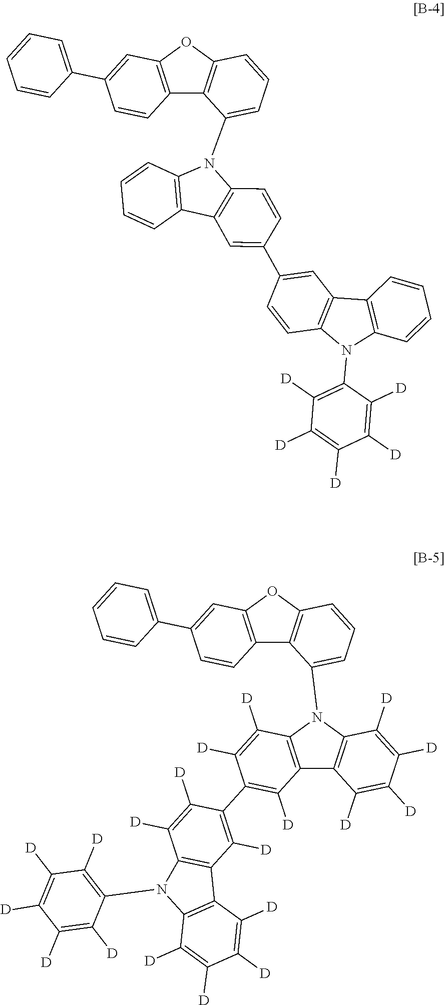 [SMILES: [2H]c1c([2H])c([2H])c(-n2c3ccccc3c3cc(-c4ccc5c(c4)c4ccccc4n5-c4cccc5oc6cc(-c7ccccc7)ccc6c45)ccc32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5oc6cc(-c7ccccc7)ccc6c45)c([2H])c([2H])c32)c([2H])c1[2H]]

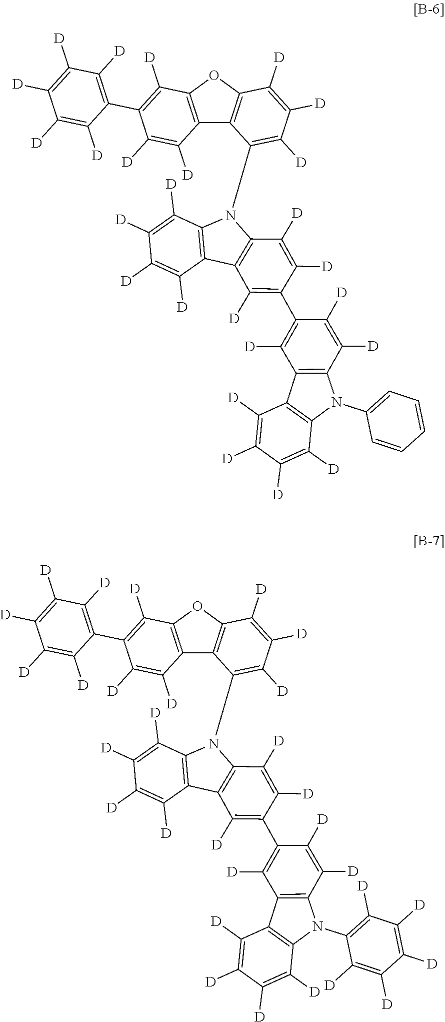 [SMILES: [2H]c1c([2H])c([2H])c(-c2c([2H])c([2H])c3c(oc4c([2H])c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)c43)c2[2H])c([2H])c1[2H] ; [2H]c1c([2H])c([2H])c(-c2c([2H])c([2H])c3c(oc4c([2H])c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c43)c2[2H])c([2H])c1[2H]]

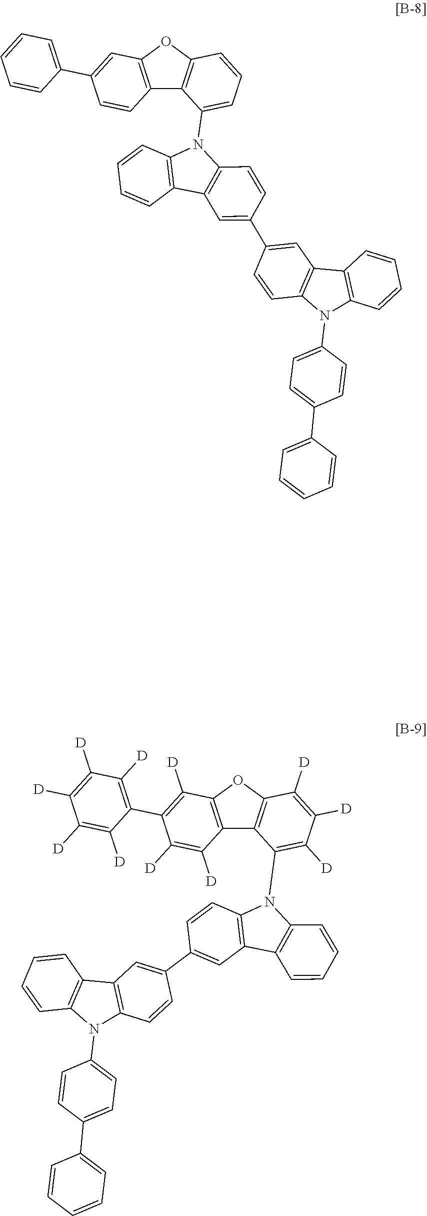 [SMILES: c1ccc(-c2ccc(-n3c4ccccc4c4cc(-c5ccc6c(c5)c5ccccc5n6-c5cccc6oc7cc(-c8ccccc8)ccc7c56)ccc43)cc2)cc1 ; [2H]c1c([2H])c([2H])c(-c2c([2H])c([2H])c3c(oc4c([2H])c([2H])c([2H])c(-n5c6ccccc6c6cc(-c7ccc8c(c7)c7ccccc7n8-c7ccc(-c8ccccc8)cc7)ccc65)c43)c2[2H])c([2H])c1[2H]]

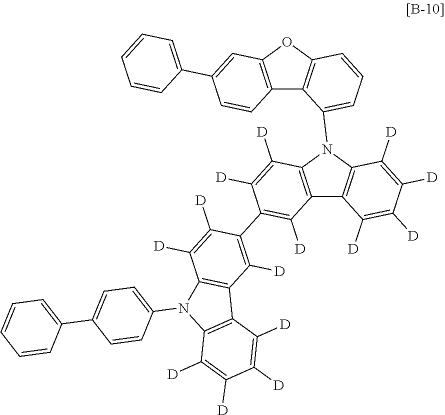 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4oc5cc(-c6ccccc6)ccc5c34)c([2H])c([2H])c1n2-c1ccc(-c2ccccc2)cc1]

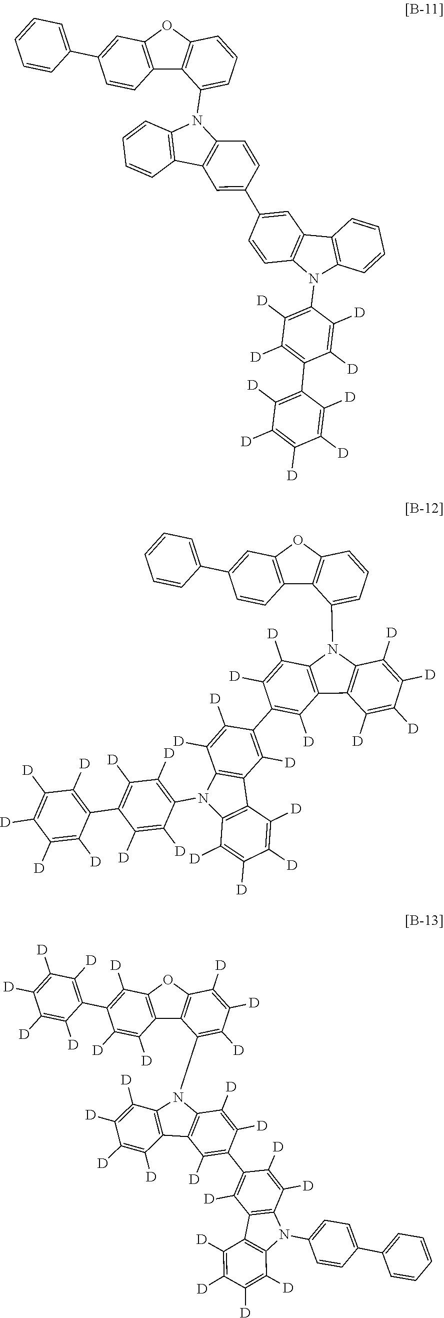

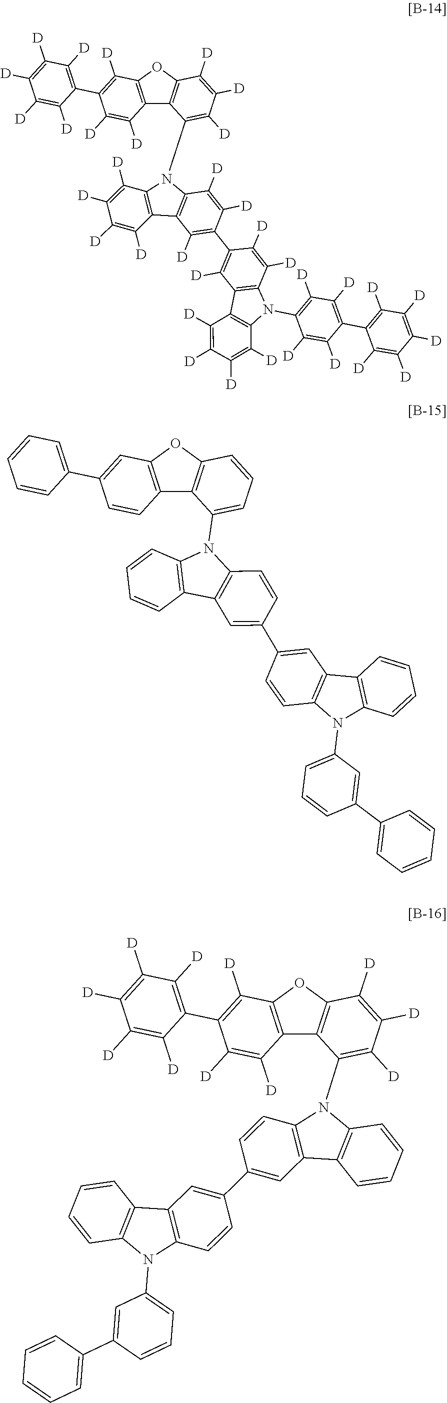

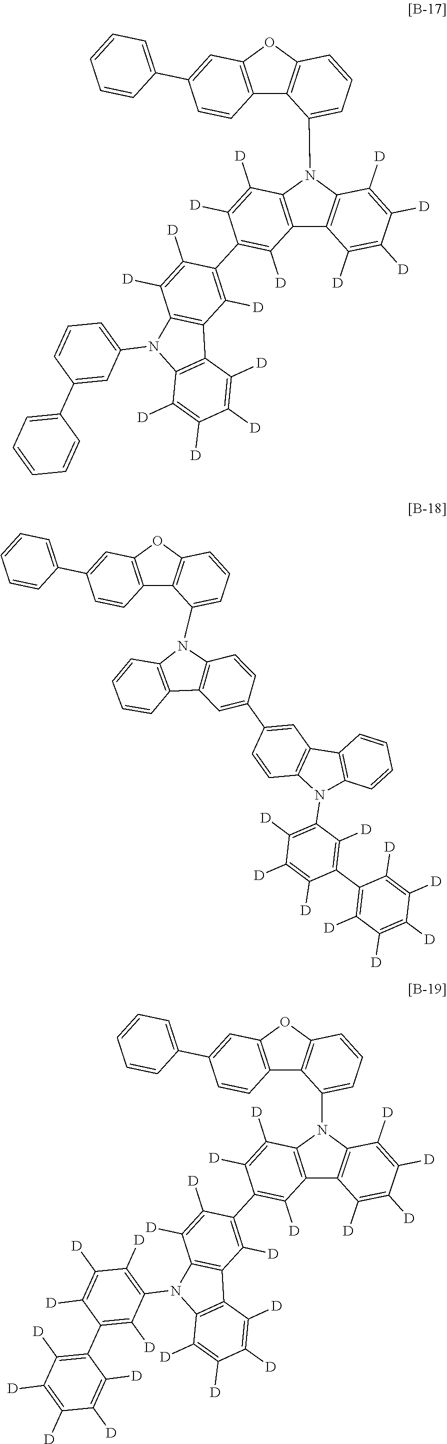

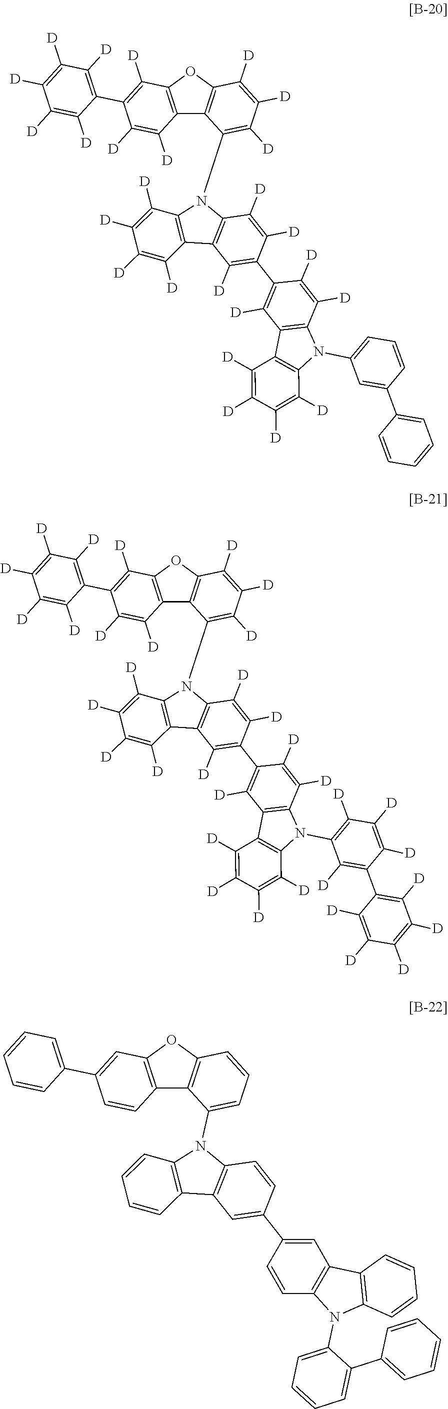

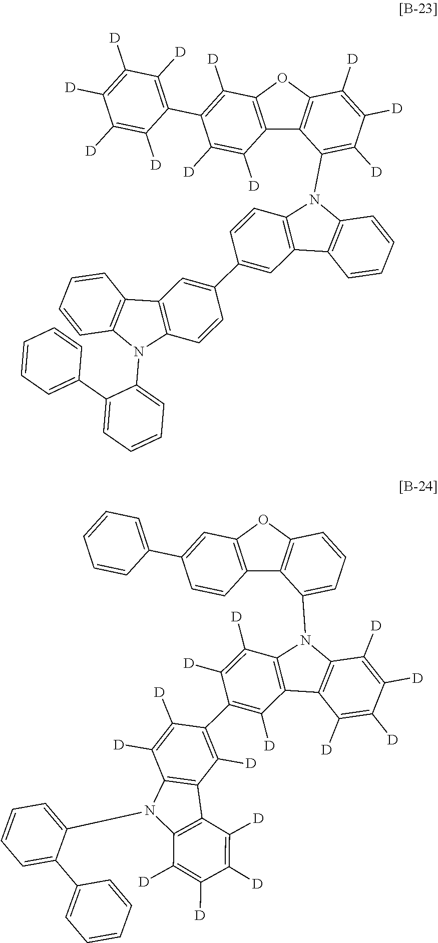 [SMILES: [2H]c1c([2H])c([2H])c(-c2c([2H])c([2H])c3c(oc4c([2H])c([2H])c([2H])c(-n5c6ccccc6c6cc(-c7ccc8c(c7)c7ccccc7n8-c7ccccc7-c7ccccc7)ccc65)c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4oc5cc(-c6ccccc6)ccc5c34)c([2H])c([2H])c1n2-c1ccccc1-c1ccccc1]

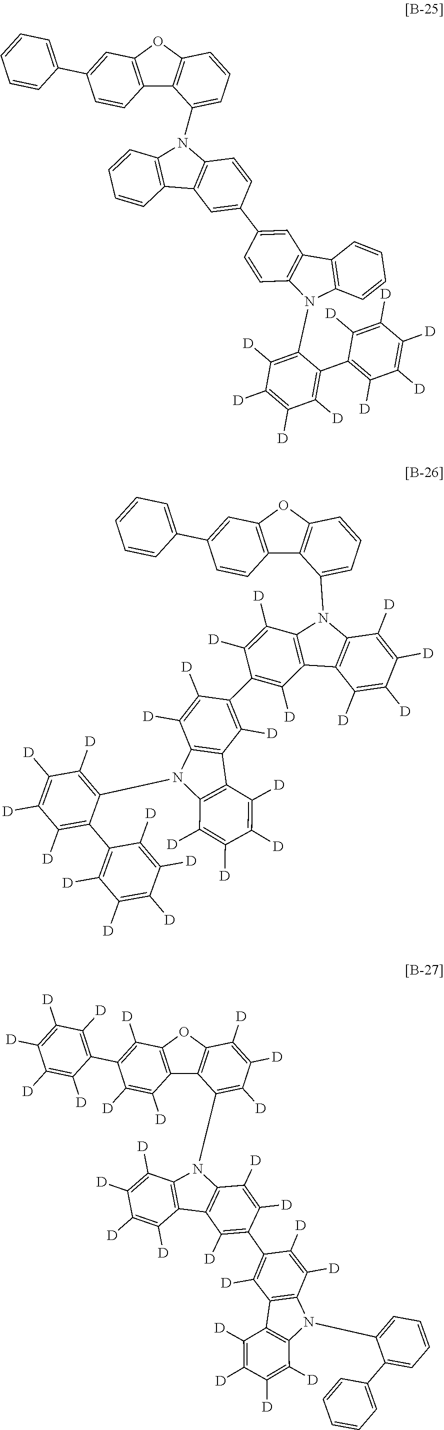

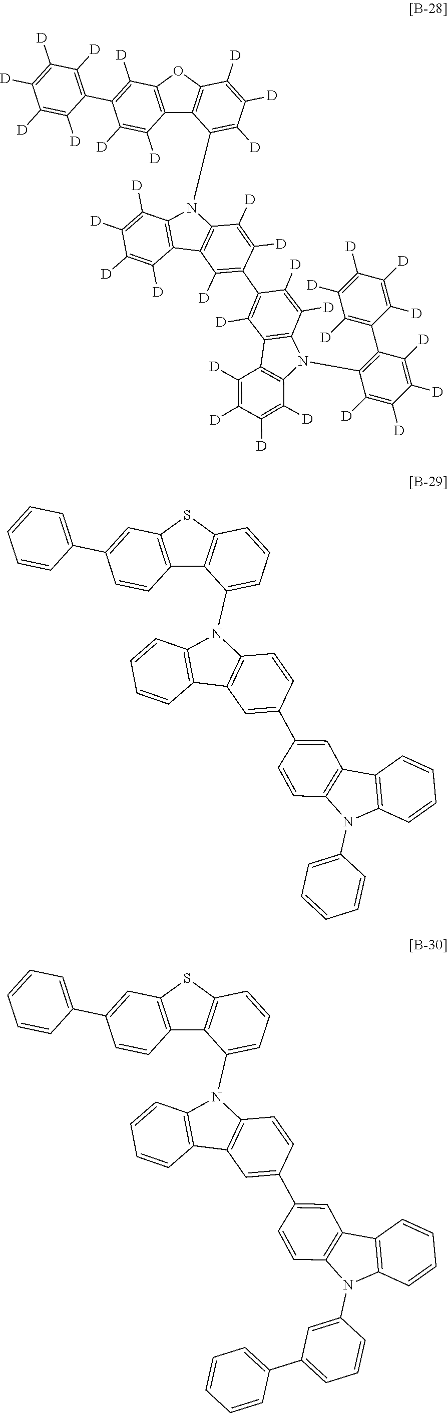 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c5oc6c([2H])c(-c7c([2H])c([2H])c([2H])c([2H])c7[2H])c([2H])c([2H])c6c45)c([2H])c([2H])c32)c([2H])c1[2H] ; c1ccc(-c2ccc3c(c2)sc2cccc(-n4c5ccccc5c5cc(-c6ccc7c(c6)c6ccccc6n7-c6ccccc6)ccc54)c23)cc1 ; c1ccc(-c2cccc(-n3c4ccccc4c4cc(-c5ccc6c(c5)c5ccccc5n6-c5cccc6sc7cc(-c8ccccc8)ccc7c56)ccc43)c2)cc1]

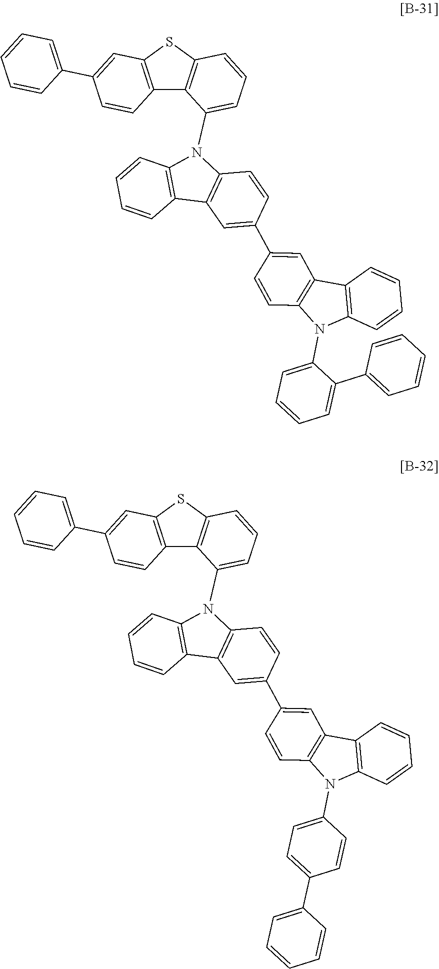 [SMILES: c1ccc(-c2ccc3c(c2)sc2cccc(-n4c5ccccc5c5cc(-c6ccc7c(c6)c6ccccc6n7-c6ccccc6-c6ccccc6)ccc54)c23)cc1 ; c1ccc(-c2ccc(-n3c4ccccc4c4cc(-c5ccc6c(c5)c5ccccc5n6-c5cccc6sc7cc(-c8ccccc8)ccc7c56)ccc43)cc2)cc1]

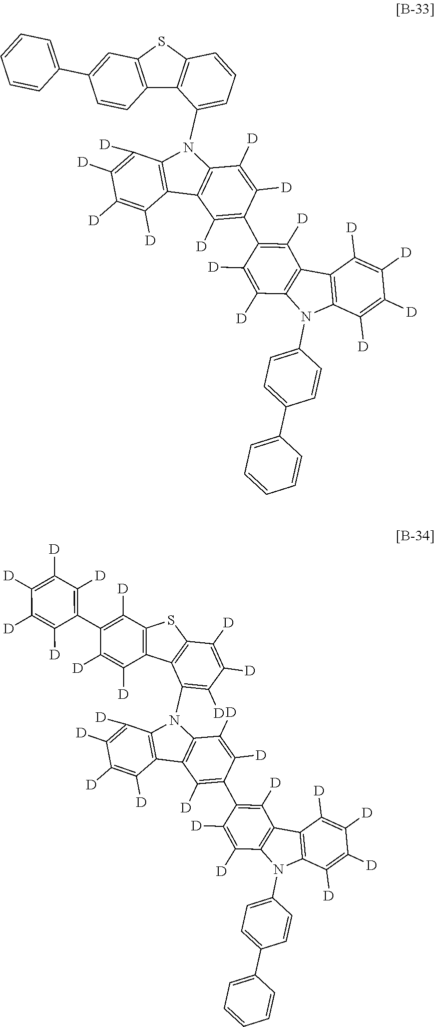 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4sc5cc(-c6ccccc6)ccc5c34)c([2H])c([2H])c1n2-c1ccc(-c2ccccc2)cc1 ; [2H]c1c([2H])c([2H])c(-c2c([2H])c([2H])c3c(sc4c([2H])c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)c43)c2[2H])c([2H])c1[2H]]

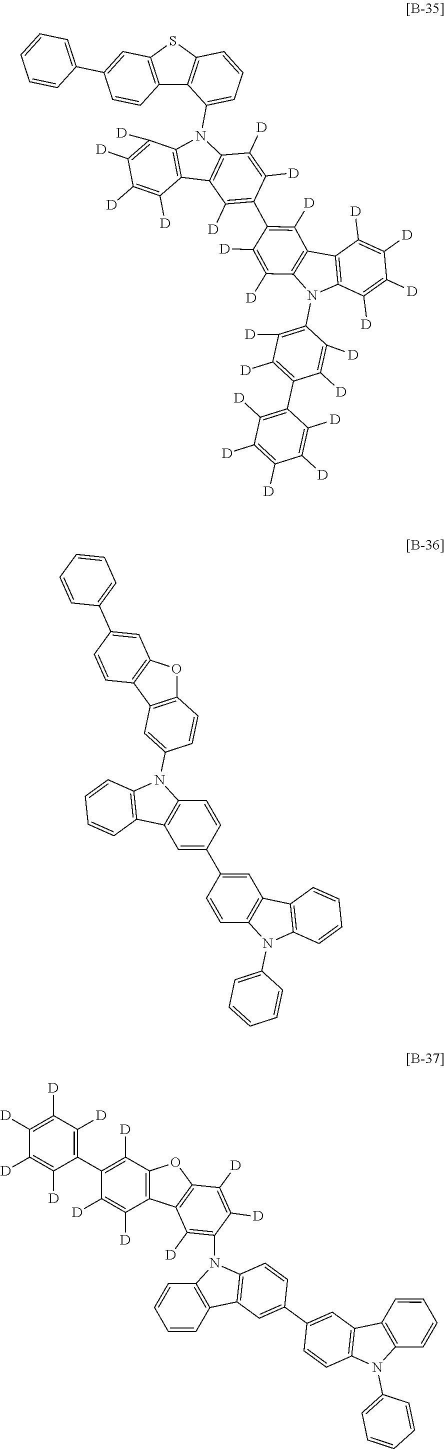 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6sc7cc(-c8ccccc8)ccc7c56)c([2H])c([2H])c43)c([2H])c2[2H])c([2H])c1[2H] ; c1ccc(-c2ccc3c(c2)oc2ccc(-n4c5ccccc5c5cc(-c6ccc7c(c6)c6ccccc6n7-c6ccccc6)ccc54)cc23)cc1 ; [2H]c1c([2H])c([2H])c(-c2c([2H])c([2H])c3c(oc4c([2H])c([2H])c(-n5c6ccccc6c6cc(-c7ccc8c(c7)c7ccccc7n8-c7ccccc7)ccc65)c([2H])c43)c2[2H])c([2H])c1[2H]]

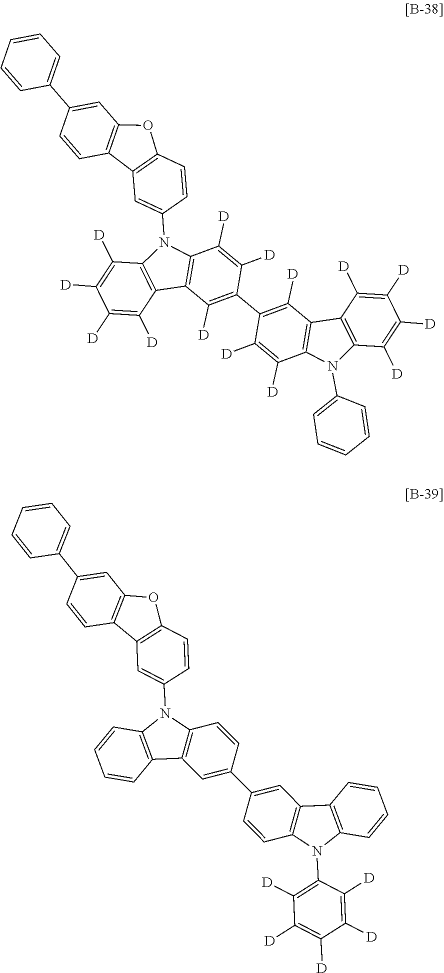 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4oc5cc(-c6ccccc6)ccc5c4c3)c([2H])c([2H])c1n2-c1ccccc1 ; [2H]c1c([2H])c([2H])c(-n2c3ccccc3c3cc(-c4ccc5c(c4)c4ccccc4n5-c4ccc5oc6cc(-c7ccccc7)ccc6c5c4)ccc32)c([2H])c1[2H]]

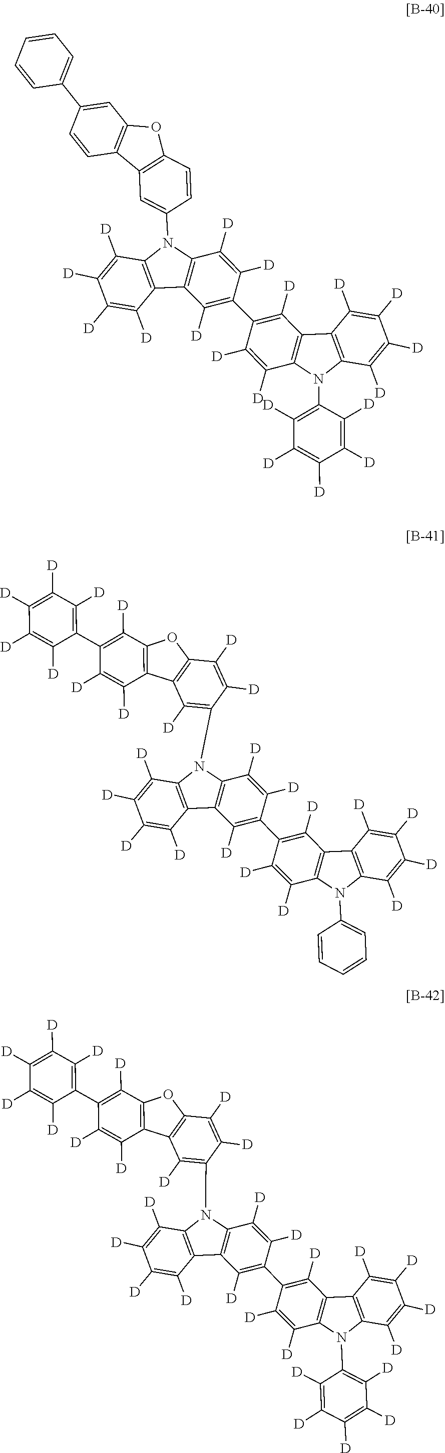

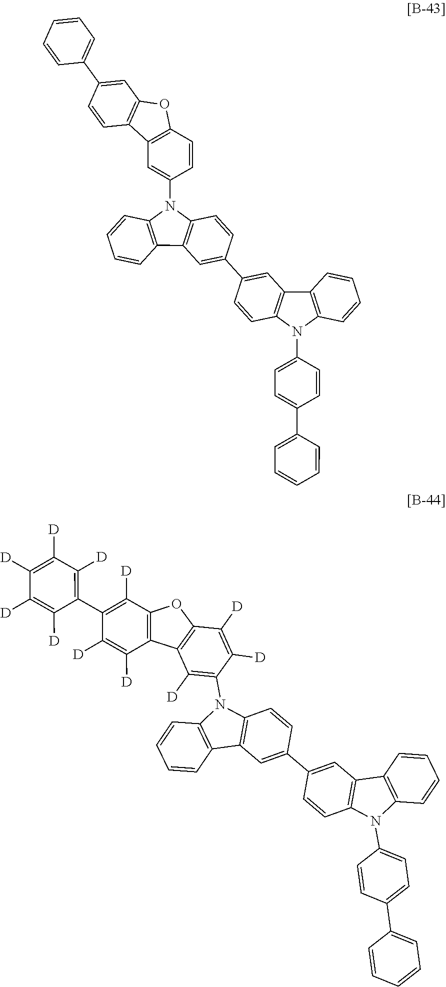 [SMILES: c1ccc(-c2ccc(-n3c4ccccc4c4cc(-c5ccc6c(c5)c5ccccc5n6-c5ccc6oc7cc(-c8ccccc8)ccc7c6c5)ccc43)cc2)cc1 ; [2H]c1c([2H])c([2H])c(-c2c([2H])c([2H])c3c(oc4c([2H])c([2H])c(-n5c6ccccc6c6cc(-c7ccc8c(c7)c7ccccc7n8-c7ccc(-c8ccccc8)cc7)ccc65)c([2H])c43)c2[2H])c([2H])c1[2H]]

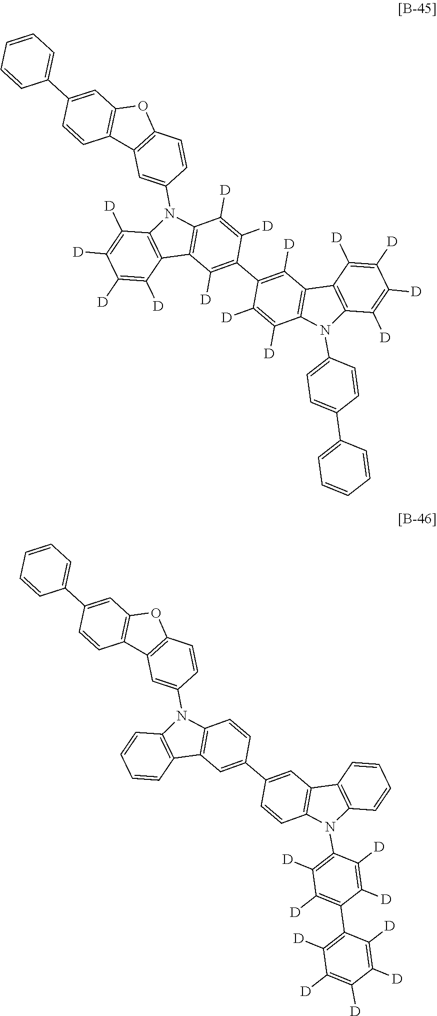 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4oc5cc(-c6ccccc6)ccc5c4c3)c([2H])c([2H])c1n2-c1ccc(-c2ccccc2)cc1 ; [2H]c1c([2H])c([2H])c(-c2c([2H])c([2H])c(-n3c4ccccc4c4cc(-c5ccc6c(c5)c5ccccc5n6-c5ccc6oc7cc(-c8ccccc8)ccc7c6c5)ccc43)c([2H])c2[2H])c([2H])c1[2H]]

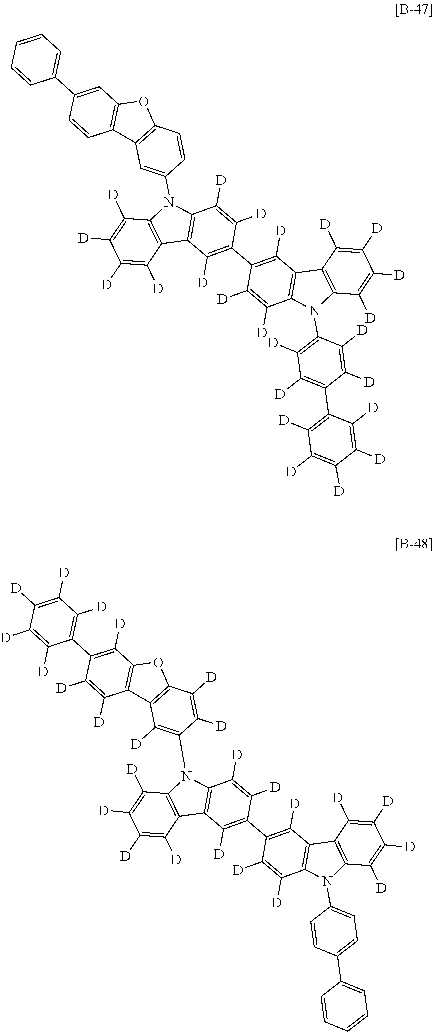 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6oc7cc(-c8ccccc8)ccc7c6c5)c([2H])c([2H])c43)c([2H])c2[2H])c([2H])c1[2H] ; [2H]c1c([2H])c([2H])c(-c2c([2H])c([2H])c3c(oc4c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)c([2H])c43)c2[2H])c([2H])c1[2H]]

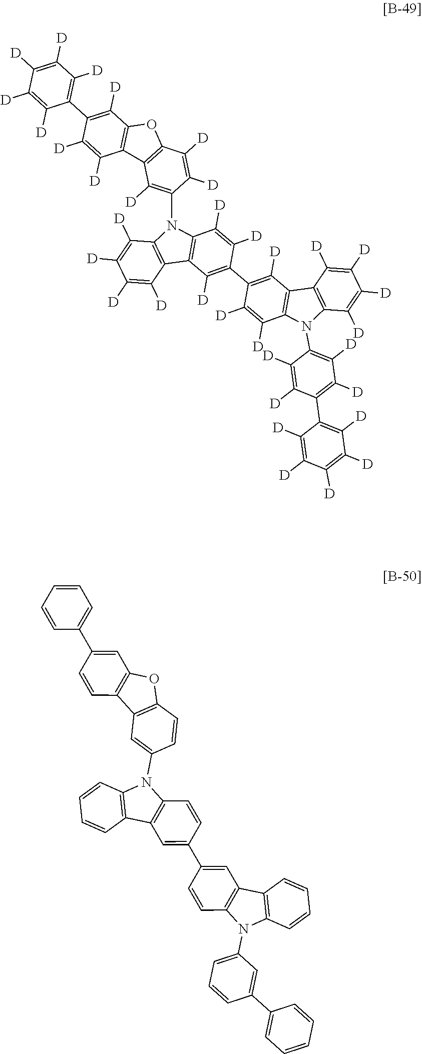 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6oc7c([2H])c(-c8c([2H])c([2H])c([2H])c([2H])c8[2H])c([2H])c([2H])c7c6c5[2H])c([2H])c([2H])c43)c([2H])c2[2H])c([2H])c1[2H] ; c1ccc(-c2cccc(-n3c4ccccc4c4cc(-c5ccc6c(c5)c5ccccc5n6-c5ccc6oc7cc(-c8ccccc8)ccc7c6c5)ccc43)c2)cc1]

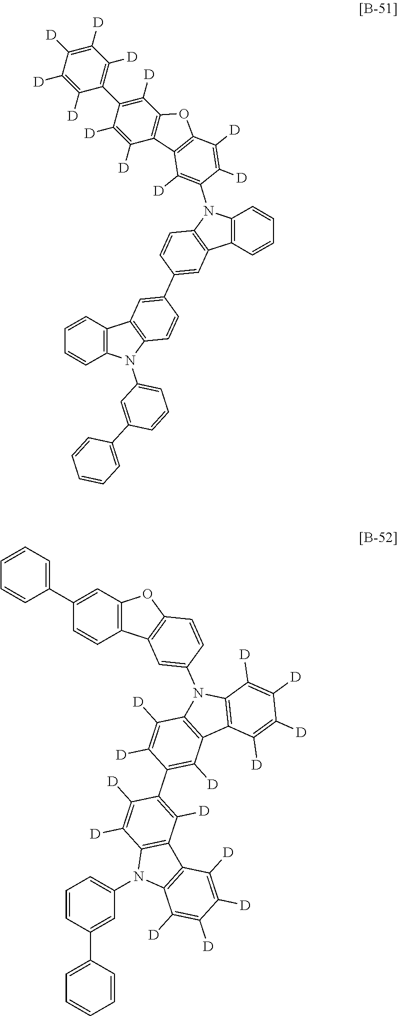 [SMILES: [2H]c1c([2H])c([2H])c(-c2c([2H])c([2H])c3c(oc4c([2H])c([2H])c(-n5c6ccccc6c6cc(-c7ccc8c(c7)c7ccccc7n8-c7cccc(-c8ccccc8)c7)ccc65)c([2H])c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4oc5cc(-c6ccccc6)ccc5c4c3)c([2H])c([2H])c1n2-c1cccc(-c2ccccc2)c1]

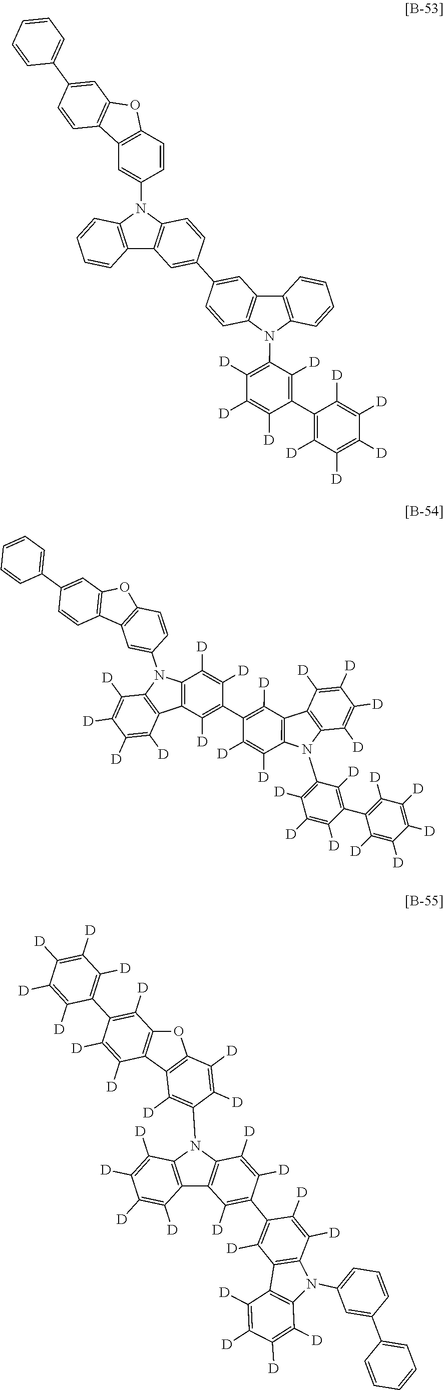

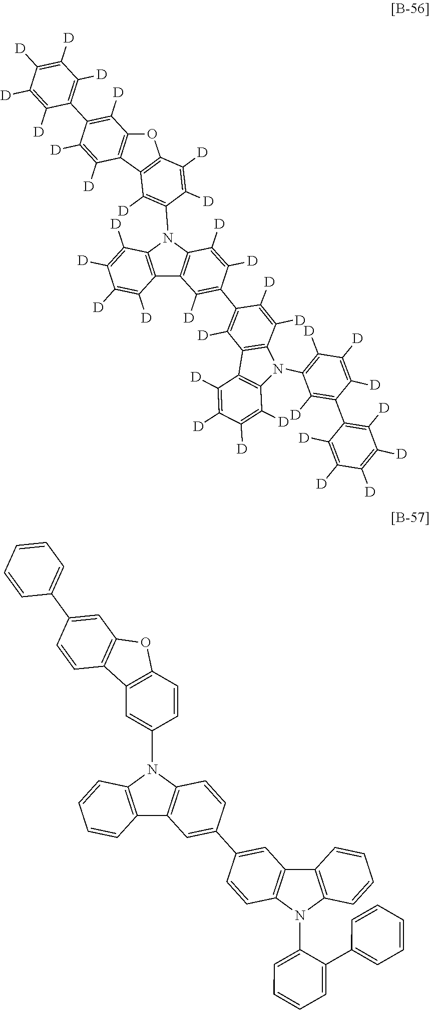 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6oc7c([2H])c(-c8c([2H])c([2H])c([2H])c([2H])c8[2H])c([2H])c([2H])c7c6c5[2H])c([2H])c([2H])c43)c2[2H])c([2H])c1[2H] ; c1ccc(-c2ccc3c(c2)oc2ccc(-n4c5ccccc5c5cc(-c6ccc7c(c6)c6ccccc6n7-c6ccccc6-c6ccccc6)ccc54)cc23)cc1]

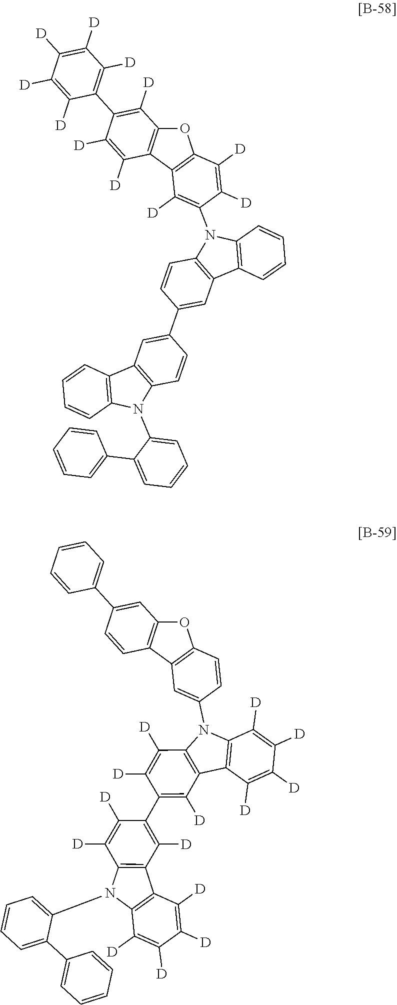 [SMILES: [2H]c1c([2H])c([2H])c(-c2c([2H])c([2H])c3c(oc4c([2H])c([2H])c(-n5c6ccccc6c6cc(-c7ccc8c(c7)c7ccccc7n8-c7ccccc7-c7ccccc7)ccc65)c([2H])c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3-c3ccccc3)c([2H])c([2H])c1n2-c1ccc2oc3cc(-c4ccccc4)ccc3c2c1]

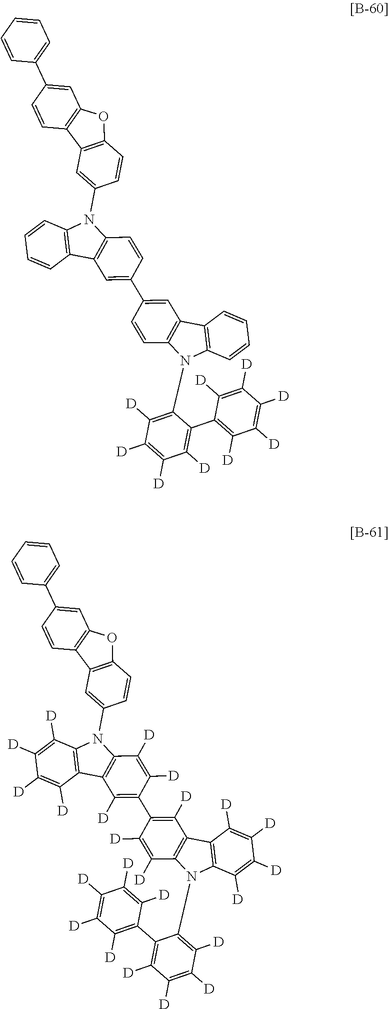 [SMILES: [2H]c1c([2H])c([2H])c(-c2c([2H])c([2H])c([2H])c([2H])c2-n2c3ccccc3c3cc(-c4ccc5c(c4)c4ccccc4n5-c4ccc5oc6cc(-c7ccccc7)ccc6c5c4)ccc32)c([2H])c1[2H] ; [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5oc6cc(-c7ccccc7)ccc6c5c4)c([2H])c([2H])c32)c([2H])c1[2H]]

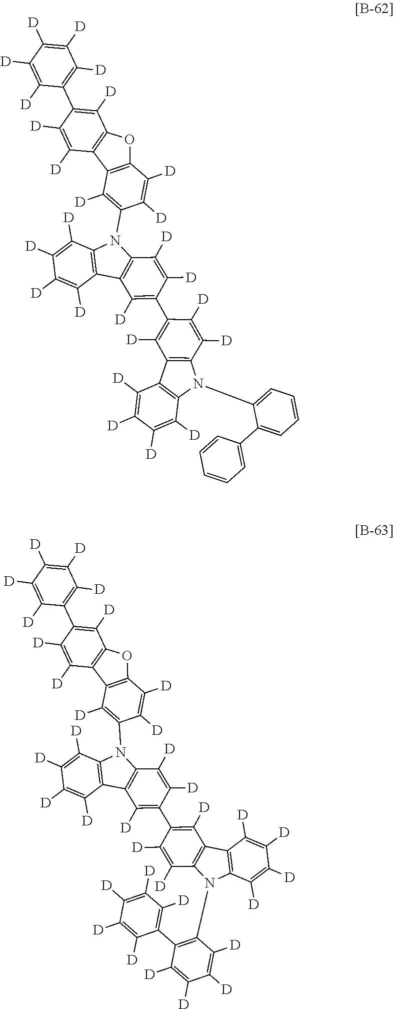

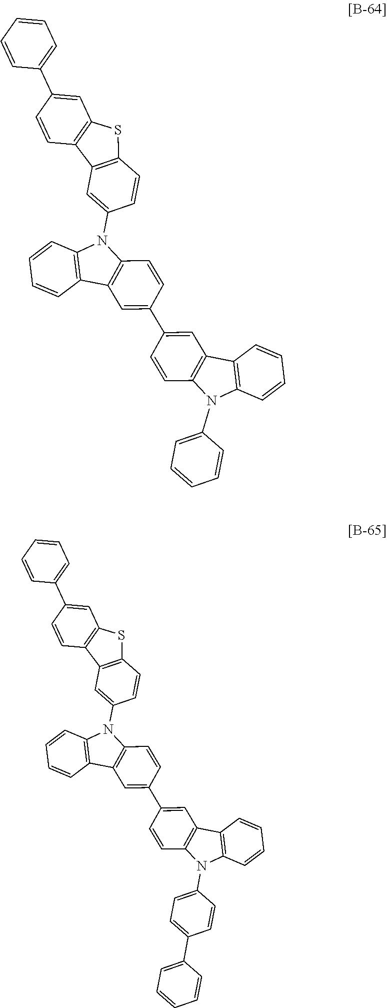 [SMILES: c1ccc(-c2ccc3c(c2)sc2ccc(-n4c5ccccc5c5cc(-c6ccc7c(c6)c6ccccc6n7-c6ccccc6)ccc54)cc23)cc1 ; c1ccc(-c2ccc(-n3c4ccccc4c4cc(-c5ccc6c(c5)c5ccccc5n6-c5ccc6sc7cc(-c8ccccc8)ccc7c6c5)ccc43)cc2)cc1]

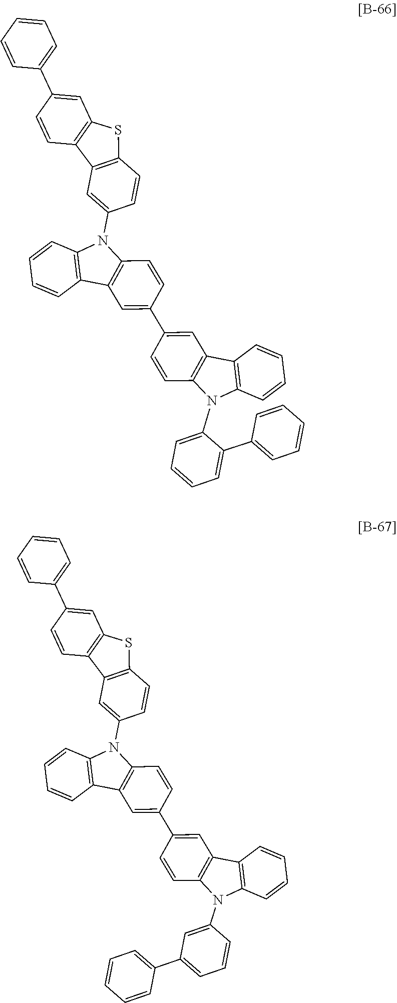 [SMILES: c1ccc(-c2ccc3c(c2)sc2ccc(-n4c5ccccc5c5cc(-c6ccc7c(c6)c6ccccc6n7-c6ccccc6-c6ccccc6)ccc54)cc23)cc1 ; c1ccc(-c2cccc(-n3c4ccccc4c4cc(-c5ccc6c(c5)c5ccccc5n6-c5ccc6sc7cc(-c8ccccc8)ccc7c6c5)ccc43)c2)cc1]

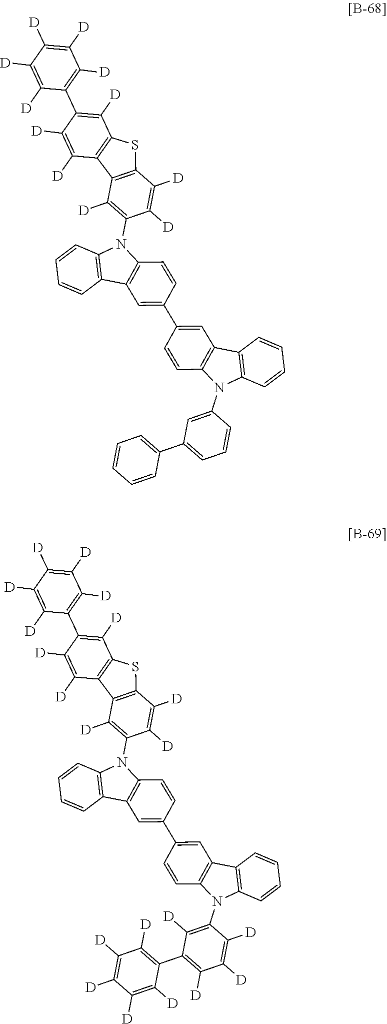 [SMILES: [2H]c1c([2H])c([2H])c(-c2c([2H])c([2H])c3c(sc4c([2H])c([2H])c(-n5c6ccccc6c6cc(-c7ccc8c(c7)c7ccccc7n8-c7cccc(-c8ccccc8)c7)ccc65)c([2H])c43)c2[2H])c([2H])c1[2H] ; [2H]c1c([2H])c([2H])c(-c2c([2H])c([2H])c([2H])c(-n3c4ccccc4c4cc(-c5ccc6c(c5)c5ccccc5n6-c5c([2H])c([2H])c6sc7c([2H])c(-c8c([2H])c([2H])c([2H])c([2H])c8[2H])c([2H])c([2H])c7c6c5[2H])ccc43)c2[2H])c([2H])c1[2H]]

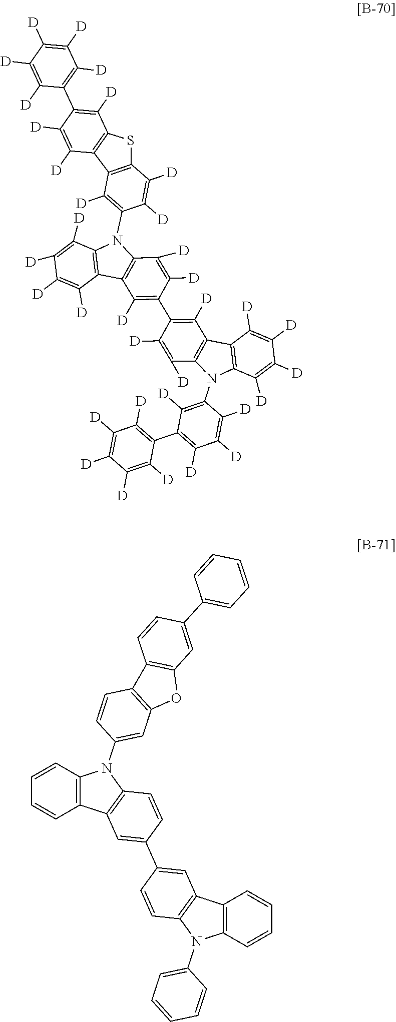 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6sc7c([2H])c(-c8c([2H])c([2H])c([2H])c([2H])c8[2H])c([2H])c([2H])c7c6c5[2H])c([2H])c([2H])c43)c2[2H])c([2H])c1[2H] ; c1ccc(-c2ccc3c(c2)oc2cc(-n4c5ccccc5c5cc(-c6ccc7c(c6)c6ccccc6n7-c6ccccc6)ccc54)ccc23)cc1]

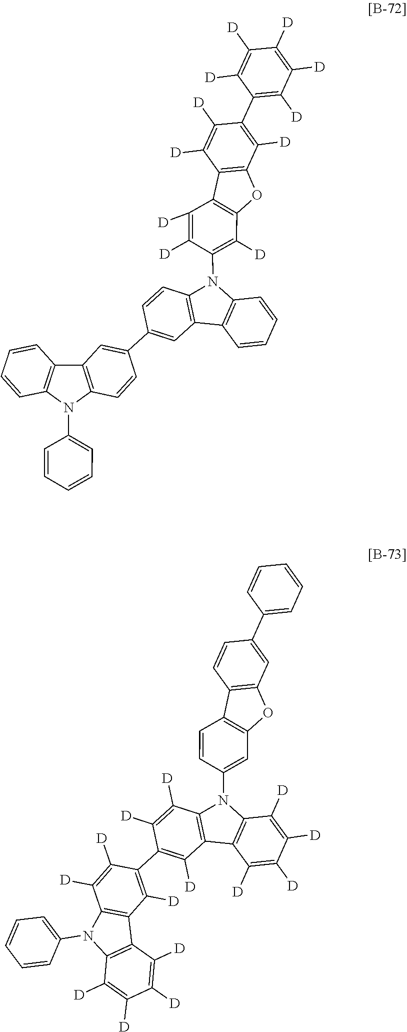 [SMILES: [2H]c1c([2H])c([2H])c(-c2c([2H])c([2H])c3c(oc4c([2H])c(-n5c6ccccc6c6cc(-c7ccc8c(c7)c7ccccc7n8-c7ccccc7)ccc65)c([2H])c([2H])c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c(c3)oc3cc(-c5ccccc5)ccc34)c([2H])c([2H])c1n2-c1ccccc1]

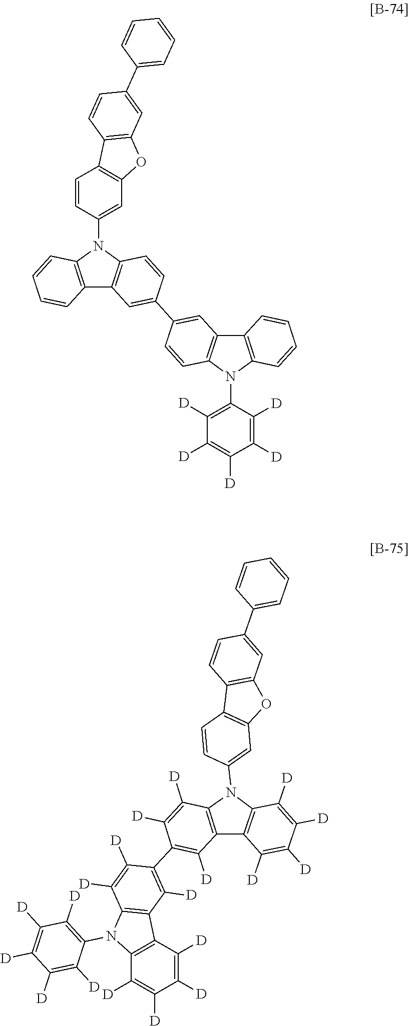 [SMILES: [2H]c1c([2H])c([2H])c(-n2c3ccccc3c3cc(-c4ccc5c(c4)c4ccccc4n5-c4ccc5c(c4)oc4cc(-c6ccccc6)ccc45)ccc32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c(c4)oc4cc(-c6ccccc6)ccc45)c([2H])c([2H])c32)c([2H])c1[2H]]

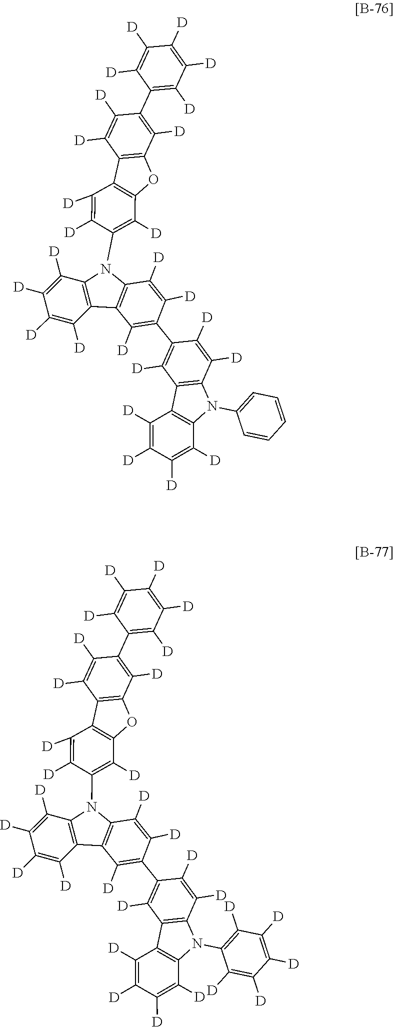 [SMILES: [2H]c1c([2H])c([2H])c(-c2c([2H])c([2H])c3c(oc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)c([2H])c([2H])c43)c2[2H])c([2H])c1[2H] ; [2H]c1c([2H])c([2H])c(-c2c([2H])c([2H])c3c(oc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c([2H])c([2H])c43)c2[2H])c([2H])c1[2H]]

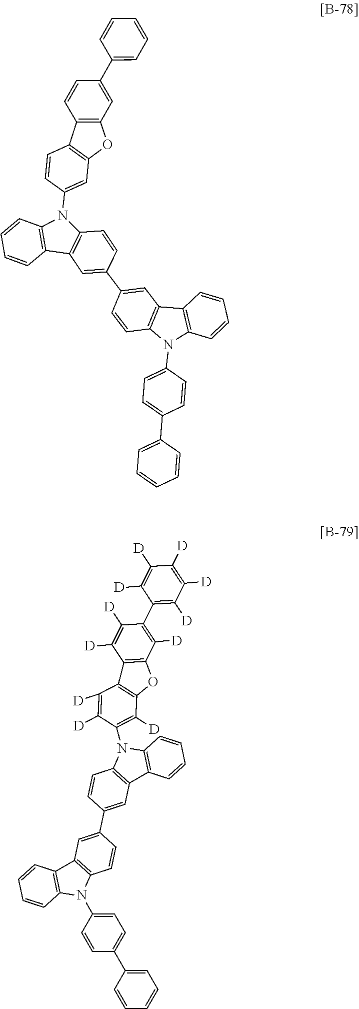 [SMILES: c1ccc(-c2ccc(-n3c4ccccc4c4cc(-c5ccc6c(c5)c5ccccc5n6-c5ccc6c(c5)oc5cc(-c7ccccc7)ccc56)ccc43)cc2)cc1 ; [2H]c1c([2H])c([2H])c(-c2c([2H])c([2H])c3c(oc4c([2H])c(-n5c6ccccc6c6cc(-c7ccc8c(c7)c7ccccc7n8-c7ccc(-c8ccccc8)cc7)ccc65)c([2H])c([2H])c43)c2[2H])c([2H])c1[2H]]

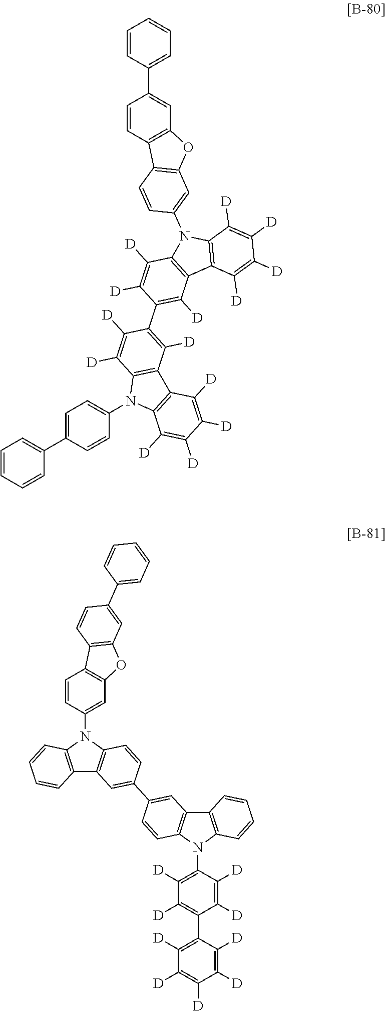 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c(c3)oc3cc(-c5ccccc5)ccc34)c([2H])c([2H])c1n2-c1ccc(-c2ccccc2)cc1 ; [2H]c1c([2H])c([2H])c(-c2c([2H])c([2H])c(-n3c4ccccc4c4cc(-c5ccc6c(c5)c5ccccc5n6-c5ccc6c(c5)oc5cc(-c7ccccc7)ccc56)ccc43)c([2H])c2[2H])c([2H])c1[2H]]

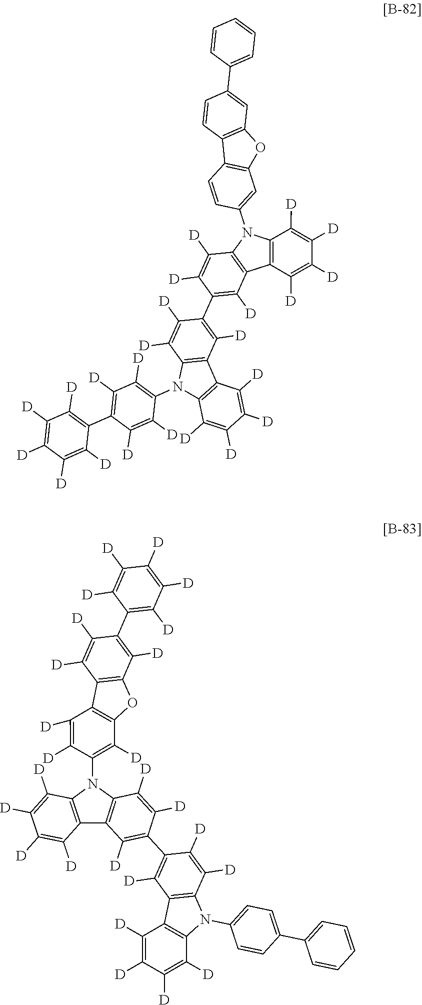 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)oc5cc(-c7ccccc7)ccc56)c([2H])c([2H])c43)c([2H])c2[2H])c([2H])c1[2H] ; [2H]c1c([2H])c([2H])c(-c2c([2H])c([2H])c3c(oc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)c([2H])c([2H])c43)c2[2H])c([2H])c1[2H]]

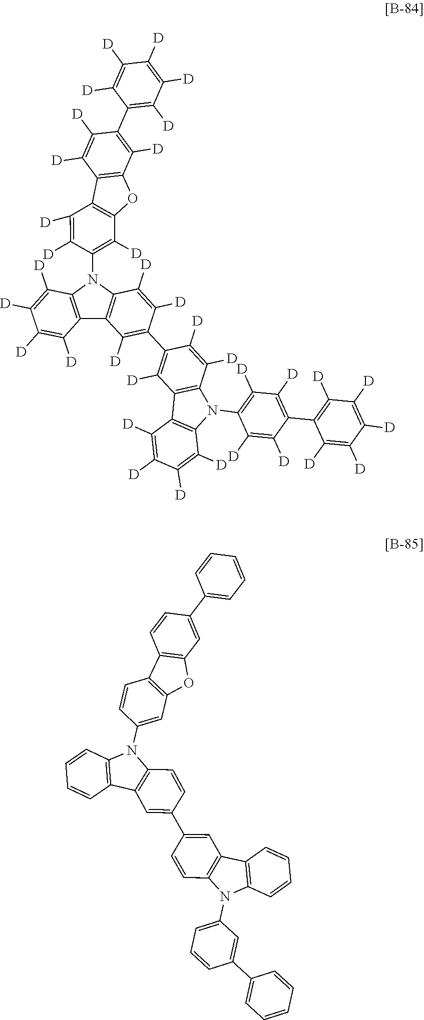 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c(oc7c([2H])c(-c8c([2H])c([2H])c([2H])c([2H])c8[2H])c([2H])c([2H])c76)c5[2H])c([2H])c([2H])c43)c([2H])c2[2H])c([2H])c1[2H] ; c1ccc(-c2cccc(-n3c4ccccc4c4cc(-c5ccc6c(c5)c5ccccc5n6-c5ccc6c(c5)oc5cc(-c7ccccc7)ccc56)ccc43)c2)cc1]

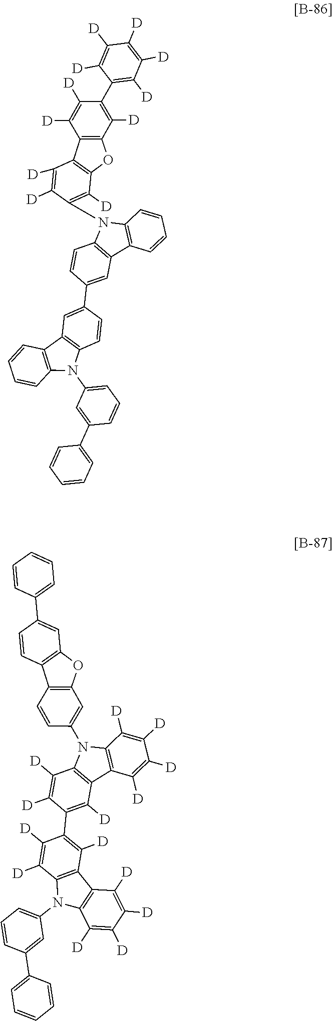 [SMILES: [2H]c1c([2H])c([2H])c(-c2c([2H])c([2H])c3c(oc4c([2H])c(-n5c6ccccc6c6cc(-c7ccc8c(c7)c7ccccc7n8-c7cccc(-c8ccccc8)c7)ccc65)c([2H])c([2H])c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c(c3)oc3cc(-c5ccccc5)ccc34)c([2H])c([2H])c1n2-c1cccc(-c2ccccc2)c1]

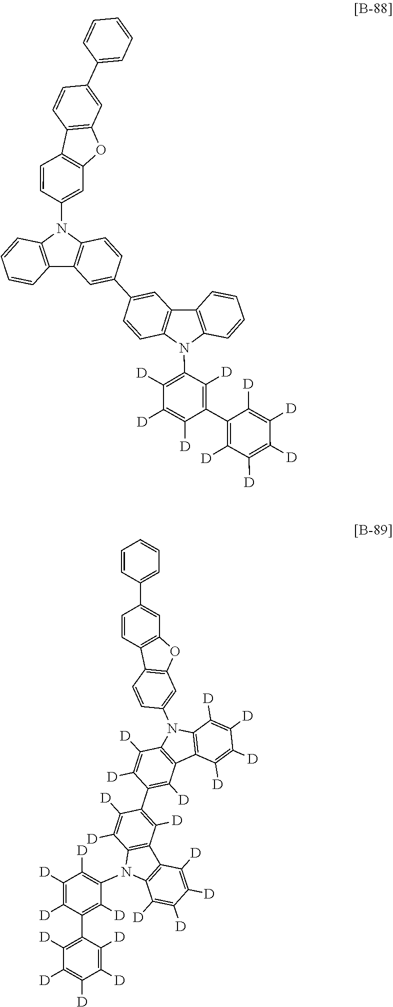 [SMILES: [2H]c1c([2H])c([2H])c(-c2c([2H])c([2H])c([2H])c(-n3c4ccccc4c4cc(-c5ccc6c(c5)c5ccccc5n6-c5ccc6c(c5)oc5cc(-c7ccccc7)ccc56)ccc43)c2[2H])c([2H])c1[2H] ; [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)oc5cc(-c7ccccc7)ccc56)c([2H])c([2H])c43)c2[2H])c([2H])c1[2H]]

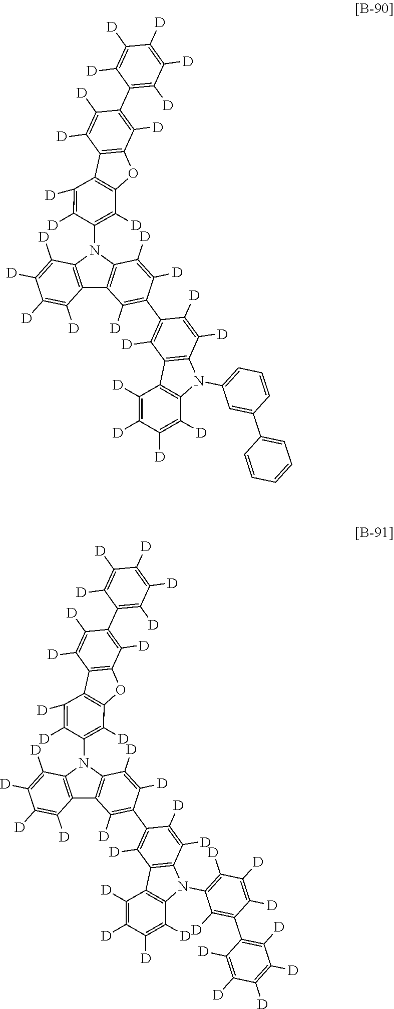

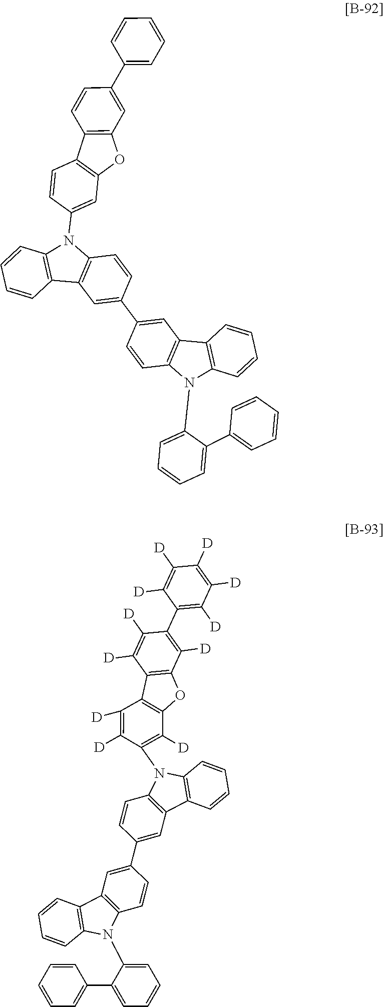 [SMILES: c1ccc(-c2ccc3c(c2)oc2cc(-n4c5ccccc5c5cc(-c6ccc7c(c6)c6ccccc6n7-c6ccccc6-c6ccccc6)ccc54)ccc23)cc1 ; [2H]c1c([2H])c([2H])c(-c2c([2H])c([2H])c3c(oc4c([2H])c(-n5c6ccccc6c6cc(-c7ccc8c(c7)c7ccccc7n8-c7ccccc7-c7ccccc7)ccc65)c([2H])c([2H])c43)c2[2H])c([2H])c1[2H]]

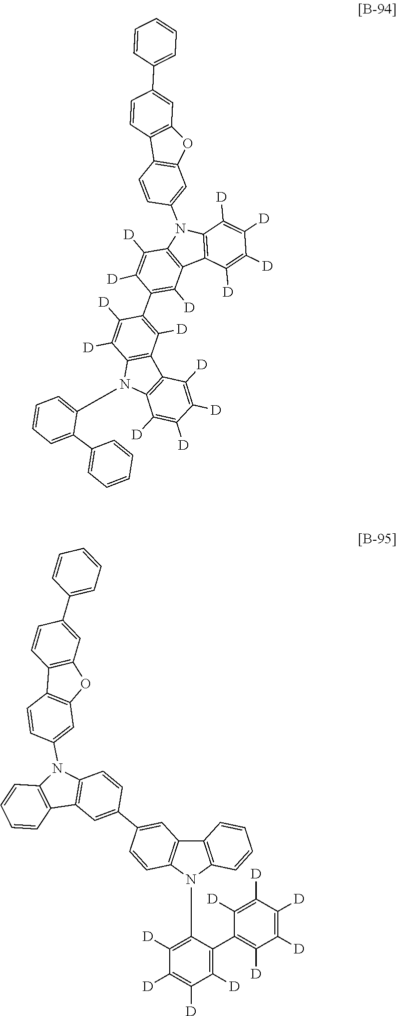 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3-c3ccccc3)c([2H])c([2H])c1n2-c1ccc2c(c1)oc1cc(-c3ccccc3)ccc12 ; [2H]c1c([2H])c([2H])c(-c2c([2H])c([2H])c([2H])c([2H])c2-n2c3ccccc3c3cc(-c4ccc5c(c4)c4ccccc4n5-c4ccc5c(c4)oc4cc(-c6ccccc6)ccc45)ccc32)c([2H])c1[2H]]

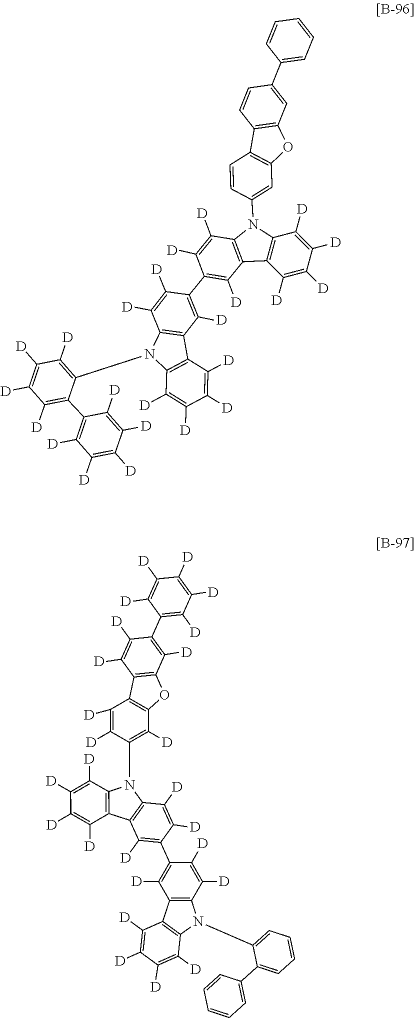 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c(c4)oc4cc(-c6ccccc6)ccc45)c([2H])c([2H])c32)c([2H])c1[2H] ; [2H]c1c([2H])c([2H])c(-c2c([2H])c([2H])c3c(oc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7-c7ccccc7)c([2H])c([2H])c65)c([2H])c([2H])c43)c2[2H])c([2H])c1[2H]]

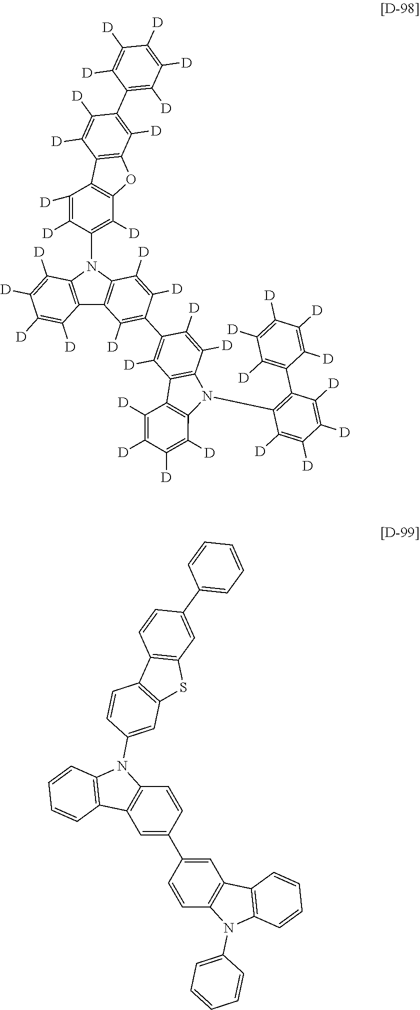 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5c(oc6c([2H])c(-c7c([2H])c([2H])c([2H])c([2H])c7[2H])c([2H])c([2H])c65)c4[2H])c([2H])c([2H])c32)c([2H])c1[2H] ; c1ccc(-c2ccc3c(c2)sc2cc(-n4c5ccccc5c5cc(-c6ccc7c(c6)c6ccccc6n7-c6ccccc6)ccc54)ccc23)cc1]

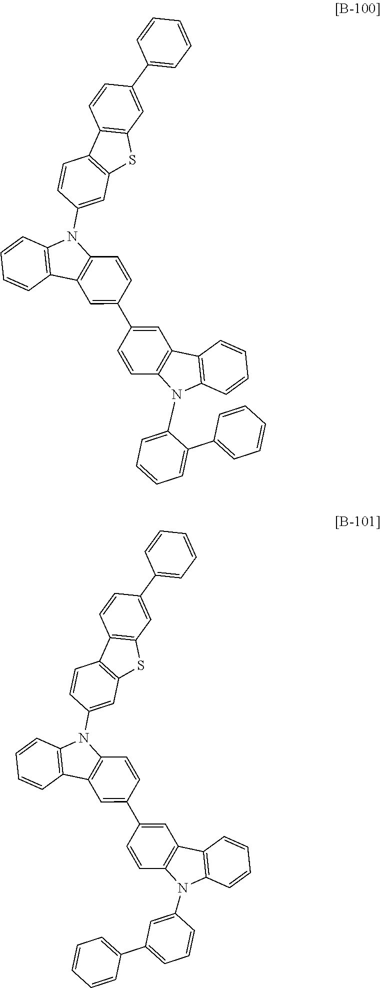 [SMILES: c1ccc(-c2ccc3c(c2)sc2cc(-n4c5ccccc5c5cc(-c6ccc7c(c6)c6ccccc6n7-c6ccccc6-c6ccccc6)ccc54)ccc23)cc1 ; c1ccc(-c2cccc(-n3c4ccccc4c4cc(-c5ccc6c(c5)c5ccccc5n6-c5ccc6c(c5)sc5cc(-c7ccccc7)ccc56)ccc43)c2)cc1]

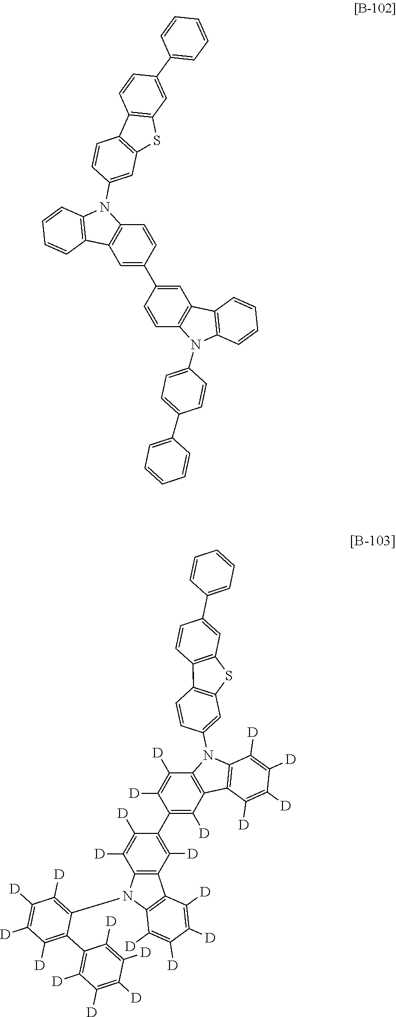 [SMILES: c1ccc(-c2ccc(-n3c4ccccc4c4cc(-c5ccc6c(c5)c5ccccc5n6-c5ccc6c(c5)sc5cc(-c7ccccc7)ccc56)ccc43)cc2)cc1 ; [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c(c4)sc4cc(-c6ccccc6)ccc45)c([2H])c([2H])c32)c([2H])c1[2H]]

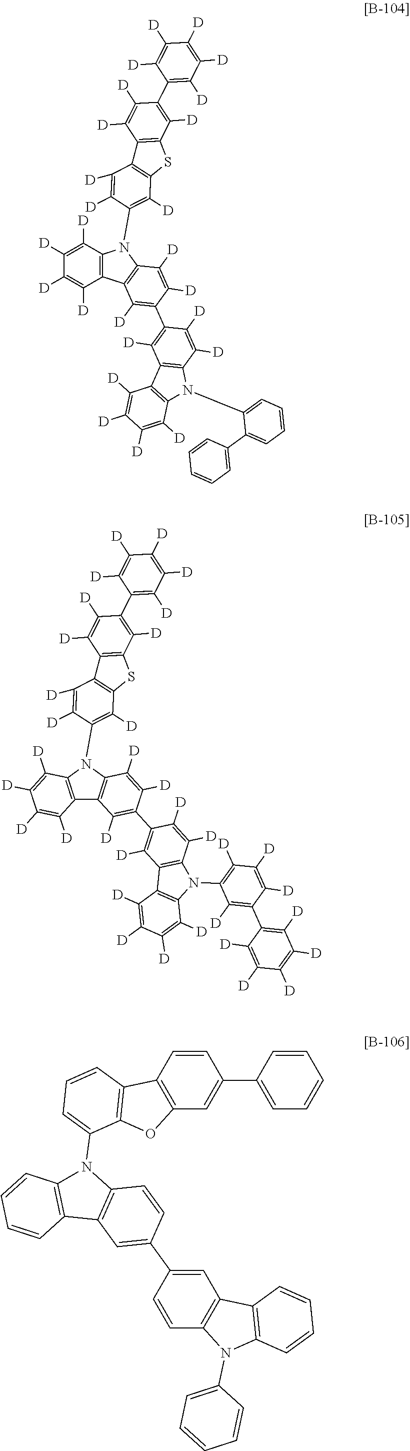

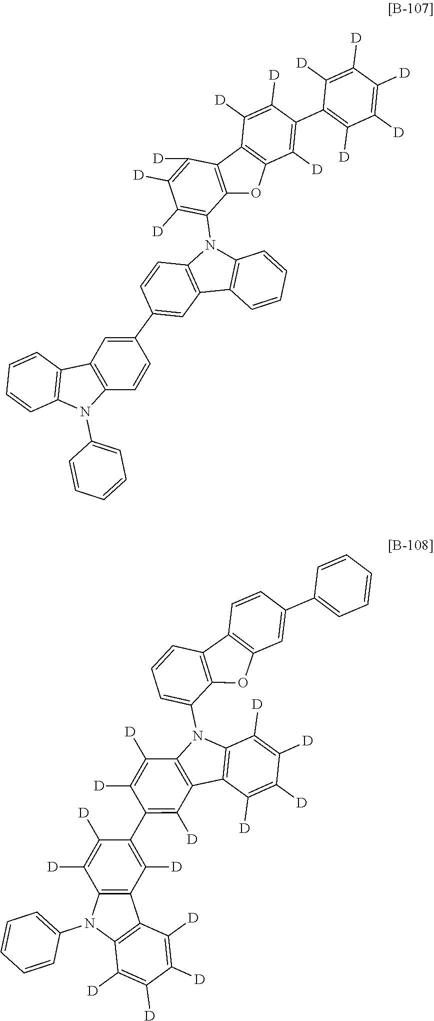 [SMILES: [2H]c1c([2H])c([2H])c(-c2c([2H])c([2H])c3c(oc4c(-n5c6ccccc6c6cc(-c7ccc8c(c7)c7ccccc7n8-c7ccccc7)ccc65)c([2H])c([2H])c([2H])c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3oc3cc(-c5ccccc5)ccc34)c([2H])c([2H])c1n2-c1ccccc1]

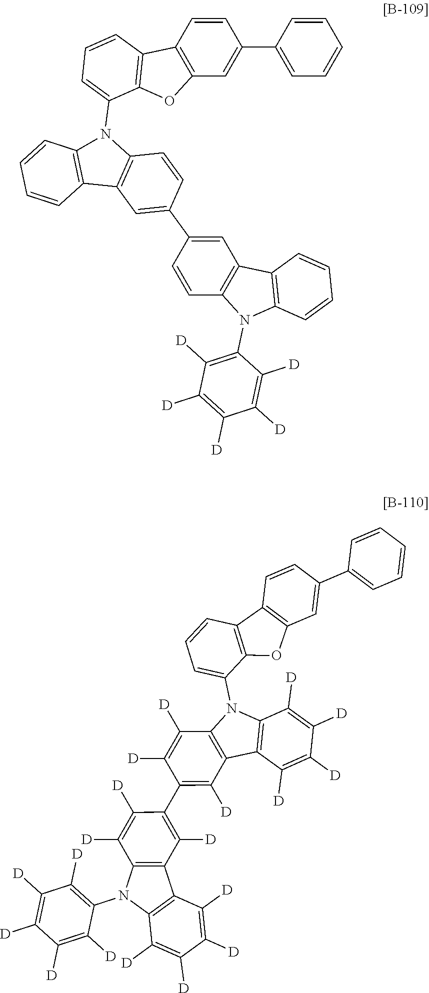 [SMILES: [2H]c1c([2H])c([2H])c(-n2c3ccccc3c3cc(-c4ccc5c(c4)c4ccccc4n5-c4cccc5c4oc4cc(-c6ccccc6)ccc45)ccc32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5c4oc4cc(-c6ccccc6)ccc45)c([2H])c([2H])c32)c([2H])c1[2H]]

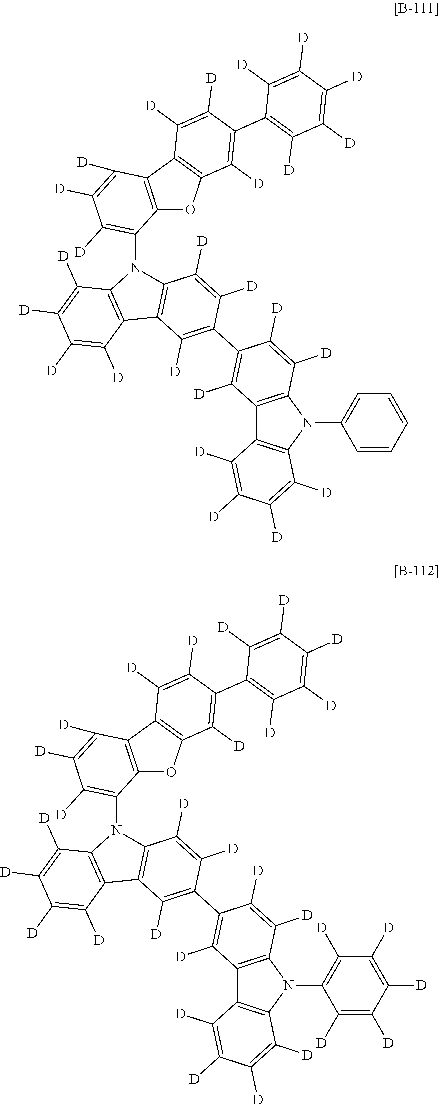 [SMILES: [2H]c1c([2H])c([2H])c(-c2c([2H])c([2H])c3c(oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)c([2H])c([2H])c([2H])c43)c2[2H])c([2H])c1[2H] ; [2H]c1c([2H])c([2H])c(-c2c([2H])c([2H])c3c(oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c([2H])c([2H])c([2H])c43)c2[2H])c([2H])c1[2H]]

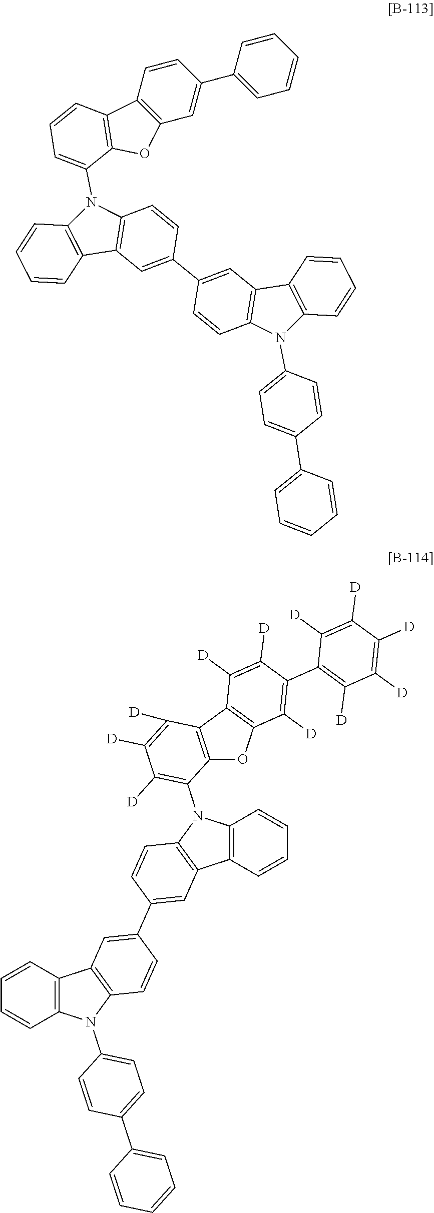 [SMILES: c1ccc(-c2ccc(-n3c4ccccc4c4cc(-c5ccc6c(c5)c5ccccc5n6-c5cccc6c5oc5cc(-c7ccccc7)ccc56)ccc43)cc2)cc1 ; [2H]c1c([2H])c([2H])c(-c2c([2H])c([2H])c3c(oc4c(-n5c6ccccc6c6cc(-c7ccc8c(c7)c7ccccc7n8-c7ccc(-c8ccccc8)cc7)ccc65)c([2H])c([2H])c([2H])c43)c2[2H])c([2H])c1[2H]]

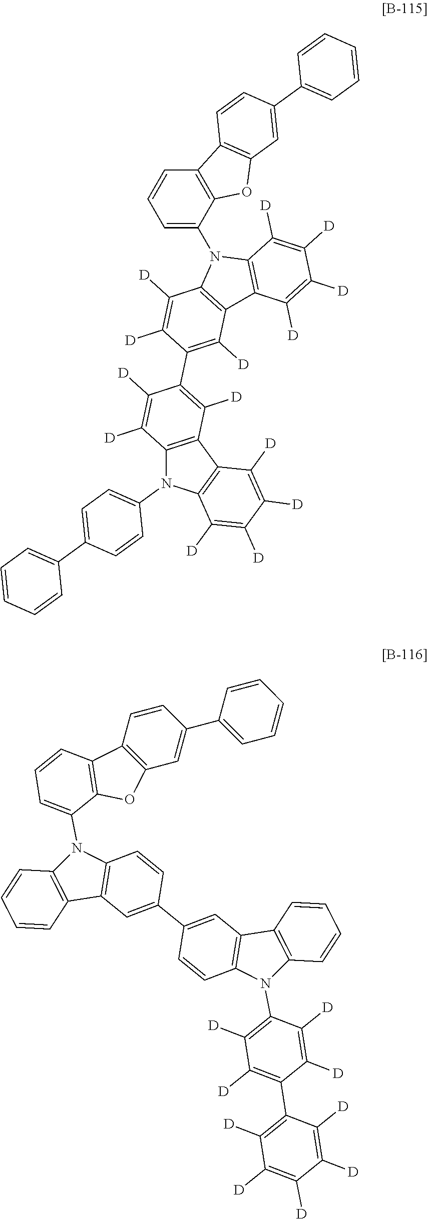 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3oc3cc(-c5ccccc5)ccc34)c([2H])c([2H])c1n2-c1ccc(-c2ccccc2)cc1 ; [2H]c1c([2H])c([2H])c(-c2c([2H])c([2H])c(-n3c4ccccc4c4cc(-c5ccc6c(c5)c5ccccc5n6-c5cccc6c5oc5cc(-c7ccccc7)ccc56)ccc43)c([2H])c2[2H])c([2H])c1[2H]]

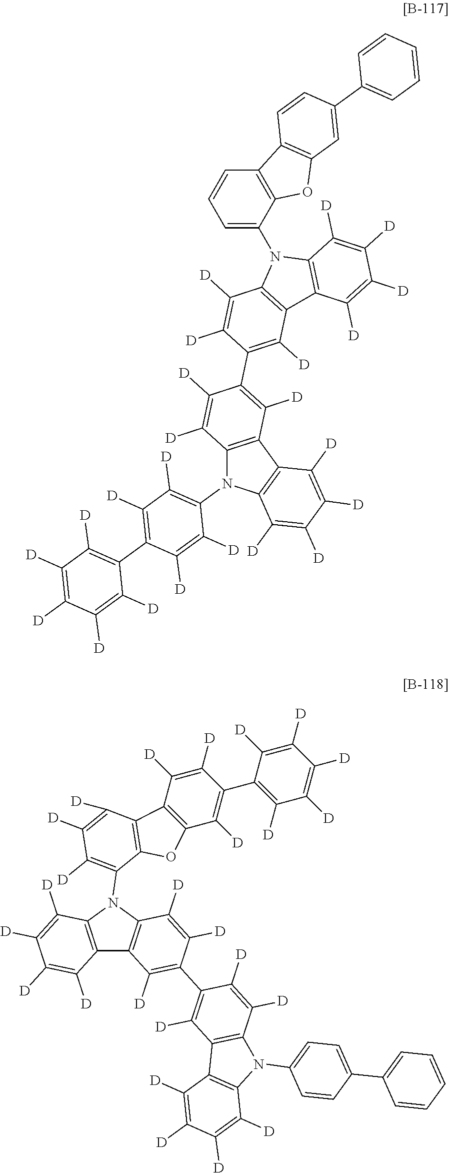 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c5oc5cc(-c7ccccc7)ccc56)c([2H])c([2H])c43)c([2H])c2[2H])c([2H])c1[2H] ; [2H]c1c([2H])c([2H])c(-c2c([2H])c([2H])c3c(oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)c([2H])c([2H])c([2H])c43)c2[2H])c([2H])c1[2H]]

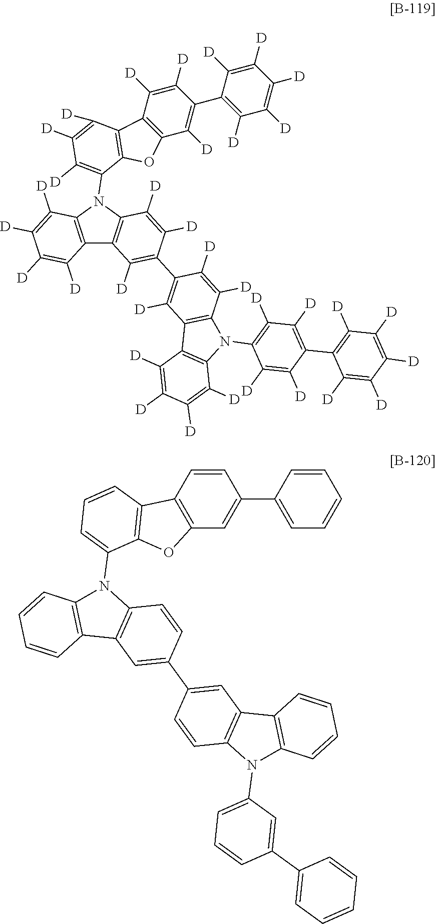 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c5oc5c([2H])c(-c7c([2H])c([2H])c([2H])c([2H])c7[2H])c([2H])c([2H])c56)c([2H])c([2H])c43)c([2H])c2[2H])c([2H])c1[2H] ; c1ccc(-c2cccc(-n3c4ccccc4c4cc(-c5ccc6c(c5)c5ccccc5n6-c5cccc6c5oc5cc(-c7ccccc7)ccc56)ccc43)c2)cc1]

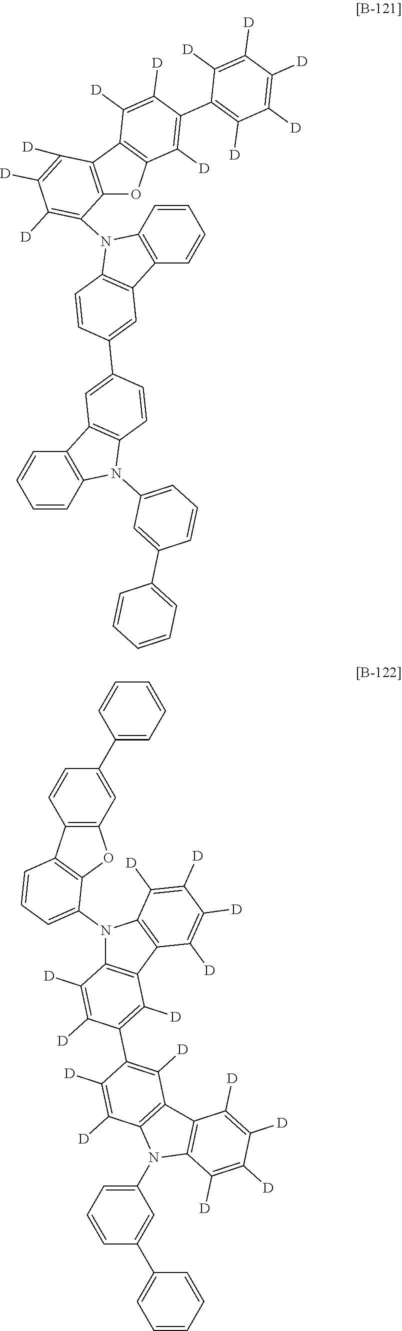

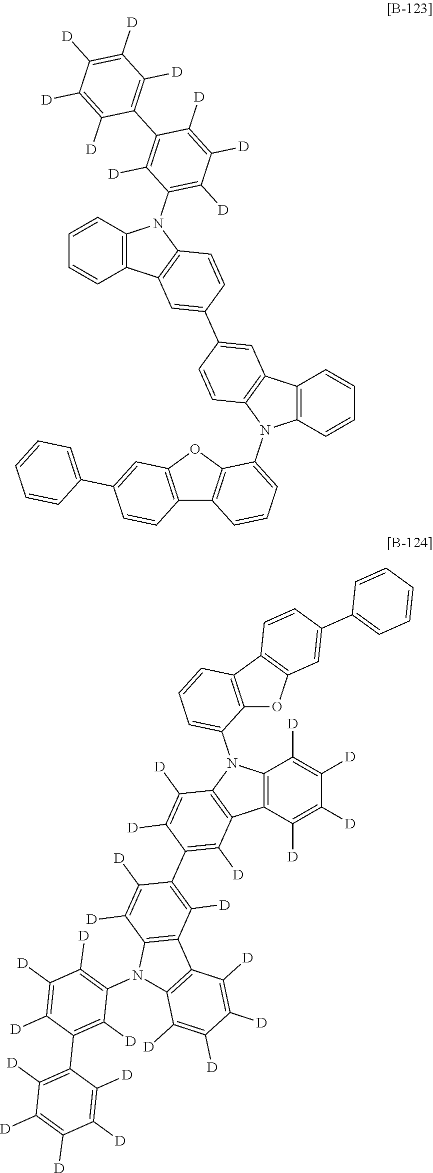 [SMILES: [2H]c1c([2H])c([2H])c(-c2c([2H])c([2H])c([2H])c(-n3c4ccccc4c4cc(-c5ccc6c(c5)c5ccccc5n6-c5cccc6c5oc5cc(-c7ccccc7)ccc56)ccc43)c2[2H])c([2H])c1[2H] ; [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c5oc5cc(-c7ccccc7)ccc56)c([2H])c([2H])c43)c2[2H])c([2H])c1[2H]]

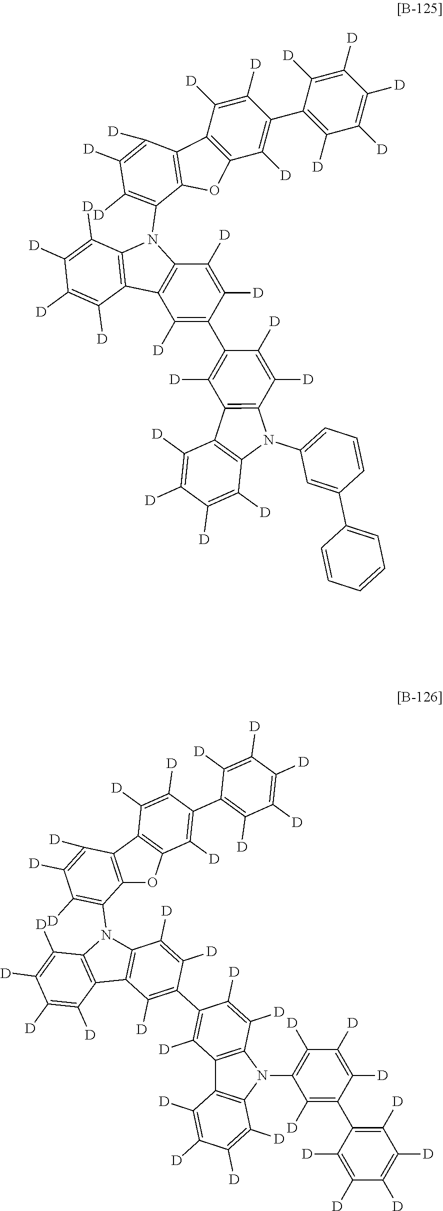

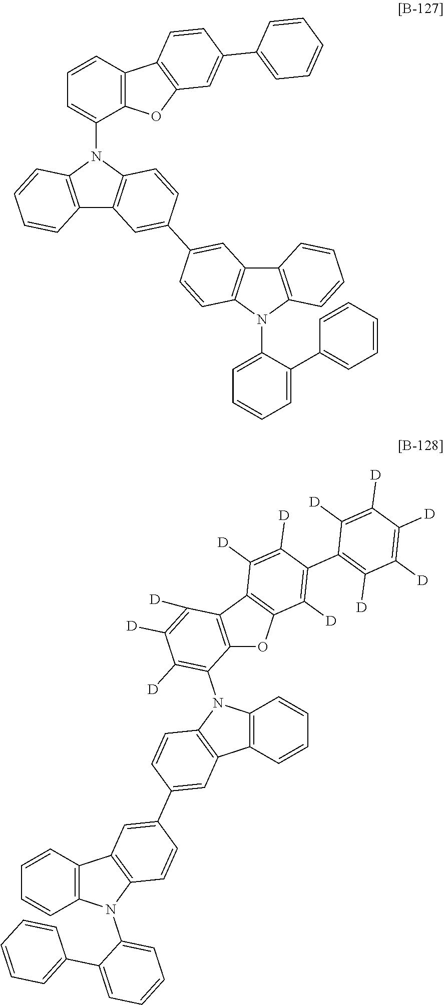 [SMILES: c1ccc(-c2ccc3c(c2)oc2c(-n4c5ccccc5c5cc(-c6ccc7c(c6)c6ccccc6n7-c6ccccc6-c6ccccc6)ccc54)cccc23)cc1 ; [2H]c1c([2H])c([2H])c(-c2c([2H])c([2H])c3c(oc4c(-n5c6ccccc6c6cc(-c7ccc8c(c7)c7ccccc7n8-c7ccccc7-c7ccccc7)ccc65)c([2H])c([2H])c([2H])c43)c2[2H])c([2H])c1[2H]]

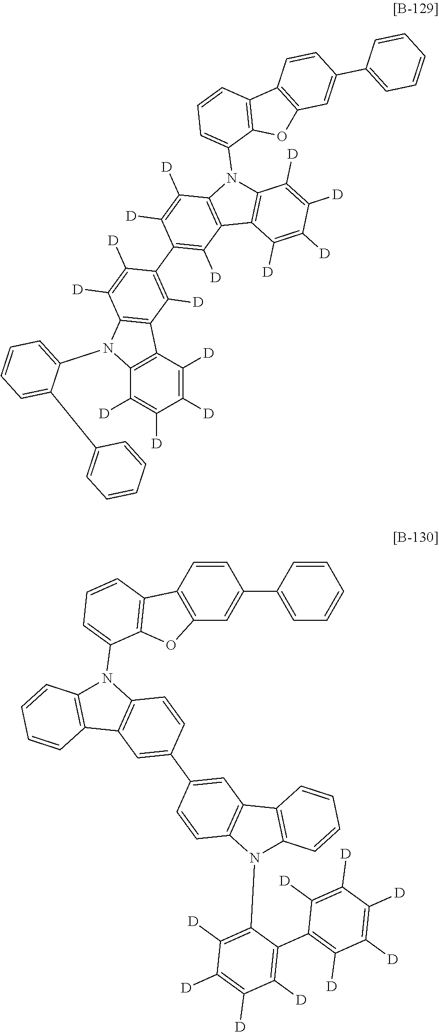 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3oc3cc(-c5ccccc5)ccc34)c([2H])c([2H])c1n2-c1ccccc1-c1ccccc1 ; [2H]c1c([2H])c([2H])c(-c2c([2H])c([2H])c([2H])c([2H])c2-n2c3ccccc3c3cc(-c4ccc5c(c4)c4ccccc4n5-c4cccc5c4oc4cc(-c6ccccc6)ccc45)ccc32)c([2H])c1[2H]]

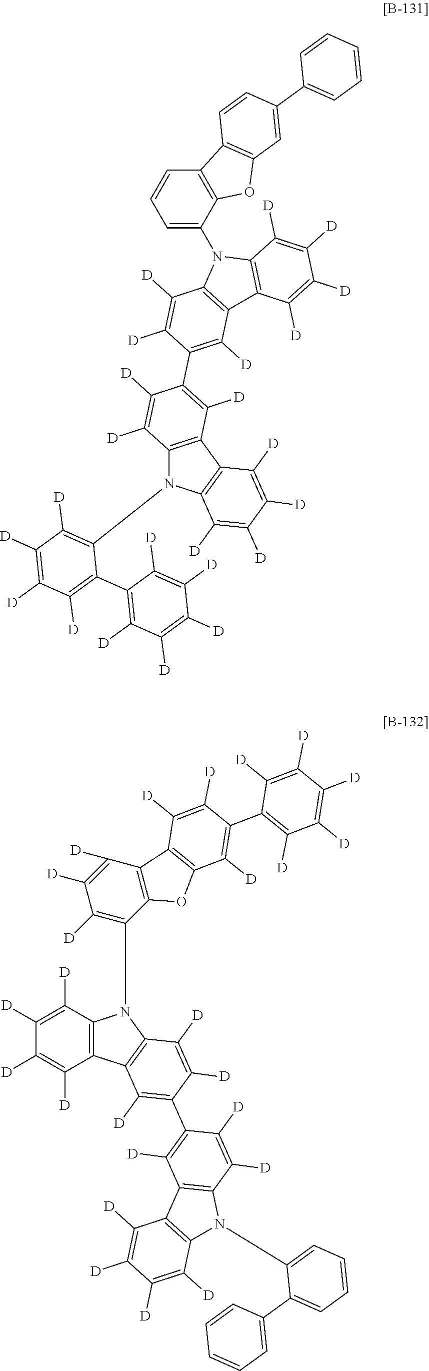 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5c4oc4cc(-c6ccccc6)ccc45)c([2H])c([2H])c32)c([2H])c1[2H] ; [2H]c1c([2H])c([2H])c(-c2c([2H])c([2H])c3c(oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7-c7ccccc7)c([2H])c([2H])c65)c([2H])c([2H])c([2H])c43)c2[2H])c([2H])c1[2H]]

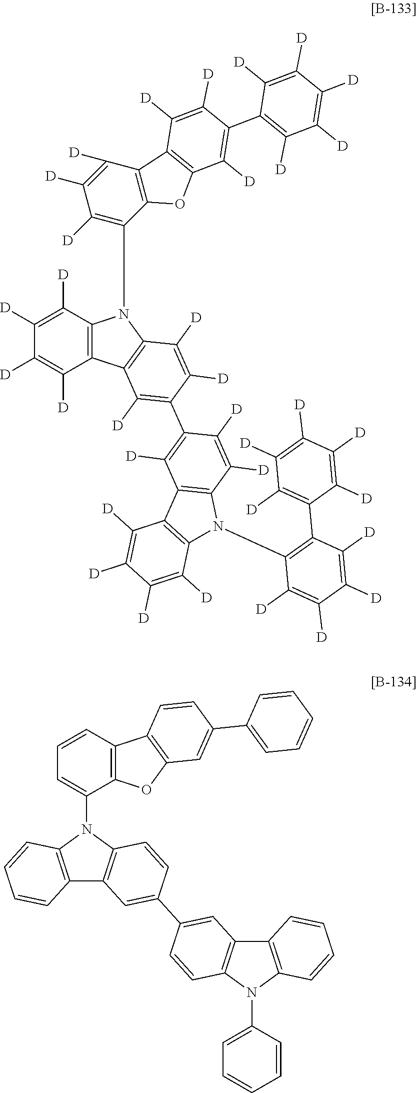 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c([2H])c5c4oc4c([2H])c(-c6c([2H])c([2H])c([2H])c([2H])c6[2H])c([2H])c([2H])c45)c([2H])c([2H])c32)c([2H])c1[2H] ; c1ccc(-c2ccc3c(c2)oc2c(-n4c5ccccc5c5cc(-c6ccc7c(c6)c6ccccc6n7-c6ccccc6)ccc54)cccc23)cc1]

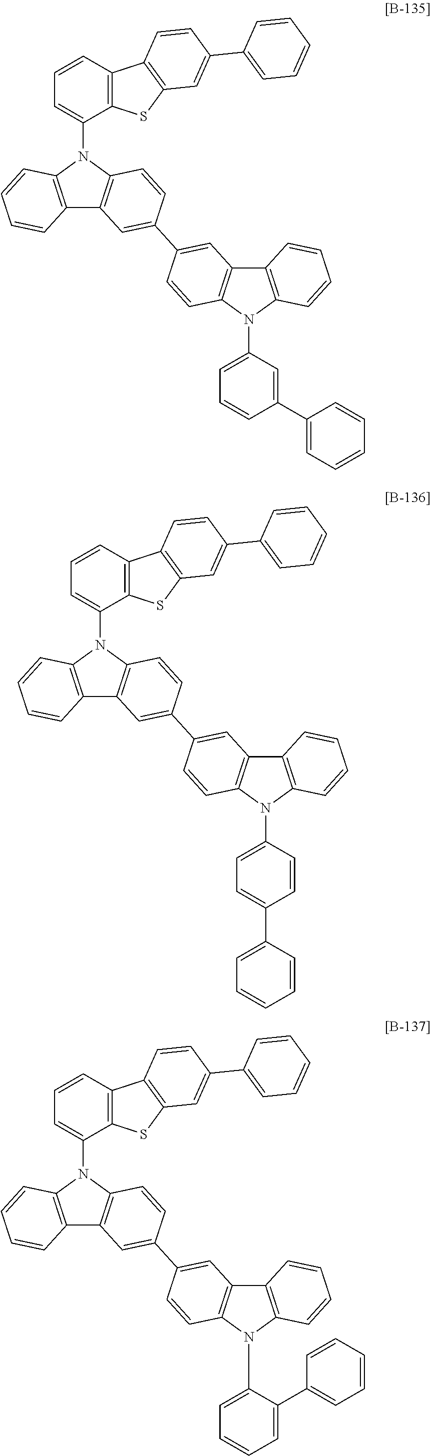

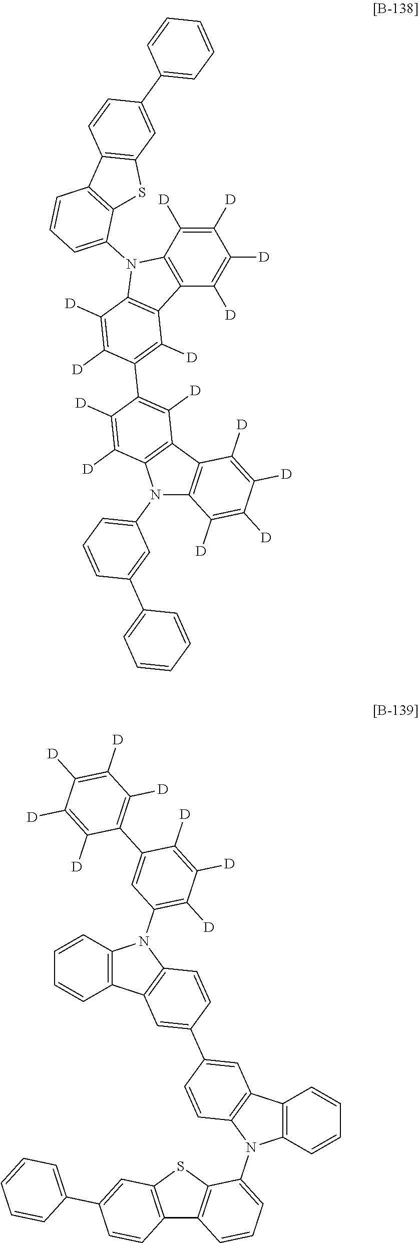 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3sc3cc(-c5ccccc5)ccc34)c([2H])c([2H])c1n2-c1cccc(-c2ccccc2)c1 ; [2H]c1c(-c2c([2H])c([2H])c([2H])c([2H])c2[2H])cc(-n2c3ccccc3c3cc(-c4ccc5c(c4)c4ccccc4n5-c4cccc5c4sc4cc(-c6ccccc6)ccc45)ccc32)c([2H])c1[2H]]

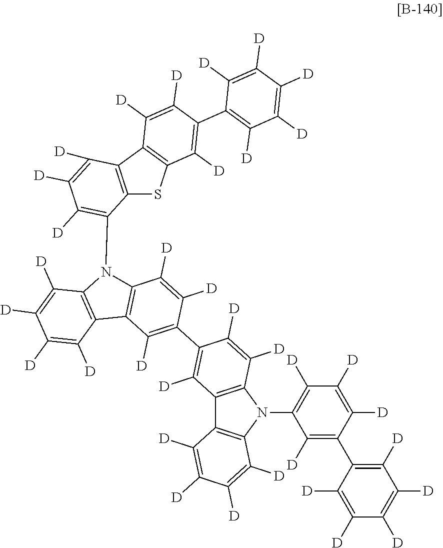 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c5sc5c([2H])c(-c7c([2H])c([2H])c([2H])c([2H])c7[2H])c([2H])c([2H])c56)c([2H])c([2H])c43)c2[2H])c([2H])c1[2H]]

The first compound and the second compound may be included in a weight ratio of, for example, 1:99 to 99:1. Within the above range, a desirable weight ratio may be adjusted using an electron transport capability of the first compound and a hole transport capability of the second compound to realize bipolar characteristics and thus to improve efficiency and life-span. Within the above range, for example, they may be included in a weight ratio of about 10:90 to 90:10, about 20:80 to 80:20, for example, about 20:80 to about 70:30, about 20:80 to about 60:40, and about 30:70 to about 60:40. As a specific example, they may be included in a weight ratio of 40:60, 50:50, or 60:40.

In addition to the aforementioned first compound and second compound, one or more compounds may be further included.

The aforementioned composition for the organic optoelectronic device may further include a dopant.

The dopant may be, for example, a phosphorescent dopant, for example, a red, green or blue phosphorescent dopant, and may be, for example, a red phosphorescent dopant.

The dopant is a material mixed with the composition for the organic optoelectronic device in a small amount to cause light emission, and may be generally a material such as a metal complex that emits light by multiple excitation into a triplet or more. The dopant may be, for example, an inorganic, organic, or organic-inorganic compound, and one or more types thereof may be used.

Examples of the dopant may be a phosphorescent dopant and examples of the phosphorescent dopant may be an organic metal compound including Ir, Pt, Os, Ti, Zr, Hf, Eu, Tb, Tm, Fe, Co, Ni, Ru, Rh, Pd, or a combination thereof. The phosphorescent dopant may be, for example, a compound represented by Chemical Formula Z, but is not limited thereto.

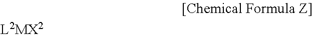

In Chemical Formula Z, M is a metal, and L² and X² are the same or different, and are a ligand to form a complex compound with M.

The M may be, for example, Ir, Pt, Os, Ti, Zr, Hf, Eu, Tb, Tm, Fe, Co, Ni, Ru, Rh, Pd, or a combination thereof and L² and X² may be, for example, a bidentate ligand.

Examples of ligands represented by L² and X² may be selected from the chemical formulas listed in Group A, but are not limited thereto.

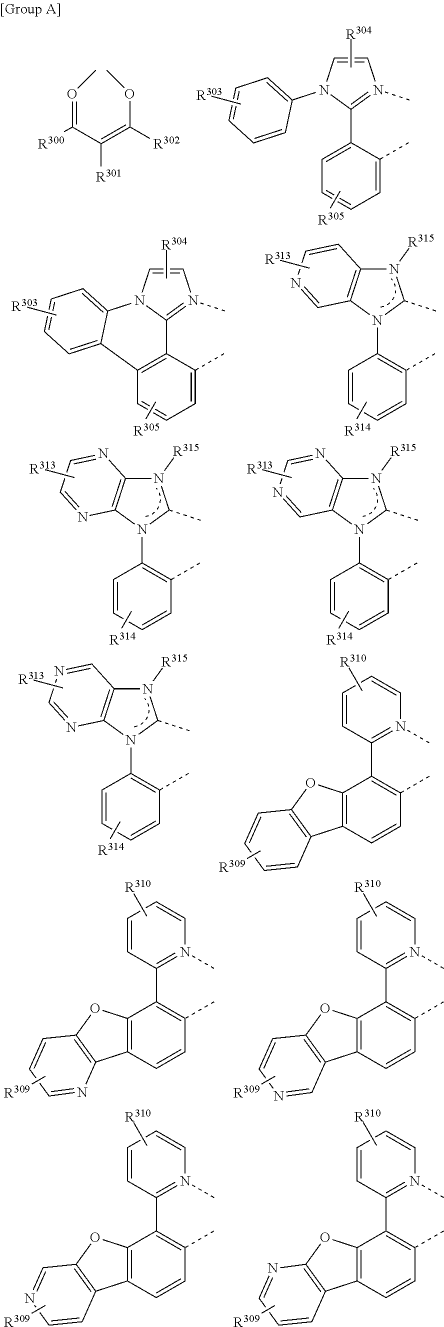

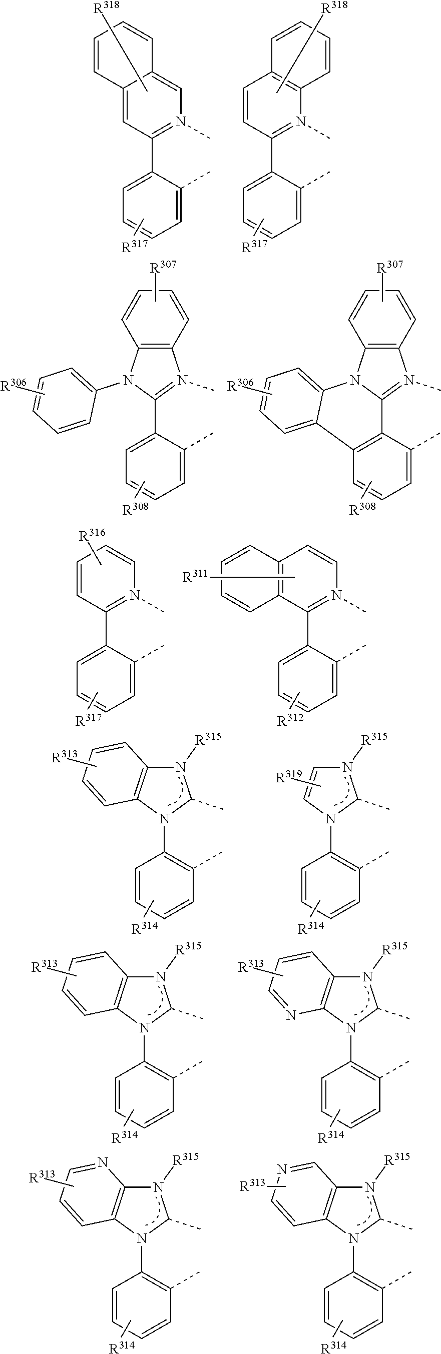

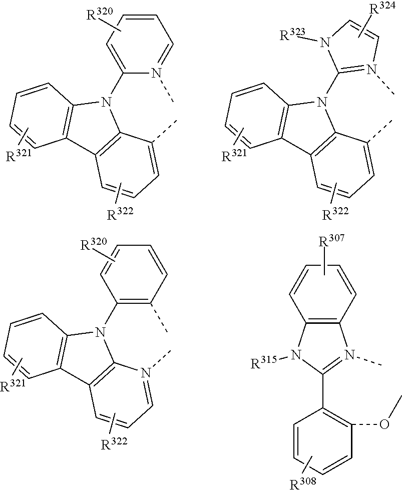

In Group A,
R³⁰⁰ to R³⁰² are each independently hydrogen, deuterium, a C1 to C30 alkyl group substituted or unsubstituted with halogen, or a C6 to C30 aryl group substituted or unsubstituted with C1 to C30 alkyl or a halogen, and
R³⁰³ to R³²⁴ are each independently hydrogen, deuterium, a halogen, a substituted or unsubstituted C1 to C30 alkyl group, a substituted or unsubstituted C1 to C30 alkoxy group, a substituted or unsubstituted C3 to C30 cycloalkyl group, a substituted or unsubstituted C2 to C30 alkenyl group, a substituted or unsubstituted C6 to C30 aryl group, a substituted or unsubstituted C1 to C30 heteroaryl group, a substituted or unsubstituted C1 to C30 amino group, a substituted or unsubstituted C6 to C30 arylamino group, SF₅, a trialkylsilyl group having a substituted or unsubstituted C1 to C30 alkyl group, a dialkylarylsilyl group having a substituted or unsubstituted C1 to C30 alkyl group and a C6 to C30 aryl group, or a triarylsilyl group having a substituted or unsubstituted C6 to C30 aryl group.

The dopant according to an embodiment may be an iridium complex, and may be, for example, represented by Chemical Formula IV-1 or Chemical Formula IV-2.

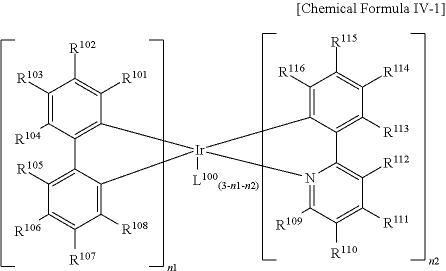

In Chemical Formula IV-1,
R¹⁰¹ to R¹¹⁶ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a substituted or unsubstituted C1 to C6 alkyl group, at least one of R¹⁰¹ to R¹¹⁶ is a functional group represented by Chemical Formula IV,
L¹⁰⁰ is a bidentate ligand of a monovalent anion, and is a ligand that coordinates to iridium through a lone pair of carbons or heteroatoms, and
n1 and n2 are each independently any one of integers of 0 to 3, and n1+n2 is any one of integers of 1 to 3,

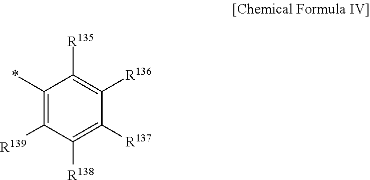

wherein, in Chemical Formula IV,
R¹³⁵ to R¹³⁹ are each independently hydrogen, deuterium, a substituted or unsubstituted C1 to C10 alkyl group, a substituted or unsubstituted C6 to C20 aryl group, or —SiR¹³²R¹³³R¹³⁴, and
* refers to a portion linked to a carbon atom.

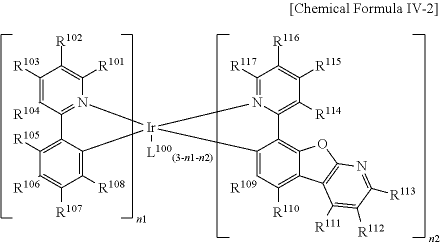

In Chemical Formula IV-2,
R¹⁰¹ to R¹¹⁷ are each independently hydrogen, deuterium, a substituted or unsubstituted C1 to C10 alkyl group, a substituted or unsubstituted C6 to C20 aryl group, or —SiR¹³³R¹³⁴R¹³⁵,
R¹³³ to R¹³⁵ are each independently a substituted or unsubstituted C1 to C6 alkyl group,
L¹⁰⁰ is a bidentate ligand of a monovalent anion, and is a ligand that coordinates to iridium through a lone pair of carbons or heteroatoms, and
n1 and n2 are each independently any one of integers of 0 to 3, and n1+n2 is any one of integers of 1 to 3.

The dopant according to another embodiment may be a platinum complex, and may be represented by, for example, Chemical Formula Z-1.

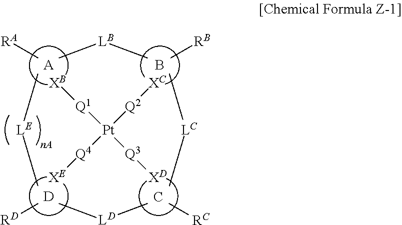

In Chemical Formula Z-1, rings A, B, C, and D are each independently a 5-membered or 6-membered carbocyclic or heterocyclic ring;
R^{A}, R^{B}, R^{C}, and R^{D} are each independently mono-, di-, tri-, or tetra-substitution, or unsubstitution;
L^{B}, L^{C}, and L^{D} are each independently a direct bond, BR, NR, PR, O, S, Se, C═O, S═O, SO₂, CRR′, SiRR′, GeRR′, or a combination thereof,
when nA is 1, L^{E} may be a direct bond, BR, NR, PR, O, S, Se, C═O, S═O, SO₂, CRR′, SiRR′, GeRR′, or a combination thereof, and when nA is 0, L^{E} does not exist;
R^{A}, R^{B}, R^{C}, R^{D}, R, and R′ are each independently hydrogen, deuterium, a halogen, alkyl group, a cycloalkyl group, a heteroalkyl group, an arylalkyl group, an alkoxy group, an aryloxy group, an amino group, a silyl group, an alkenyl group, a cycloalkenyl group, a heteroalkenyl group, an alkynyl group, an aryl group, a heteroaryl group, an acyl group, a carbonyl group, a carboxylic acid group, an ester group, a nitrile group, an isonitrile group, a sulfanyl group, a sulfinyl group, a sulfonyl group, a phosphino group, or a combination thereof, any adjacent R^{A}R^{B}, R^{C}, R^{D}, R, and R′ are optionally linked to each other to provide a ring; X^{B}, X^{C}, X^{D}, and X^{E} are each independently selected from carbon and nitrogen; and Q¹, Q², Q³, and Q⁴ each represent oxygen or a direct bond.

The dopant according to an embodiment may be a platinum complex, and may be represented by, for example, Chemical Formula V-1 or Chemical Formula V-2.

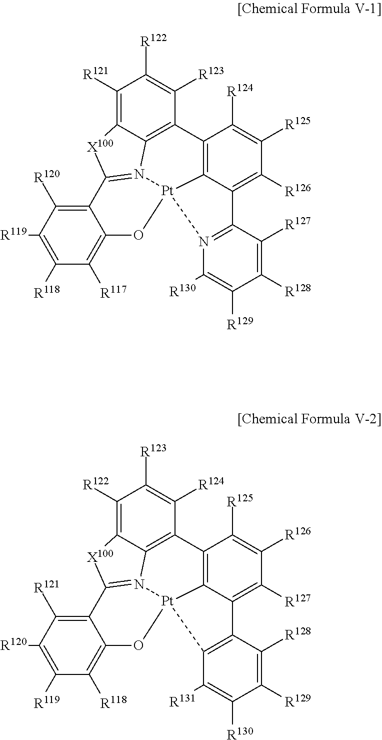

In Chemical Formula V-1 and Chemical Formula V-2,
X¹⁰⁰ is selected from O, S, and NR¹³¹,
R¹¹⁷ to R¹³¹ are each independently hydrogen, deuterium, a substituted or unsubstituted C1 to C10 alkyl group, a substituted or unsubstituted C6 to C20 aryl group, or —SiR¹³²R¹³³R¹³⁴,
R¹³² to R¹³⁴ are each independently a substituted or unsubstituted C1 to C6 alkyl group, and
at least one of R¹¹⁷ to R¹³¹ is —SiR¹³²R¹³³R¹³⁴ or a tert-butyl group.

Hereinafter, an organic optoelectronic device including the aforementioned composition for the organic optoelectronic device is described.

The organic optoelectronic device may be a suitable device to convert electrical energy into photoenergy and vice versa, e.g., an organic photoelectric device, an organic light emitting diode, an organic solar cell, or an organic photoconductor drum.

Herein, an organic light emitting diode as one example of an organic optoelectronic device is described referring to drawings.

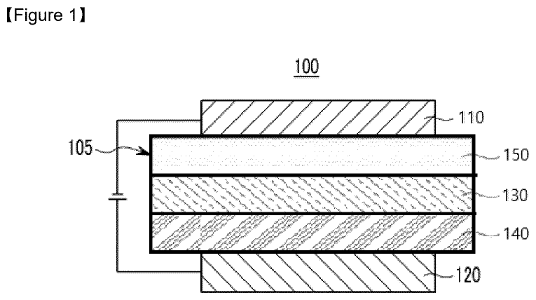

FIG. 1 is a cross-sectional view showing an organic light emitting diode according to an embodiment.

Referring to FIG. 1, an organic light emitting diode 100 according to an embodiment includes an anode 120 and a cathode 110 facing each other and an organic layer 105 disposed between the anode 120 and cathode 110.

The anode 120 may be made of a conductor having a large work function to help hole injection, and may be for example a metal, a metal oxide and/or a conductive polymer. The anode 120 may be, for example a metal such as nickel, platinum, vanadium, chromium, copper, zinc, gold, and the like or an alloy thereof; a metal oxide such as zinc oxide, indium oxide, indium tin oxide (ITO), indium zinc oxide (IZO), and the like; a combination of a metal and an oxide such as ZnO and Al or SnO₂ and Sb; a conductive polymer such as poly(3-methylthiophene), poly(3,4-(ethylene-1,2-dioxy)thiophene) (PEDOT), polypyrrole, and polyaniline, but is not limited thereto.

The cathode 110 may be made of a conductor having a small work function to help electron injection, and may be for example a metal, a metal oxide, and/or a conductive polymer. The cathode 110 may be for example a metal such as magnesium, calcium, sodium, potassium, titanium, indium, yttrium, lithium, gadolinium, aluminum silver, tin, lead, cesium, barium, and the like, or an alloy thereof; a multi-layer structure material such as LiF/Al, LiO₂/Al, LiF/Ca, and BaF₂/Ca, but is not limited thereto.

The organic layer 105 may include the aforementioned composition for the organic optoelectronic device.

The organic layer 105 may include a light emitting layer 130, and the light emitting layer 130 may include the aforementioned composition for the organic optoelectronic device.

The composition for the organic optoelectronic device further including a dopant may be, for example, a green light emitting composition.

The light emitting layer 130 may include, for example, the aforementioned composition for an organic optoelectronic device as a phosphorescent host.

The organic layer may further include a charge transport region in addition to the light emitting layer.

The charge transport region may be, for example, a hole transport region 140.

The hole transport region 140 can further increase hole injection and/or hole mobility and block electrons between the anode 120 and the light emitting layer 130.

Specifically, the hole transport region 140 may include a hole transport layer between the anode 120 and the light emitting layer 130, and a hole transport auxiliary layer between the light emitting layer 130 and the hole transport layer, and at least one of the compounds listed in Group B may be included in at least one layer of the hole transport layer and the hole transport auxiliary layer.

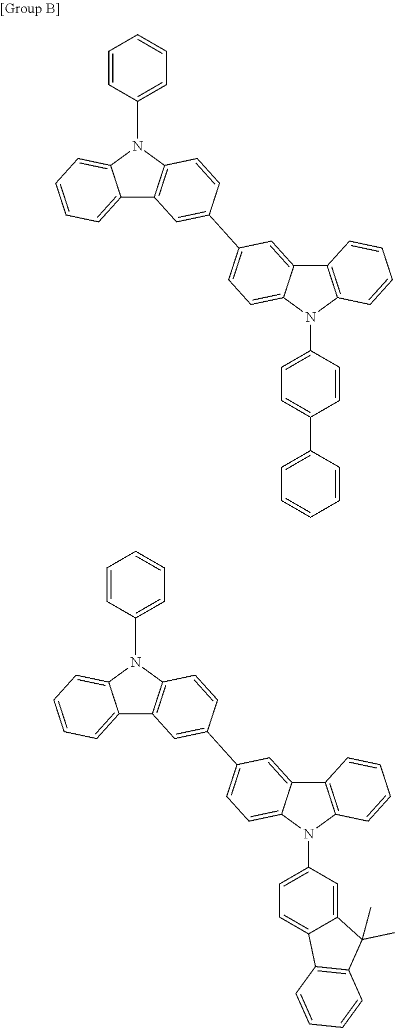 [SMILES: c1ccc(-c2ccc(-n3c4ccccc4c4cc(-c5ccc6c(c5)c5ccccc5n6-c5ccccc5)ccc43)cc2)cc1 ; CC1(C)c2ccccc2-c2ccc(-n3c4ccccc4c4cc(-c5ccc6c(c5)c5ccccc5n6-c5ccccc5)ccc43)cc21]

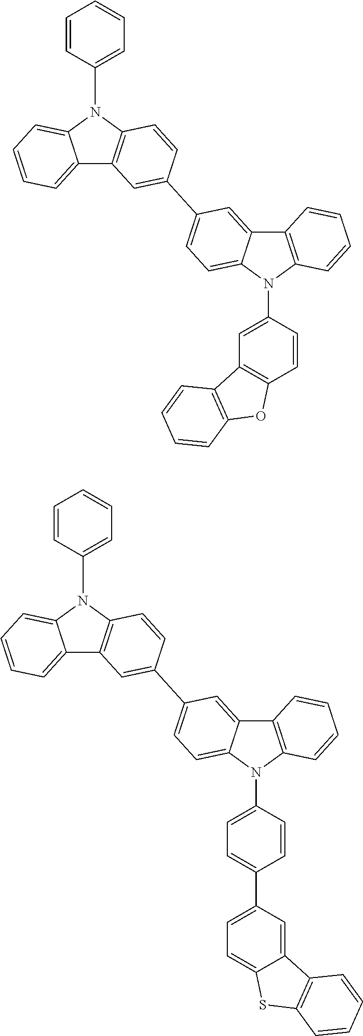 [SMILES: c1ccc(-n2c3ccccc3c3cc(-c4ccc5c(c4)c4ccccc4n5-c4ccc5oc6ccccc6c5c4)ccc32)cc1 ; c1ccc(-n2c3ccccc3c3cc(-c4ccc5c(c4)c4ccccc4n5-c4ccc(-c5ccc6sc7ccccc7c6c5)cc4)ccc32)cc1]

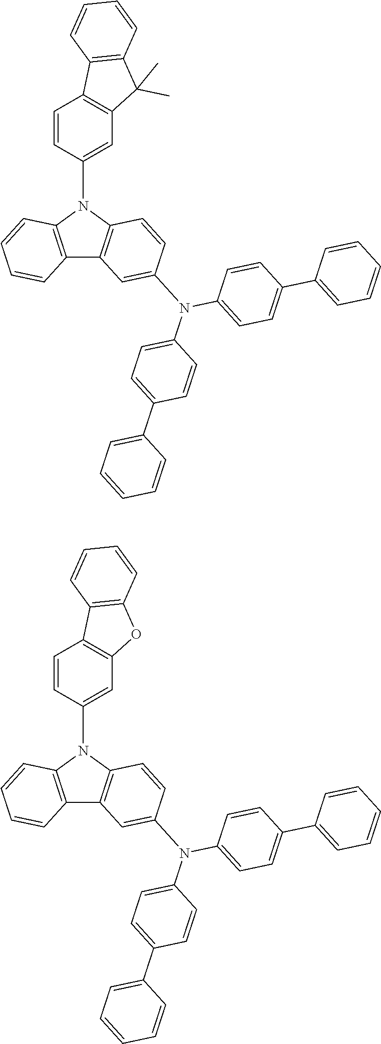 [SMILES: CC1(C)c2ccccc2-c2ccc(-n3c4ccccc4c4cc(N(c5ccc(-c6ccccc6)cc5)c5ccc(-c6ccccc6)cc5)ccc43)cc21 ; c1ccc(-c2ccc(N(c3ccc(-c4ccccc4)cc3)c3ccc4c(c3)c3ccccc3n4-c3ccc4c(c3)oc3ccccc34)cc2)cc1]

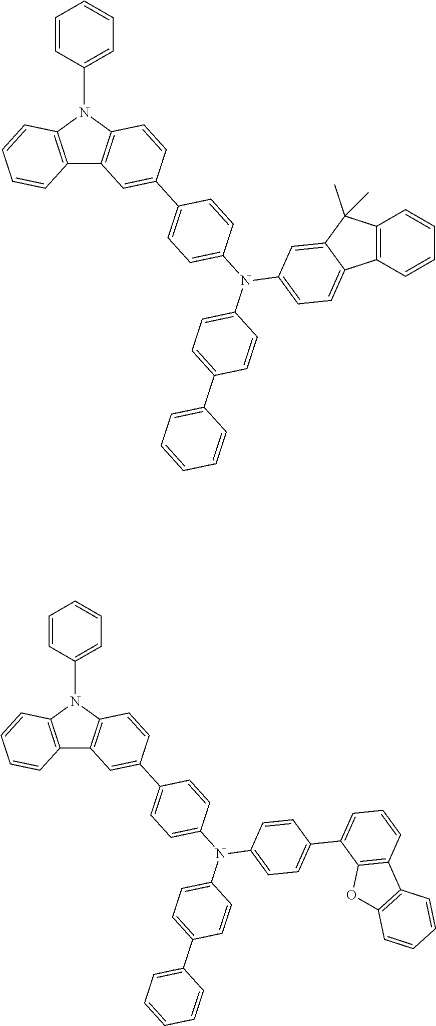 [SMILES: CC1(C)c2ccccc2-c2ccc(N(c3ccc(-c4ccccc4)cc3)c3ccc(-c4ccc5c(c4)c4ccccc4n5-c4ccccc4)cc3)cc21 ; c1ccc(-c2ccc(N(c3ccc(-c4ccc5c(c4)c4ccccc4n5-c4ccccc4)cc3)c3ccc(-c4cccc5c4oc4ccccc45)cc3)cc2)cc1]

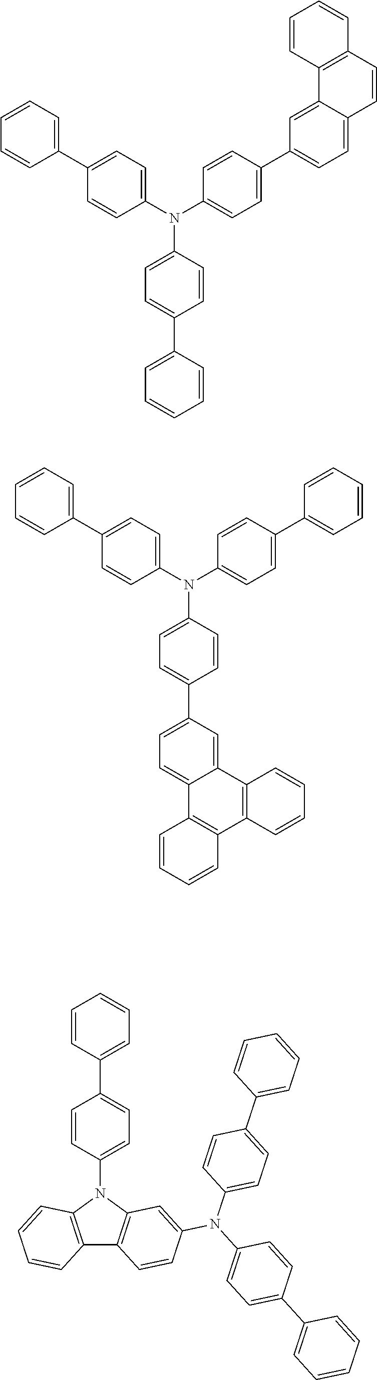 [SMILES: c1ccc(-c2ccc(N(c3ccc(-c4ccccc4)cc3)c3ccc(-c4ccc5ccc6ccccc6c5c4)cc3)cc2)cc1 ; c1ccc(-c2ccc(N(c3ccc(-c4ccccc4)cc3)c3ccc(-c4ccc5c6ccccc6c6ccccc6c5c4)cc3)cc2)cc1 ; c1ccc(-c2ccc(N(c3ccc(-c4ccccc4)cc3)c3ccc4c5ccccc5n(-c5ccc(-c6ccccc6)cc5)c4c3)cc2)cc1]

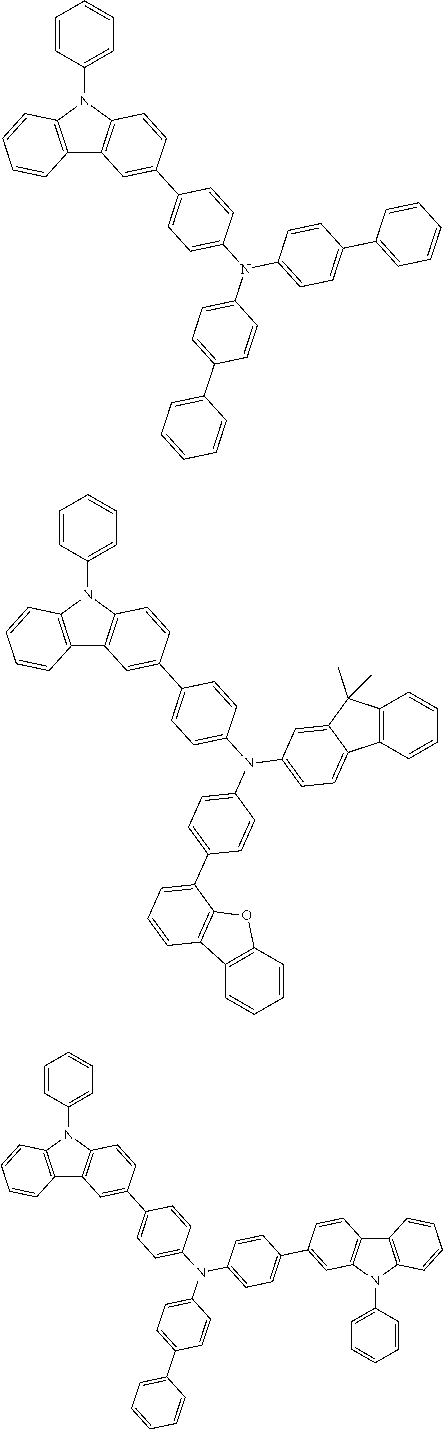 [SMILES: c1ccc(-c2ccc(N(c3ccc(-c4ccccc4)cc3)c3ccc(-c4ccc5c(c4)c4ccccc4n5-c4ccccc4)cc3)cc2)cc1 ; CC1(C)c2ccccc2-c2ccc(N(c3ccc(-c4ccc5c(c4)c4ccccc4n5-c4ccccc4)cc3)c3ccc(-c4cccc5c4oc4ccccc45)cc3)cc21 ; c1ccc(-c2ccc(N(c3ccc(-c4ccc5c(c4)c4ccccc4n5-c4ccccc4)cc3)c3ccc(-c4ccc5c6ccccc6n(-c6ccccc6)c5c4)cc3)cc2)cc1]

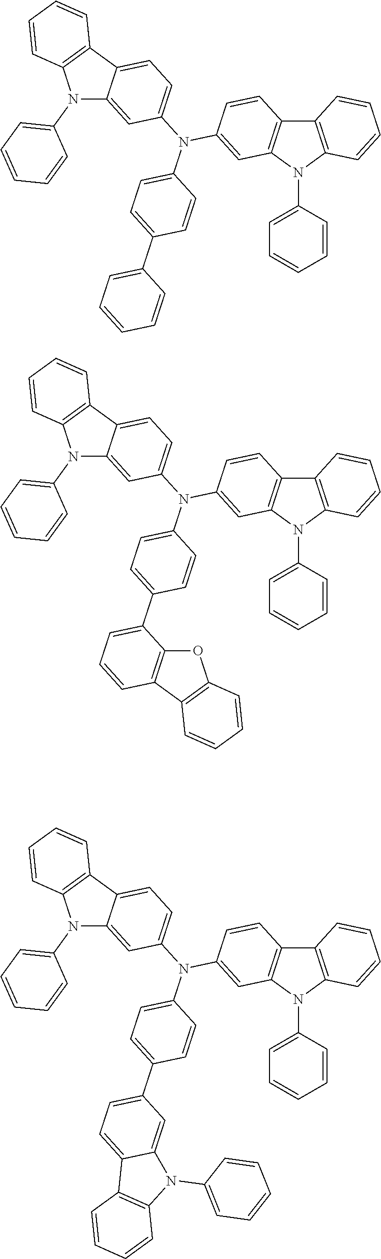 [SMILES: c1ccc(-c2ccc(N(c3ccc4c5ccccc5n(-c5ccccc5)c4c3)c3ccc4c5ccccc5n(-c5ccccc5)c4c3)cc2)cc1 ; c1ccc(-n2c3ccccc3c3ccc(N(c4ccc(-c5cccc6c5oc5ccccc56)cc4)c4ccc5c6ccccc6n(-c6ccccc6)c5c4)cc32)cc1 ; c1ccc(-n2c3ccccc3c3ccc(-c4ccc(N(c5ccc6c7ccccc7n(-c7ccccc7)c6c5)c5ccc6c7ccccc7n(-c7ccccc7)c6c5)cc4)cc32)cc1]

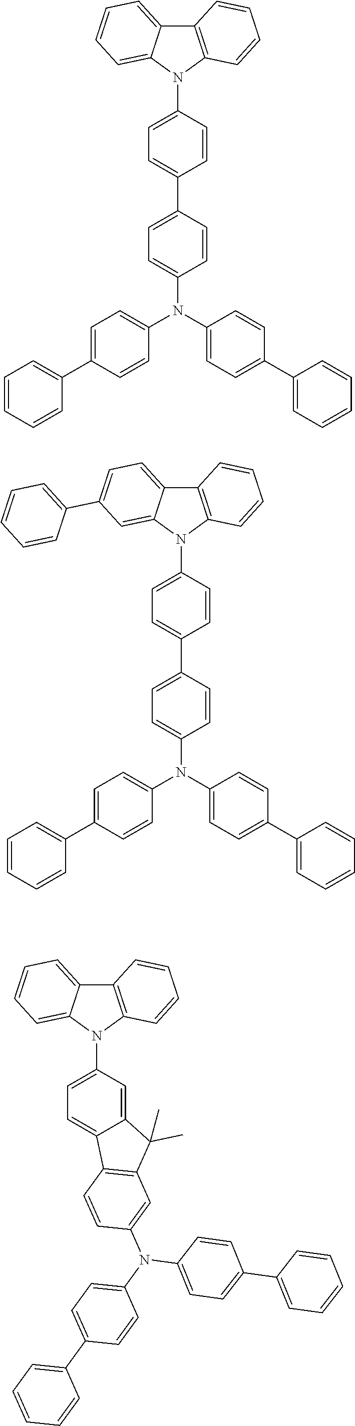 [SMILES: c1ccc(-c2ccc(N(c3ccc(-c4ccccc4)cc3)c3ccc(-c4ccc(-n5c6ccccc6c6ccccc65)cc4)cc3)cc2)cc1 ; c1ccc(-c2ccc(N(c3ccc(-c4ccccc4)cc3)c3ccc(-c4ccc(-n5c6ccccc6c6ccc(-c7ccccc7)cc65)cc4)cc3)cc2)cc1 ; CC1(C)c2cc(N(c3ccc(-c4ccccc4)cc3)c3ccc(-c4ccccc4)cc3)ccc2-c2ccc(-n3c4ccccc4c4ccccc43)cc21]

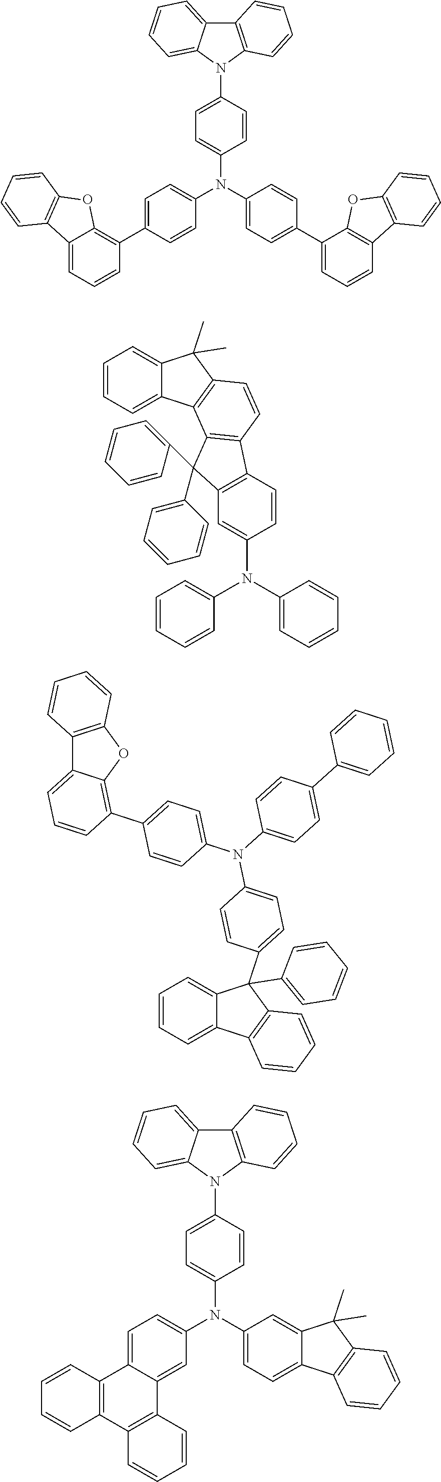

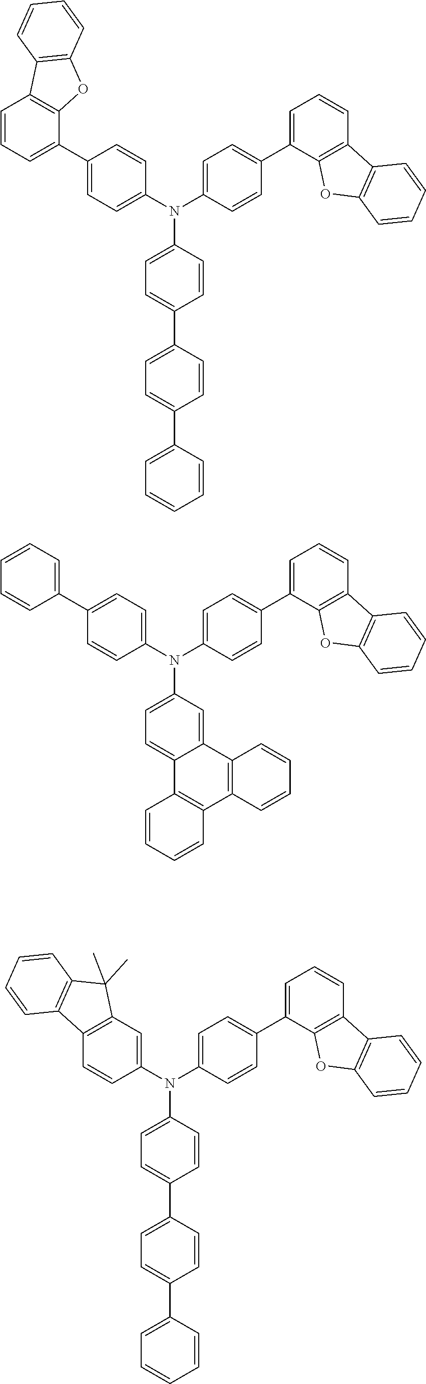 [SMILES: c1ccc(-c2ccc(-c3ccc(N(c4ccc(-c5cccc6c5oc5ccccc56)cc4)c4ccc(-c5cccc6c5oc5ccccc56)cc4)cc3)cc2)cc1 ; c1ccc(-c2ccc(N(c3ccc(-c4cccc5c4oc4ccccc45)cc3)c3ccc4c5ccccc5c5ccccc5c4c3)cc2)cc1 ; CC1(C)c2ccccc2-c2ccc(N(c3ccc(-c4ccc(-c5ccccc5)cc4)cc3)c3ccc(-c4cccc5c4oc4ccccc45)cc3)cc21]

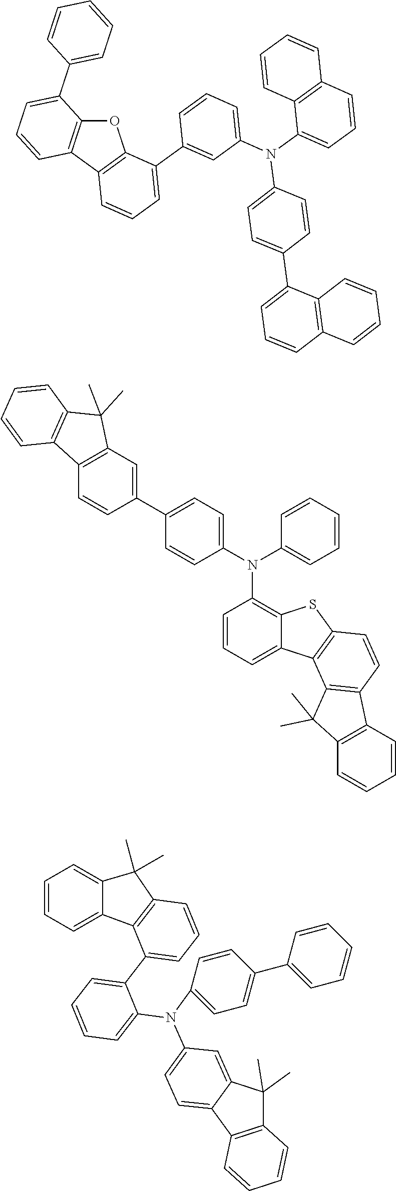 [SMILES: c1ccc(-c2cccc3c2oc2c(-c4cccc(N(c5ccc(-c6cccc7ccccc67)cc5)c5cccc6ccccc56)c4)cccc23)cc1 ; CC1(C)c2ccccc2-c2ccc(-c3ccc(N(c4ccccc4)c4cccc5c4sc4ccc6c(c45)C(C)(C)c4ccccc4-6)cc3)cc21 ; CC1(C)c2ccccc2-c2ccc(N(c3ccc(-c4ccccc4)cc3)c3ccccc3-c3cccc4c3-c3ccccc3C4(C)C)cc21]

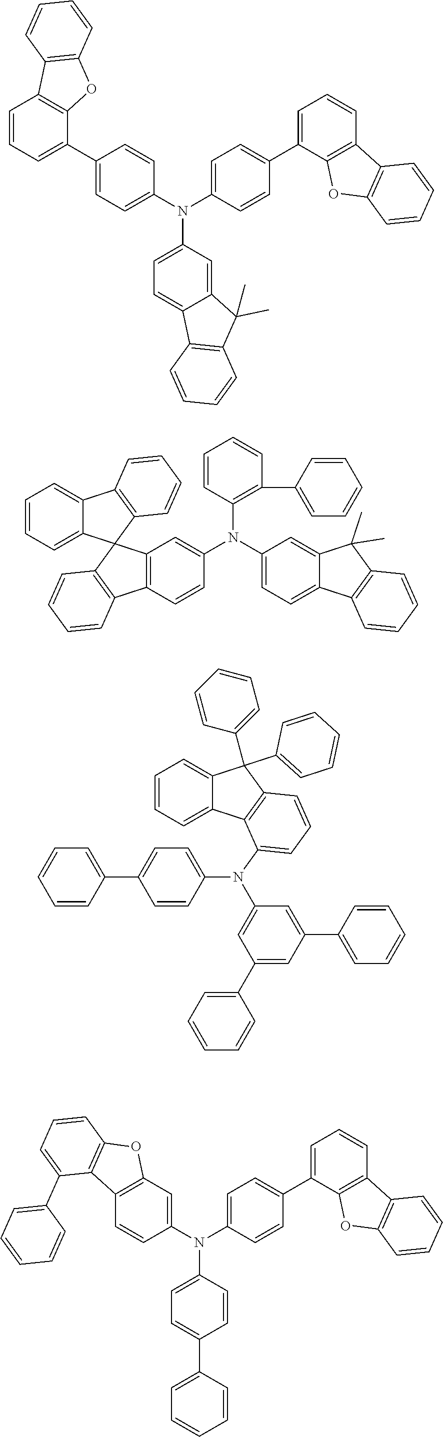

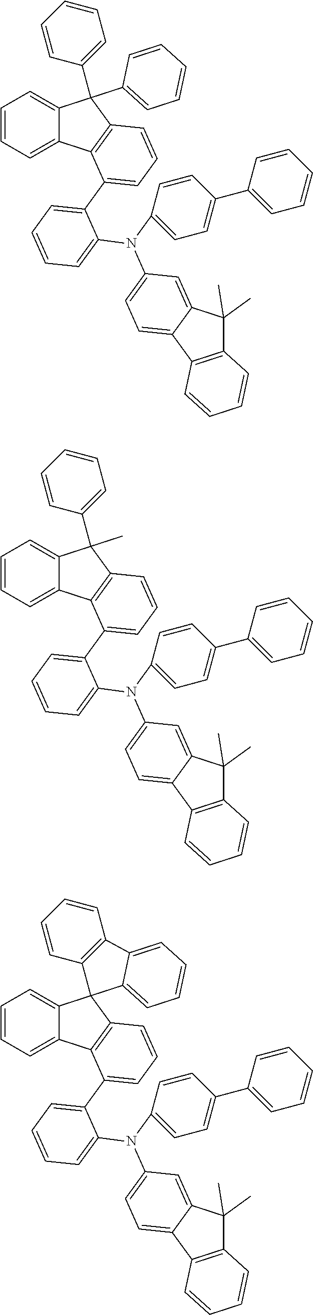 [SMILES: CC1(C)c2ccccc2-c2ccc(N(c3ccc(-c4ccccc4)cc3)c3ccccc3-c3cccc4c3-c3ccccc3C4(c3ccccc3)c3ccccc3)cc21 ; CC1(C)c2ccccc2-c2ccc(N(c3ccc(-c4ccccc4)cc3)c3ccccc3-c3cccc4c3-c3ccccc3C4(C)c3ccccc3)cc21 ; CC1(C)c2ccccc2-c2ccc(N(c3ccc(-c4ccccc4)cc3)c3ccccc3-c3cccc4c3-c3ccccc3C43c4ccccc4-c4ccccc43)cc21]

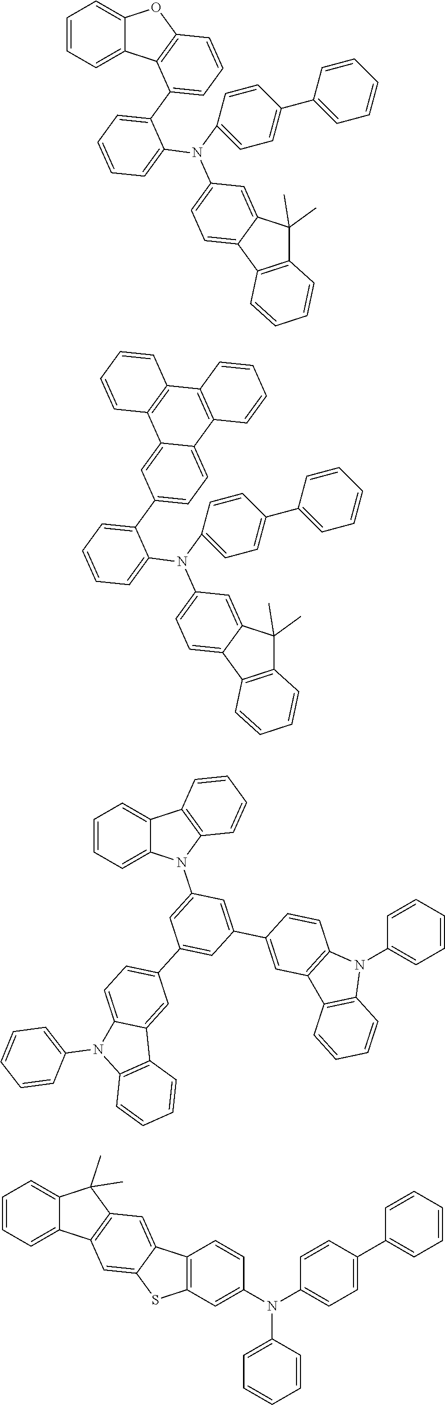

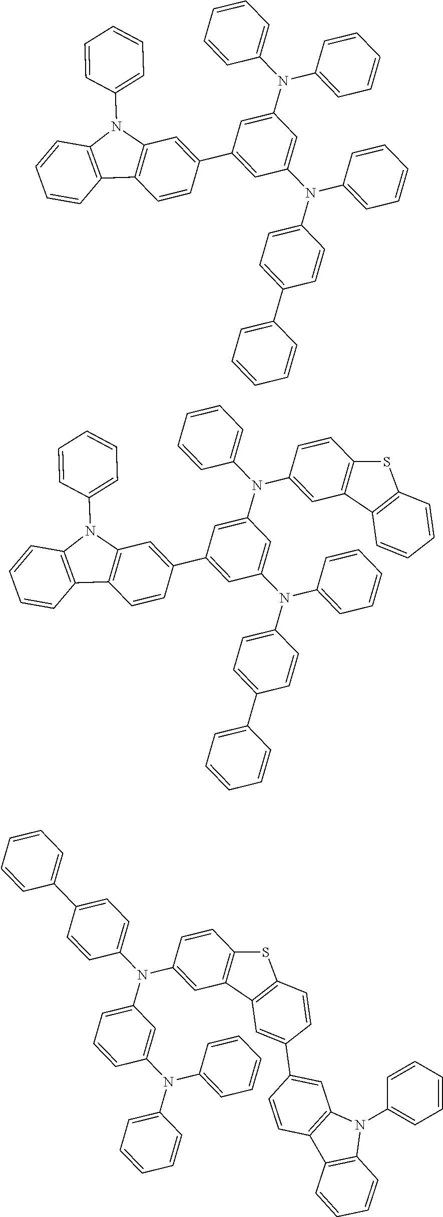

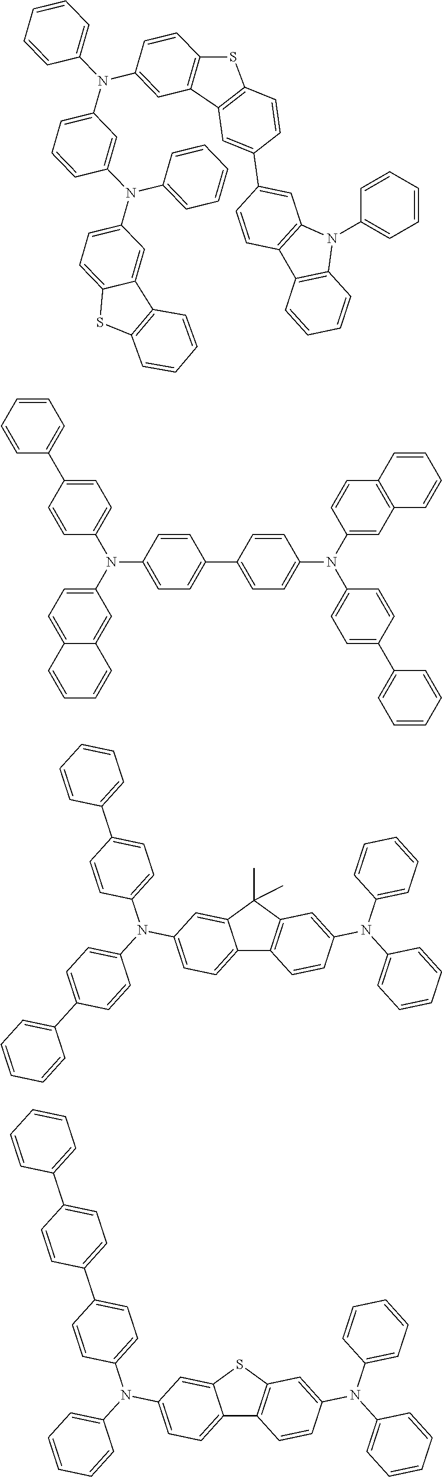

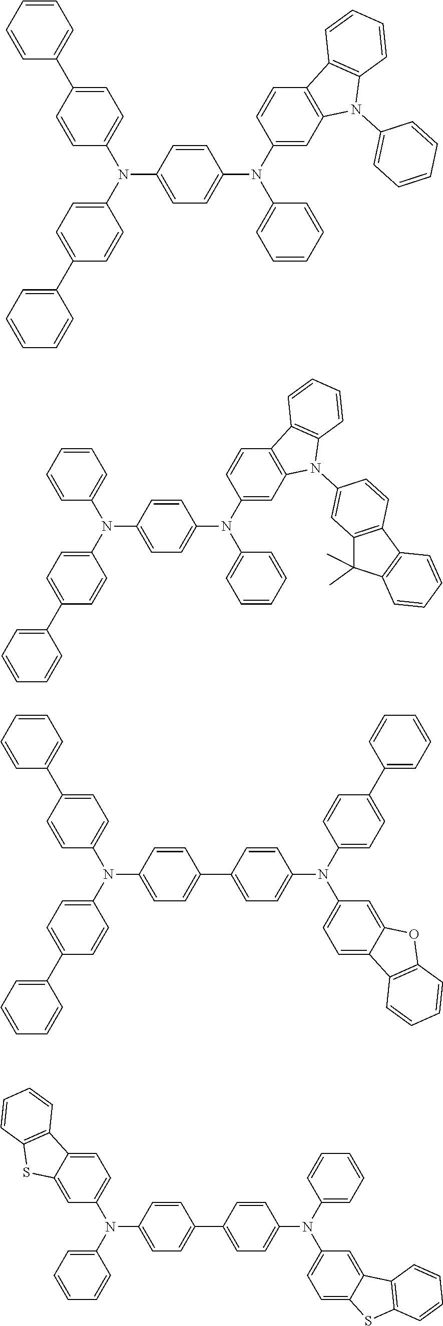

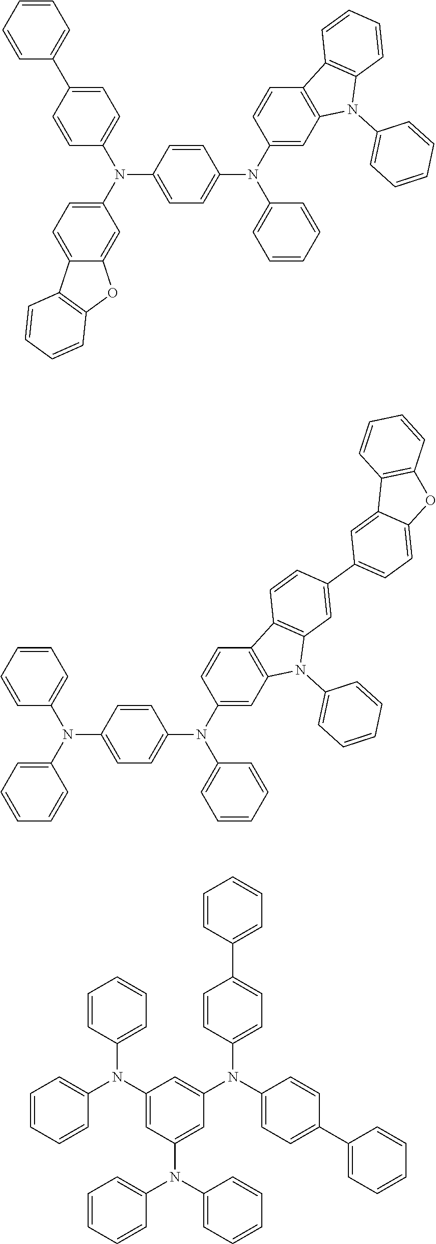 [SMILES: c1ccc(-c2ccc(N(c3ccc(N(c4ccccc4)c4ccc5c6ccccc6n(-c6ccccc6)c5c4)cc3)c3ccc4c(c3)oc3ccccc34)cc2)cc1 ; c1ccc(N(c2ccccc2)c2ccc(N(c3ccccc3)c3ccc4c5ccc(-c6ccc7oc8ccccc8c7c6)cc5n(-c5ccccc5)c4c3)cc2)cc1 ; c1ccc(-c2ccc(N(c3ccc(-c4ccccc4)cc3)c3cc(N(c4ccccc4)c4ccccc4)cc(N(c4ccccc4)c4ccccc4)c3)cc2)cc1]

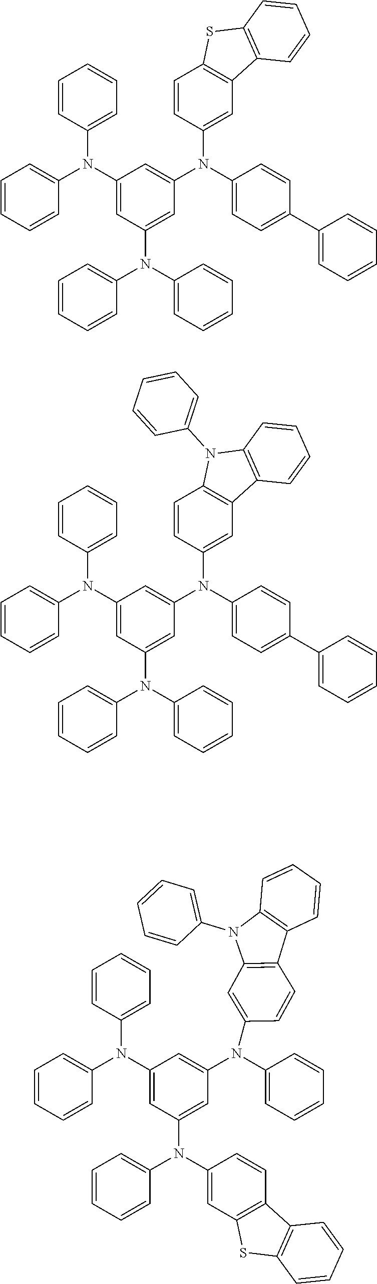 [SMILES: c1ccc(-c2ccc(N(c3cc(N(c4ccccc4)c4ccccc4)cc(N(c4ccccc4)c4ccccc4)c3)c3ccc4sc5ccccc5c4c3)cc2)cc1 ; c1ccc(-c2ccc(N(c3cc(N(c4ccccc4)c4ccccc4)cc(N(c4ccccc4)c4ccccc4)c3)c3ccc4c(c3)c3ccccc3n4-c3ccccc3)cc2)cc1 ; c1ccc(N(c2ccccc2)c2cc(N(c3ccccc3)c3ccc4c(c3)sc3ccccc34)cc(N(c3ccccc3)c3ccc4c5ccccc5n(-c5ccccc5)c4c3)c2)cc1]

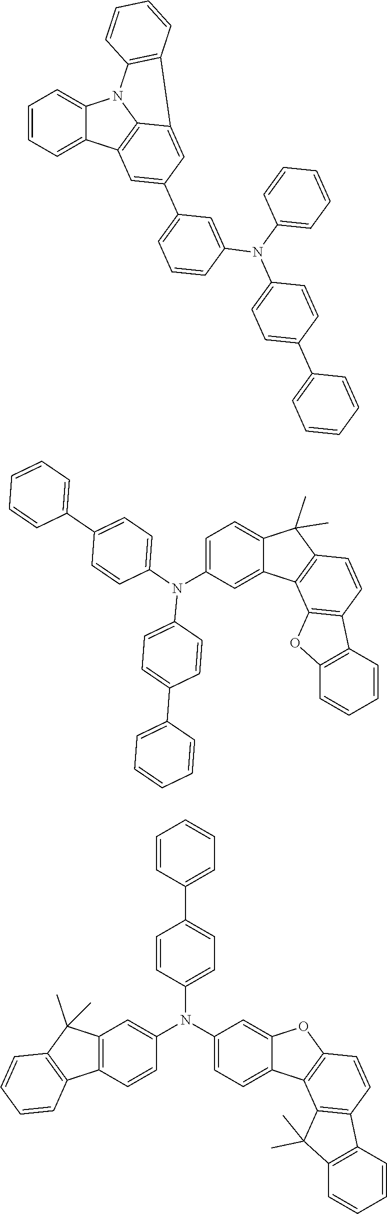

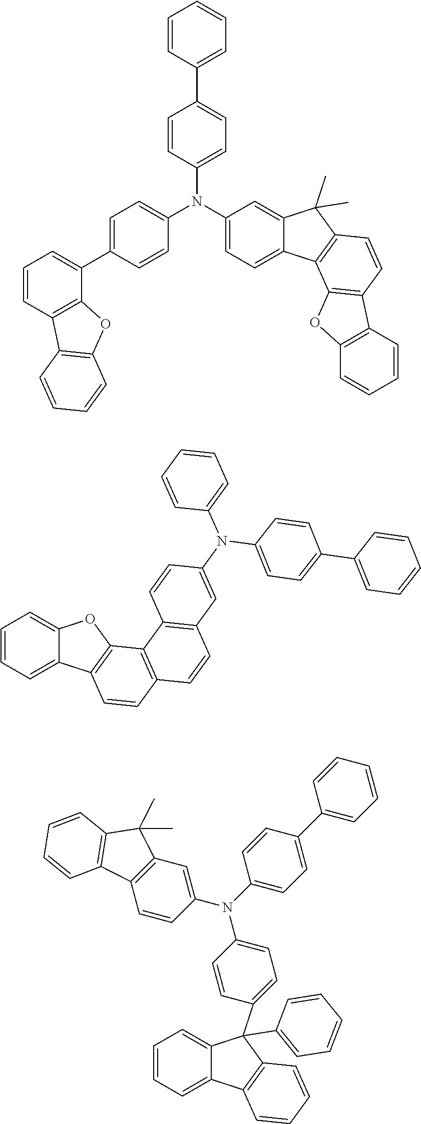 [SMILES: CC1(C)c2cc(N(c3ccc(-c4ccccc4)cc3)c3ccc(-c4cccc5c4oc4ccccc45)cc3)ccc2-c2c1ccc1c2oc2ccccc21 ; c1ccc(-c2ccc(N(c3ccccc3)c3ccc4c(ccc5ccc6c7ccccc7oc6c54)c3)cc2)cc1 ; CC1(C)c2ccccc2-c2ccc(N(c3ccc(-c4ccccc4)cc3)c3ccc(C4(c5ccccc5)c5ccccc5-c5ccccc54)cc3)cc21]

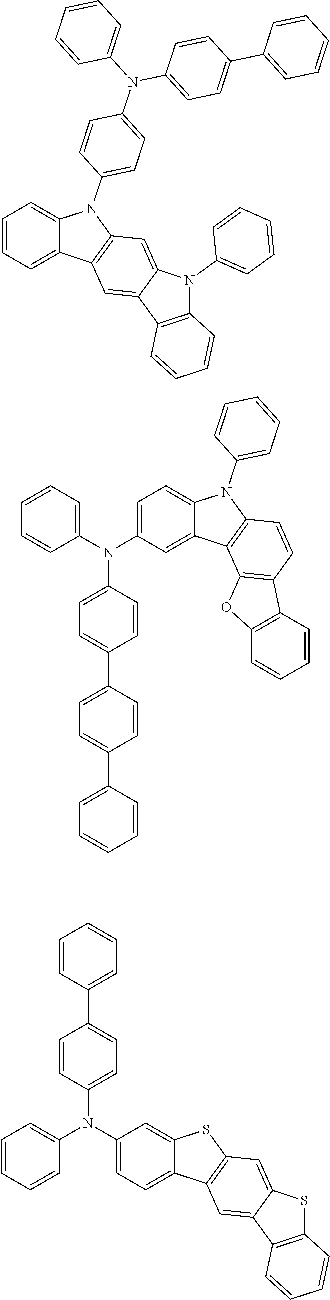

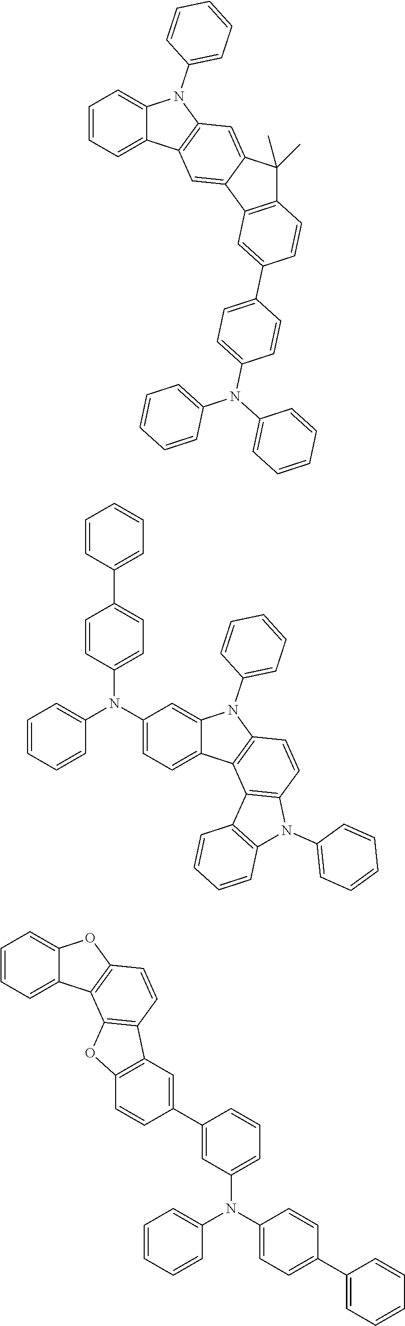 [SMILES: CC1(C)c2ccc(-c3ccc(N(c4ccccc4)c4ccccc4)cc3)cc2-c2cc3c4ccccc4n(-c4ccccc4)c3cc21 ; c1ccc(-c2ccc(N(c3ccccc3)c3ccc4c5c6c7ccccc7n(-c7ccccc7)c6ccc5n(-c5ccccc5)c4c3)cc2)cc1 ; c1ccc(-c2ccc(N(c3ccccc3)c3cccc(-c4ccc5oc6c(ccc7oc8ccccc8c76)c5c4)c3)cc2)cc1]

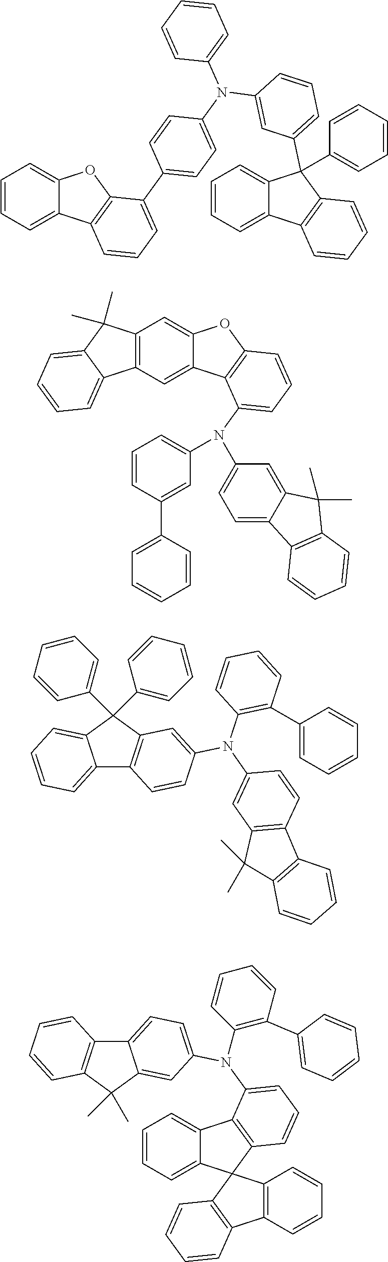

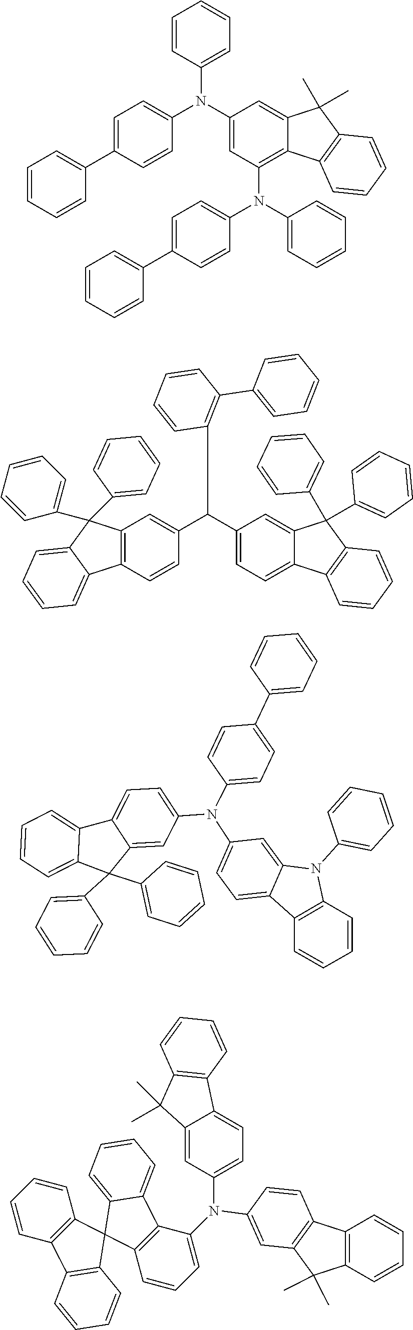

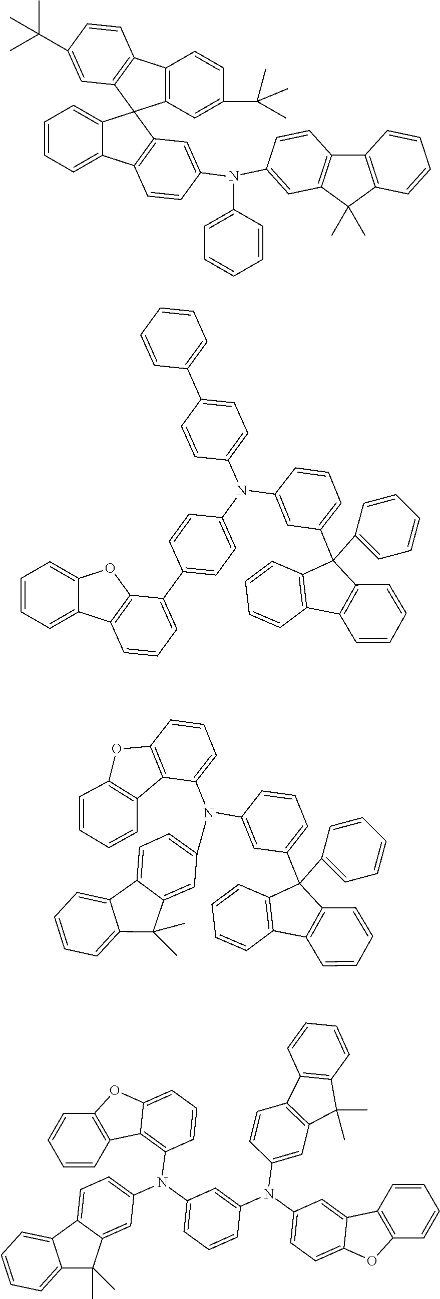 [SMILES: CC(C)(C)c1ccc2c(c1)C1(c3ccccc3-c3ccc(N(c4ccccc4)c4ccc5c(c4)C(C)(C)c4ccccc4-5)cc31)c1cc(C(C)(C)C)ccc1-2 ; c1ccc(-c2ccc(N(c3ccc(-c4cccc5c4oc4ccccc45)cc3)c3cccc(C4(c5ccccc5)c5ccccc5-c5ccccc54)c3)cc2)cc1 ; CC1(C)c2ccccc2-c2ccc(N(c3cccc(C4(c5ccccc5)c5ccccc5-c5ccccc54)c3)c3cccc4oc5ccccc5c34)cc21 ; CC1(C)c2ccccc2-c2ccc(N(c3cccc(N(c4ccc5c(c4)C(C)(C)c4ccccc4-5)c4cccc5oc6ccccc6c45)c3)c3ccc4oc5ccccc5c4c3)cc21]

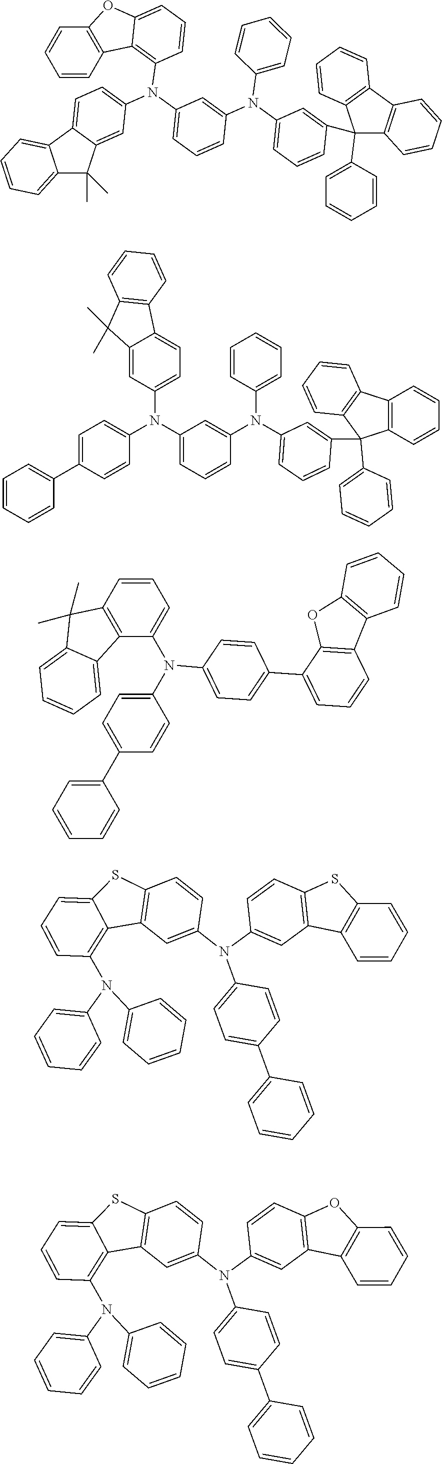

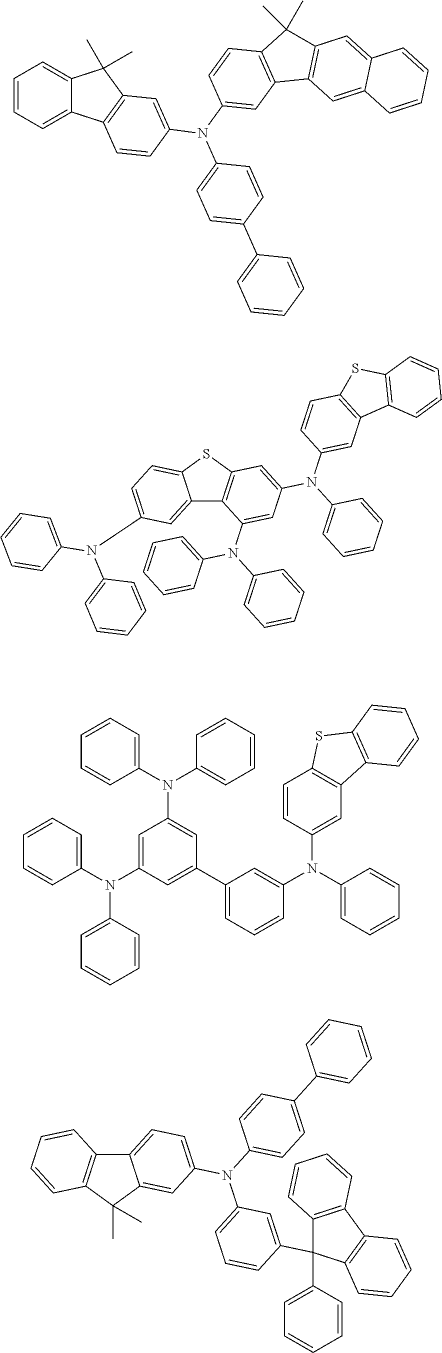 [SMILES: CC1(C)c2ccccc2-c2ccc(N(c3ccc(-c4ccccc4)cc3)c3ccc4c(c3)-c3cc5ccccc5cc3C4(C)C)cc21 ; c1ccc(N(c2cc(N(c3ccccc3)c3ccccc3)c3c(c2)sc2ccc(N(c4ccccc4)c4ccccc4)cc23)c2ccc3sc4ccccc4c3c2)cc1 ; c1ccc(N(c2ccccc2)c2cc(-c3cccc(N(c4ccccc4)c4ccc5sc6ccccc6c5c4)c3)cc(N(c3ccccc3)c3ccccc3)c2)cc1 ; CC1(C)c2ccccc2-c2ccc(N(c3ccc(-c4ccccc4)cc3)c3cccc(C4(c5ccccc5)c5ccccc5-c5ccccc54)c3)cc21]

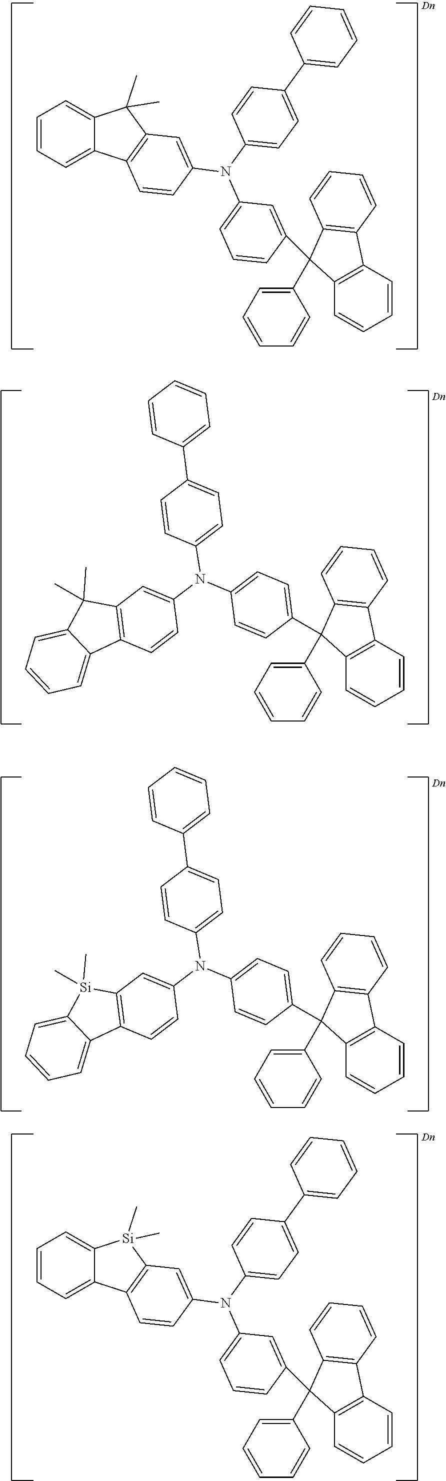

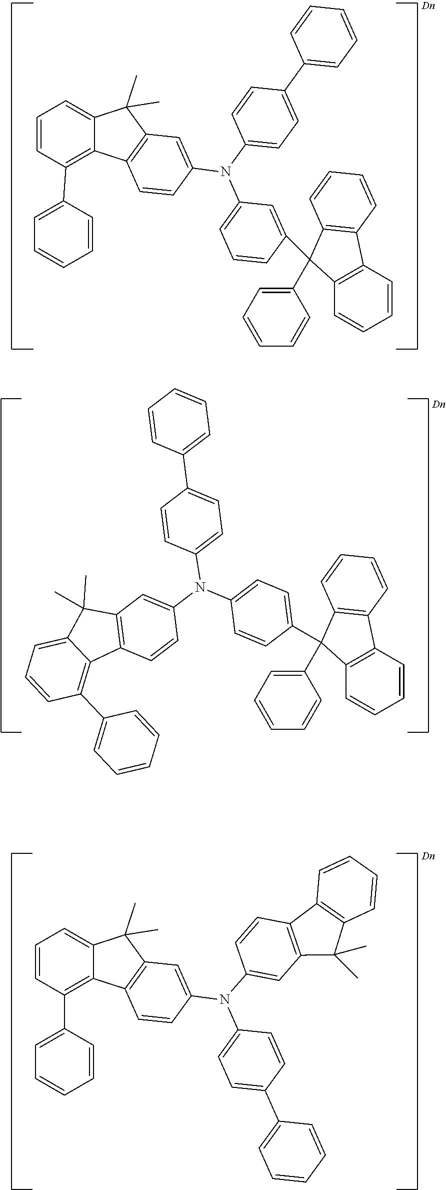

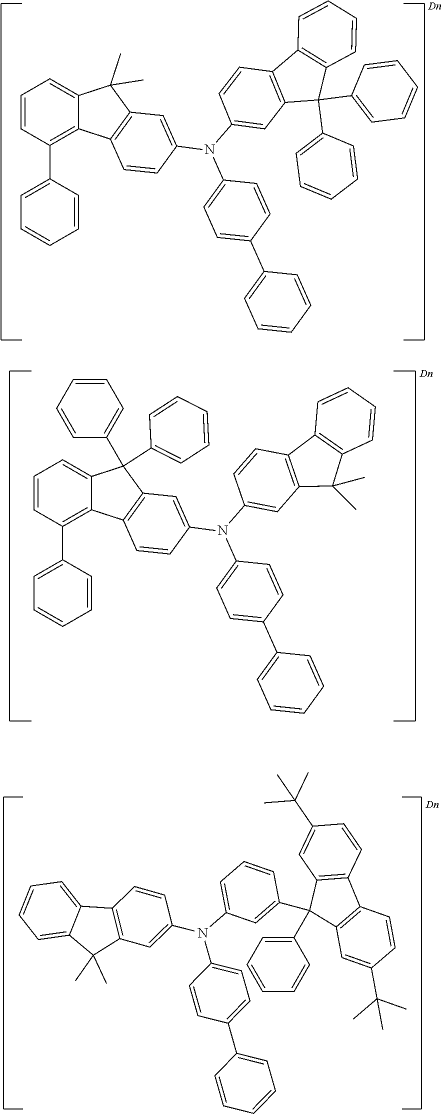

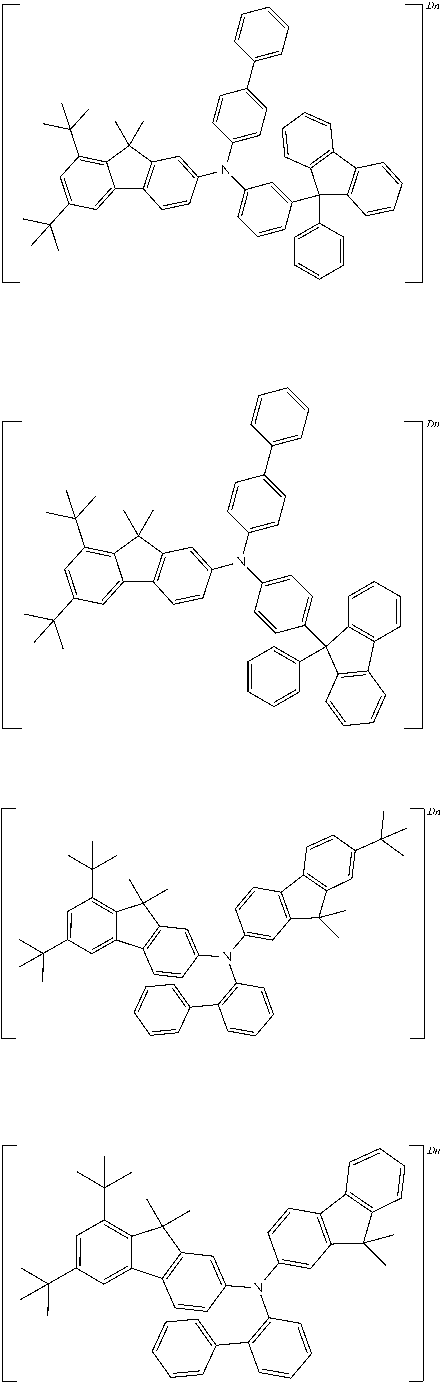

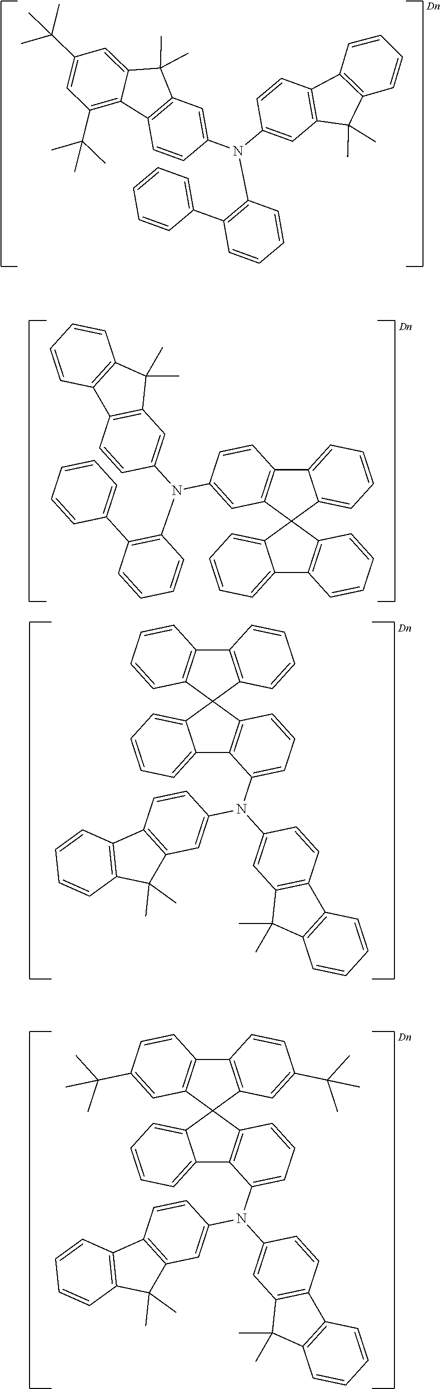

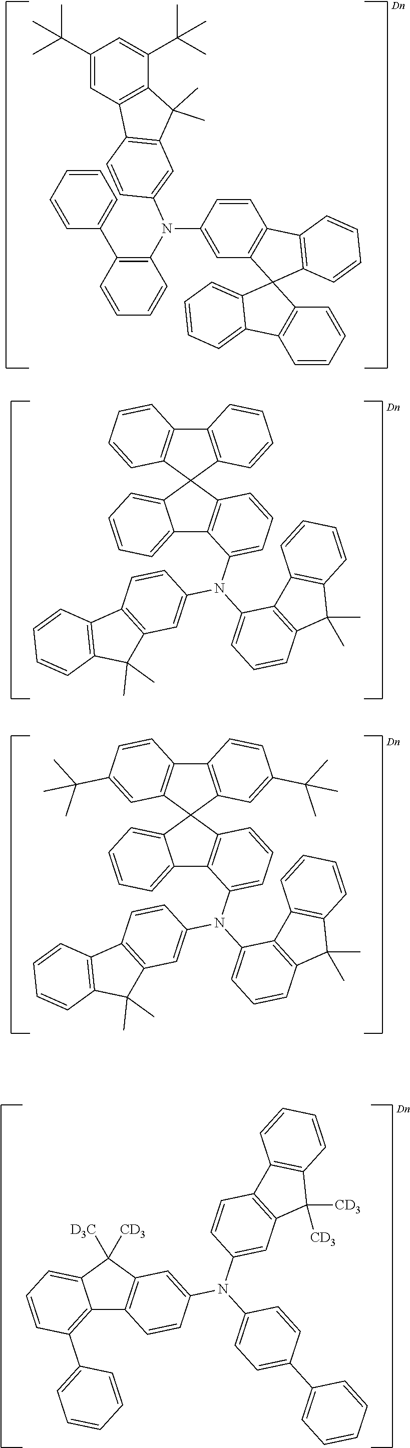

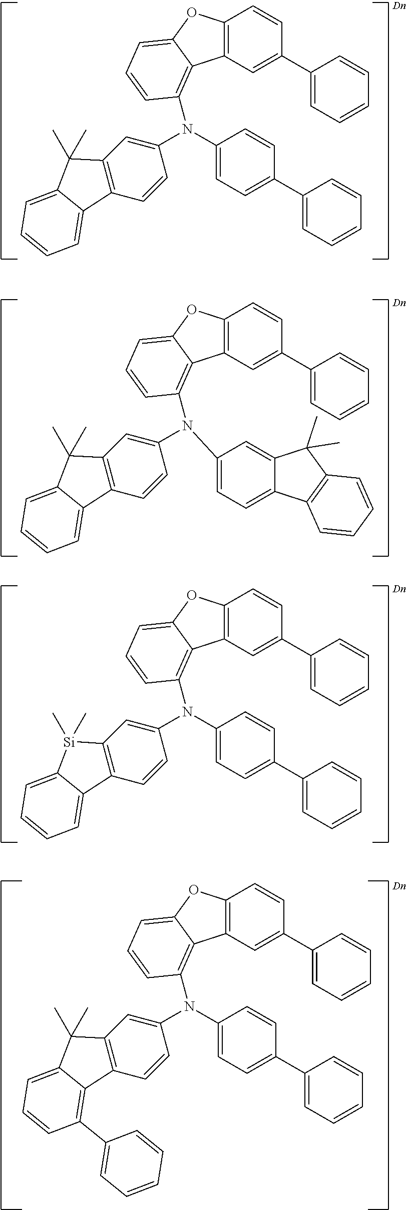

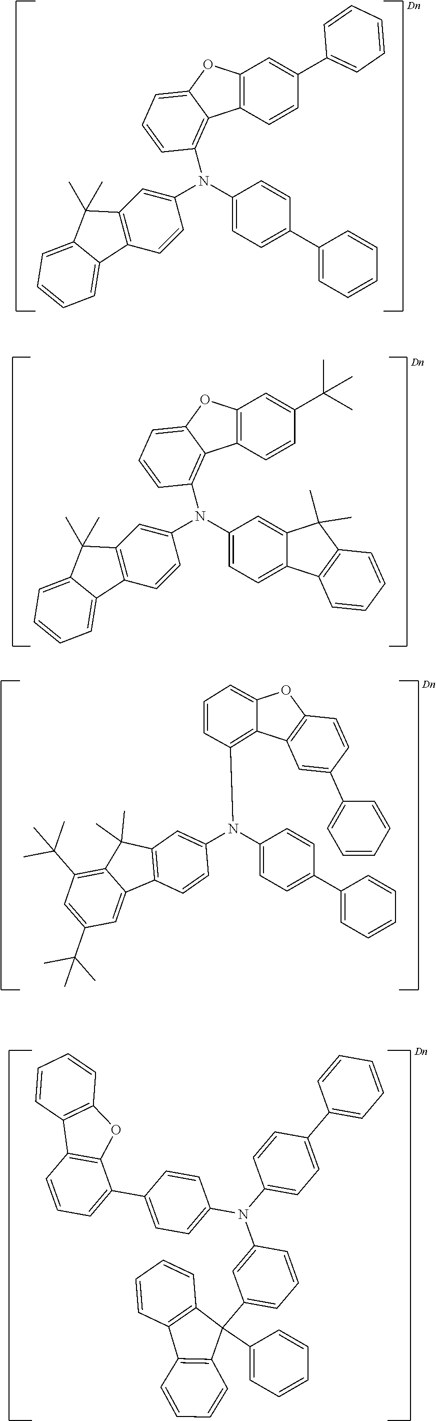

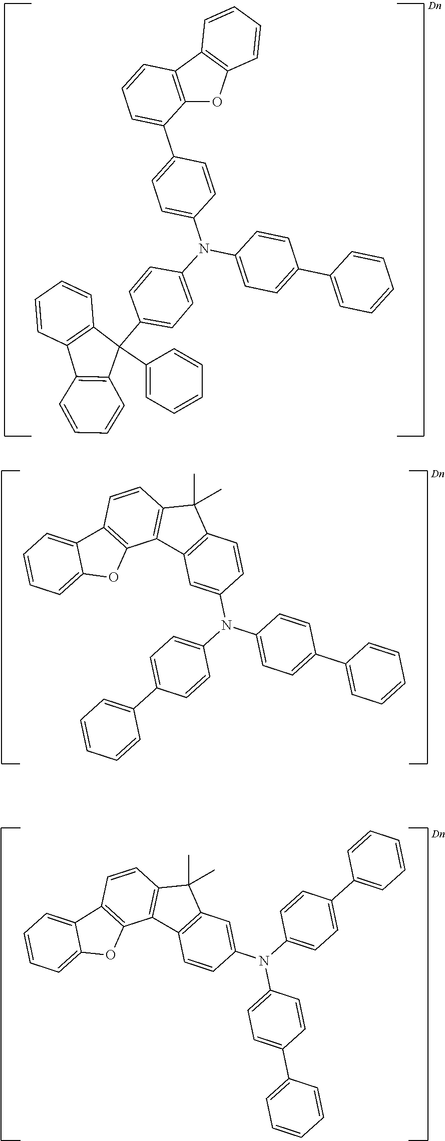

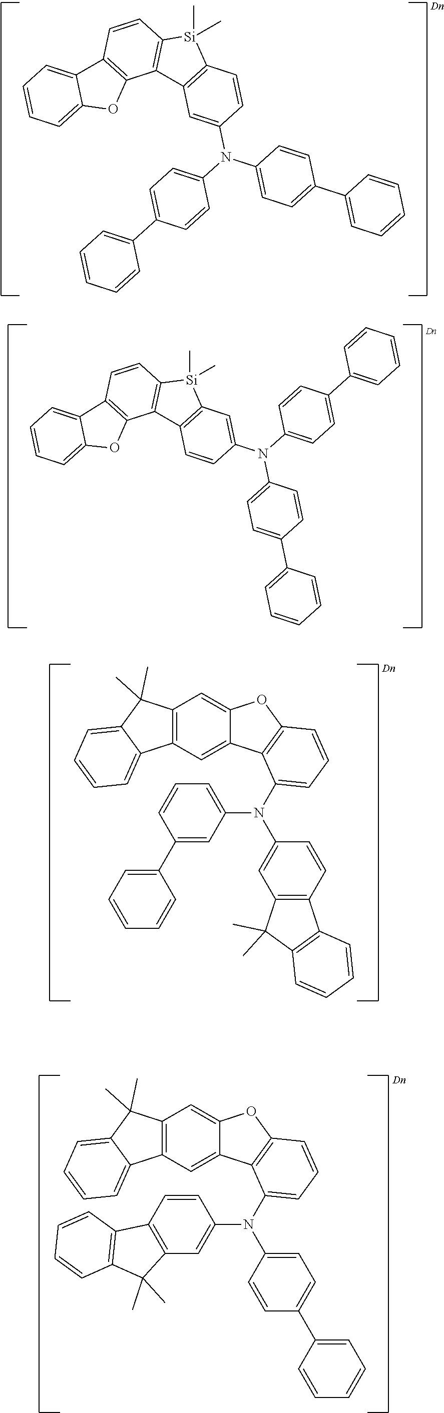

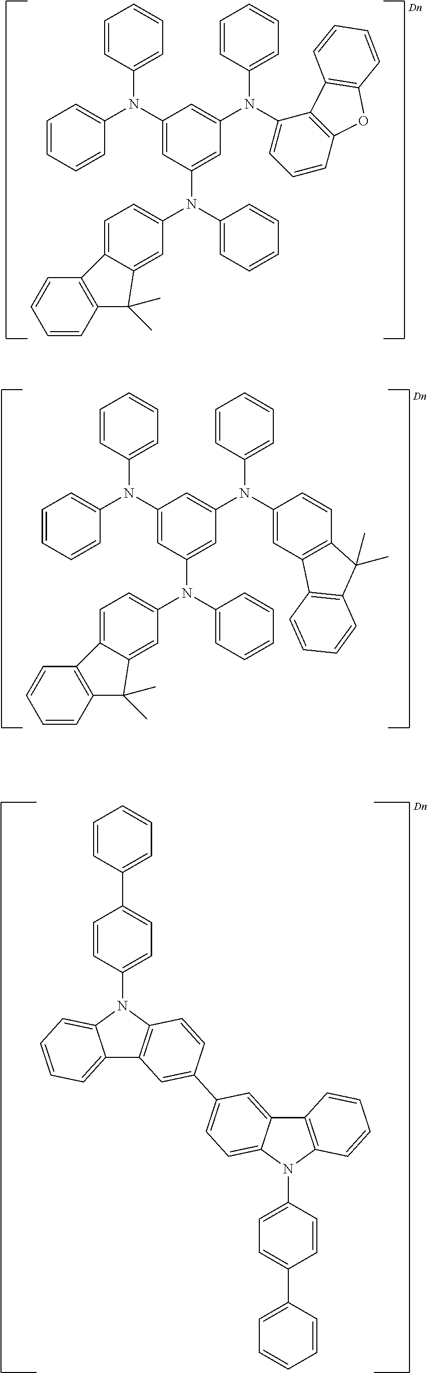

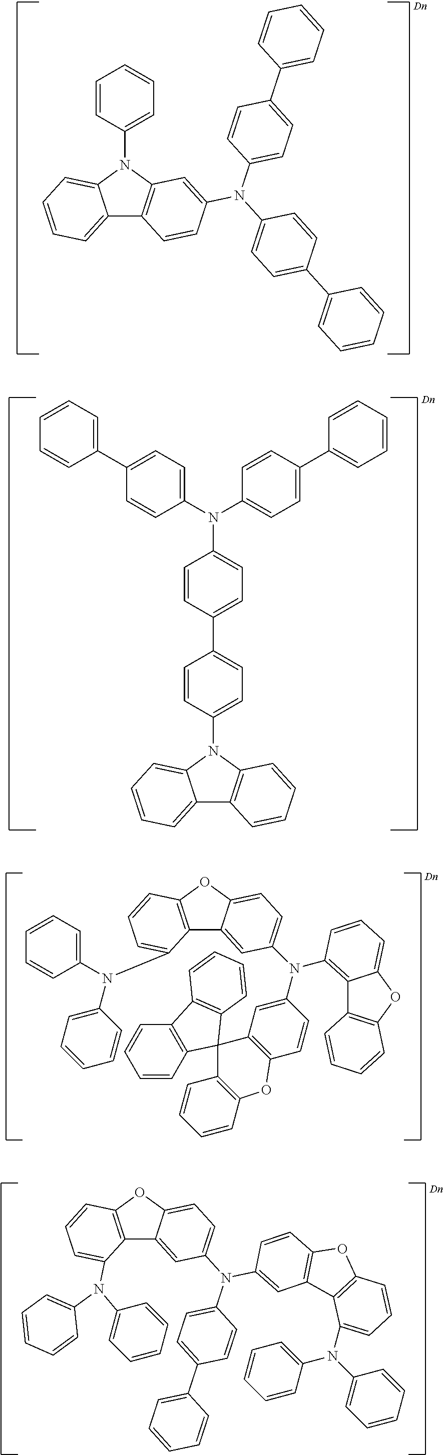

(Dn refers to the number of deuterium atoms substituted, and indicates a structure in which one or more deuterium atoms are substituted.)

In the hole transport region 140, in addition to the compounds described above, known compounds disclosed in U.S. Pat. No. 5,061,569A, JP1993-009471A, WO1995-009147A1, JP1995-126615A, JP1998-095973A, etc. and compounds having a similar structure may also be used.

Also, the charge transport region may be, for example, the electron transport region 150.

The electron transport region 150 may further increase electron injection and/or electron mobility and block holes between the cathode 110 and the light emitting layer 130.

Specifically, the electron transport region 150 may include an electron transport layer between the cathode 110 and the light emitting layer 130, and an electron transport auxiliary layer between the light emitting layer 130 and the electron transport layer, and at least one of the compounds of Group C may be included in at least one of the electron transport layer and the electron transport auxiliary layer.

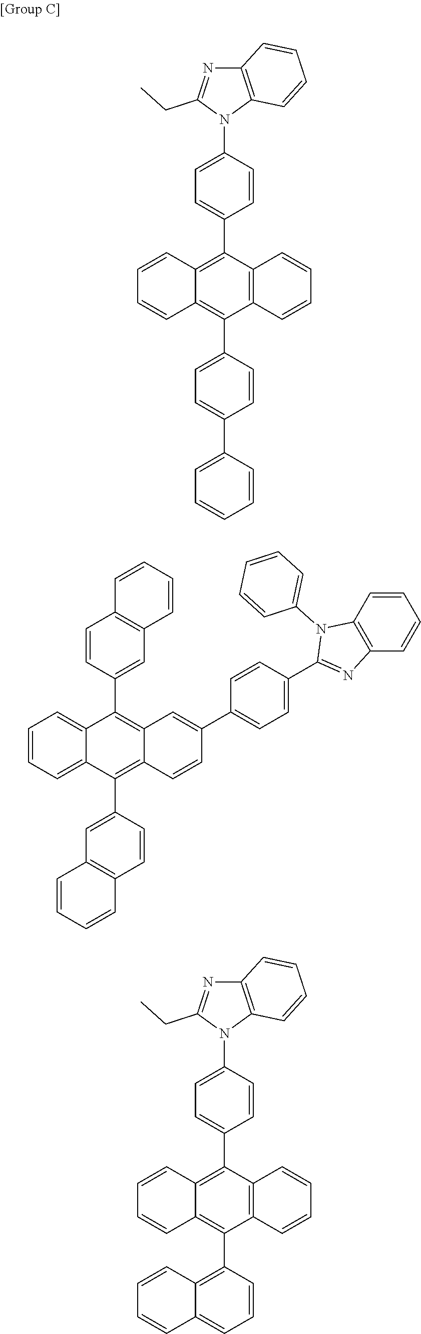 [SMILES: CCc1nc2ccccc2n1-c1ccc(-c2c3ccccc3c(-c3ccc(-c4ccccc4)cc3)c3ccccc23)cc1 ; c1ccc(-n2c(-c3ccc(-c4ccc5c(-c6ccc7ccccc7c6)c6ccccc6c(-c6ccc7ccccc7c6)c5c4)cc3)nc3ccccc32)cc1 ; CCc1nc2ccccc2n1-c1ccc(-c2c3ccccc3c(-c3cccc4ccccc34)c3ccccc23)cc1]

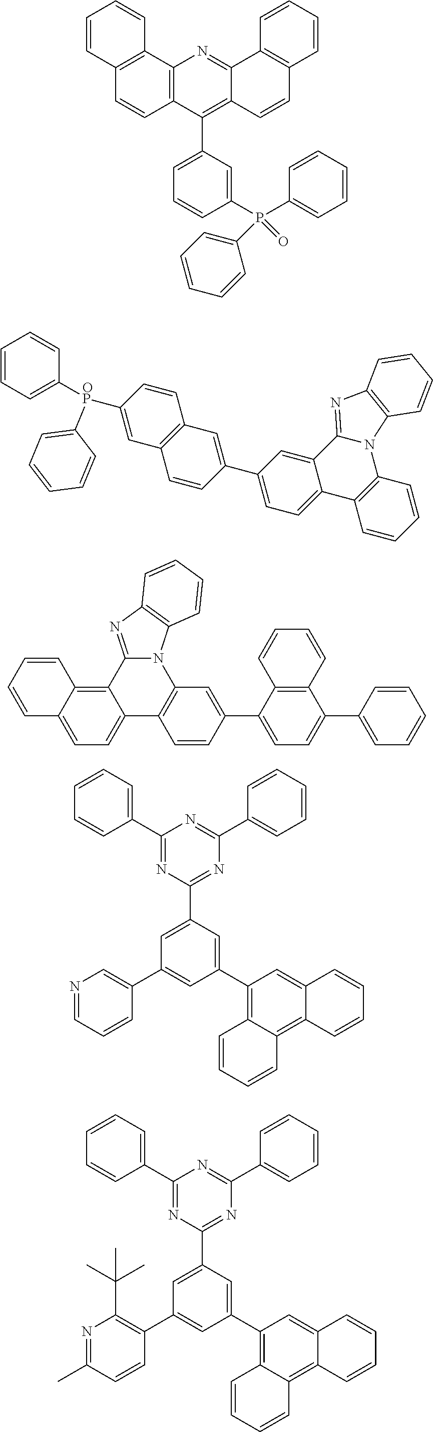

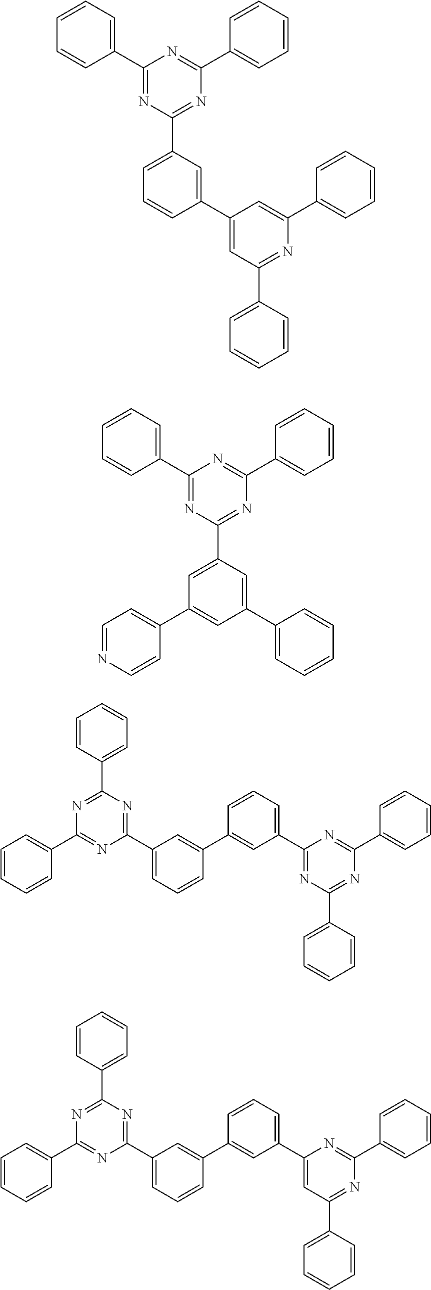

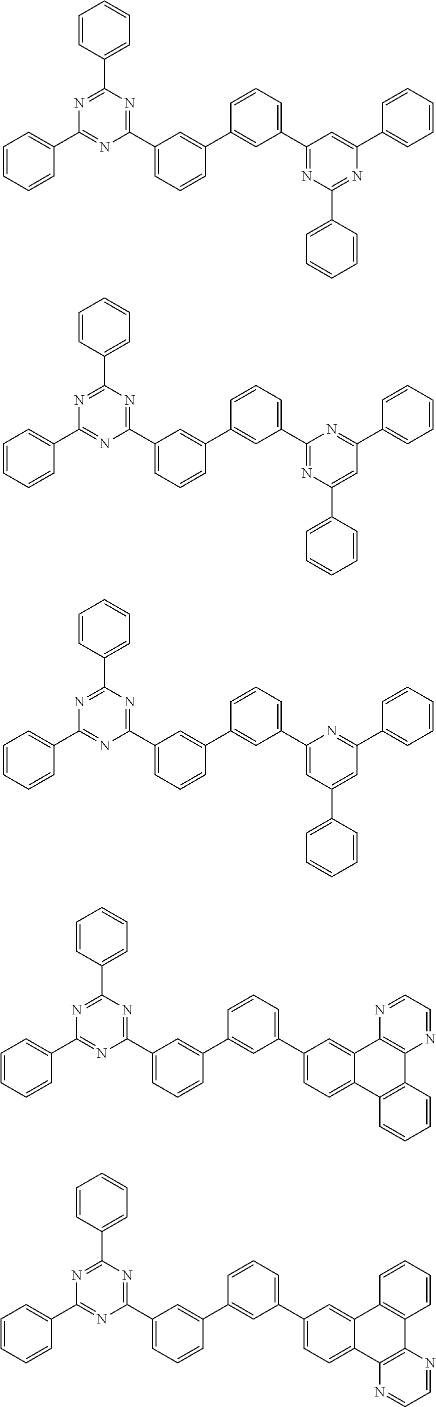 [SMILES: c1ccc(-c2cc(-c3cccc(-c4cccc(-c5nc(-c6ccccc6)nc(-c6ccccc6)n5)c4)c3)nc(-c3ccccc3)n2)cc1 ; c1ccc(-c2cc(-c3ccccc3)nc(-c3cccc(-c4cccc(-c5nc(-c6ccccc6)nc(-c6ccccc6)n5)c4)c3)n2)cc1 ; c1ccc(-c2cc(-c3ccccc3)nc(-c3cccc(-c4cccc(-c5nc(-c6ccccc6)nc(-c6ccccc6)n5)c4)c3)c2)cc1 ; c1ccc(-c2nc(-c3ccccc3)nc(-c3cccc(-c4cccc(-c5ccc6c7ccccc7c7nccnc7c6c5)c4)c3)n2)cc1 ; c1ccc(-c2nc(-c3ccccc3)nc(-c3cccc(-c4cccc(-c5ccc6c(c5)c5ccccc5c5nccnc65)c4)c3)n2)cc1]

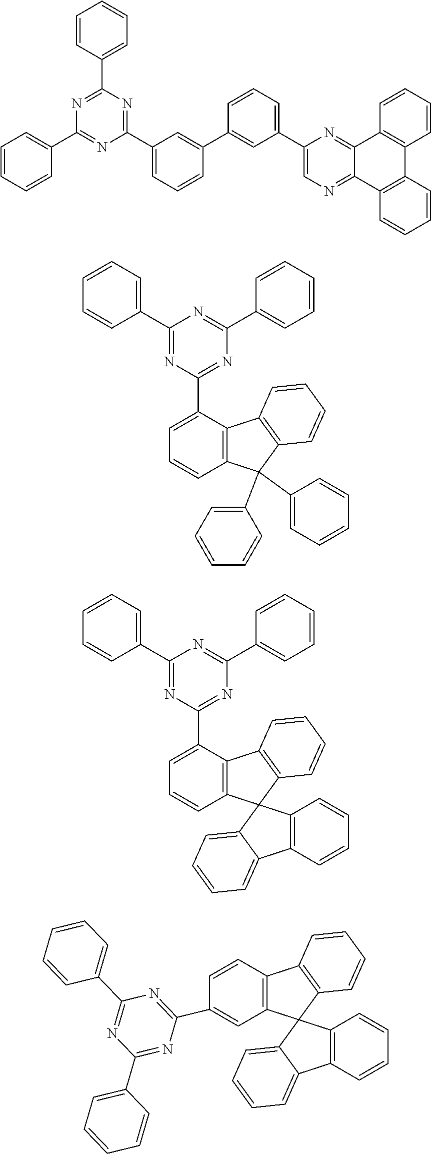 [SMILES: c1ccc(-c2nc(-c3ccccc3)nc(-c3cccc(-c4cccc(-c5cnc6c7ccccc7c7ccccc7c6n5)c4)c3)n2)cc1 ; c1ccc(-c2nc(-c3ccccc3)nc(-c3cccc4c3-c3ccccc3C4(c3ccccc3)c3ccccc3)n2)cc1 ; c1ccc(-c2nc(-c3ccccc3)nc(-c3cccc4c3-c3ccccc3C43c4ccccc4-c4ccccc43)n2)cc1 ; c1ccc(-c2nc(-c3ccccc3)nc(-c3ccc4c(c3)C3(c5ccccc5-c5ccccc53)c3ccccc3-4)n2)cc1]

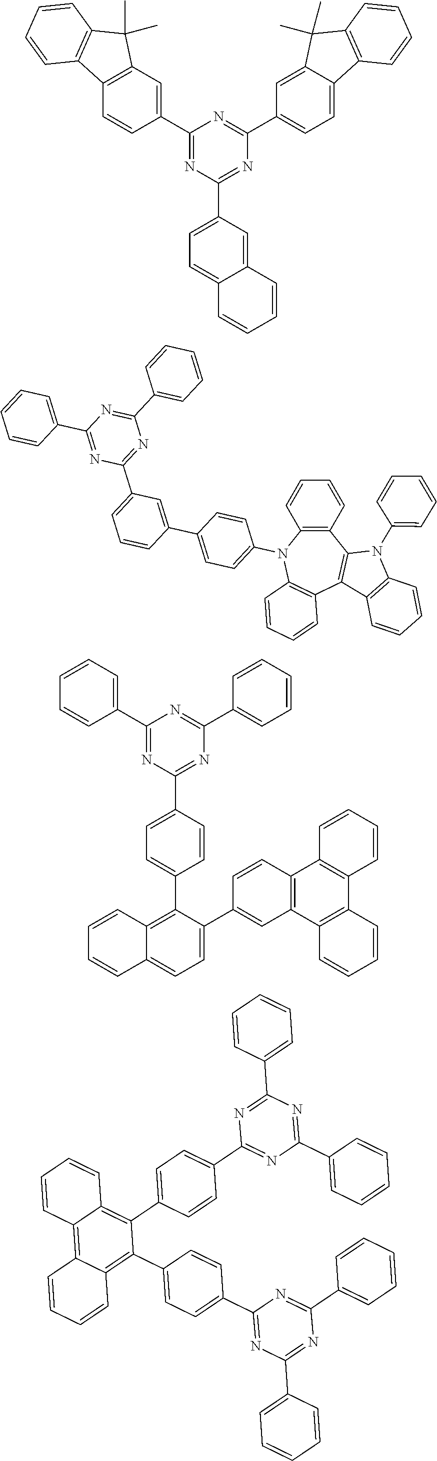

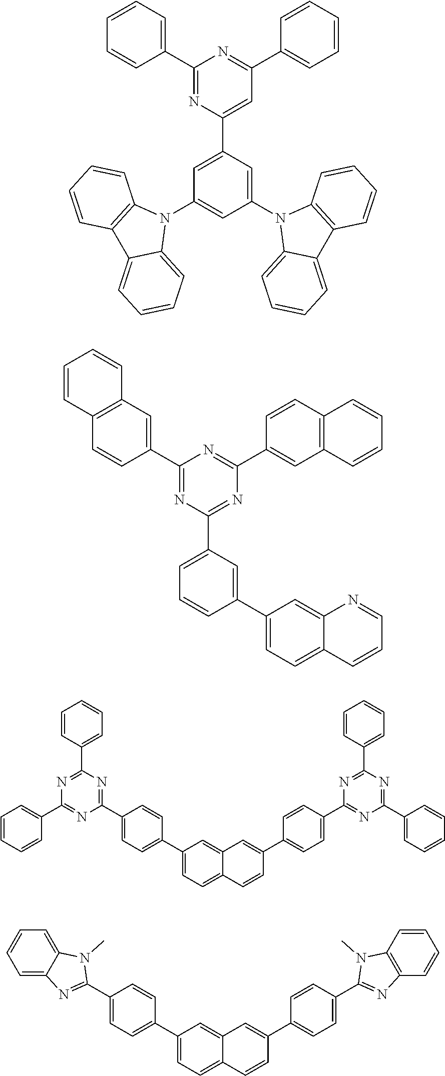

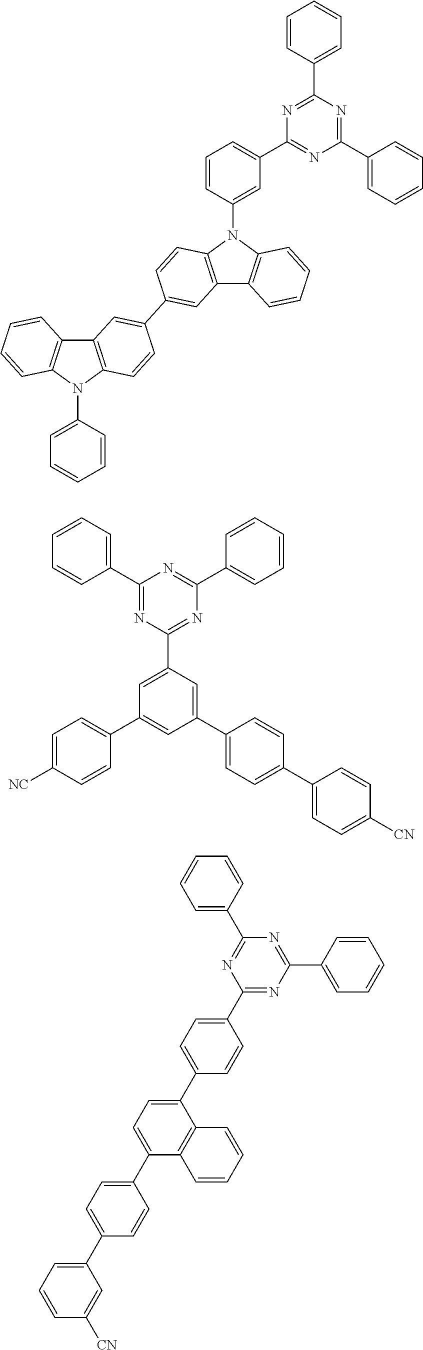

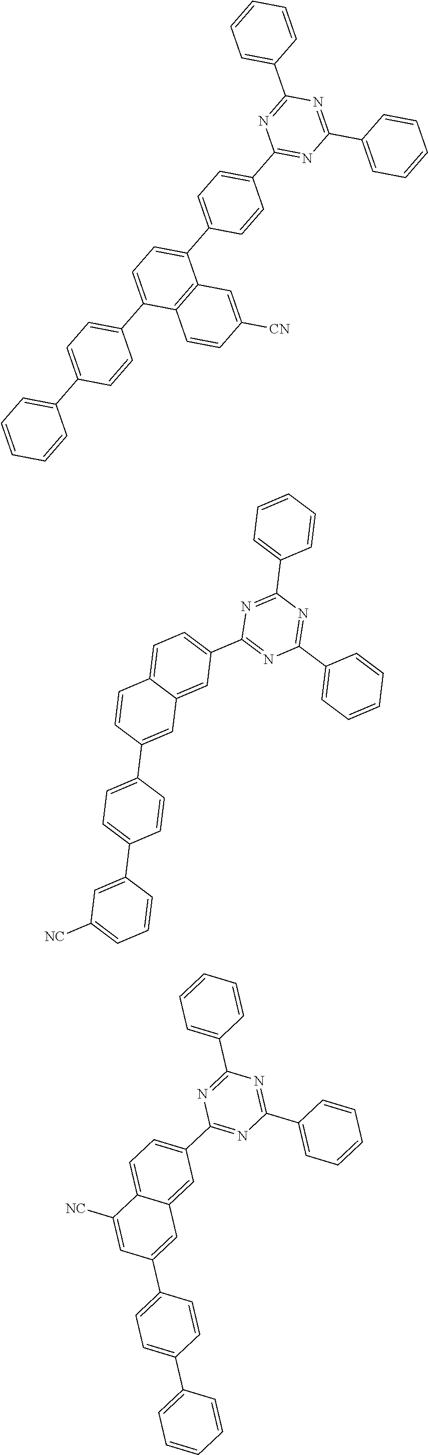 [SMILES: N#Cc1ccc2c(-c3ccc(-c4ccccc4)cc3)ccc(-c3ccc(-c4nc(-c5ccccc5)nc(-c5ccccc5)n4)cc3)c2c1 ; N#Cc1cccc(-c2ccc(-c3ccc4ccc(-c5nc(-c6ccccc6)nc(-c6ccccc6)n5)cc4c3)cc2)c1 ; N#Cc1cc(-c2ccc(-c3ccccc3)cc2)cc2cc(-c3nc(-c4ccccc4)nc(-c4ccccc4)n3)ccc12]

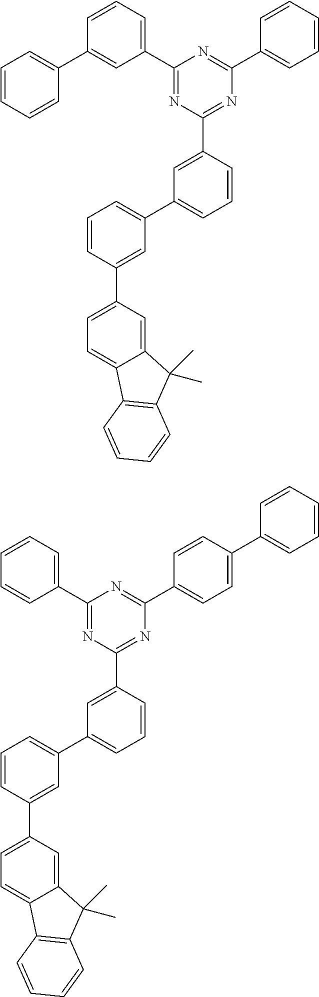 [SMILES: CC1(C)c2ccccc2-c2ccc(-c3cccc(-c4cccc(-c5nc(-c6ccccc6)nc(-c6cccc(-c7ccccc7)c6)n5)c4)c3)cc21 ; CC1(C)c2ccccc2-c2ccc(-c3cccc(-c4cccc(-c5nc(-c6ccccc6)nc(-c6ccc(-c7ccccc7)cc6)n5)c4)c3)cc21]

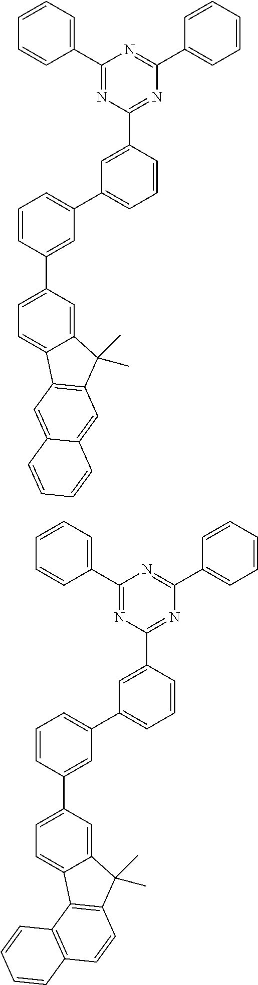

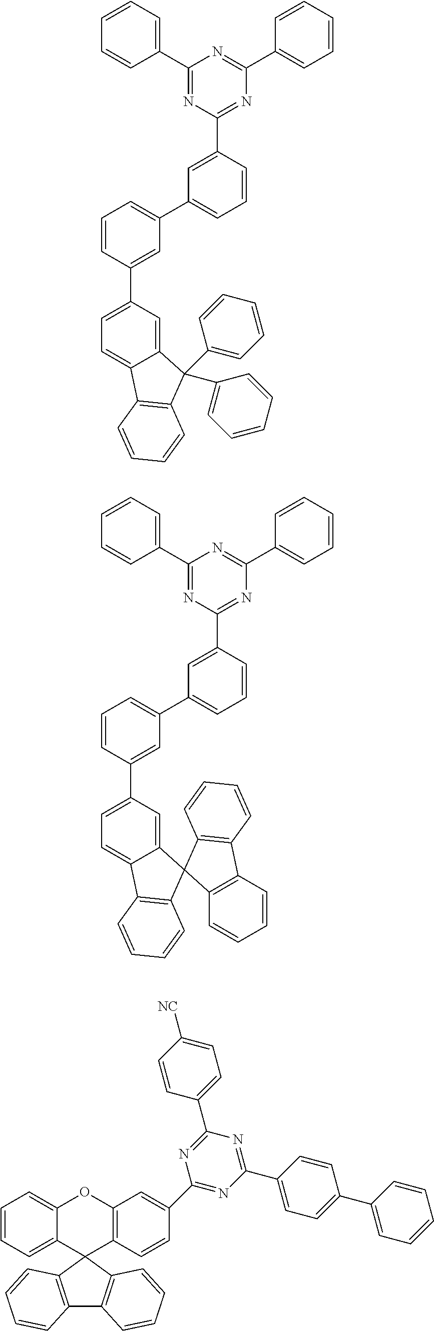

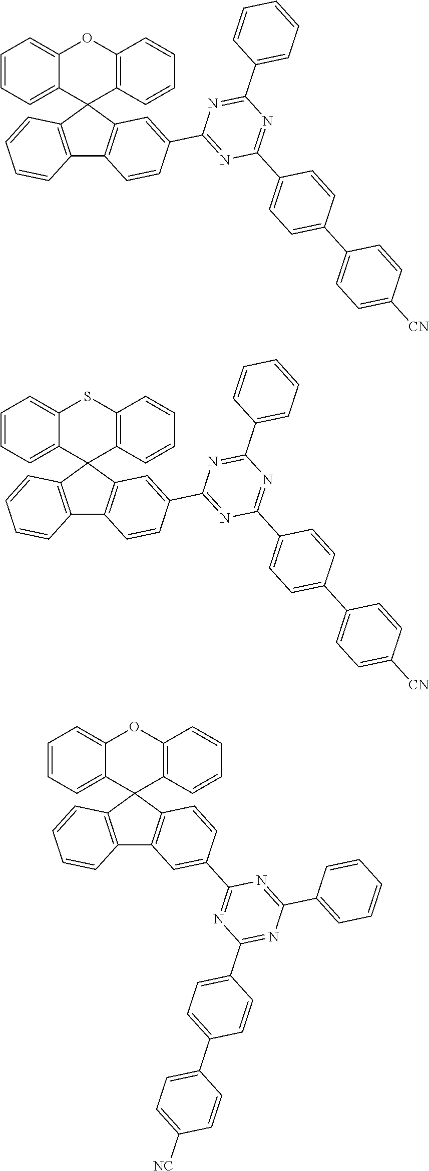 [SMILES: N#Cc1ccc(-c2ccc(-c3nc(-c4ccccc4)nc(-c4ccc5c(c4)C4(c6ccccc6Oc6ccccc64)c4ccccc4-5)n3)cc2)cc1 ; N#Cc1ccc(-c2ccc(-c3nc(-c4ccccc4)nc(-c4ccc5c(c4)C4(c6ccccc6Sc6ccccc64)c4ccccc4-5)n3)cc2)cc1 ; N#Cc1ccc(-c2ccc(-c3nc(-c4ccccc4)nc(-c4ccc5c(c4)-c4ccccc4C54c5ccccc5Oc5ccccc54)n3)cc2)cc1]

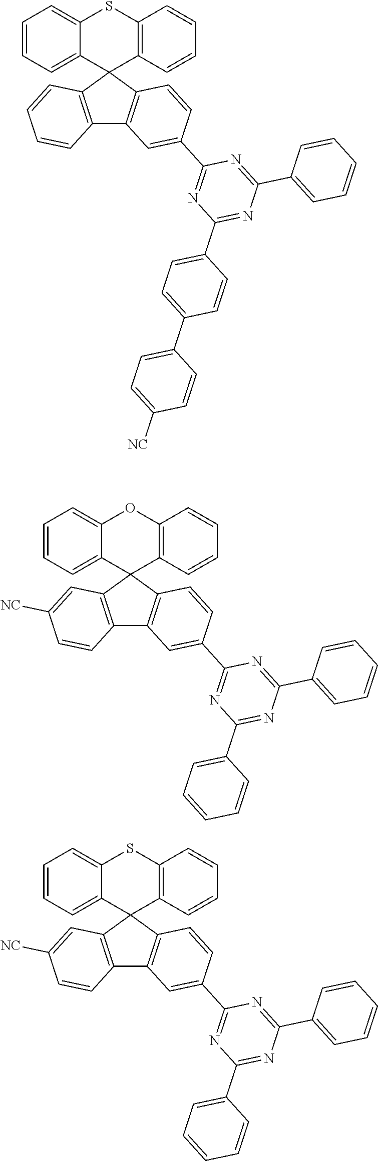

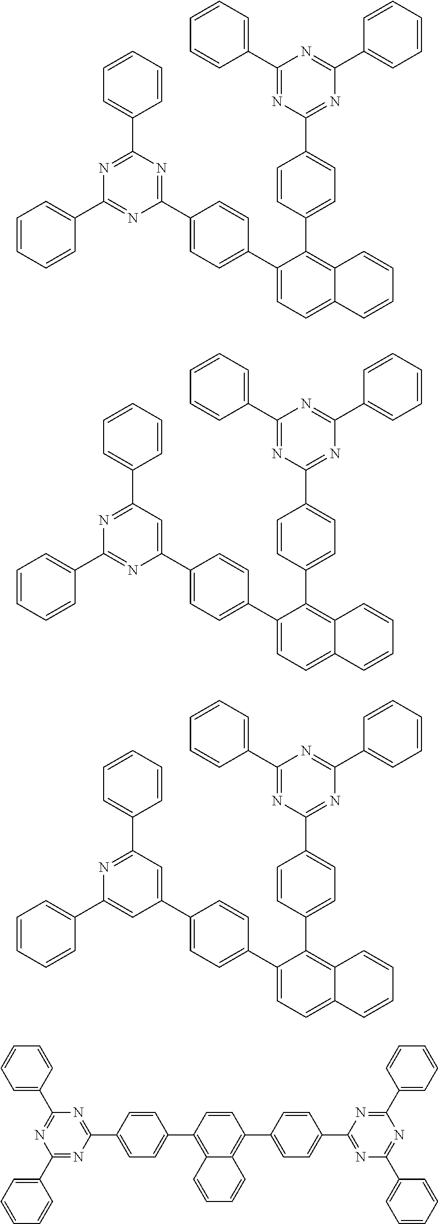

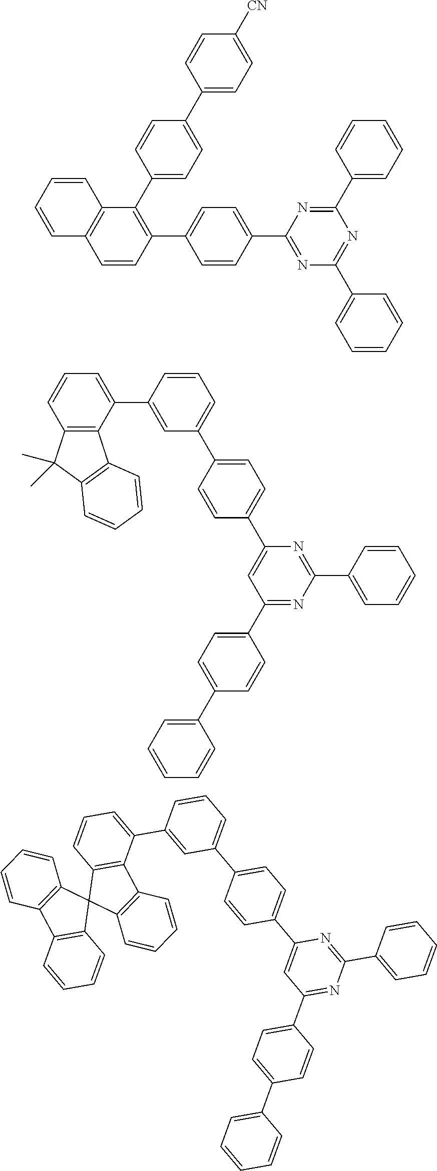

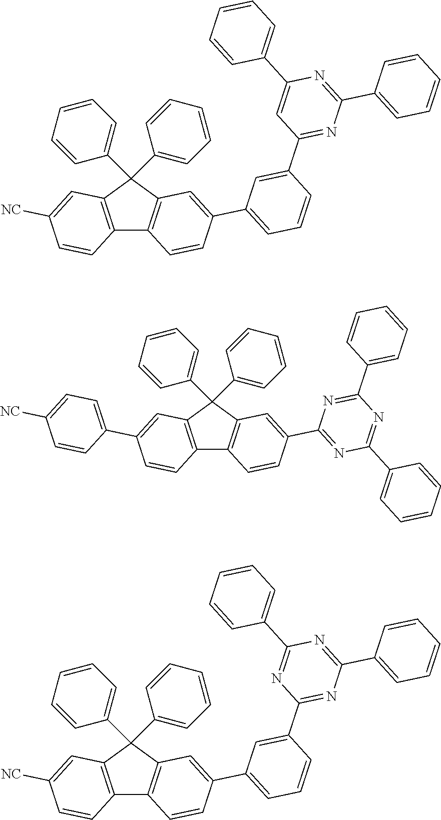 [SMILES: N#Cc1ccc2c(c1)C(c1ccccc1)(c1ccccc1)c1cc(-c3cccc(-c4cc(-c5ccccc5)nc(-c5ccccc5)n4)c3)ccc1-2 ; N#Cc1ccc(-c2ccc3c(c2)C(c2ccccc2)(c2ccccc2)c2cc(-c4nc(-c5ccccc5)nc(-c5ccccc5)n4)ccc2-3)cc1 ; N#Cc1ccc2c(c1)C(c1ccccc1)(c1ccccc1)c1cc(-c3cccc(-c4nc(-c5ccccc5)nc(-c5ccccc5)n4)c3)ccc1-2]

An embodiment may be an organic light emitting diode including the light emitting layer as the organic layer.

Another embodiment may be an organic light emitting diode including a light emitting layer and a hole transport region as the organic layer.

Another embodiment may be an organic light emitting diode including a light emitting layer and an electron transport region as the organic layer.

An organic light emitting diode according to an embodiment includes a hole transport region 140 and an electron transport region 150 in addition to the light emitting layer 130 as the organic layer 105, as shown in FIG. 1.

On the other hand, an organic light emitting diode may further include an electron injection layer (not shown), a hole injection layer (not shown), etc. in addition to the light emitting layer as the organic layer.

The organic light emitting diodes 100 may be manufactured by forming an anode or a cathode on a substrate, and then forming an organic layer by a dry film method such as vacuum deposition, sputtering, plasma plating and ion plating, and forming a cathode or an anode thereon.

The organic light emitting diode may be applied to an organic light emitting display device.

While this invention has been described in connection with what is presently considered to be practical embodiments, it is to be understood that the invention is not limited to the disclosed embodiments, but, on the contrary, is intended to cover various modifications and equivalent arrangements included within the spirit and scope of the appended claims.

### MODE FOR INVENTION

Hereinafter, the embodiments are illustrated in more detail with reference to examples. However, these examples are exemplary, and the present scope is not limited thereto.

### (Synthesis of First Compound)

### Synthesis Example 1: Synthesis of Compound A-1

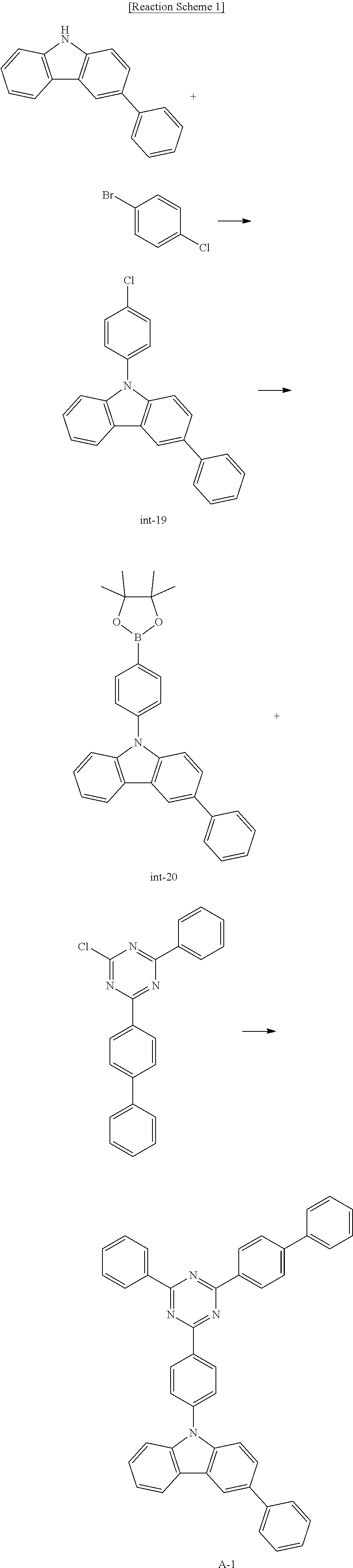

1st Step. Synthesis of Intermediate Int-19

3-Phenyl-9H-carbazole (30 g, 123.3 mmol), 1-bromo-4-chlorobenzene (23.6 g, 123.3 mmol), sodium tert-butoxide (23.7 g, 246.6 mmol), tri-tert-butylphosphine (2.5 g, 12.3 mmol), and Pd₂(dba)₃ (5.6 g, 6.2 mmol) were added in a round-bottomed flask, dissolved in xylene (600 ml), and stirred under reflux at 150° C. for 8 hours. When a reaction was completed, after removing a salt by filtration, a filtrate therefrom was adsorbed. Column chromatography (Hexane:DCM (25%)) was used to obtain 28.4 g (65%) of Intermediate int-19.

### 2nd Step: Synthesis of Intermediate Int-20

Intermediate int-19 (28 g, 79.1 mmol), bis(pinacolato)diboron (24.1 g, 94.9 mmol), tricyclohexylphosphine (3.8 g, 15.8 mmol), potassium acetate (15.5 g, 158.3 mmol), and Pd(dppf)Cl₂ (1.9 g, 2.4 mmol) were added in a round-bottomed flask and dissolved in 250 ml of xylene. The mixture was stirred under reflux at 120° C. for 8 hours. When a reaction was completed, after cooling to room temperature and removing a salt by filtration, an excessive amount of DCM and distilled water were added thereto for extraction. Column chromatography (Hexane:DCM (30%)) was used to obtain 20.4 g (82%) of Intermediate int-20.

### 3rd Step: Synthesis of Compound A-1

2-chloro-4-(biphenyl-4-yl)-6-phenyl-1,3,5-triazine (14.1 g, 41.0 mmol), Intermediate int-20 (20.1 g, 45.1 mmol), K₂CO₃ (11.3 g, 82.0 mmol), and Pd(PPh₃)₄(2.4 g, 2.1 mmol) were added in a round-bottomed flask, dissolved in THF (200 ml) and distilled water (40 ml), and stirred under reflux at 70° C. for 12 hours. After the reaction was completed, the solid was separated by filtration and recrystallized with monochlorobenzene to obtain 18.5 g (72%) of Compound A-1.

### Synthesis Example 2: Synthesis of Compound A-27

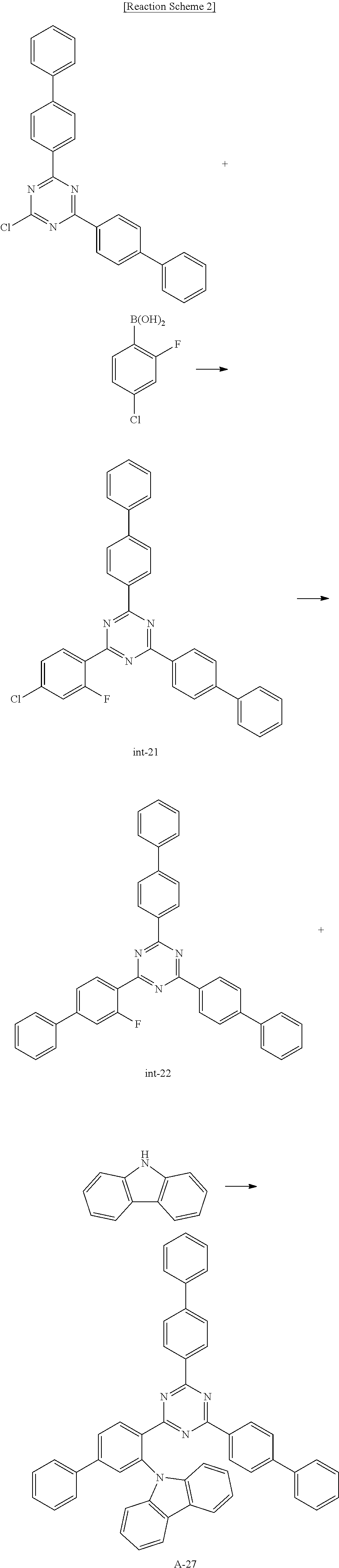

### 1st Step: Synthesis of Intermediate Int-21

2,4-bis([1,1′-biphenyl]-4-yl)-6-chloro-1,3,5-triazine (40 g, 95.3 mmol), 4-chloro-2-fluorophenylboronic acid (15.8 g, 90.5 mmol), K₂CO₃ (26.3 g, 190.5 mmol), and Pd(PPh₃)₄(5.5 g, 4.8 mmol) were added in a round-bottomed flask, dissolved in THF (320 ml) and distilled water (100 ml), and stirred under reflux at 70° C. for 12 hours. After the reaction was completed, the precipitated solid was filtered and then a silica hot filter was performed using monochlorobenzene. The filtrate was distilled under reduced pressure and recrystallized with monochlorobenzene to obtain 38.6 g (83%) of Intermediate int-21.

### 2nd Step: Synthesis of Intermediate Int-22

Intermediate int-21 (21.7 g, 42.2 mmol), phenylboronic acid (15.4 g, 126.7 mmol), Cs₂CO₃ (27.5 g, 84.4 mmol), tri-tert-butylphosphine (1.7 g, 8.4 mmol), and Pd₂(dba)₃ (1.9 g, 2.1 mmol) were added in a round-bottomed flask, dissolved in 1,4-dioxane (200 ml), and stirred under reflux at 120° C. for 8 hours. When a reaction was completed, an excessive amount of distilled water was added to the reactant and then, stirred for 30 minutes, and a solid precipitated therein was filtered. After performing silica hot filter using monochlorobenzene, the filtrate was distilled under reduced pressure and recrystallized with monochlorobenzene to obtain 22.1 g (94%) of Intermediate int-22.

3rd step: Synthesis of Compound A-27

Intermediate int-22 (22.1 g, 39.8 mmol), 9H-carbazole (8.0 g, 47.7 mmol), and K₃PO₄ (16.9 g, 79.6 mmol) were added in a round bottom flask, dissolved in DMF (120 ml), and stirred under reflux at 150° C. for 4 hours. When a reaction was completed, the reactant was slowly added in a dropwise fashion to an excessive amount of was to precipitate a solid, and the solid was filtered therefrom. Column chromatography (hexane:DCM (30%)) was used to obtain 24.6 g (88%) of Compound A-27.

### (Synthesis of Second Compound)

### Synthesis Example 3: Synthesis of Compound B-106

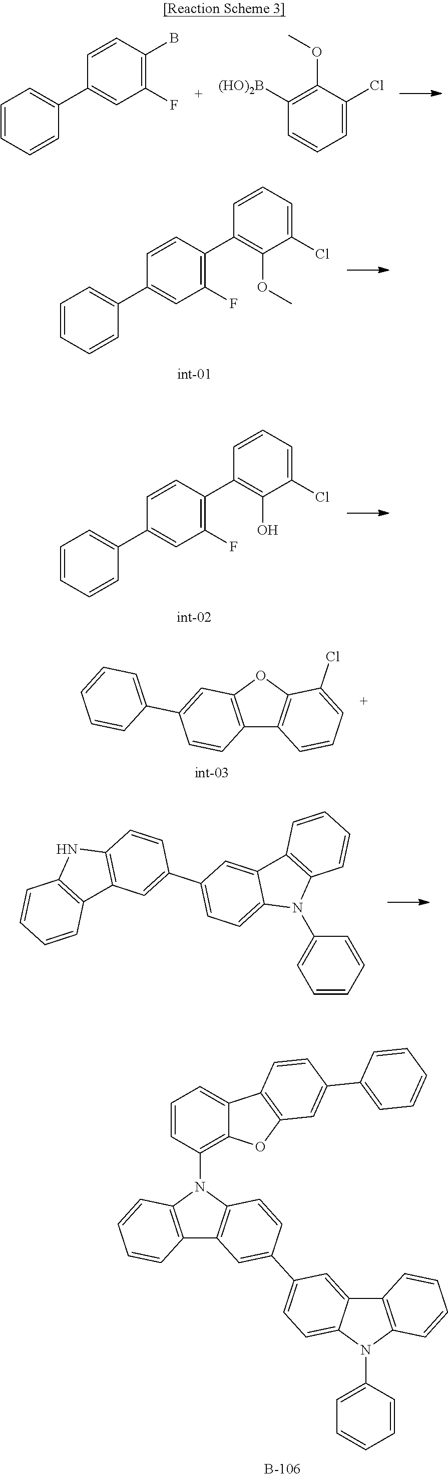

### 1st Step: Synthesis of Intermediate Int-01

4-bromo-3-fluoro-1,1′-biphenyl (20 g, 79.7 mmol), (3-chloro-2-methoxyphenyl)boronic acid (16.3 g, 87.6 mmol), K₂CO₃ (22.1 g, 159.3 mmol), and Pd(PPh₃)₄(4.6 g, 4.0 mmol) were added to a round-bottomed flask, dissolved in 320 ml of THF and 80 ml of distilled water, and then, stirred under reflux at 70° C. for 8 hours. When a reaction was completed, the resultant was cooled to room temperature, filtered to remove a salt, and then, extracted by adding an excessive amount of DCM and distilled water thereto. 24.7 g (99%) of Intermediate int-01 was obtained by using column chromatography (hexane:DCM 30%).

### 2nd Step: Synthesis of Intermediate Int-02

Intermediate int-01 (23.7 g, 75.8 mmol) and pyridine hydrochloride (52.5 g, 454.6 mmol) were added to a round-bottomed flask and then, stirred under reflux at 200° C. for 24 hours. When a reaction was completed, the resultant was cooled to room temperature, slowly poured into distilled water, and then, stirred for 1 hour. A solid was filtered therefrom, obtaining 22.6 g (100%) of Intermediate int-02.

### 3rd Step: Synthesis of Intermediate Int-03

Intermediate int-02 (21 g, 70.3 mmol) and K₂CO₃ (19.4 g, 140.6 mmol) were added to a round-bottomed flask, dissolved in 100 ml of NMP, and then, stirred under reflux at 180° C. for 12 hours. When a reaction was completed, the mixture was poured into an excessive amount of distilled water. A solid was filtered therefrom, dissolved in ethylacetate, and dried with MgSO₄, and then, an organic layer was removed therefrom under a reduced pressure. 13.8 g (70%) of Intermediate int-03 was obtained therefrom through column chromatography (hexane:DCM (20%)).

4th step: Synthesis of Compound B-106

Intermediate int-03 (13 g, 46.6 mmol), 9-phenyl-3,3′-bicarbazole (19.1 g, 46.6 mmol), sodium tert-butoxide (8.9 g, 93.2 mmol), SPhos (1.9 g, 4.7 mmol), and Pd₂(dba)₃ (2.1 g, 2.3 mmol) were added to a round-bottomed flask, dissolved in 150 ml of xylene, and then, stirred under reflux at 150° C. for 6 hours. When a reaction was completed, the resultant was filtered to remove a salt, and a filtrate therefrom was adsorbed with silica gel. 22.8 g (75%) of Compound B-106 was obtained by using column chromatography (hexane:DCM (30%)).

### Synthesis Example 4: Synthesis of Compound B-36

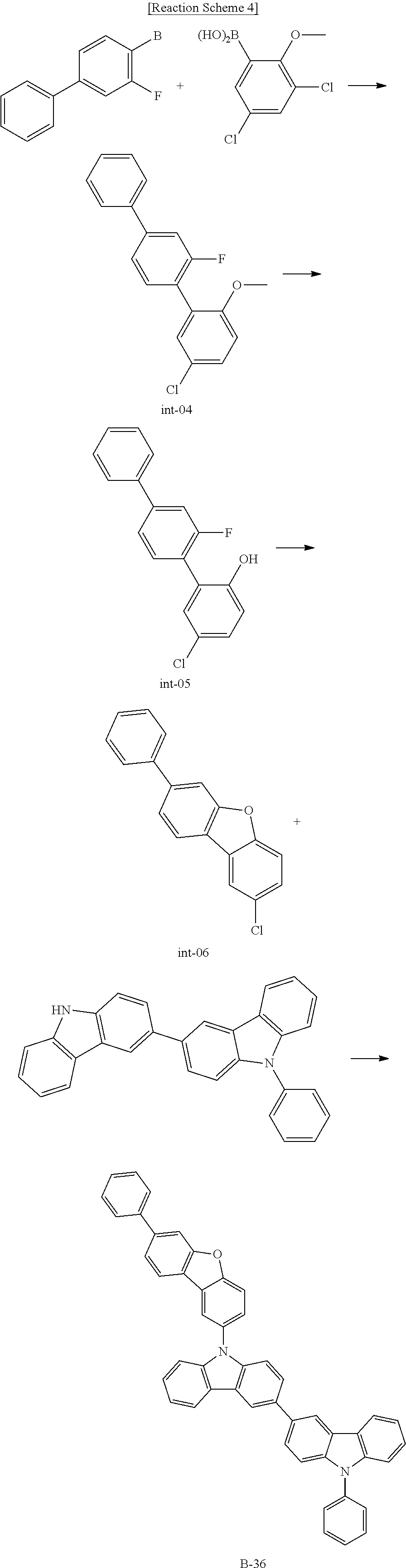

### 1st Step: Synthesis of Intermediate Int-04

4-bromo-3-fluoro-1,1′-biphenyl (20 g, 79.7 mmol), (5-chloro-2-methoxyphenyl)boronic acid (16.3 g, 87.6 mmol), K₂CO₃ (22.1 g, 159.3 mmol), and Pd(PPh₃)₄ (4.6 g, 4.0 mmol) were added to a round-bottomed flask, dissolved in 320 ml of THF and 80 ml of distilled water, and then, stirred under reflux at 70° C. for 8 hours. When a reaction was completed, the resultant was cooled to room temperature and filtered to remove a salt, and an excessive amount of DCM and distilled water were added thereto for extraction. 20.8 g (85%) of Intermediate int-04 was obtained by using column chromatography (hexane:DCM (30%)).

### 2nd Step: Synthesis of Intermediate Int-05

Intermediate int-04 (20.8 g, 66.5 mmol) and pyridine hydrochloride (46.1 g, 399.0 mmol) were added to a round-bottomed flask and then, stirred under reflux at 200° C. for 24 hours. When a reaction was completed, the resultant was cooled to room temperature, slowly poured into distilled water, and then, stirred for 1 hours. A solid was filtered therefrom, obtaining 18.1 g (91%) of Intermediate int-05.

### 3rd Step: Synthesis of Intermediate Int-06

Intermediate int-05 (18.1 g, 60.6 mmol) and K₂CO₃ (16.8 g, 121.2 mmol) were added to a round-bottomed flask, dissolved in 80 ml of NMP, and then, stirred under reflux at 180° C. for 12 hours. When a reaction was completed, the mixture was poured into an excessive amount of distilled water. A solid was filtered therefrom, dissolved in ethylacetate, and dried with MgSO₄, and then, an organic layer was removed under a reduced pressure. 16.1 g (95%) of Intermediate int-06 was obtained by using column chromatography (hexane:DCM (20%)).

4th step: Synthesis of Compound B-36

Intermediate int-06 (16.1 g, 57.8 mmol), 9-phenyl-3,3′-bicarbazole (23.6 g, 57.8 mmol), sodium tert-butoxide (11.1 g, 115.5 mmol), SPhos (2.4 g, 5.8 mmol), and Pd₂(dba)₃ (2.6 g, 2.9 mmol) were added to a round-bottomed flask and dissolved in 200 ml of xylene and then, stirred under reflux at 150° C. for 6 hours. When a reaction was completed, the resultant was filtered to remove a salt, and then, a filtrate therefrom was adsorbed with silica gel. 25.6 g (68%) of Compound B-36 was obtained by using column chromatography (hexane:DCM (30%)).

### Comparative Synthesis Example 1: Synthesis of Compound C-1

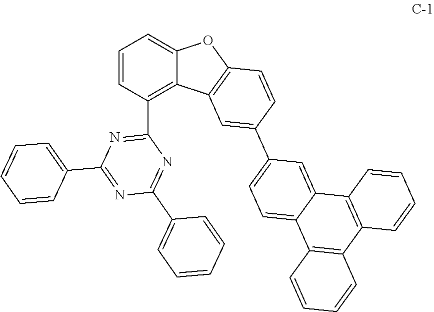 [SMILES: c1ccc(-c2nc(-c3ccccc3)nc(-c3cccc4oc5ccc(-c6ccc7c8ccccc8c8ccccc8c7c6)cc5c34)n2)cc1]

Compound C-1 was synthesized by referring to the synthetic method known in registered patent No. KR 1849747 B1.

### Comparative Synthesis Example 2: Synthesis of Compound C-2

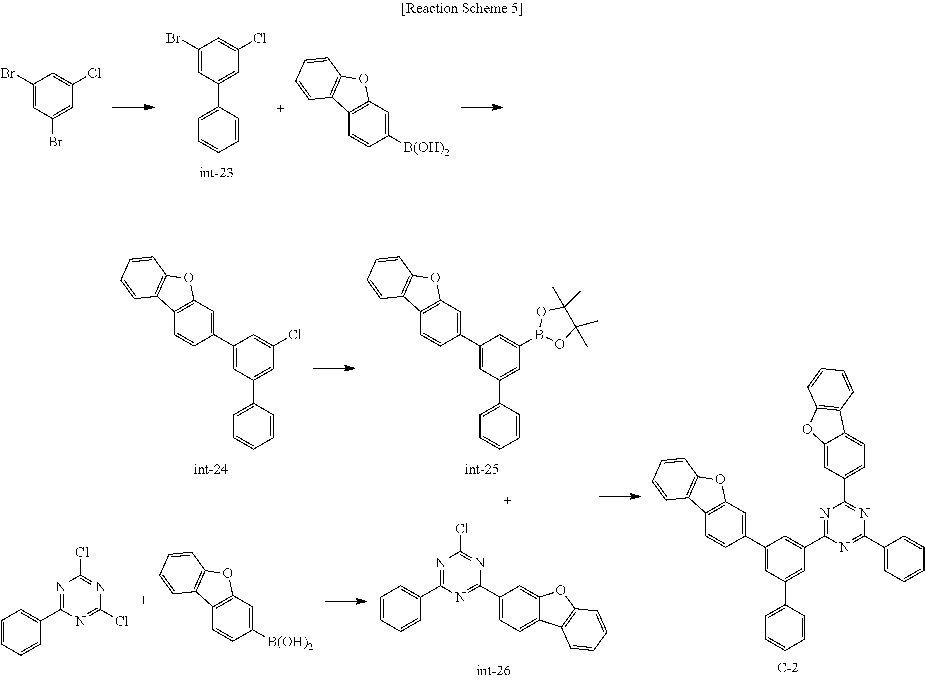

### 1st Step: Synthesis of Intermediate Int-23

1,3-dibromo-5-chlorobenzene (45 g, 166.5 mmol), phenylboronic acid (19.3 g, 158.1 mmol), K₂CO₃ (41.4 g, 299.6 mmol), and Pd(PPh₃)₄ (9.6 g, 8.3 mmol) were added to a round-bottomed flask, dissolved in 600 ml of THF and 150 ml of distilled water, and then, stirred under reflux at 70° C. for 8 hours. When a reaction was completed, after removing an aqueous layer, 25 g (59%) of Intermediate int-23 was obtained by using column chromatography (hexane:DCM (15%)).

### 2nd Step: Synthesis of Intermediate Int-24

Intermediate int-23 (25 g, 93.4 mmol), 3-dibenzofuranylboronic acid (21.8 g, 102.8 mmol), K₂CO₃ (25.8 g, 186.9 mmol), and Pd(PPh₃)₄ (5.4 g, 4.7 mmol) were added to a round-bottomed flask, dissolved in 400 ml of THF and 100 ml of distilled water, and then, stirred under reflux at 70° C. for 8 hours. When a reaction was completed, after removing an aqueous layer, 24.4 g (67%) of Intermediate int-24 was obtained therefrom by using column chromatography (hexane:DCM (30%)).

### 3rd Step: Synthesis of Intermediate Int-25

Intermediate int-24 (24 g, 67.6 mmol), bis(pinacolato)diboron (20.6 g, 81.2 mmol), tricyclohexylphosphine (3.3 g, 13.5 mmol), potassium acetate (13.3 g, 135.3 mmol), and Pd(dppf)Cl₂ (1.7 g, 2.0 mmol) were added to a round-bottomed flask and dissolved in 250 ml of xylene. The mixture was stirred under reflux at 150° C. for 8 hours. When a reaction was completed, the resultant was cooled to room temperature, filtered to remove a salt, and then, extracted by adding an excessive amount of DCM and distilled water thereto. 23.6 g (78%) of Intermediate int-25 was obtained by using column chromatography (hexane:DCM (40%)).

### 4th Step: Synthesis of Intermediate Int-26

2,4-dichloro-6-phenyl-1,3,5-triazine (20 g, 88.5 mmol), 3-dibenzofuranylboronic acid (17.8 g, 84.1 mmol), K₂CO₃ (24.5 g, 176.9 mmol), and Pd(dppf)Cl₂ (3.6 g, 4.4 mmol) were added to a round-bottomed flask, dissolved in 250 ml of toluene and 90 ml of distilled water, and then, stirred at 60° C. for 6 hours. When a reaction was completed, after separating an aqueous layer by using a separatory funnel, an organic layer therefrom was distilled under a reduced pressure. The obtained product was dissolved by heating with monochlorobenzene, silica-filtered, and recrystallized, obtaining 14.4 g (48%) of Intermediate int-26.

5th step: Synthesis of Compound C-2

Intermediate int-26 (14.1 g, 39.4 mmol), Intermediate int-25 (18.5 g, 14.4 mmol), K₂CO₃ (10.9 g, 78.8 mmol), and Pd(PPh₃)₄ (2.3 g, 2.0 mmol) were added to a round-bottomed flask, dissolved in 200 ml of THF and 40 ml of distilled water, and then, stirred under reflux at 70° C. for 8 hours. When a reaction was completed, after removing an aqueous layer, a solid extracted therein was filtered. The solid was dissolved by heating with monochlorobenzene, silica-filtered, and then, recrystallized, obtaining 18.2 g (72%) of Compound C-2.

### Comparative Synthesis Example 3: Synthesis of Compound C-3

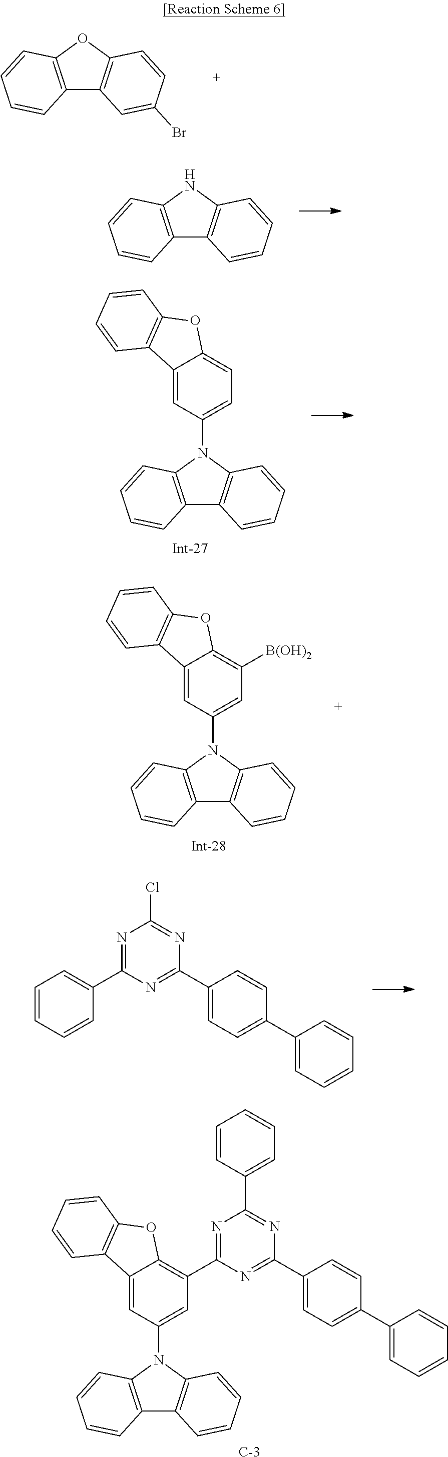

### 1st Step: Synthesis of Intermediate Int-27

2-bromo-4-chlorodibenzofuran (30.3 g, 107.6 mmol), 9H-carbazole (18.0 g, 107.6 mmol), sodium tert-butoxide (20.7 g, 215.3 mmol), tri-tert-butylphosphine (2.2 g, 10.8 mmol), and Pd₂(dba)₃ (4.9 g, 5.4 mmol) were added to a round-bottomed flask, dissolved in 550 ml of xylene, and then, stirred under reflux at 150° C. for 8 hours. When a reaction was completed, after removing a salt by filtering, a filtrate therefrom was adsorbed. 21.4 g (54%) of Intermediate int-27 was obtained by using column chromatography (hexane:DCM (40%)).

### 2nd Step: Synthesis of Intermediate Int-28

Intermediate int-27 (21.2 g, 57.6 mmol), bis(pinacolato)diboron (17.6 g, 69.2 mmol), tricyclohexylphosphine (2.8 g, 11.5 mmol), potassium acetate (11.3 g, 115.3 mmol), and Pd₂(dba)₃ (1.6 g, 1.7 mmol) were added to a round-bottomed flask and dissolved in 200 ml of xylene. The mixture was stirred under reflux at 160° C. for 8 hours. When a reaction was completed, the resultant was cooled to room temperature, filtered to remove a salt, and then, extracted by adding an excessive amount of DCM and distilled water thereto. 23.3 g (88%) of Intermediate int-28 was obtained by using column chromatography (hexane:DCM (40%)).

### 3rd Step: Synthesis of Compound C-3

2-chloro-4-(biphenyl-4-yl)-6-phenyl-1,3,5-triazine (13.5 g, 39.3 mmol), Intermediate int-28 (18.9 g, 41.2 mmol), K₂CO₃ (10.9 g, 78.5 mmol), and Pd(PPh₃)₄ (2.3 g, 2.0 mmol) were added to a round-bottomed flask, dissolved in 150 ml of THF and 40 ml of distilled water, and then, stirred under reflux at 70° C. for 12 hours. When reaction was completed, a solid was separated therefrom by filtering and recrystallized with monochlorobenzene, to obtain 18.1 g (72%) of Compound C-3.

### Comparative Synthesis Example 4: Synthesis of Compound C-4

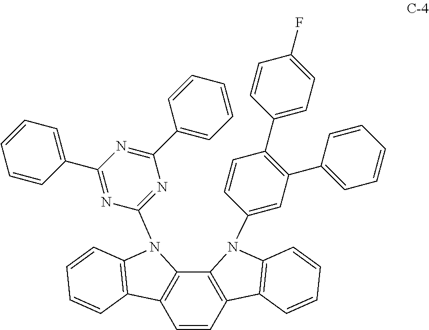 [SMILES: Fc1ccc(-c2ccc(-n3c4ccccc4c4ccc5c6ccccc6n(-c6nc(-c7ccccc7)nc(-c7ccccc7)n6)c5c43)cc2-c2ccccc2)cc1]

Compound C-4 was synthesized by referring to the synthetic method known in published patent No. CN 114075204 A.

### Comparative Synthesis Example 5: Synthesis of Compound C-5

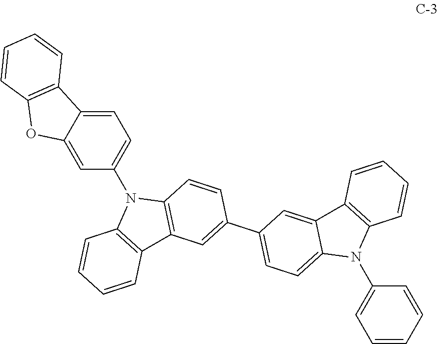 [SMILES: c1ccc(-n2c3ccccc3c3cc(-c4ccc5c(c4)c4ccccc4n5-c4ccc5c(c4)oc4ccccc45)ccc32)cc1]

Compound C-5 was synthesized by referring to the synthetic method known in registered patent No. KR 2322795 B1.

### Comparative Synthesis Example 6: Synthesis of Compound C-6

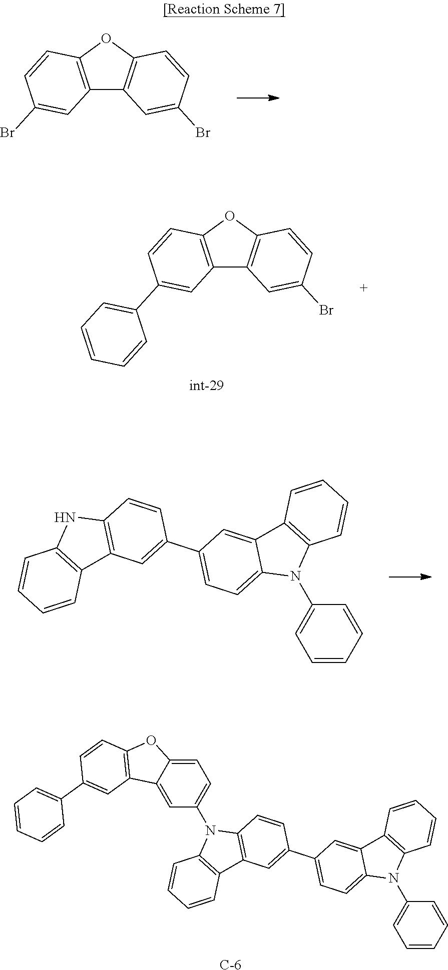

### 1st Step: Synthesis of Intermediate Int-29

Intermediate int-29 was synthesized by referring to the synthetic method disclosed in registered patent No. KR 1862881 B1.

2nd step: Synthesis of Compound C-6

Compound C-6 was synthesized using the same method as 4th step of Synthesis Example 4.

### Comparative Synthesis Example 7: Synthesis of Compound C-7

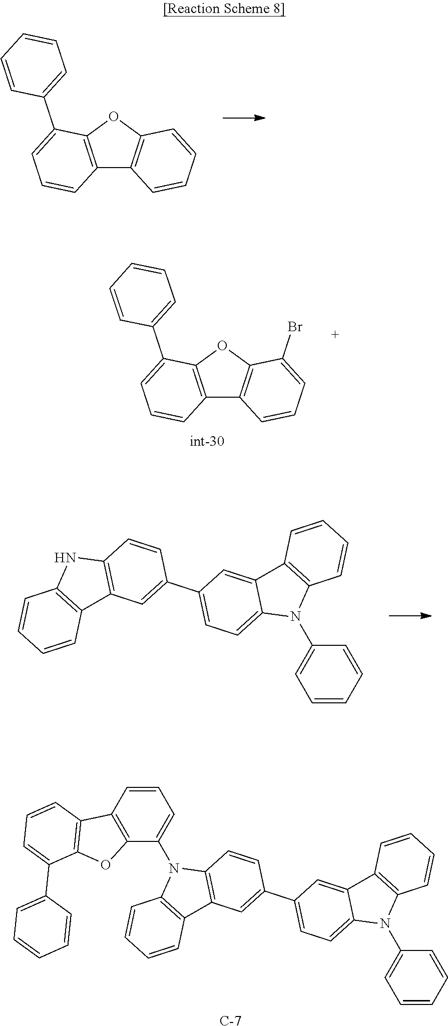

### 1st Step: Synthesis of Intermediate Int-30

Intermediate int-30 was synthesized by referring to the synthetic method disclosed in published patent application WO 2017-100967 A1.

2nd step: Synthesis of Compound C-7

Compound C-7 was synthesized using the same method as 4th step of Synthesis Example 4.

### Comparative Synthesis Example 8: Synthesis of Compound C-8

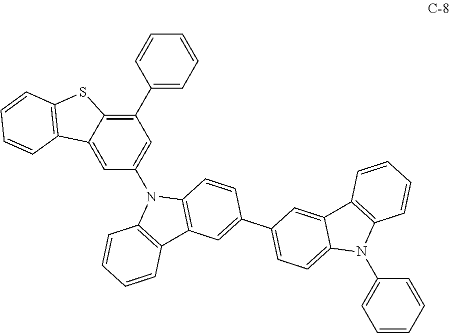 [SMILES: c1ccc(-c2cc(-n3c4ccccc4c4cc(-c5ccc6c(c5)c5ccccc5n6-c5ccccc5)ccc43)cc3c2sc2ccccc23)cc1]

Compound C-8 was synthesized by referring to the synthetic method known in registered patent KR 2290362 B1.

### Comparative Synthesis Example 9: Synthesis of Compound C-9

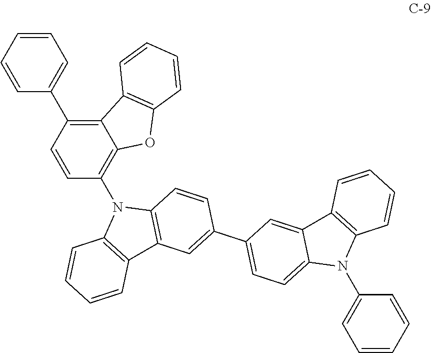 [SMILES: c1ccc(-c2ccc(-n3c4ccccc4c4cc(-c5ccc6c(c5)c5ccccc5n6-c5ccccc5)ccc43)c3oc4ccccc4c23)cc1]

Compound C-9 was synthesized by referring to the synthetic method known in registered patent No. KR 2247294 B1.

### Comparative Synthesis Example 10: Synthesis of Compound C-10

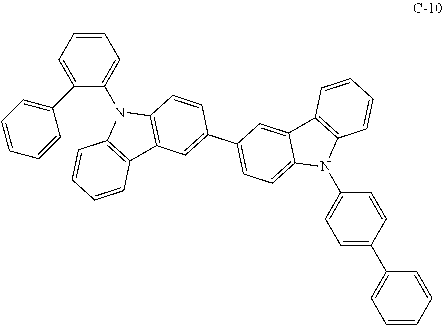 [SMILES: c1ccc(-c2ccc(-n3c4ccccc4c4cc(-c5ccc6c(c5)c5ccccc5n6-c5ccccc5-c5ccccc5)ccc43)cc2)cc1]

Compound C-10 was synthesized by referring to the synthetic method known in published patent application KR 2021-0152819.

### (Manufacturing of Organic Light Emitting Diode)

### Example 1

A glass substrate coated with ITO (Indium tin oxide) was washed with distilled water and ultrasonic waves. After washing with the distilled water, the glass substrate was washed with a solvent such as isopropyl alcohol, acetone, methanol, and the like ultrasonically and dried and then, moved to a plasma cleaner, cleaned by using oxygen plasma for 10 minutes, and moved to a vacuum depositor. This obtained ITO transparent electrode was used as an anode, Compound A doped with 3% NDP-9 (available from Novaled) was vacuum-deposited on the ITO substrate to form a 100 Å-thick hole injection layer, and Compound A was deposited to on the hole injection layer to form a 1350 Å-thick hole transport layer. Compound B was deposited on the hole transport layer to a thickness of 350 Å to form a hole transport auxiliary layer. Compound A-1 synthesized in Synthesis Example 1 and Compound B-36 synthesized in Synthesis Example 4 were simultaneously used in a weight ratio of 3:7 as a host on the hole transport auxiliary layer, and PhGD was doped at 10 wt % as a dopant to form a 380 Å-thick light emitting layer by vacuum deposition. Next, Compound C was deposited on the light emitting layer to form a 50 Å-thick electron transport auxiliary layer, and Compound D and Liq were simultaneously vacuum deposited at a weight ratio of 1:1 to form a 300 Å-thick electron transport layer. LiQ (15 Å) and Al (1200 Å) were sequentially vacuum-deposited on the electron transport layer to form a cathode, thereby manufacturing an organic light emitting diode.

The organic light emitting diode has a structure of ITO/Compound A (3% NDP-9 doping, 100 Å)/Compound A (1350 Å)/Compound B (350 Å)/EML [90 wt % of host (Compound A-1: Compound B-36=3:7 w/w): 10 wt % of PhGD] (380 Å)/Compound C (50 Å)/Compound D:LiQ (300 Å)/LiQ (15 Å)/Al (1200 Å).
Compound A: N-(biphenyl-4-yl)-9,9-dimethyl-N-(4-(9-phenyl-9H-carbazol-3-yl)phenyl)-9H-fluoren-2-amine
Compound B: N,N-bis(9,9-dimethyl-9H-fluoren-4-yl)-9,9-spirobi(fluorene)-2-amine
Compound C: 2-[3′-(9,9-Dimethyl-9H-fluoren-2-yl)[1,1′-biphenyl]-3-yl]-4,6-diphenyl-1,3,5-triazine
Compound D: 2-(Biphenyl-4-yl)-4-(9,9-diphenyl-9H-fluoren-4-yl)-6-phenyl-1,3,5-triazine

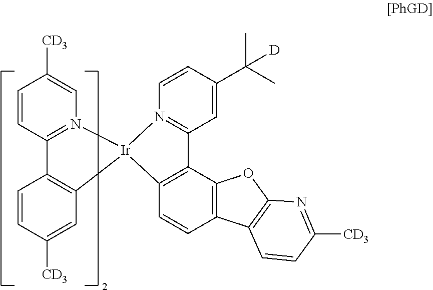

### Examples 2 to 4 and Comparative Examples 1 to 10

Diodes of Examples 2 to 4 and Comparative Examples 1 to 10 were manufactured in the same manner as in Example 1, except that the host was changed as described in Table 1.

### Evaluation

### (1) Measurement of Current Density Change Depending on Voltage Change

The obtained organic light emitting diodes were measured regarding a current value flowing in the unit diode, while increasing the voltage from 0 V to 10 V using a current-voltage meter (Keithley 2400), and the measured current value was divided by area to provide the results.

### (2) Measurement of Luminance Change Depending on Voltage Change

Luminance was measured by using a luminance meter (Minolta Cs-1000A), while the voltage of the organic light emitting diodes was increased from 0 V to 10 V.

### (3) Measurement of Luminous Efficiency

The luminous efficiency (cd/A) at the same current density (10 mA/cm²) was calculated using the luminance and current density measured from (1) and (2) above.

The luminous efficiency ratios of Examples 1 to 4 and Comparative Examples 1 to 10 were calculated as relative values based on Comparative Example 1 and are shown in Table 1.

### (4) Measurement of Driving Voltage

The results were obtained by measuring the driving voltage of each diode at 15 mA/cm using a current-voltage meter (Keithley 2400).

The driving voltage ratios of Examples 1 to 4 and Comparative Examples 1 to 10 were calculated as relative values based on Comparative Example 1 and are shown in Table 1.

TABLE 1

| | | | Driving | Luminous |
|---|---|---|---|---|
| | First | Second | voltage ratio | efficiency ratio |
| | host | host | (%) | (%) |
| Example 1 | A-1 | B-36 | 95 | 110 |
| Example 2 | A-1 | B-106 | 96 | 111 |
| Example 3 | A-27 | B-36 | 96 | 113 |
| Example 4 | A-27 | B-106 | 97 | 114 |
| Comparative | C-1 | B-36 | 100 | 100 |
| Example 1 | | | | |
| Comparative | C-2 | B-36 | 105 | 90 |
| Example 2 | | | | |
| Comparative | C-3 | B-106 | 104 | 98 |
| Example 3 | | | | |
| Comparative | C-4 | B-106 | 107 | 94 |
| Example 4 | | | | |
| Comparative | A-1 | C-5 | 99 | 93 |
| Example 5 | | | | |
| Comparative | A-1 | C-6 | 98 | 98 |
| Example 6 | | | | |
| Comparative | A-1 | C-7 | 100 | 95 |
| Example 7 | | | | |
| Comparative | A-1 | C-8 | 105 | 94 |
| Example 8 | | | | |
| Comparative | A-1 | C-9 | 105 | 94 |
| Example 9 | | | | |
| Comparative | A-1 | C-10 | 109 | 93 |
| Example 10 | | | | |

Referring to Table 1, the organic light emitting diodes according to Examples 1 to 4 have significantly improved luminous efficiency while maintaining an equivalent or higher driving voltage compared to the organic light emitting diodes according to Comparative Examples 1 to 10.

While this invention has been described in connection with what is presently considered to be practical embodiments, it is to be understood that the invention is not limited to the disclosed embodiments, but, on the contrary, is intended to cover various modifications and equivalent arrangements included within the spirit and scope of the appended claims.

1. A composition for an organic optoelectronic device, comprising
a first compound represented by Chemical Formula 1; and
a second compound represented by Chemical Formula 2: 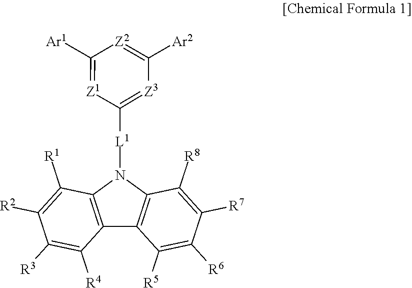
wherein, in Chemical Formula 1,
Z¹ to Z³ are each independently N or C—R^{a},
at least two of Z¹ to Z³ are N,
L¹ is a single bond or a substituted or unsubstituted C6 to C20 arylene group,
Ar¹ and Ar² are each independently a substituted or unsubstituted C6 to C30 aryl group, and
R^{a}, and R¹ to R⁸ are each independently hydrogen, deuterium, a substituted or unsubstituted C1 to C10 alkyl group, a substituted or unsubstituted C6 to C30 aryl group, a substituted or unsubstituted C2 to C30 heterocyclic group, or a combination thereof, 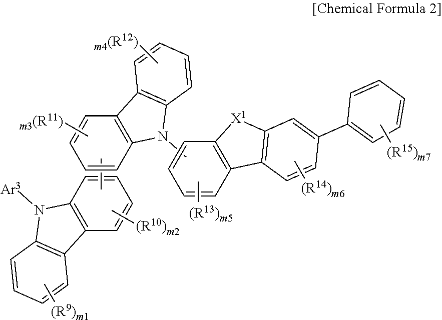
wherein, in Chemical Formula 2,
X¹ is O or S,
Ar³ is a substituted or unsubstituted C6 to C30 aryl group, a substituted or unsubstituted C2 to C30 heterocyclic group, or a combination thereof,
R⁹ to R¹² are each independently hydrogen, deuterium, a cyano group, a halogen, a substituted or unsubstituted amine group, a substituted or unsubstituted C1 to C30 alkyl group, a substituted or unsubstituted silyl group, a substituted or unsubstituted C6 to C30 aryl group, or a substituted or unsubstituted C2 to C30 heterocyclic group,
R¹³ to R¹⁵ are each independently hydrogen, deuterium, or substituted or unsubstituted phenyl group,
m2, m3, m5, and m6 are each independently one of integers of 1 to 3,
m1 and m4 are each independently one of integers of 1 to 4, and
m7 is one of integers of 1 to 5.

2. The composition for the organic optoelectronic device as claimed in claim 1, wherein
in Chemical Formula 1, Ar¹ and Ar² are each independently a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, or a substituted or unsubstituted fluorenyl group.

3. The composition for the organic optoelectronic device as claimed in claim 1, wherein
in Chemical Formula 1, R¹ to R⁸ are each independently hydrogen, deuterium, a cyano group, a substituted or unsubstituted C6 to C20 aryl group, or a substituted or unsubstituted C2 to C20 heterocyclic group.

4. The composition for the organic optoelectronic device as claimed in claim 1, wherein
the second compound is represented by any one of Chemical Formula 2-1 to Chemical Formula 2-4: 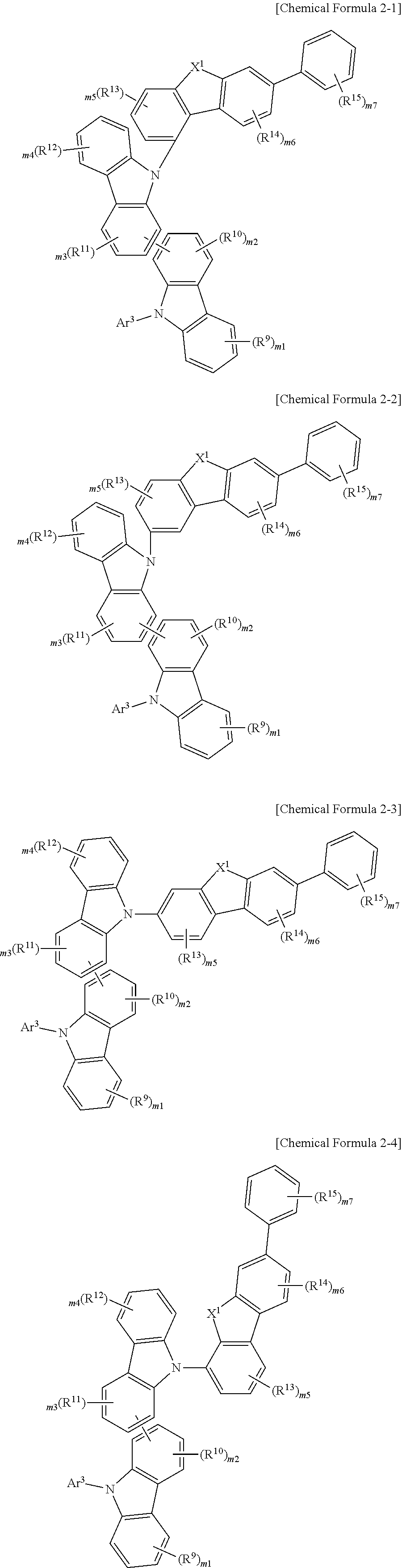
wherein, in Chemical Formula 2-1 to Chemical Formula 2-4, X¹, Ar³, R⁹ to R¹⁵, and m1 to m7 are defined the same as those of Chemical Formula 2.

5. The composition for the organic optoelectronic device as claimed in claim 4, wherein
the second compound is represented by any one of Chemical Formula 2-1-11, Chemical Formula 2-2-11, Chemical Formula 2-3-11, and Chemical Formula 2-4-11: 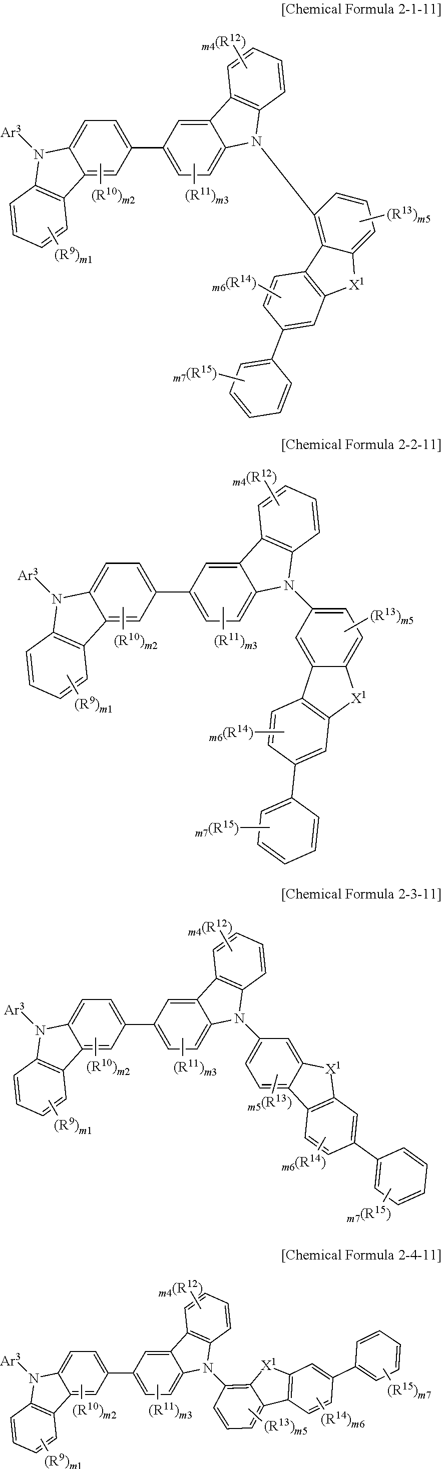
wherein, in Chemical Formula 2-1-11, Chemical Formula 2-2-11, Chemical Formula 2-3-11, and Chemical Formula 2-4-11, X¹, Ar³, R⁹ to R¹⁵, and m1 to m7 are defined the same as those of Chemical Formula 2.

6. The composition for the organic optoelectronic device as claimed in claim 1, wherein
in Chemical Formula 2, Ar³ is a substituted or unsubstituted phenyl group, a substituted or unsubstituted biphenyl group, a substituted or unsubstituted terphenyl group, a substituted or unsubstituted naphthyl group, a substituted or unsubstituted anthracenyl group, a substituted or unsubstituted phenanthrenyl group, a substituted or unsubstituted triphenylene group, or a substituted or unsubstituted fluorenyl group.

7. The composition for the organic optoelectronic device as claimed in claim 1, wherein
the first compound is selected from compounds listed in Group 1, and
the second compound is selected from compounds listed in Group 2:

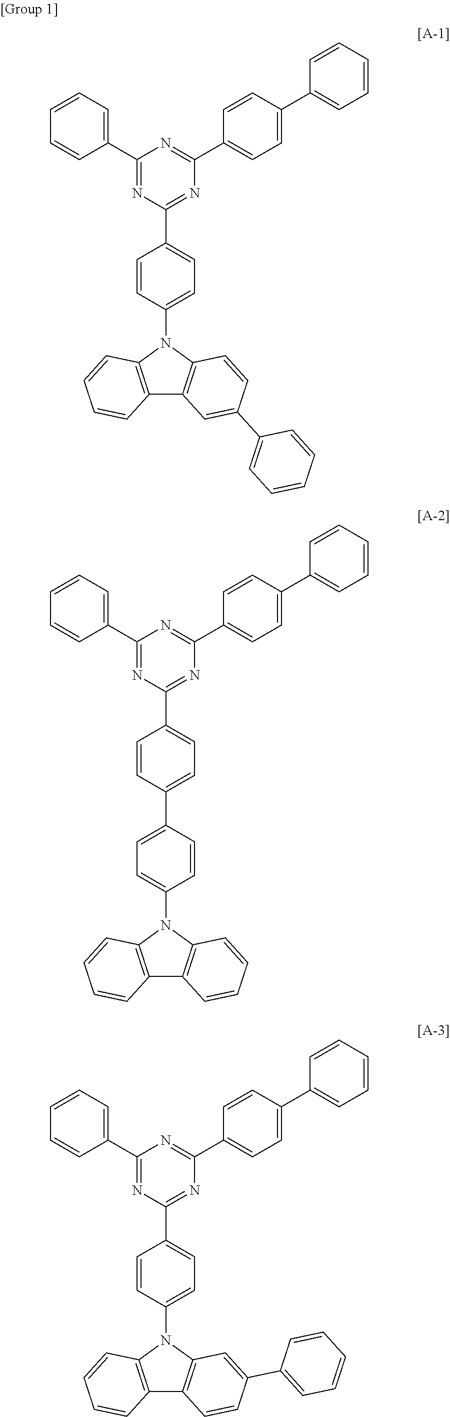 [SMILES: c1ccc(-c2ccc(-c3nc(-c4ccccc4)nc(-c4ccc(-n5c6ccccc6c6cc(-c7ccccc7)ccc65)cc4)n3)cc2)cc1 ; c1ccc(-c2ccc(-c3nc(-c4ccccc4)nc(-c4ccc(-c5ccc(-n6c7ccccc7c7ccccc76)cc5)cc4)n3)cc2)cc1 ; c1ccc(-c2ccc(-c3nc(-c4ccccc4)nc(-c4ccc(-n5c6ccccc6c6ccc(-c7ccccc7)cc65)cc4)n3)cc2)cc1]

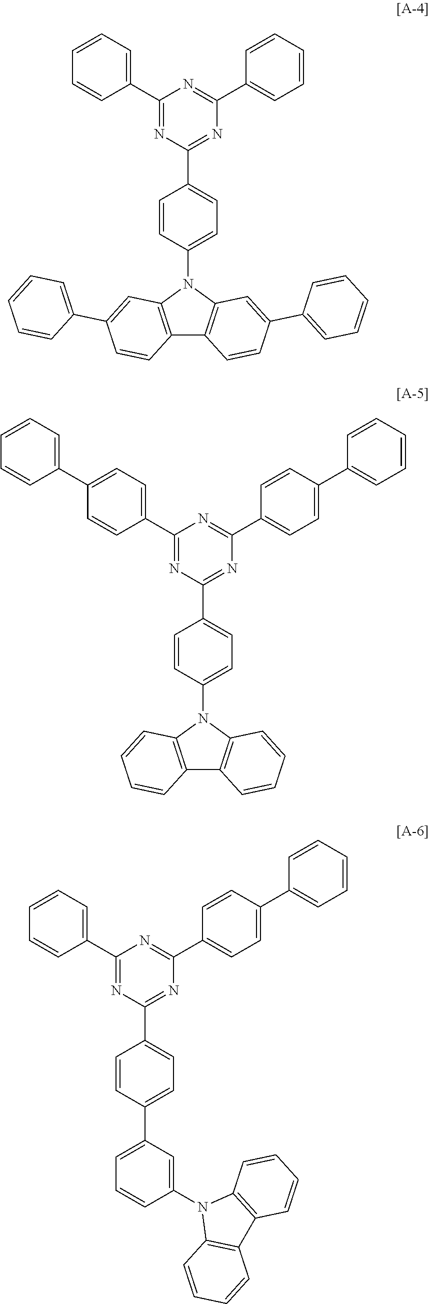 [SMILES: c1ccc(-c2ccc3c4ccc(-c5ccccc5)cc4n(-c4ccc(-c5nc(-c6ccccc6)nc(-c6ccccc6)n5)cc4)c3c2)cc1 ; c1ccc(-c2ccc(-c3nc(-c4ccc(-c5ccccc5)cc4)nc(-c4ccc(-n5c6ccccc6c6ccccc65)cc4)n3)cc2)cc1 ; c1ccc(-c2ccc(-c3nc(-c4ccccc4)nc(-c4ccc(-c5cccc(-n6c7ccccc7c7ccccc76)c5)cc4)n3)cc2)cc1]

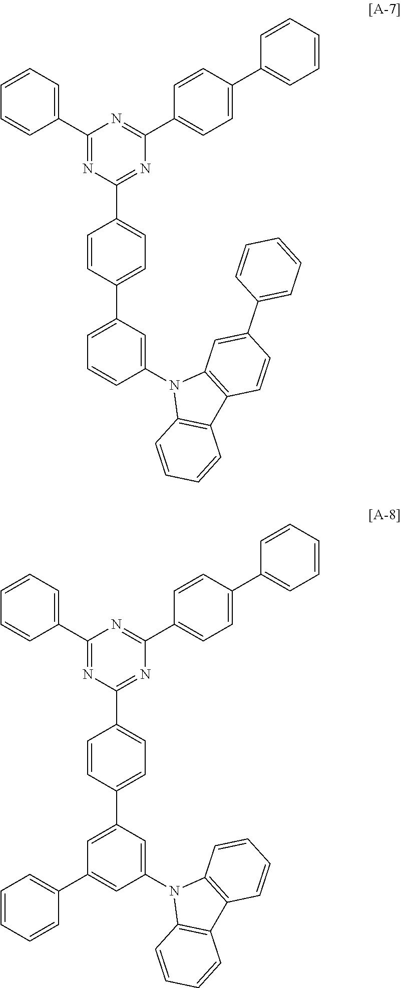 [SMILES: c1ccc(-c2ccc(-c3nc(-c4ccccc4)nc(-c4ccc(-c5cccc(-n6c7ccccc7c7ccc(-c8ccccc8)cc76)c5)cc4)n3)cc2)cc1 ; c1ccc(-c2ccc(-c3nc(-c4ccccc4)nc(-c4ccc(-c5cc(-c6ccccc6)cc(-n6c7ccccc7c7ccccc76)c5)cc4)n3)cc2)cc1]

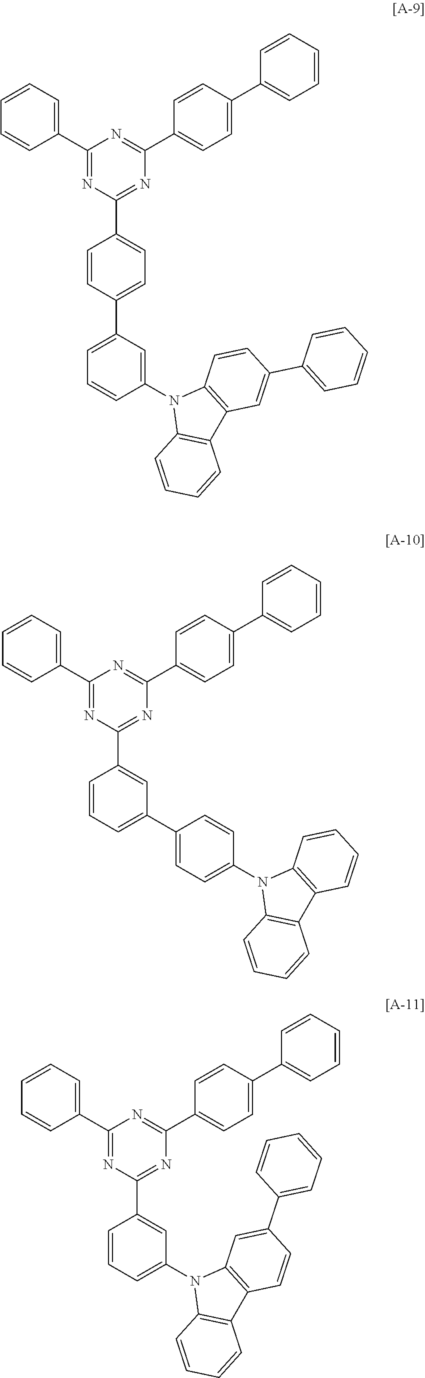

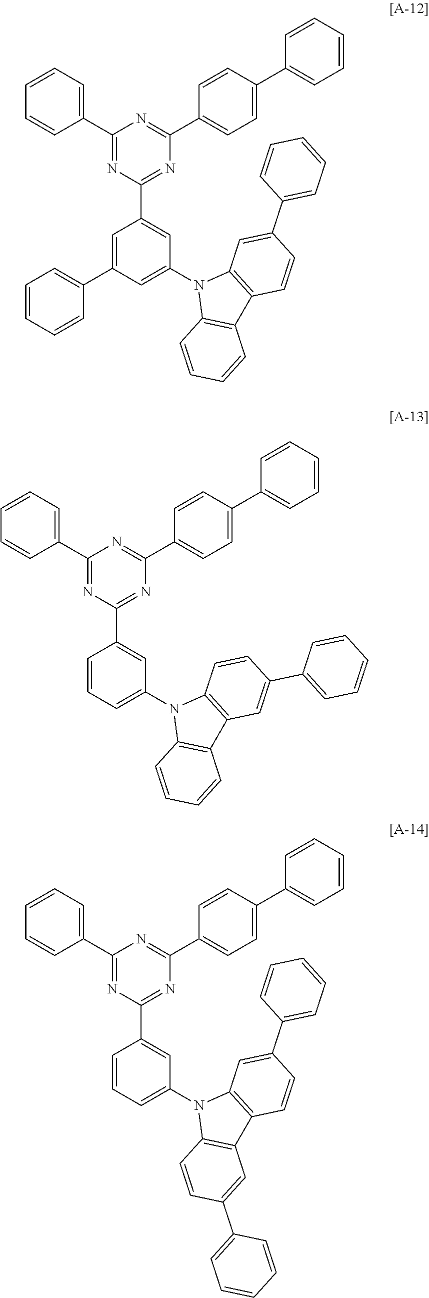 [SMILES: c1ccc(-c2ccc(-c3nc(-c4ccccc4)nc(-c4cc(-c5ccccc5)cc(-n5c6ccccc6c6ccc(-c7ccccc7)cc65)c4)n3)cc2)cc1 ; c1ccc(-c2ccc(-c3nc(-c4ccccc4)nc(-c4cccc(-n5c6ccccc6c6cc(-c7ccccc7)ccc65)c4)n3)cc2)cc1 ; c1ccc(-c2ccc(-c3nc(-c4ccccc4)nc(-c4cccc(-n5c6ccc(-c7ccccc7)cc6c6ccc(-c7ccccc7)cc65)c4)n3)cc2)cc1]

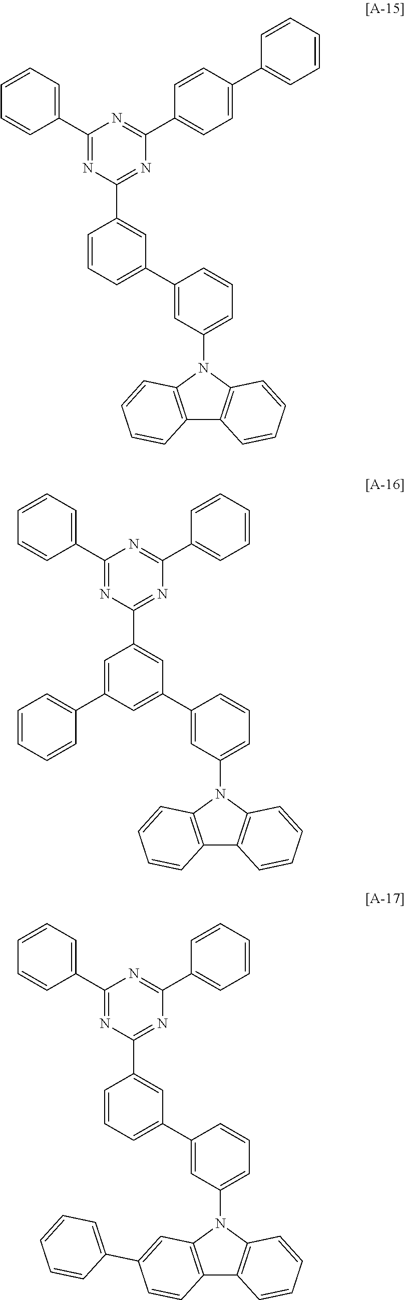 [SMILES: c1ccc(-c2ccc(-c3nc(-c4ccccc4)nc(-c4cccc(-c5cccc(-n6c7ccccc7c7ccccc76)c5)c4)n3)cc2)cc1 ; c1ccc(-c2cc(-c3cccc(-n4c5ccccc5c5ccccc54)c3)cc(-c3nc(-c4ccccc4)nc(-c4ccccc4)n3)c2)cc1 ; c1ccc(-c2ccc3c4ccccc4n(-c4cccc(-c5cccc(-c6nc(-c7ccccc7)nc(-c7ccccc7)n6)c5)c4)c3c2)cc1]

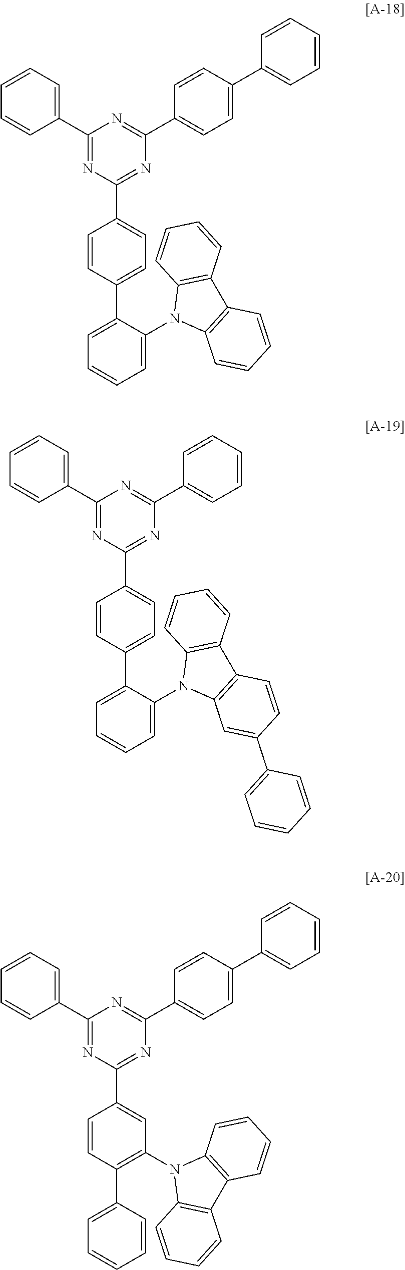 [SMILES: c1ccc(-c2ccc(-c3nc(-c4ccccc4)nc(-c4ccc(-c5ccccc5-n5c6ccccc6c6ccccc65)cc4)n3)cc2)cc1 ; c1ccc(-c2ccc3c4ccccc4n(-c4ccccc4-c4ccc(-c5nc(-c6ccccc6)nc(-c6ccccc6)n5)cc4)c3c2)cc1 ; c1ccc(-c2ccc(-c3nc(-c4ccccc4)nc(-c4ccc(-c5ccccc5)c(-n5c6ccccc6c6ccccc65)c4)n3)cc2)cc1]

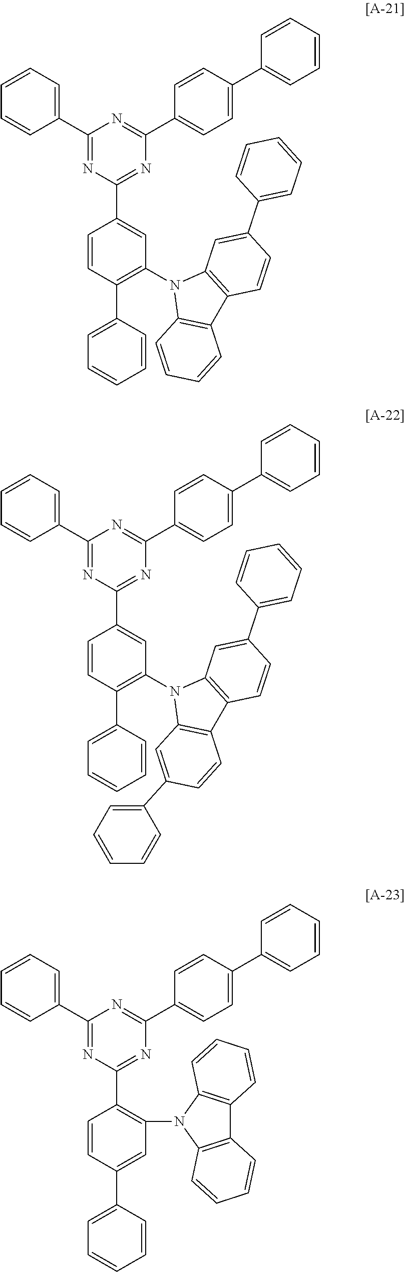 [SMILES: c1ccc(-c2ccc(-c3nc(-c4ccccc4)nc(-c4ccc(-c5ccccc5)c(-n5c6ccccc6c6ccc(-c7ccccc7)cc65)c4)n3)cc2)cc1 ; c1ccc(-c2ccc(-c3nc(-c4ccccc4)nc(-c4ccc(-c5ccccc5)c(-n5c6cc(-c7ccccc7)ccc6c6ccc(-c7ccccc7)cc65)c4)n3)cc2)cc1 ; c1ccc(-c2ccc(-c3nc(-c4ccccc4)nc(-c4ccc(-c5ccccc5)cc4-n4c5ccccc5c5ccccc54)n3)cc2)cc1]

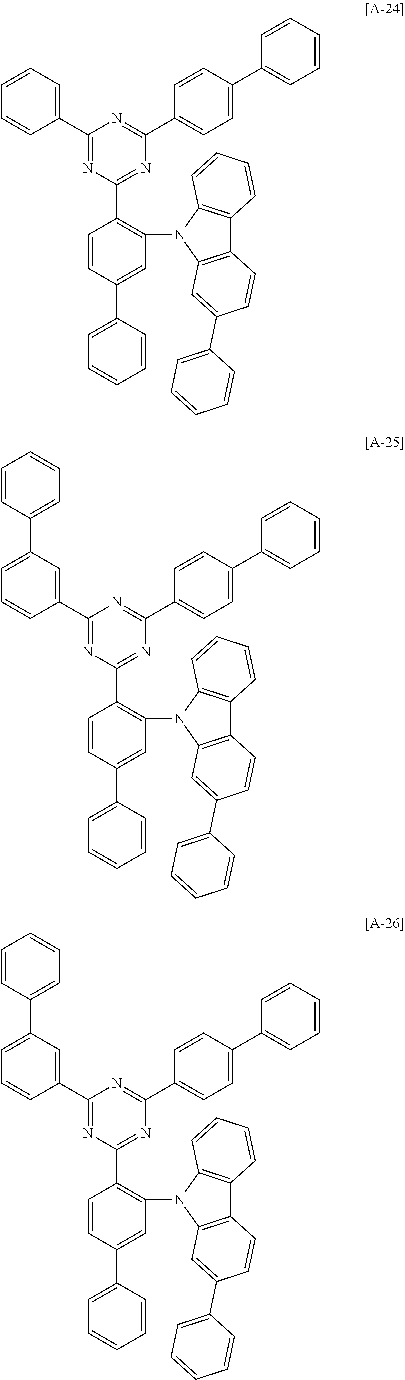 [SMILES: c1ccc(-c2ccc(-c3nc(-c4ccccc4)nc(-c4ccc(-c5ccccc5)cc4-n4c5ccccc5c5ccc(-c6ccccc6)cc54)n3)cc2)cc1 ; c1ccc(-c2ccc(-c3nc(-c4cccc(-c5ccccc5)c4)nc(-c4ccc(-c5ccccc5)cc4-n4c5ccccc5c5ccc(-c6ccccc6)cc54)n3)cc2)cc1 ; c1ccc(-c2ccc(-c3nc(-c4cccc(-c5ccccc5)c4)nc(-c4ccc(-c5ccccc5)cc4-n4c5ccccc5c5ccc(-c6ccccc6)cc54)n3)cc2)cc1]

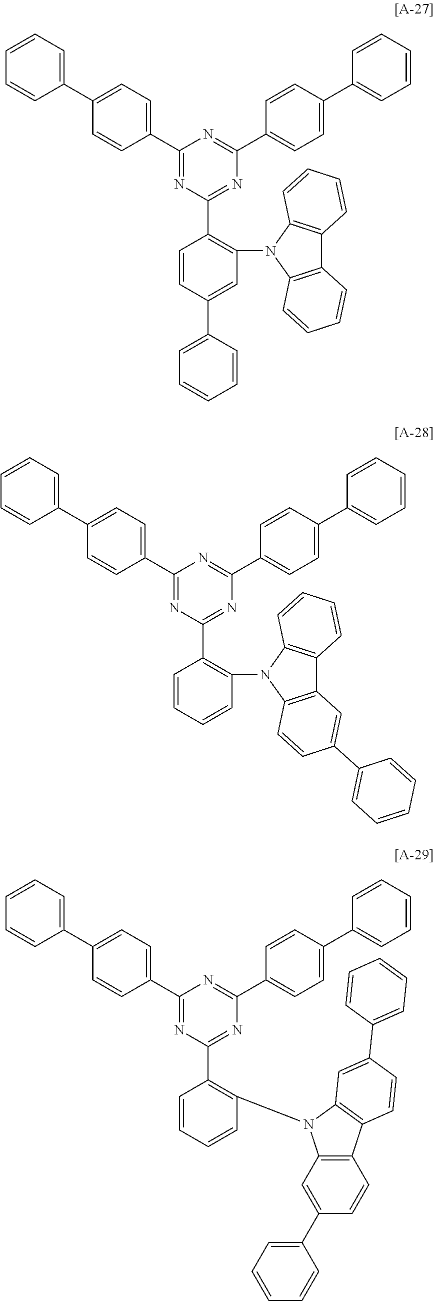 [SMILES: c1ccc(-c2ccc(-c3nc(-c4ccc(-c5ccccc5)cc4)nc(-c4ccc(-c5ccccc5)cc4-n4c5ccccc5c5ccccc54)n3)cc2)cc1 ; c1ccc(-c2ccc(-c3nc(-c4ccc(-c5ccccc5)cc4)nc(-c4ccccc4-n4c5ccccc5c5cc(-c6ccccc6)ccc54)n3)cc2)cc1 ; c1ccc(-c2ccc(-c3nc(-c4ccc(-c5ccccc5)cc4)nc(-c4ccccc4-n4c5cc(-c6ccccc6)ccc5c5ccc(-c6ccccc6)cc54)n3)cc2)cc1]

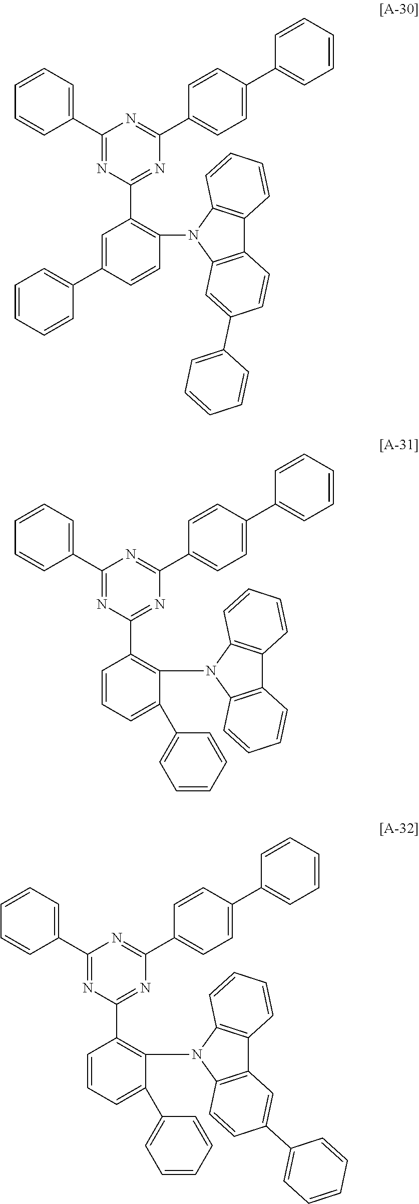 [SMILES: c1ccc(-c2ccc(-c3nc(-c4ccccc4)nc(-c4cc(-c5ccccc5)ccc4-n4c5ccccc5c5ccc(-c6ccccc6)cc54)n3)cc2)cc1 ; c1ccc(-c2ccc(-c3nc(-c4ccccc4)nc(-c4cccc(-c5ccccc5)c4-n4c5ccccc5c5ccccc54)n3)cc2)cc1 ; c1ccc(-c2ccc(-c3nc(-c4ccccc4)nc(-c4cccc(-c5ccccc5)c4-n4c5ccccc5c5cc(-c6ccccc6)ccc54)n3)cc2)cc1]

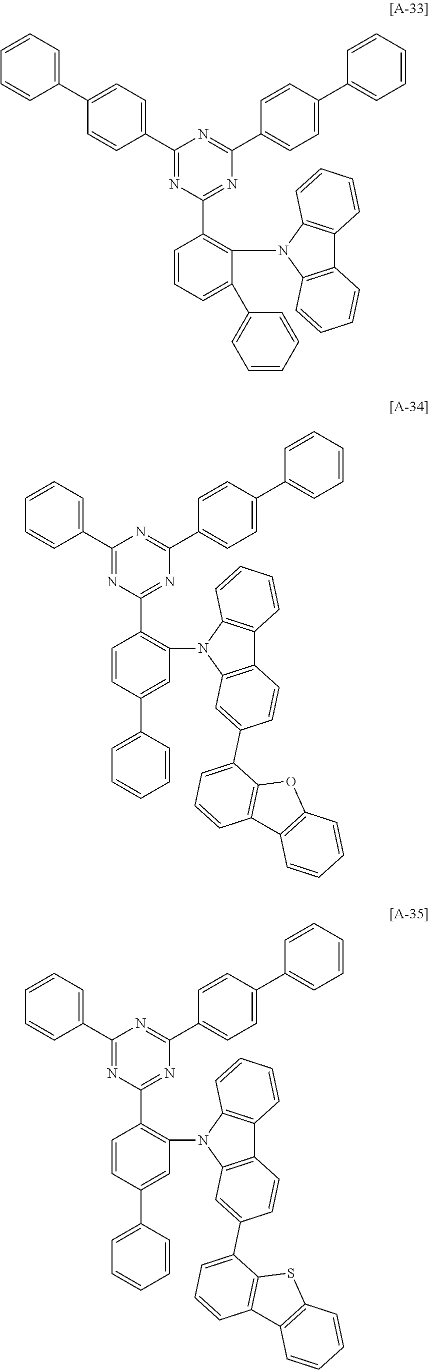

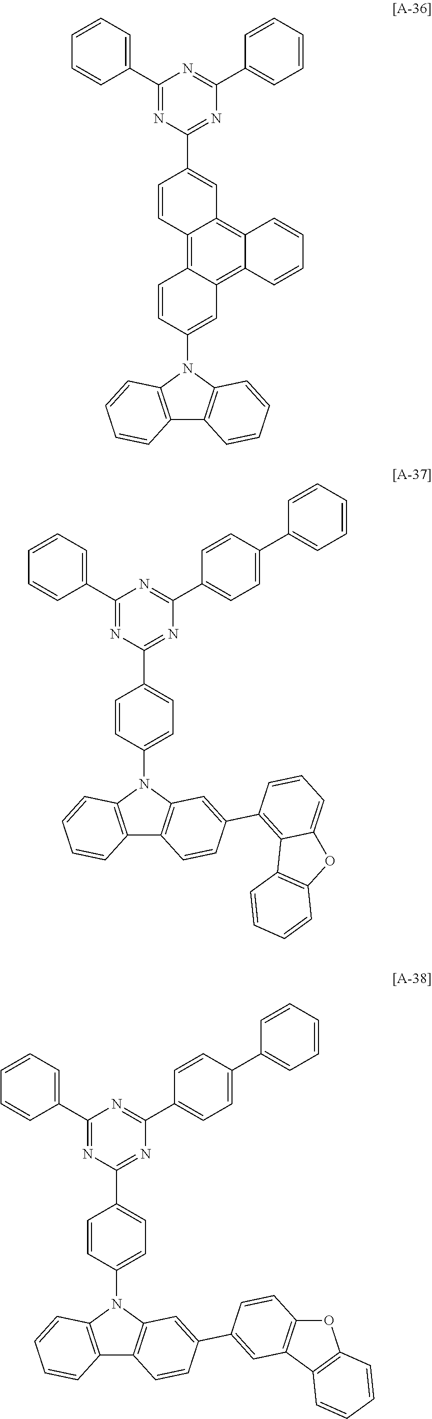 [SMILES: c1ccc(-c2nc(-c3ccccc3)nc(-c3ccc4c5ccc(-n6c7ccccc7c7ccccc76)cc5c5ccccc5c4c3)n2)cc1 ; c1ccc(-c2ccc(-c3nc(-c4ccccc4)nc(-c4ccc(-n5c6ccccc6c6ccc(-c7cccc8oc9ccccc9c78)cc65)cc4)n3)cc2)cc1 ; c1ccc(-c2ccc(-c3nc(-c4ccccc4)nc(-c4ccc(-n5c6ccccc6c6ccc(-c7ccc8oc9ccccc9c8c7)cc65)cc4)n3)cc2)cc1]

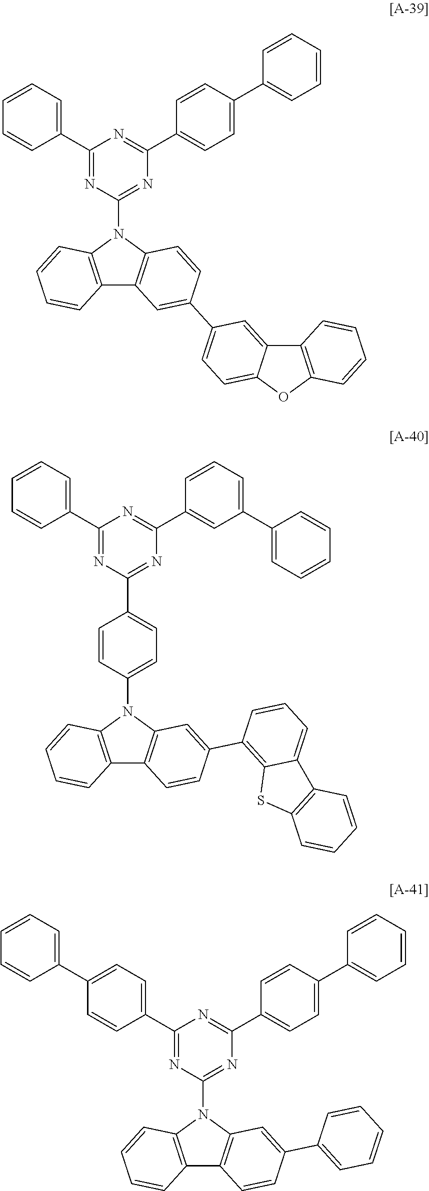 [SMILES: c1ccc(-c2ccc(-c3nc(-c4ccccc4)nc(-n4c5ccccc5c5cc(-c6ccc7oc8ccccc8c7c6)ccc54)n3)cc2)cc1 ; c1ccc(-c2cccc(-c3nc(-c4ccccc4)nc(-c4ccc(-n5c6ccccc6c6ccc(-c7cccc8c7sc7ccccc78)cc65)cc4)n3)c2)cc1 ; c1ccc(-c2ccc(-c3nc(-c4ccc(-c5ccccc5)cc4)nc(-n4c5ccccc5c5ccc(-c6ccccc6)cc54)n3)cc2)cc1]

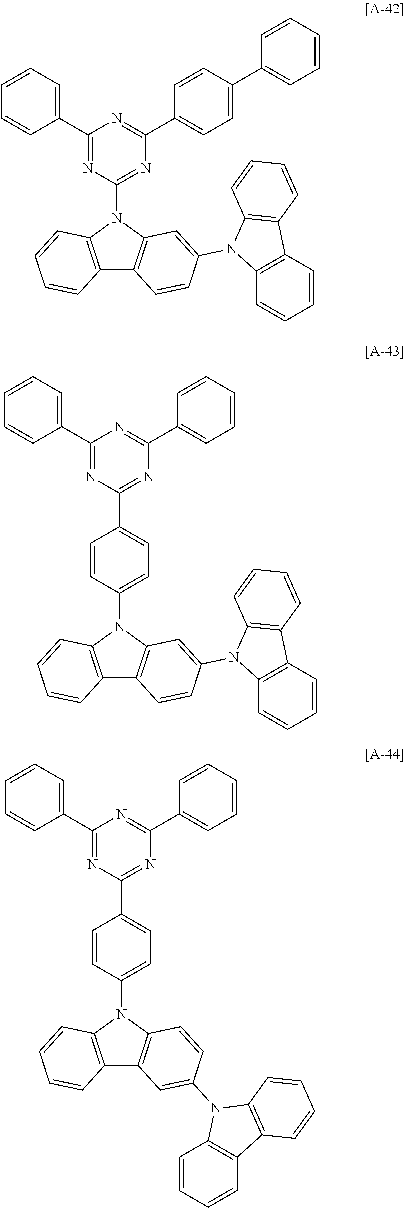 [SMILES: c1ccc(-c2ccc(-c3nc(-c4ccccc4)nc(-n4c5ccccc5c5ccc(-n6c7ccccc7c7ccccc76)cc54)n3)cc2)cc1 ; c1ccc(-c2nc(-c3ccccc3)nc(-c3ccc(-n4c5ccccc5c5ccc(-n6c7ccccc7c7ccccc76)cc54)cc3)n2)cc1 ; c1ccc(-c2nc(-c3ccccc3)nc(-c3ccc(-n4c5ccccc5c5cc(-n6c7ccccc7c7ccccc76)ccc54)cc3)n2)cc1]

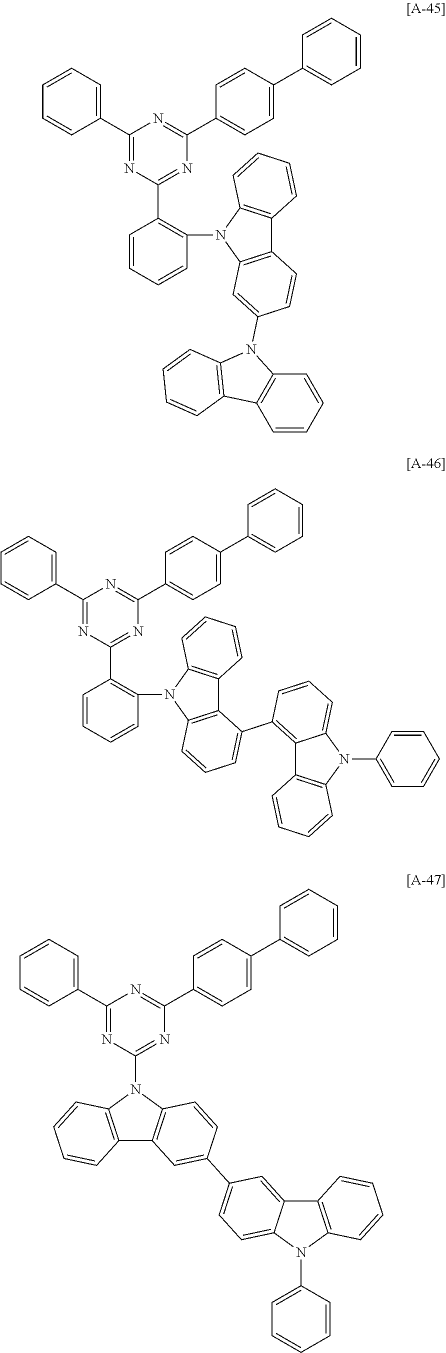 [SMILES: c1ccc(-c2ccc(-c3nc(-c4ccccc4)nc(-c4ccccc4-n4c5ccccc5c5ccc(-n6c7ccccc7c7ccccc76)cc54)n3)cc2)cc1 ; c1ccc(-c2ccc(-c3nc(-c4ccccc4)nc(-c4ccccc4-n4c5ccccc5c5c(-c6cccc7c6c6ccccc6n7-c6ccccc6)cccc54)n3)cc2)cc1 ; c1ccc(-c2ccc(-c3nc(-c4ccccc4)nc(-n4c5ccccc5c5cc(-c6ccc7c(c6)c6ccccc6n7-c6ccccc6)ccc54)n3)cc2)cc1]

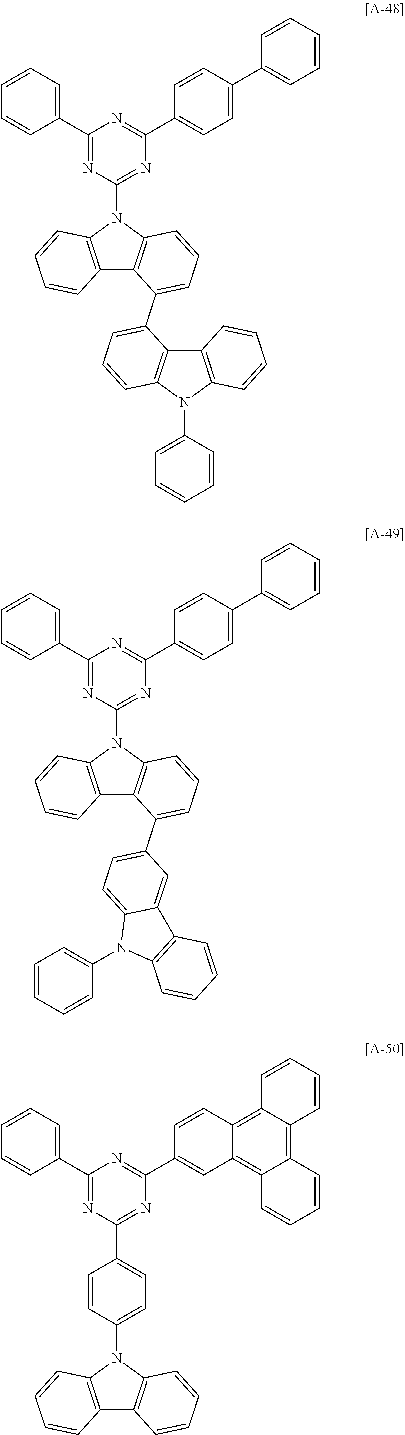 [SMILES: c1ccc(-c2ccc(-c3nc(-c4ccccc4)nc(-n4c5ccccc5c5c(-c6cccc7c6c6ccccc6n7-c6ccccc6)cccc54)n3)cc2)cc1 ; c1ccc(-c2ccc(-c3nc(-c4ccccc4)nc(-n4c5ccccc5c5c(-c6ccc7c(c6)c6ccccc6n7-c6ccccc6)cccc54)n3)cc2)cc1 ; c1ccc(-c2nc(-c3ccc(-n4c5ccccc5c5ccccc54)cc3)nc(-c3ccc4c5ccccc5c5ccccc5c4c3)n2)cc1]

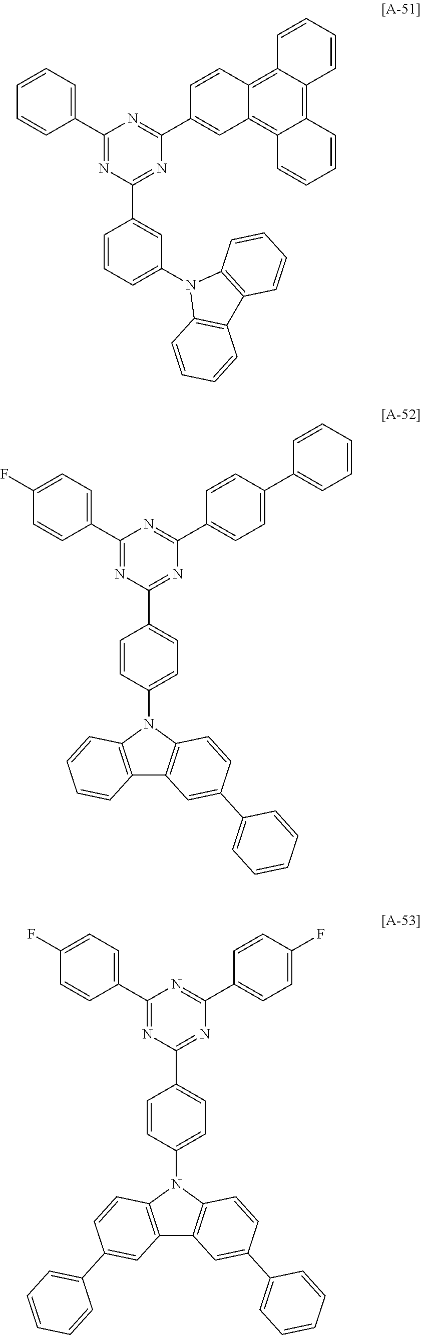 [SMILES: c1ccc(-c2nc(-c3cccc(-n4c5ccccc5c5ccccc54)c3)nc(-c3ccc4c5ccccc5c5ccccc5c4c3)n2)cc1 ; Fc1ccc(-c2nc(-c3ccc(-c4ccccc4)cc3)nc(-c3ccc(-n4c5ccccc5c5cc(-c6ccccc6)ccc54)cc3)n2)cc1 ; Fc1ccc(-c2nc(-c3ccc(F)cc3)nc(-c3ccc(-n4c5ccc(-c6ccccc6)cc5c5cc(-c6ccccc6)ccc54)cc3)n2)cc1]

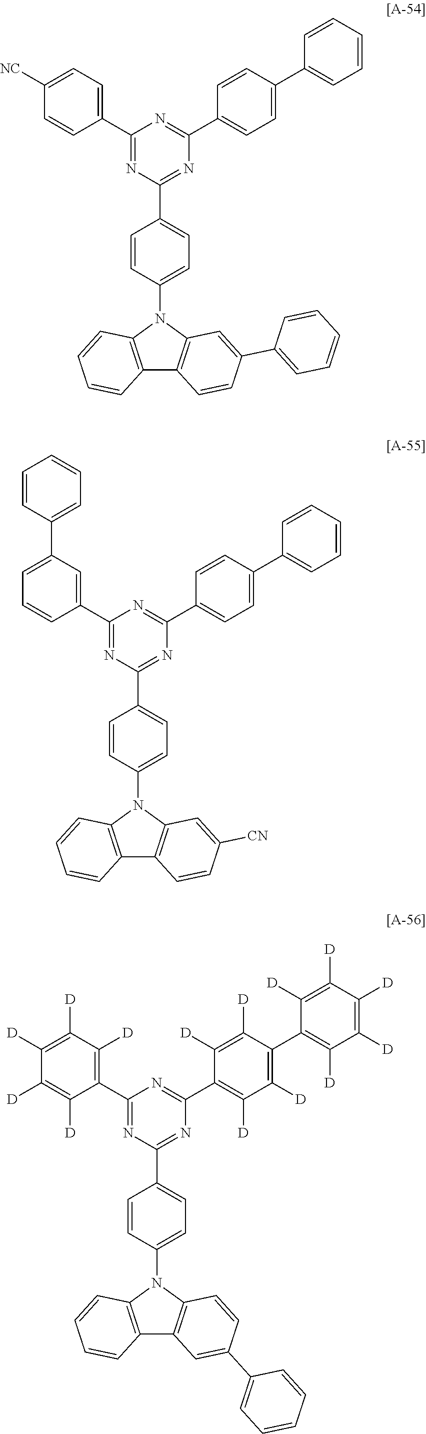 [SMILES: N#Cc1ccc(-c2nc(-c3ccc(-c4ccccc4)cc3)nc(-c3ccc(-n4c5ccccc5c5ccc(-c6ccccc6)cc54)cc3)n2)cc1 ; N#Cc1ccc2c3ccccc3n(-c3ccc(-c4nc(-c5ccc(-c6ccccc6)cc5)nc(-c5cccc(-c6ccccc6)c5)n4)cc3)c2c1 ; [2H]c1c([2H])c([2H])c(-c2nc(-c3ccc(-n4c5ccccc5c5cc(-c6ccccc6)ccc54)cc3)nc(-c3c([2H])c([2H])c(-c4c([2H])c([2H])c([2H])c([2H])c4[2H])c([2H])c3[2H])n2)c([2H])c1[2H]]

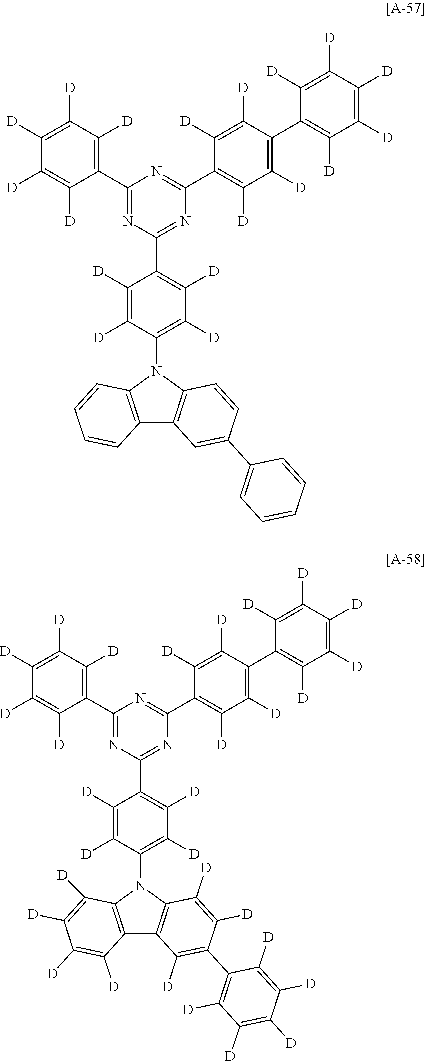 [SMILES: [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c(-n4c5ccccc5c5cc(-c6ccccc6)ccc54)c([2H])c3[2H])n2)c([2H])c1[2H] ; [2H]c1c([2H])c([2H])c(-c2nc(-c3c([2H])c([2H])c(-c4c([2H])c([2H])c([2H])c([2H])c4[2H])c([2H])c3[2H])nc(-c3c([2H])c([2H])c(-n4c5c([2H])c([2H])c([2H])c([2H])c5c5c([2H])c(-c6c([2H])c([2H])c([2H])c([2H])c6[2H])c([2H])c([2H])c54)c([2H])c3[2H])n2)c([2H])c1[2H]]

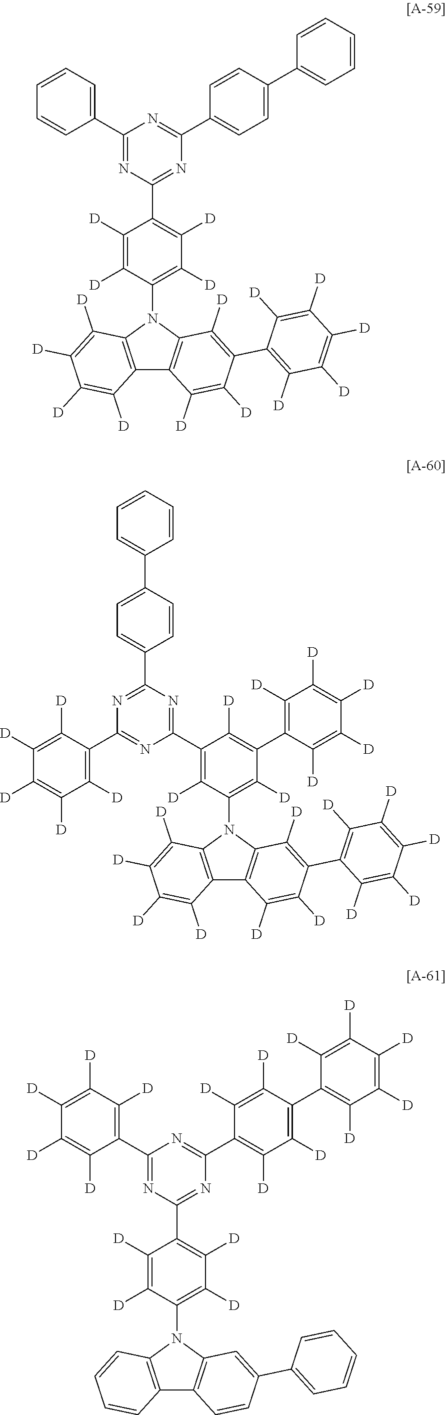

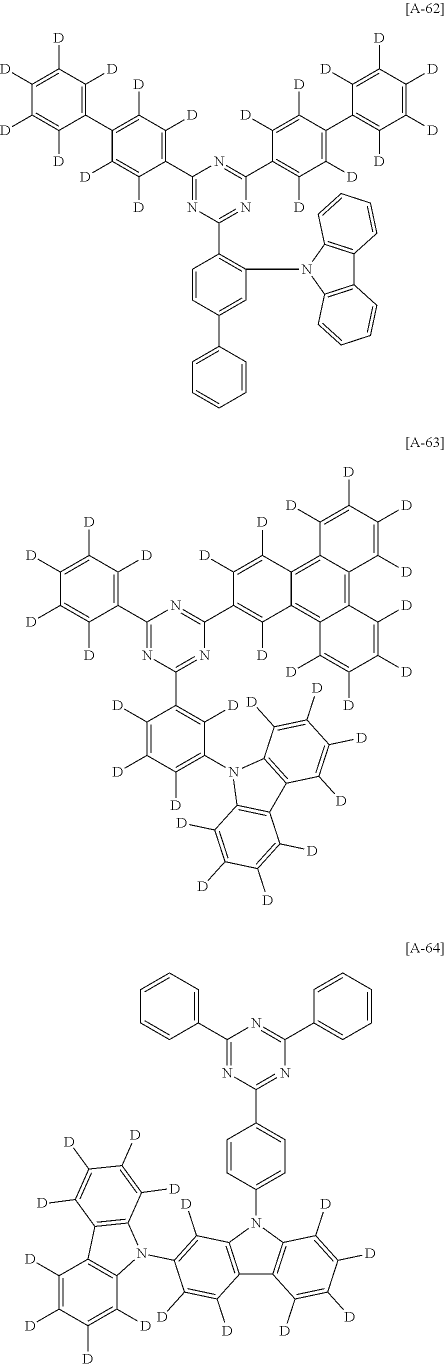

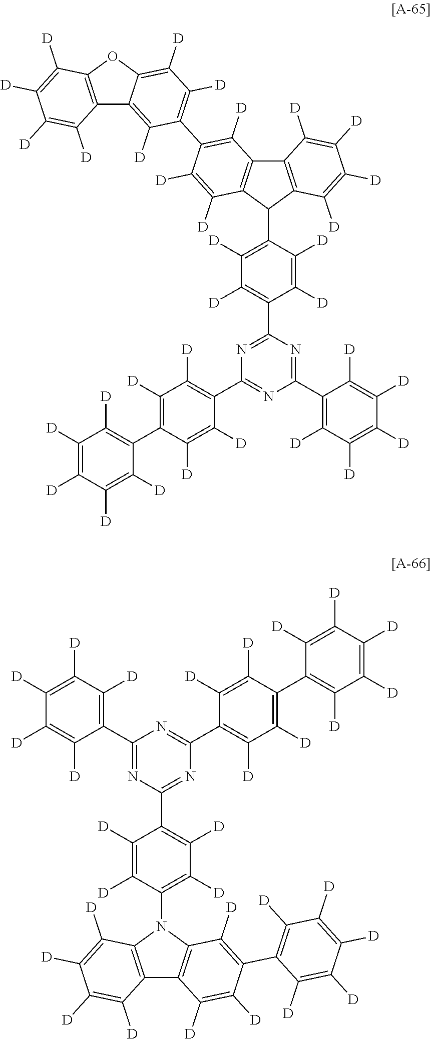

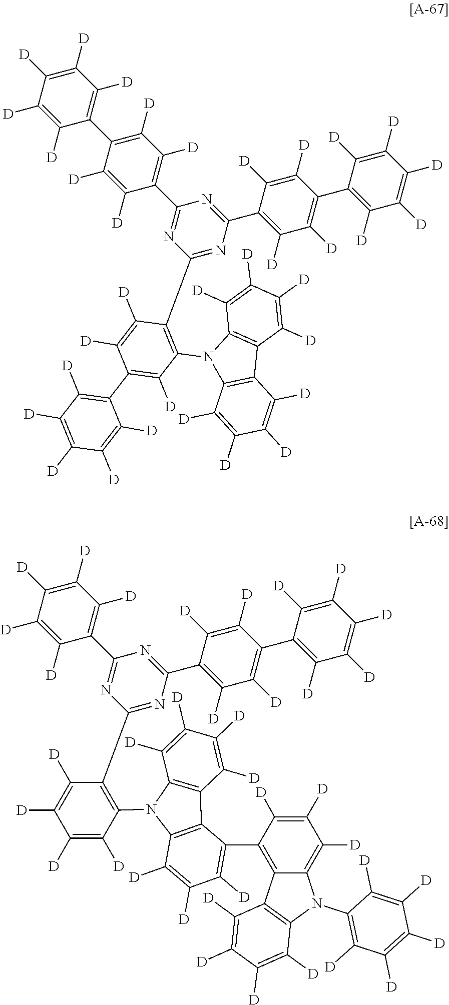

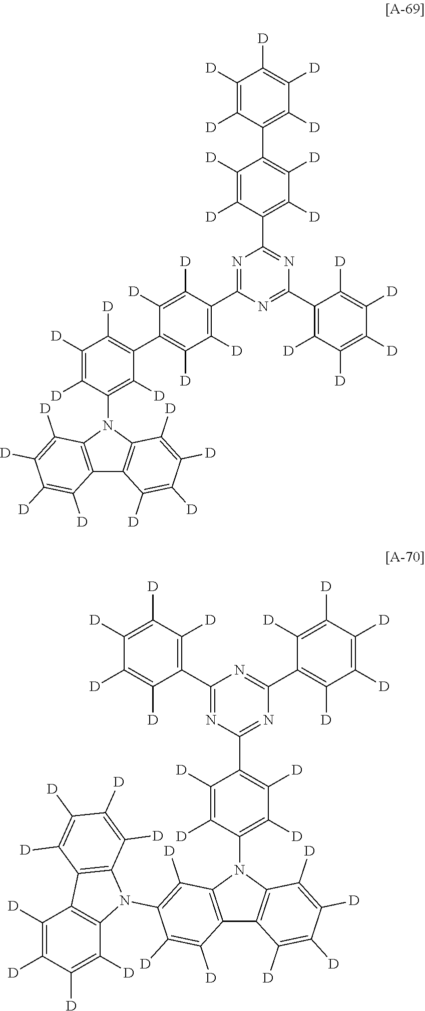

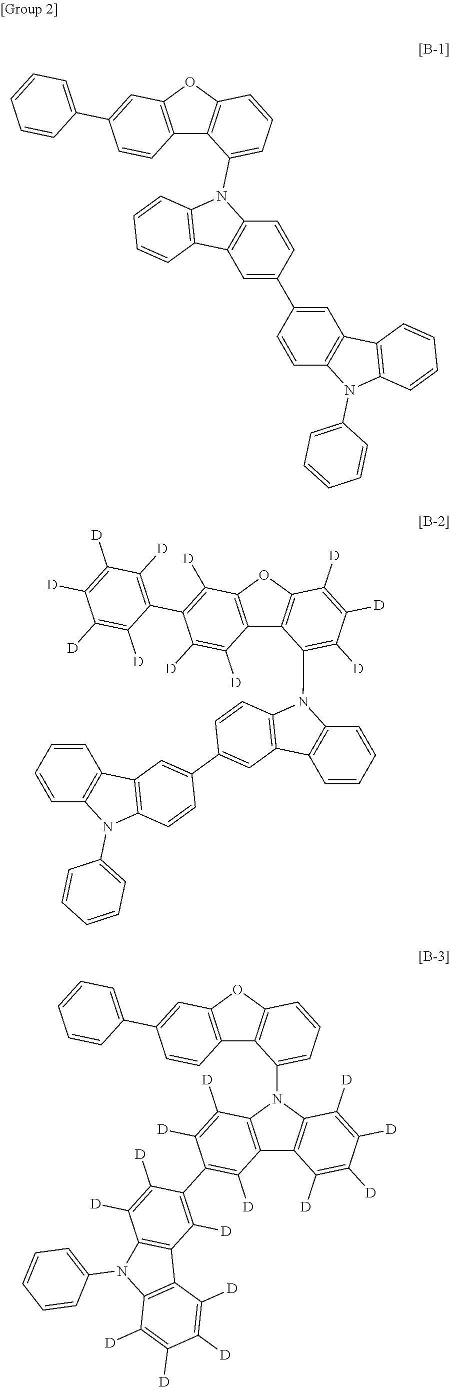 [SMILES: c1ccc(-c2ccc3c(c2)oc2cccc(-n4c5ccccc5c5cc(-c6ccc7c(c6)c6ccccc6n7-c6ccccc6)ccc54)c23)cc1 ; [2H]c1c([2H])c([2H])c(-c2c([2H])c([2H])c3c(oc4c([2H])c([2H])c([2H])c(-n5c6ccccc6c6cc(-c7ccc8c(c7)c7ccccc7n8-c7ccccc7)ccc65)c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4oc5cc(-c6ccccc6)ccc5c34)c([2H])c([2H])c1n2-c1ccccc1]

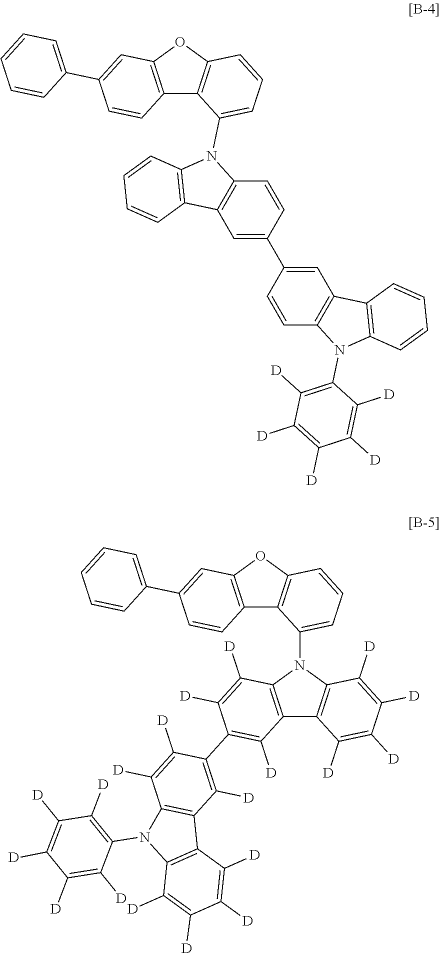 [SMILES: [2H]c1c([2H])c([2H])c(-n2c3ccccc3c3cc(-c4ccc5c(c4)c4ccccc4n5-c4cccc5oc6cc(-c7ccccc7)ccc6c45)ccc32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5oc6cc(-c7ccccc7)ccc6c45)c([2H])c([2H])c32)c([2H])c1[2H]]

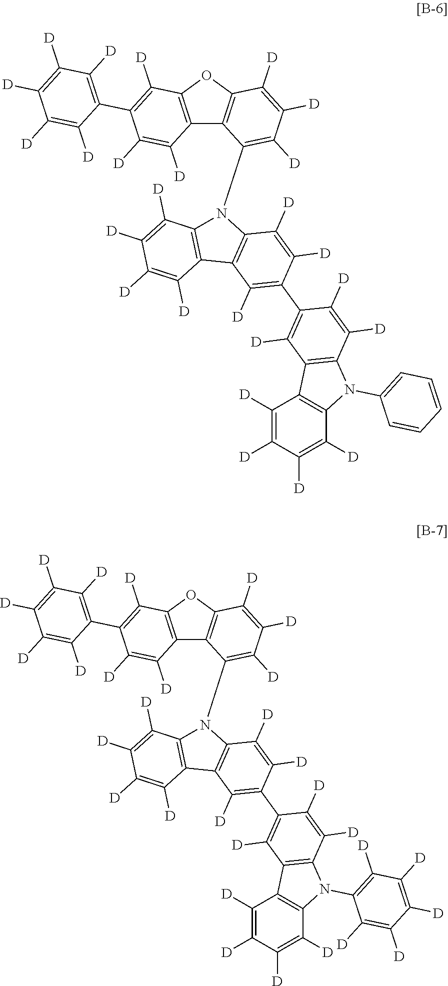 [SMILES: [2H]c1c([2H])c([2H])c(-c2c([2H])c([2H])c3c(oc4c([2H])c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)c43)c2[2H])c([2H])c1[2H] ; [2H]c1c([2H])c([2H])c(-c2c([2H])c([2H])c3c(oc4c([2H])c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c43)c2[2H])c([2H])c1[2H]]

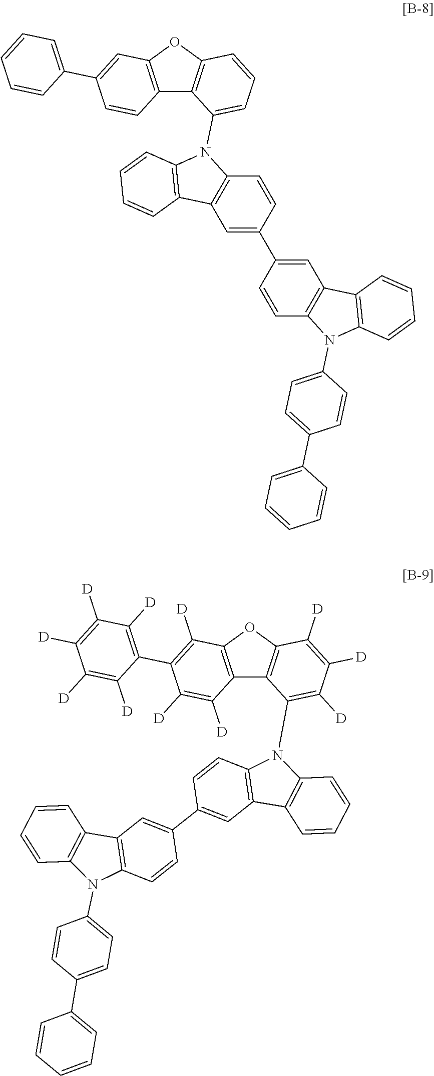 [SMILES: c1ccc(-c2ccc(-n3c4ccccc4c4cc(-c5ccc6c(c5)c5ccccc5n6-c5cccc6oc7cc(-c8ccccc8)ccc7c56)ccc43)cc2)cc1 ; [2H]c1c([2H])c([2H])c(-c2c([2H])c([2H])c3c(oc4c([2H])c([2H])c([2H])c(-n5c6ccccc6c6cc(-c7ccc8c(c7)c7ccccc7n8-c7ccc(-c8ccccc8)cc7)ccc65)c43)c2[2H])c([2H])c1[2H]]

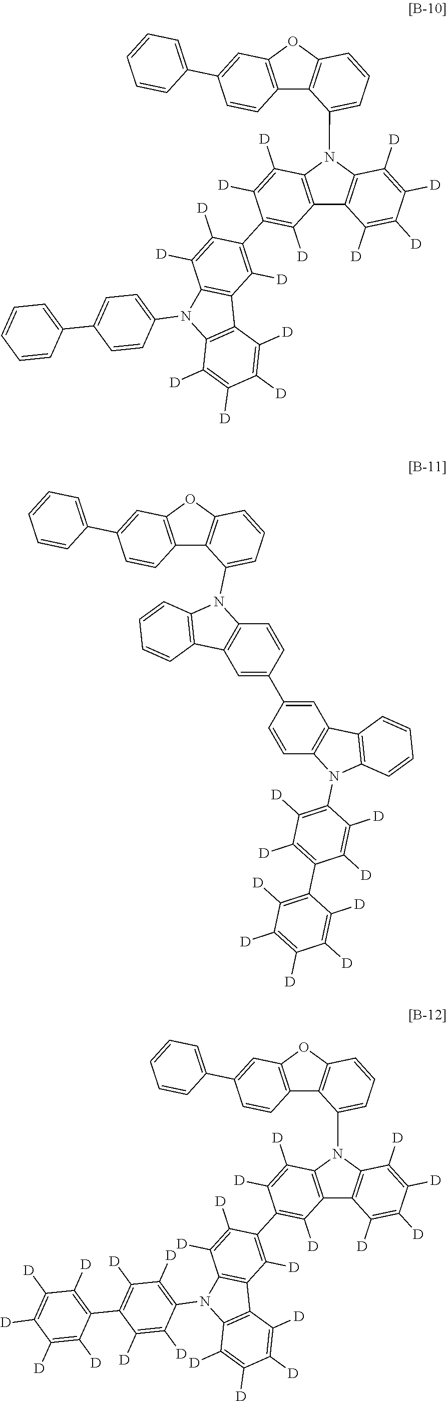

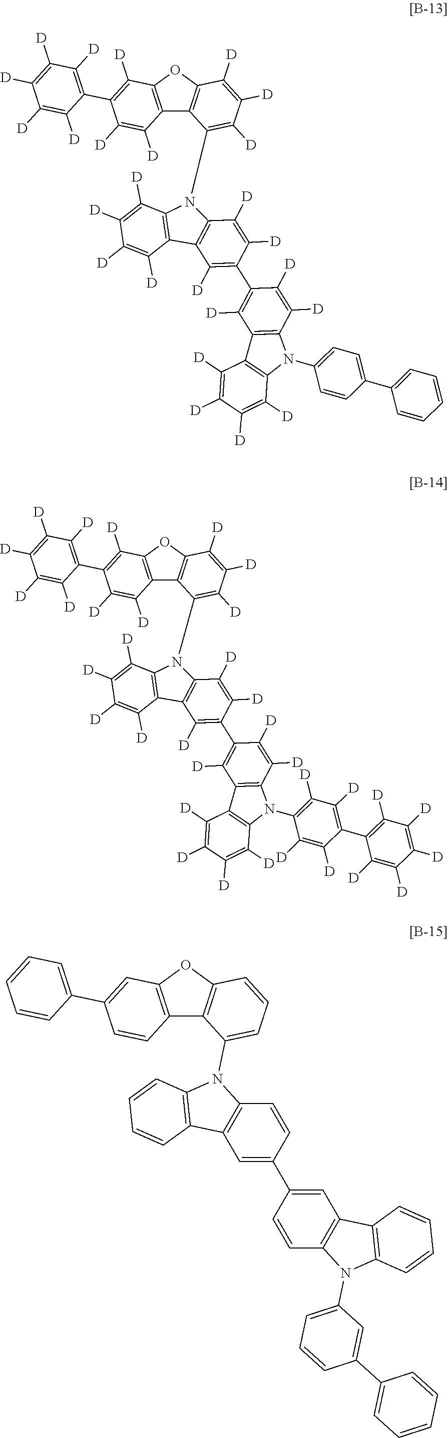

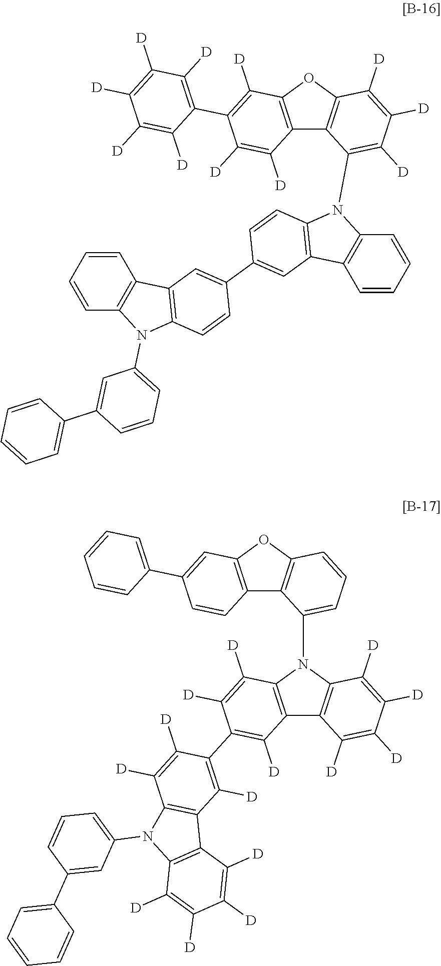 [SMILES: [2H]c1c([2H])c([2H])c(-c2c([2H])c([2H])c3c(oc4c([2H])c([2H])c([2H])c(-n5c6ccccc6c6cc(-c7ccc8c(c7)c7ccccc7n8-c7cccc(-c8ccccc8)c7)ccc65)c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4oc5cc(-c6ccccc6)ccc5c34)c([2H])c([2H])c1n2-c1cccc(-c2ccccc2)c1]

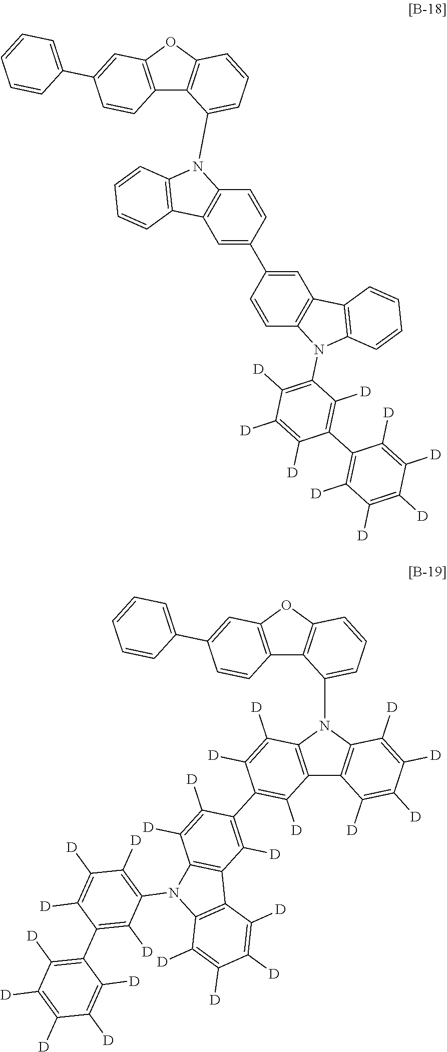 [SMILES: [2H]c1c([2H])c([2H])c(-c2c([2H])c([2H])c([2H])c(-n3c4ccccc4c4cc(-c5ccc6c(c5)c5ccccc5n6-c5cccc6oc7cc(-c8ccccc8)ccc7c56)ccc43)c2[2H])c([2H])c1[2H] ; [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6oc7cc(-c8ccccc8)ccc7c56)c([2H])c([2H])c43)c2[2H])c([2H])c1[2H]]

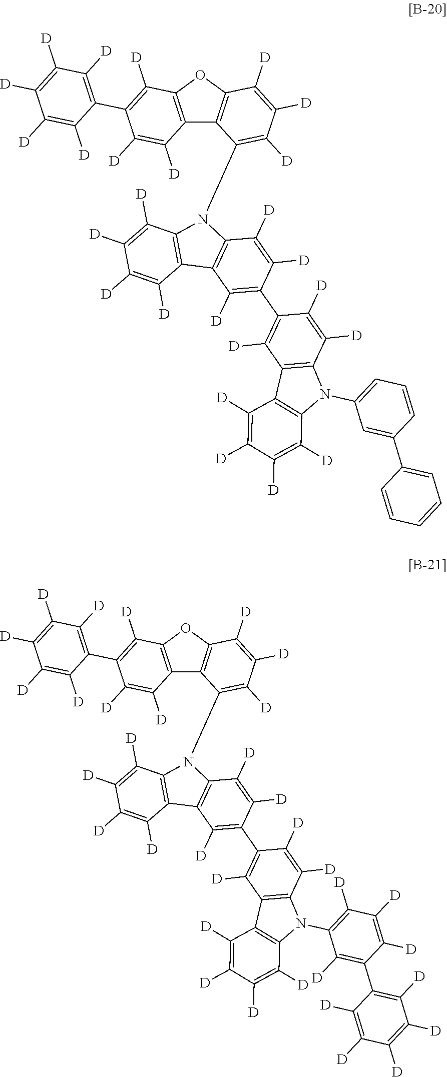

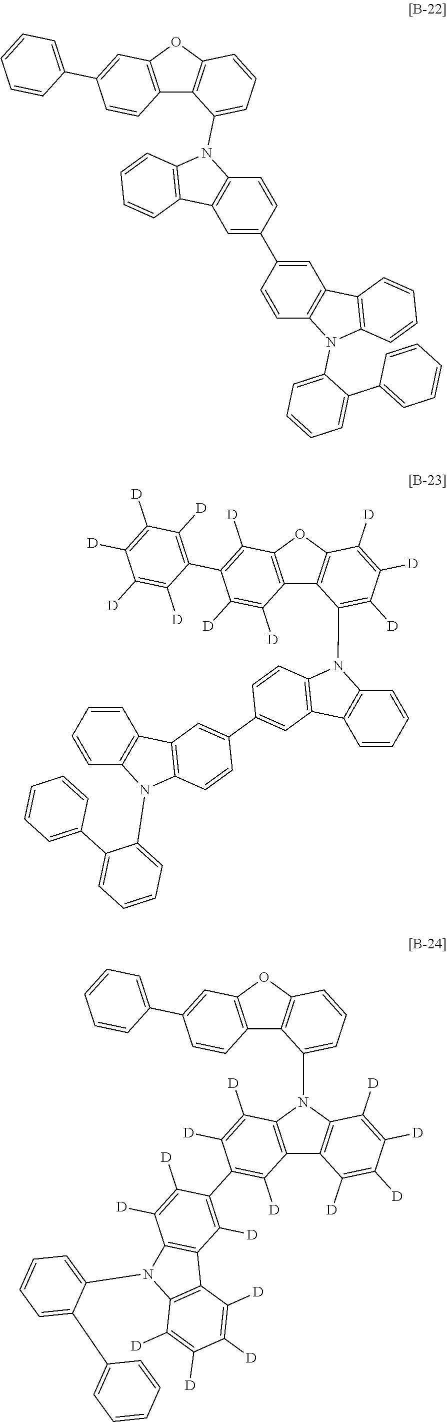 [SMILES: c1ccc(-c2ccc3c(c2)oc2cccc(-n4c5ccccc5c5cc(-c6ccc7c(c6)c6ccccc6n7-c6ccccc6-c6ccccc6)ccc54)c23)cc1 ; [2H]c1c([2H])c([2H])c(-c2c([2H])c([2H])c3c(oc4c([2H])c([2H])c([2H])c(-n5c6ccccc6c6cc(-c7ccc8c(c7)c7ccccc7n8-c7ccccc7-c7ccccc7)ccc65)c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4oc5cc(-c6ccccc6)ccc5c34)c([2H])c([2H])c1n2-c1ccccc1-c1ccccc1]

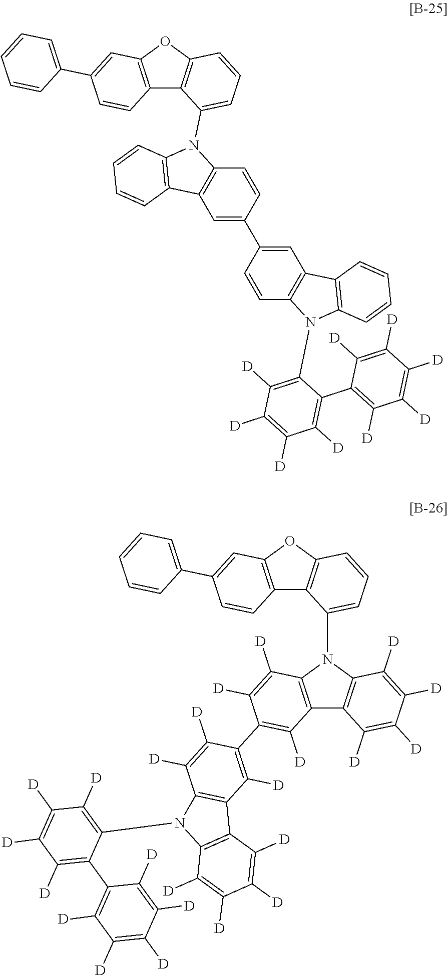 [SMILES: [2H]c1c([2H])c([2H])c(-c2c([2H])c([2H])c([2H])c([2H])c2-n2c3ccccc3c3cc(-c4ccc5c(c4)c4ccccc4n5-c4cccc5oc6cc(-c7ccccc7)ccc6c45)ccc32)c([2H])c1[2H] ; [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5oc6cc(-c7ccccc7)ccc6c45)c([2H])c([2H])c32)c([2H])c1[2H]]

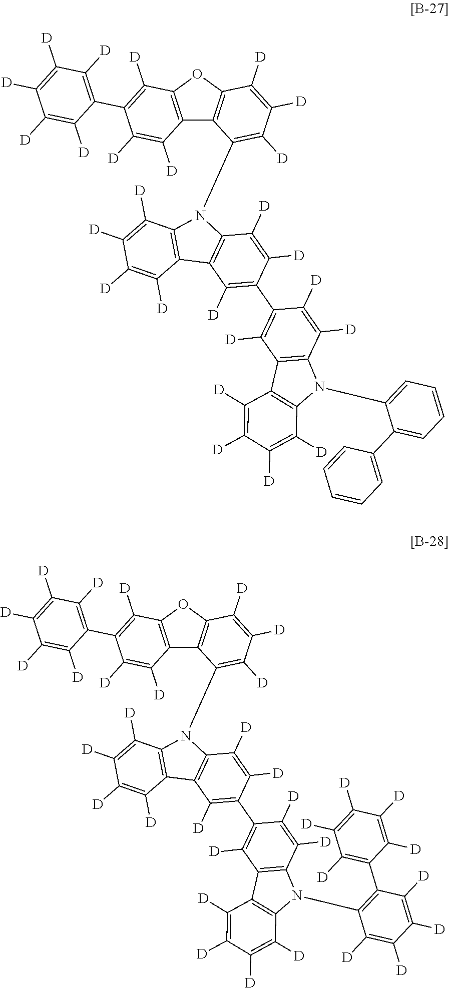

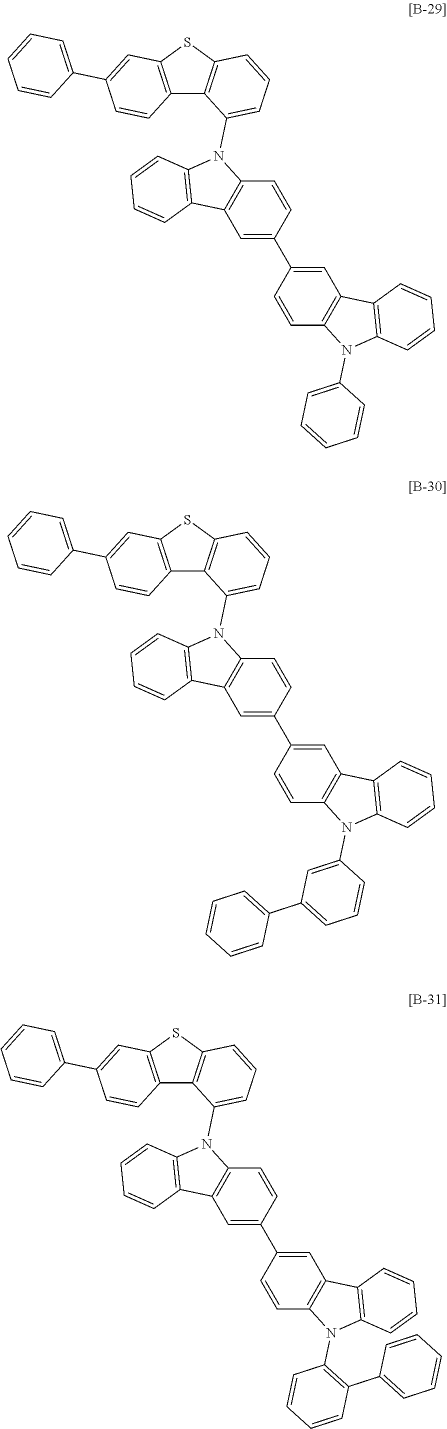 [SMILES: c1ccc(-c2ccc3c(c2)sc2cccc(-n4c5ccccc5c5cc(-c6ccc7c(c6)c6ccccc6n7-c6ccccc6)ccc54)c23)cc1 ; c1ccc(-c2cccc(-n3c4ccccc4c4cc(-c5ccc6c(c5)c5ccccc5n6-c5cccc6sc7cc(-c8ccccc8)ccc7c56)ccc43)c2)cc1 ; c1ccc(-c2ccc3c(c2)sc2cccc(-n4c5ccccc5c5cc(-c6ccc7c(c6)c6ccccc6n7-c6ccccc6-c6ccccc6)ccc54)c23)cc1]

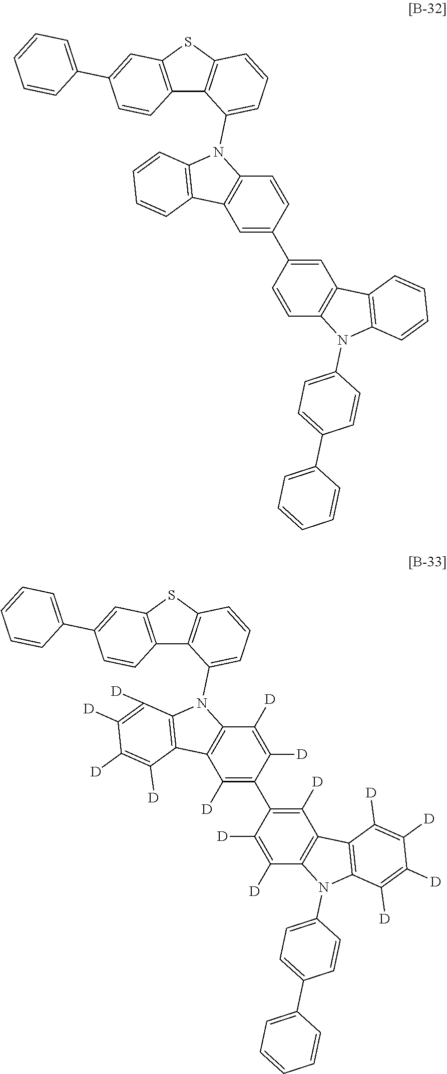 [SMILES: c1ccc(-c2ccc(-n3c4ccccc4c4cc(-c5ccc6c(c5)c5ccccc5n6-c5cccc6sc7cc(-c8ccccc8)ccc7c56)ccc43)cc2)cc1 ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4sc5cc(-c6ccccc6)ccc5c34)c([2H])c([2H])c1n2-c1ccc(-c2ccccc2)cc1]

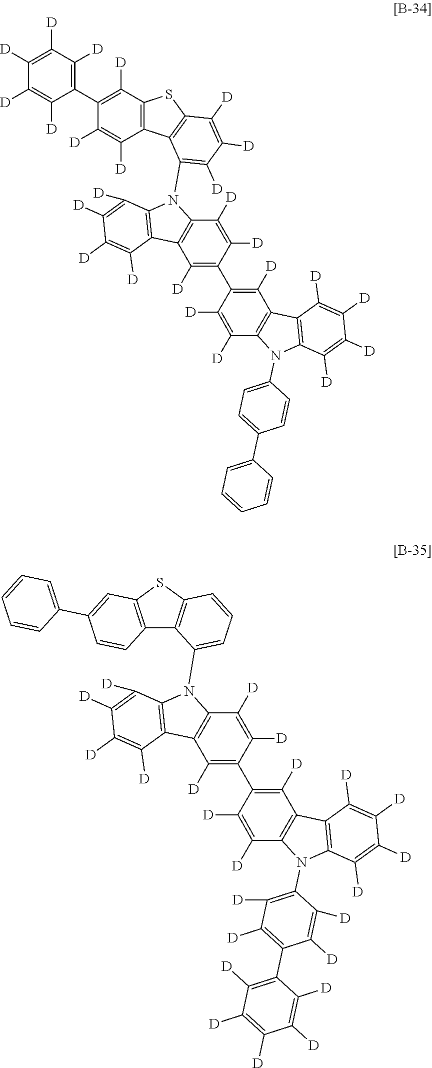 [SMILES: [2H]c1c([2H])c([2H])c(-c2c([2H])c([2H])c3c(sc4c([2H])c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)c43)c2[2H])c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6sc7cc(-c8ccccc8)ccc7c56)c([2H])c([2H])c43)c([2H])c2[2H])c([2H])c1[2H]]

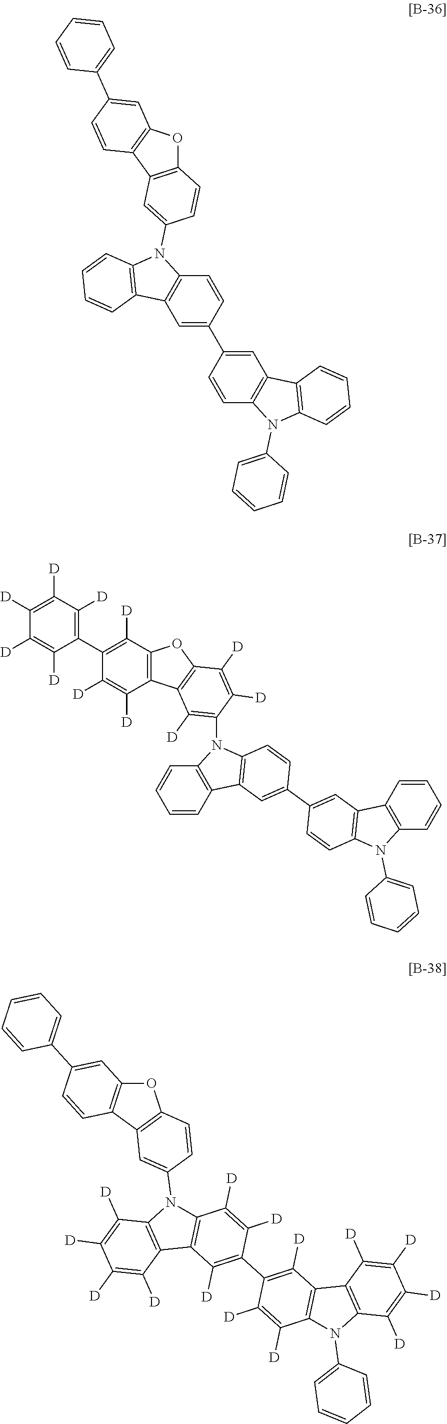 [SMILES: c1ccc(-c2ccc3c(c2)oc2ccc(-n4c5ccccc5c5cc(-c6ccc7c(c6)c6ccccc6n7-c6ccccc6)ccc54)cc23)cc1 ; [2H]c1c([2H])c([2H])c(-c2c([2H])c([2H])c3c(oc4c([2H])c([2H])c(-n5c6ccccc6c6cc(-c7ccc8c(c7)c7ccccc7n8-c7ccccc7)ccc65)c([2H])c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4oc5cc(-c6ccccc6)ccc5c4c3)c([2H])c([2H])c1n2-c1ccccc1]

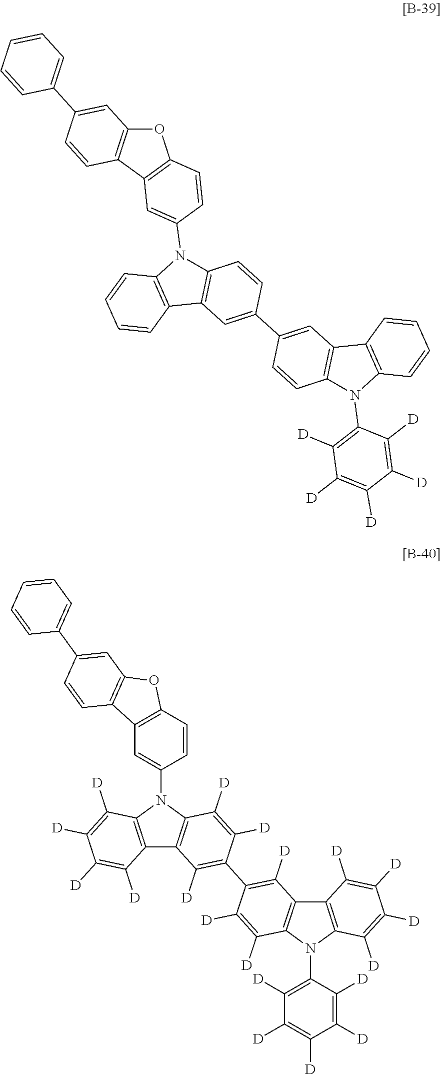 [SMILES: [2H]c1c([2H])c([2H])c(-n2c3ccccc3c3cc(-c4ccc5c(c4)c4ccccc4n5-c4ccc5oc6cc(-c7ccccc7)ccc6c5c4)ccc32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5oc6cc(-c7ccccc7)ccc6c5c4)c([2H])c([2H])c32)c([2H])c1[2H]]

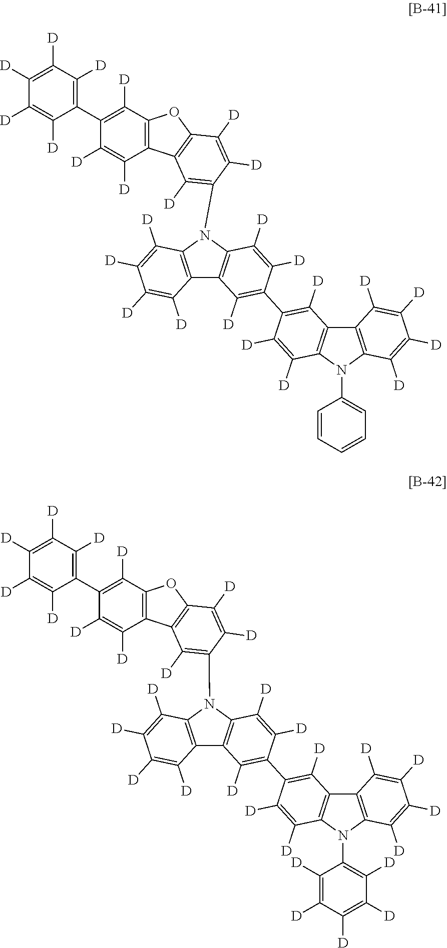 [SMILES: [2H]c1c([2H])c([2H])c(-c2c([2H])c([2H])c3c(oc4c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)c([2H])c43)c2[2H])c([2H])c1[2H] ; [2H]c1c([2H])c([2H])c(-c2c([2H])c([2H])c3c(oc4c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c([2H])c43)c2[2H])c([2H])c1[2H]]

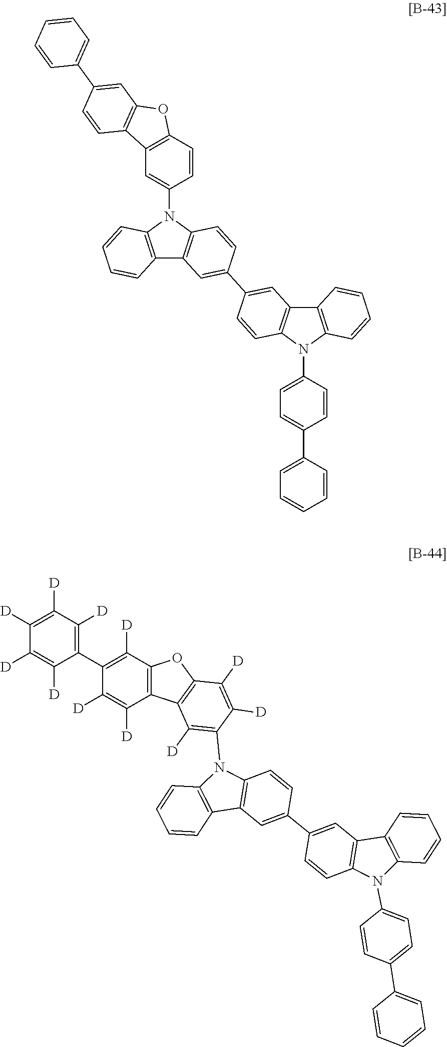 [SMILES: c1ccc(-c2ccc(-n3c4ccccc4c4cc(-c5ccc6c(c5)c5ccccc5n6-c5ccc6oc7cc(-c8ccccc8)ccc7c6c5)ccc43)cc2)cc1 ; [2H]c1c([2H])c([2H])c(-c2c([2H])c([2H])c3c(oc4c([2H])c([2H])c(-n5c6ccccc6c6cc(-c7ccc8c(c7)c7ccccc7n8-c7ccc(-c8ccccc8)cc7)ccc65)c([2H])c43)c2[2H])c([2H])c1[2H]]

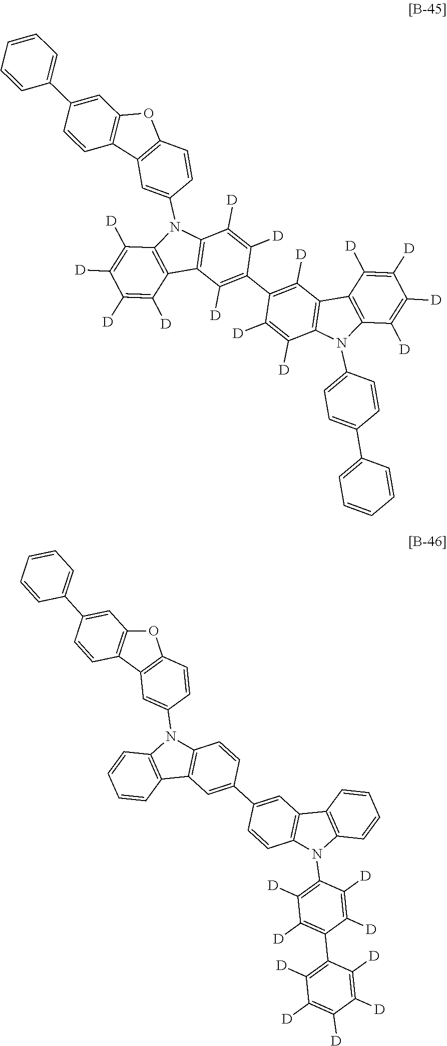 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4oc5cc(-c6ccccc6)ccc5c4c3)c([2H])c([2H])c1n2-c1ccc(-c2ccccc2)cc1 ; [2H]c1c([2H])c([2H])c(-c2c([2H])c([2H])c(-n3c4ccccc4c4cc(-c5ccc6c(c5)c5ccccc5n6-c5ccc6oc7cc(-c8ccccc8)ccc7c6c5)ccc43)c([2H])c2[2H])c([2H])c1[2H]]

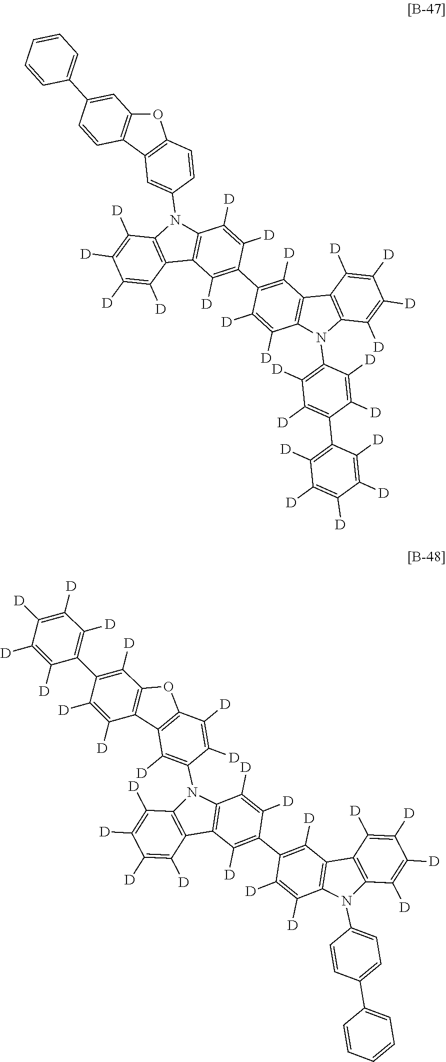 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6oc7cc(-c8ccccc8)ccc7c6c5)c([2H])c([2H])c43)c([2H])c2[2H])c([2H])c1[2H] ; [2H]c1c([2H])c([2H])c(-c2c([2H])c([2H])c3c(oc4c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)c([2H])c43)c2[2H])c([2H])c1[2H]]

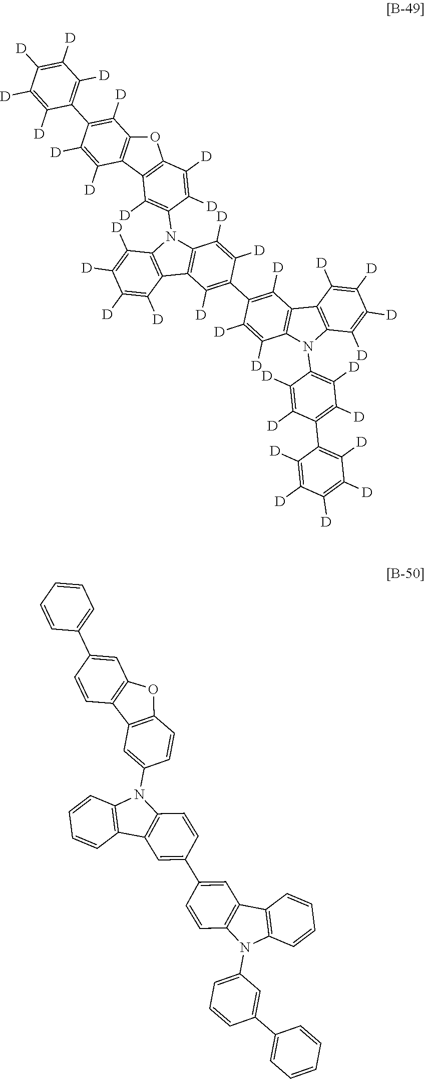 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6oc7c([2H])c(-c8c([2H])c([2H])c([2H])c([2H])c8[2H])c([2H])c([2H])c7c6c5[2H])c([2H])c([2H])c43)c([2H])c2[2H])c([2H])c1[2H] ; c1ccc(-c2cccc(-n3c4ccccc4c4cc(-c5ccc6c(c5)c5ccccc5n6-c5ccc6oc7cc(-c8ccccc8)ccc7c6c5)ccc43)c2)cc1]

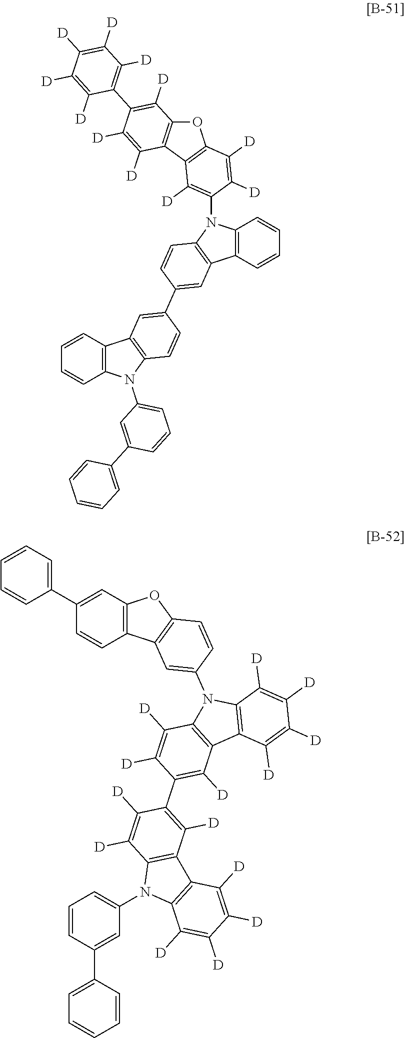 [SMILES: [2H]c1c([2H])c([2H])c(-c2c([2H])c([2H])c3c(oc4c([2H])c([2H])c(-n5c6ccccc6c6cc(-c7ccc8c(c7)c7ccccc7n8-c7cccc(-c8ccccc8)c7)ccc65)c([2H])c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4oc5cc(-c6ccccc6)ccc5c4c3)c([2H])c([2H])c1n2-c1cccc(-c2ccccc2)c1]

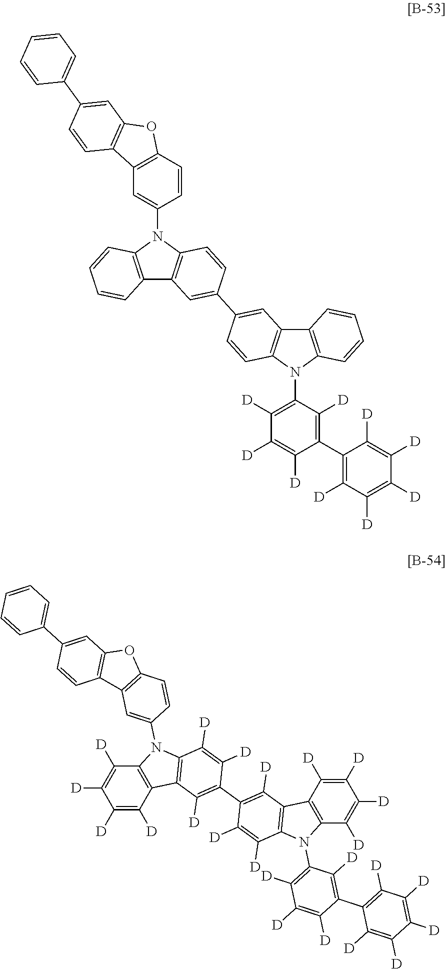 [SMILES: [2H]c1c([2H])c([2H])c(-c2c([2H])c([2H])c([2H])c(-n3c4ccccc4c4cc(-c5ccc6c(c5)c5ccccc5n6-c5ccc6oc7cc(-c8ccccc8)ccc7c6c5)ccc43)c2[2H])c([2H])c1[2H] ; [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6oc7cc(-c8ccccc8)ccc7c6c5)c([2H])c([2H])c43)c2[2H])c([2H])c1[2H]]

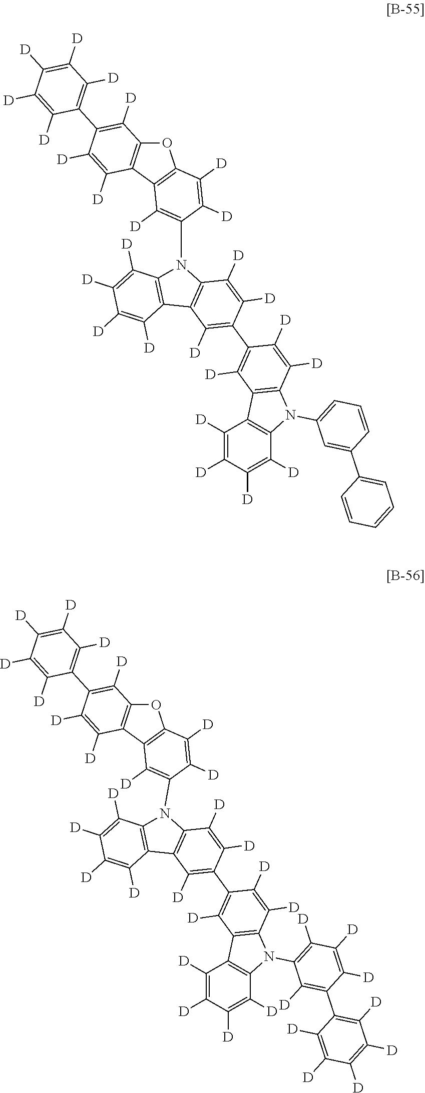

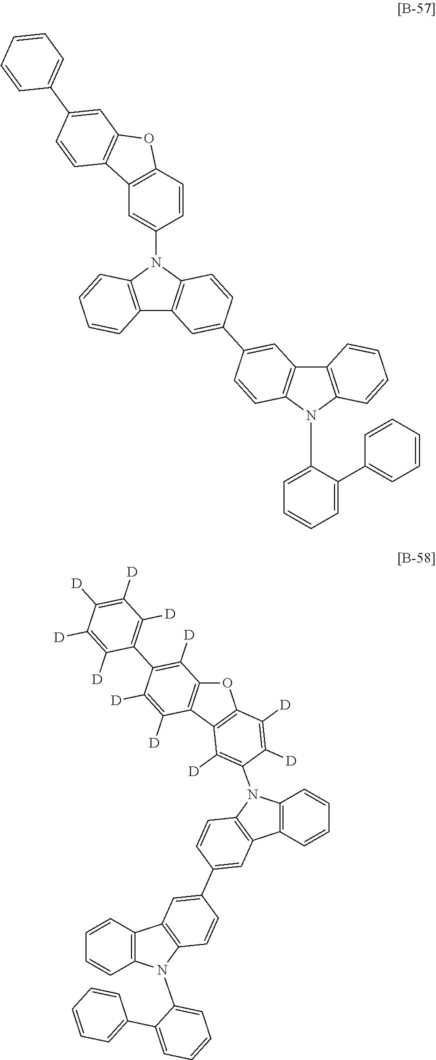 [SMILES: c1ccc(-c2ccc3c(c2)oc2ccc(-n4c5ccccc5c5cc(-c6ccc7c(c6)c6ccccc6n7-c6ccccc6-c6ccccc6)ccc54)cc23)cc1 ; [2H]c1c([2H])c([2H])c(-c2c([2H])c([2H])c3c(oc4c([2H])c([2H])c(-n5c6ccccc6c6cc(-c7ccc8c(c7)c7ccccc7n8-c7ccccc7-c7ccccc7)ccc65)c([2H])c43)c2[2H])c([2H])c1[2H]]

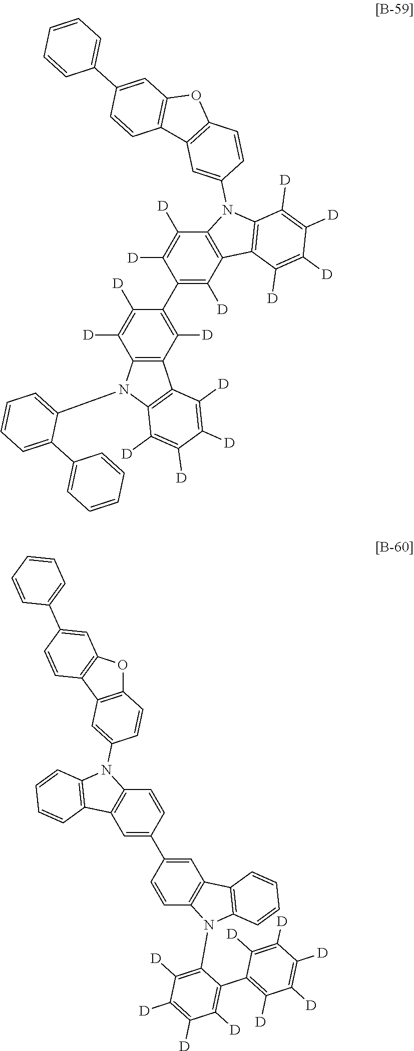 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3-c3ccccc3)c([2H])c([2H])c1n2-c1ccc2oc3cc(-c4ccccc4)ccc3c2c1 ; [2H]c1c([2H])c([2H])c(-c2c([2H])c([2H])c([2H])c([2H])c2-n2c3ccccc3c3cc(-c4ccc5c(c4)c4ccccc4n5-c4ccc5oc6cc(-c7ccccc7)ccc6c5c4)ccc32)c([2H])c1[2H]]

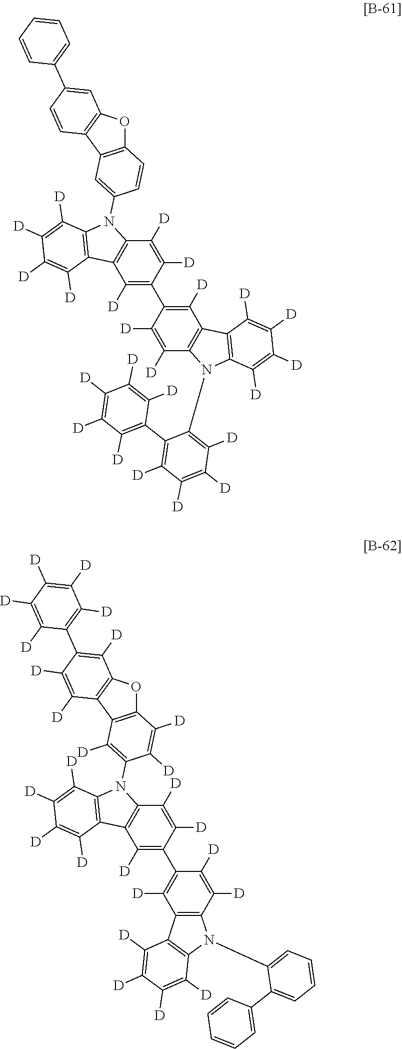 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5oc6cc(-c7ccccc7)ccc6c5c4)c([2H])c([2H])c32)c([2H])c1[2H] ; [2H]c1c([2H])c([2H])c(-c2c([2H])c([2H])c3c(oc4c([2H])c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7-c7ccccc7)c([2H])c([2H])c65)c([2H])c43)c2[2H])c([2H])c1[2H]]

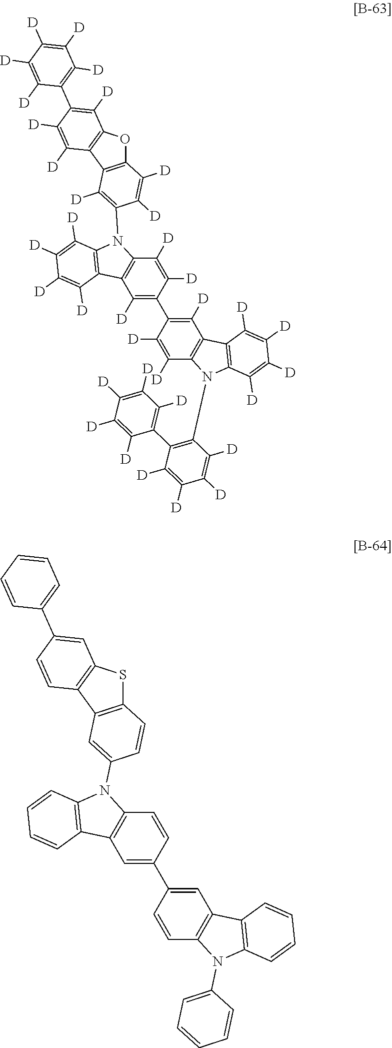 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4c([2H])c([2H])c5oc6c([2H])c(-c7c([2H])c([2H])c([2H])c([2H])c7[2H])c([2H])c([2H])c6c5c4[2H])c([2H])c([2H])c32)c([2H])c1[2H] ; c1ccc(-c2ccc3c(c2)sc2ccc(-n4c5ccccc5c5cc(-c6ccc7c(c6)c6ccccc6n7-c6ccccc6)ccc54)cc23)cc1]

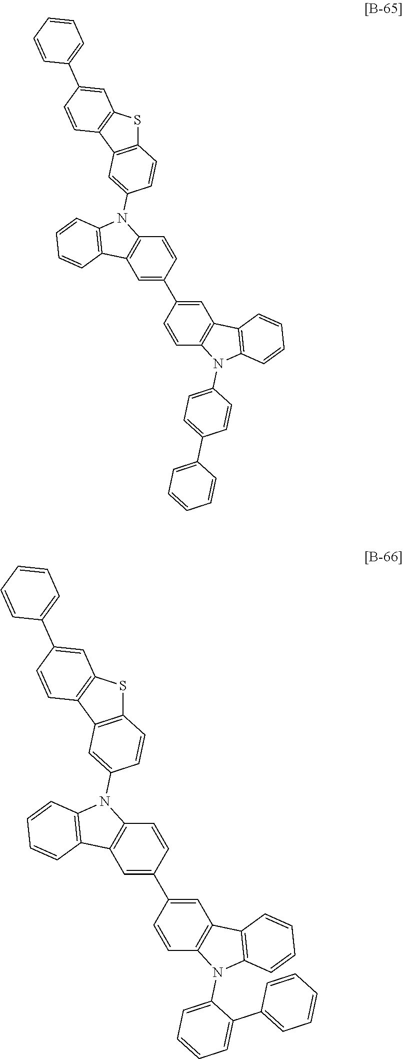 [SMILES: c1ccc(-c2ccc(-n3c4ccccc4c4cc(-c5ccc6c(c5)c5ccccc5n6-c5ccc6sc7cc(-c8ccccc8)ccc7c6c5)ccc43)cc2)cc1 ; c1ccc(-c2ccc3c(c2)sc2ccc(-n4c5ccccc5c5cc(-c6ccc7c(c6)c6ccccc6n7-c6ccccc6-c6ccccc6)ccc54)cc23)cc1]

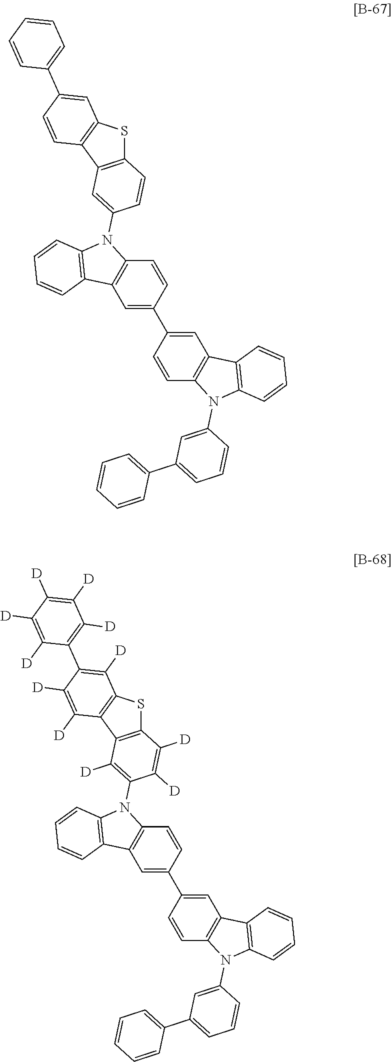 [SMILES: c1ccc(-c2cccc(-n3c4ccccc4c4cc(-c5ccc6c(c5)c5ccccc5n6-c5ccc6sc7cc(-c8ccccc8)ccc7c6c5)ccc43)c2)cc1 ; [2H]c1c([2H])c([2H])c(-c2c([2H])c([2H])c3c(sc4c([2H])c([2H])c(-n5c6ccccc6c6cc(-c7ccc8c(c7)c7ccccc7n8-c7cccc(-c8ccccc8)c7)ccc65)c([2H])c43)c2[2H])c([2H])c1[2H]]

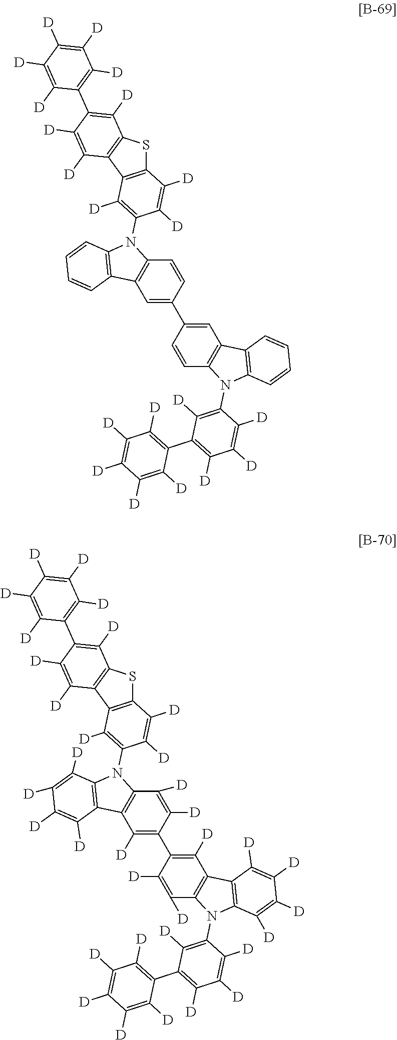 [SMILES: [2H]c1c([2H])c([2H])c(-c2c([2H])c([2H])c([2H])c(-n3c4ccccc4c4cc(-c5ccc6c(c5)c5ccccc5n6-c5c([2H])c([2H])c6sc7c([2H])c(-c8c([2H])c([2H])c([2H])c([2H])c8[2H])c([2H])c([2H])c7c6c5[2H])ccc43)c2[2H])c([2H])c1[2H] ; [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6sc7c([2H])c(-c8c([2H])c([2H])c([2H])c([2H])c8[2H])c([2H])c([2H])c7c6c5[2H])c([2H])c([2H])c43)c2[2H])c([2H])c1[2H]]

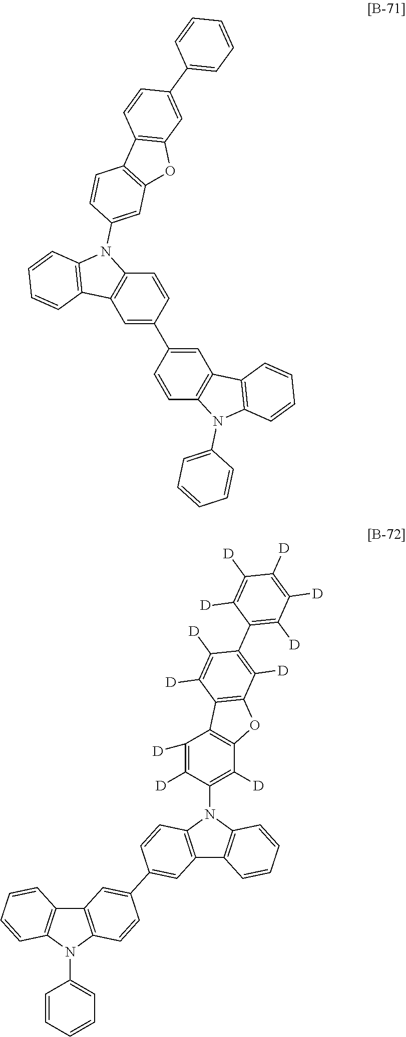 [SMILES: c1ccc(-c2ccc3c(c2)oc2cc(-n4c5ccccc5c5cc(-c6ccc7c(c6)c6ccccc6n7-c6ccccc6)ccc54)ccc23)cc1 ; [2H]c1c([2H])c([2H])c(-c2c([2H])c([2H])c3c(oc4c([2H])c(-n5c6ccccc6c6cc(-c7ccc8c(c7)c7ccccc7n8-c7ccccc7)ccc65)c([2H])c([2H])c43)c2[2H])c([2H])c1[2H]]

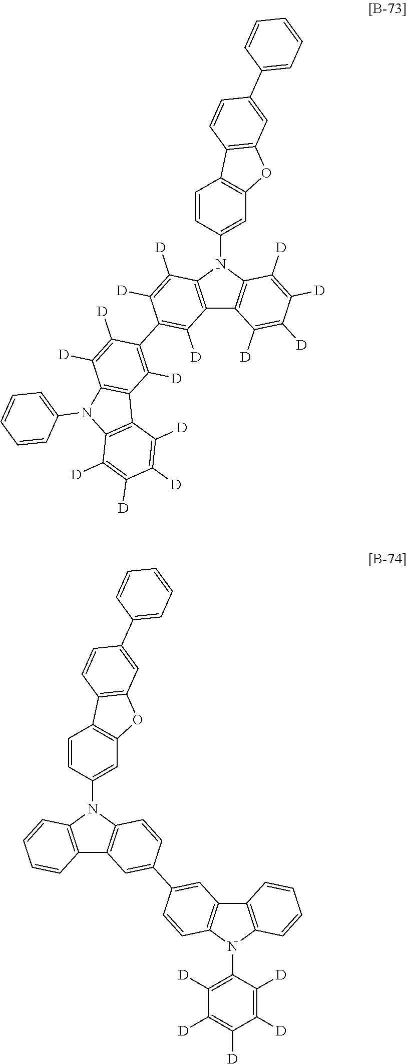 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c(c3)oc3cc(-c5ccccc5)ccc34)c([2H])c([2H])c1n2-c1ccccc1 ; [2H]c1c([2H])c([2H])c(-n2c3ccccc3c3cc(-c4ccc5c(c4)c4ccccc4n5-c4ccc5c(c4)oc4cc(-c6ccccc6)ccc45)ccc32)c([2H])c1[2H]]

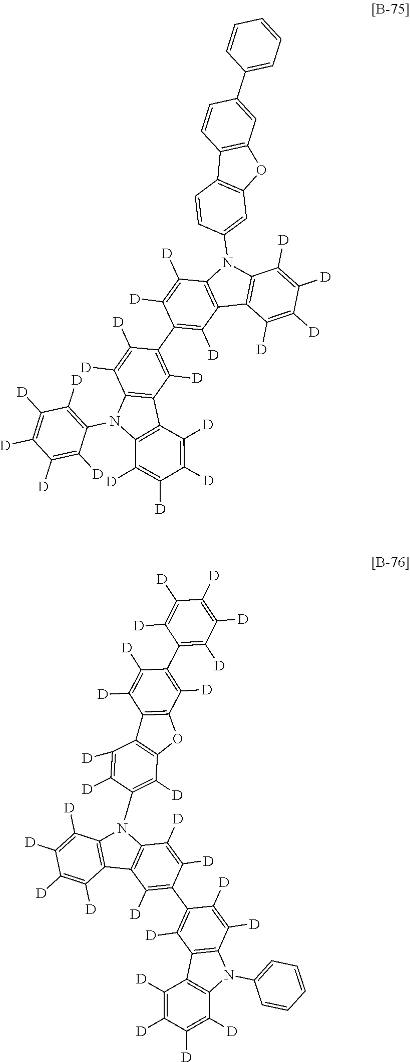 [SMILES: [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c(c4)oc4cc(-c6ccccc6)ccc45)c([2H])c([2H])c32)c([2H])c1[2H] ; [2H]c1c([2H])c([2H])c(-c2c([2H])c([2H])c3c(oc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)c([2H])c([2H])c43)c2[2H])c([2H])c1[2H]]

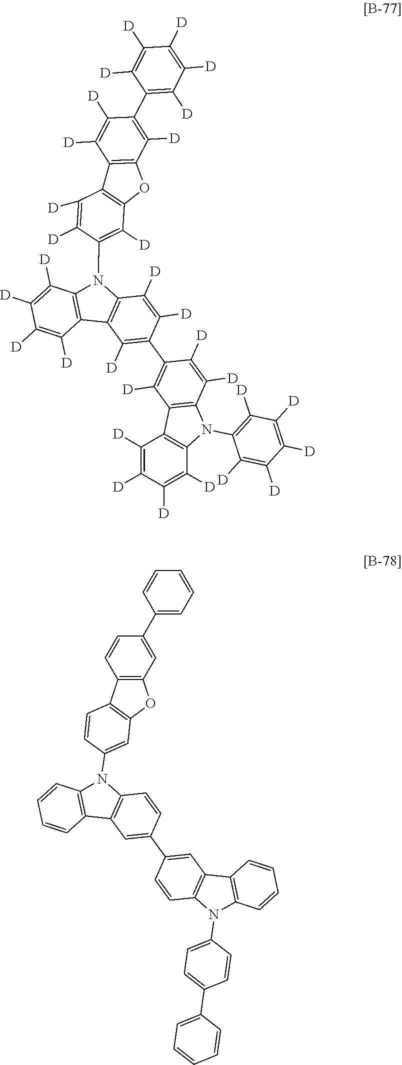 [SMILES: [2H]c1c([2H])c([2H])c(-c2c([2H])c([2H])c3c(oc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c([2H])c([2H])c43)c2[2H])c([2H])c1[2H] ; c1ccc(-c2ccc(-n3c4ccccc4c4cc(-c5ccc6c(c5)c5ccccc5n6-c5ccc6c(c5)oc5cc(-c7ccccc7)ccc56)ccc43)cc2)cc1]

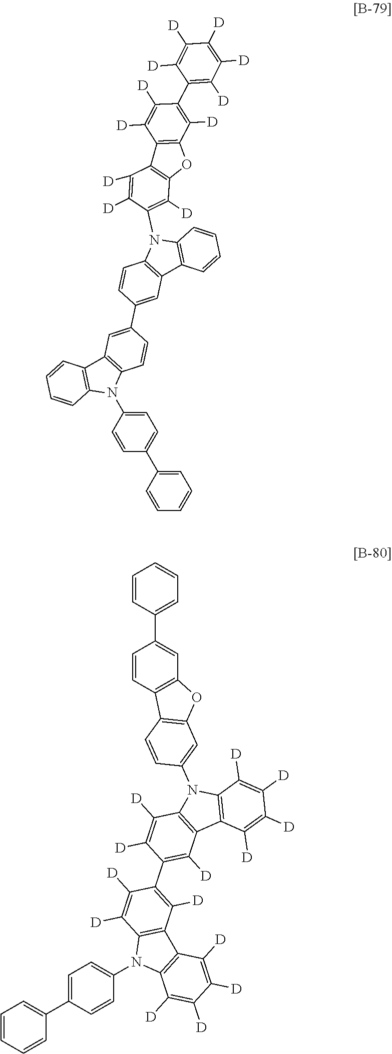 [SMILES: [2H]c1c([2H])c([2H])c(-c2c([2H])c([2H])c3c(oc4c([2H])c(-n5c6ccccc6c6cc(-c7ccc8c(c7)c7ccccc7n8-c7ccc(-c8ccccc8)cc7)ccc65)c([2H])c([2H])c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c(c3)oc3cc(-c5ccccc5)ccc34)c([2H])c([2H])c1n2-c1ccc(-c2ccccc2)cc1]

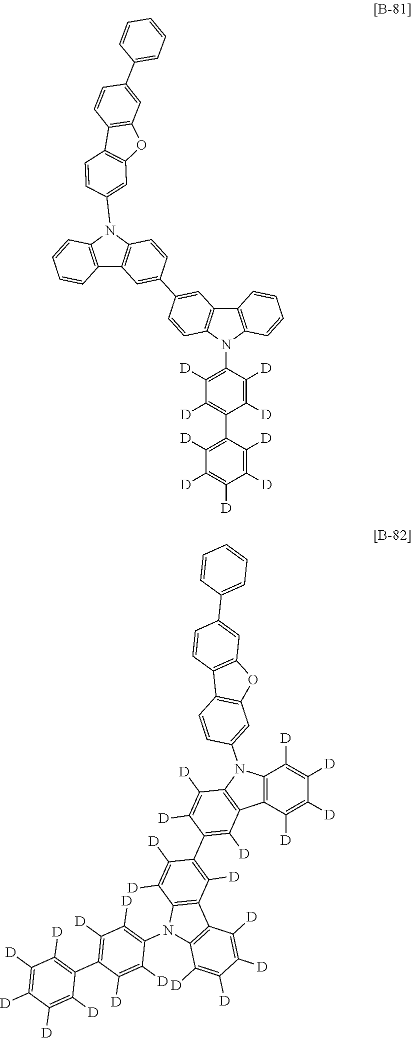 [SMILES: [2H]c1c([2H])c([2H])c(-c2c([2H])c([2H])c(-n3c4ccccc4c4cc(-c5ccc6c(c5)c5ccccc5n6-c5ccc6c(c5)oc5cc(-c7ccccc7)ccc56)ccc43)c([2H])c2[2H])c([2H])c1[2H] ; [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)oc5cc(-c7ccccc7)ccc56)c([2H])c([2H])c43)c([2H])c2[2H])c([2H])c1[2H]]

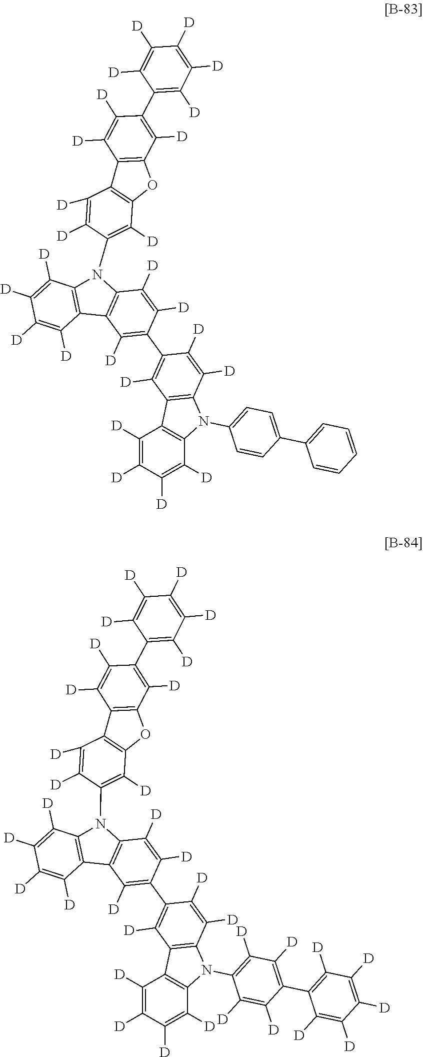

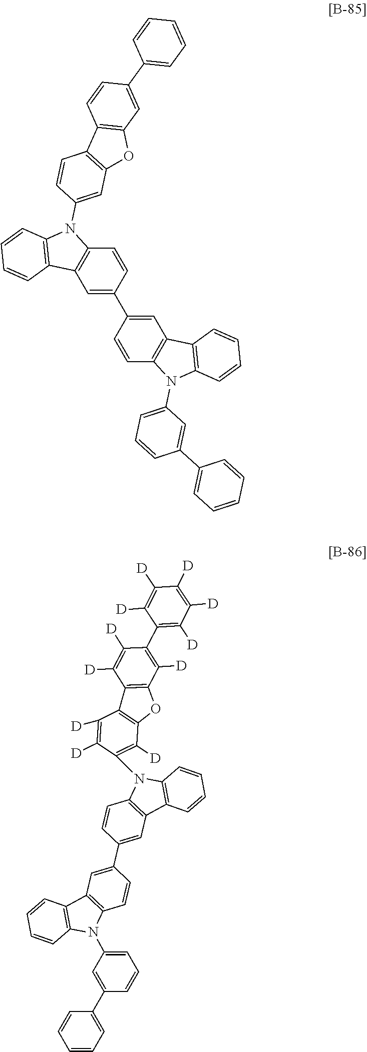 [SMILES: c1ccc(-c2cccc(-n3c4ccccc4c4cc(-c5ccc6c(c5)c5ccccc5n6-c5ccc6c(c5)oc5cc(-c7ccccc7)ccc56)ccc43)c2)cc1 ; [2H]c1c([2H])c([2H])c(-c2c([2H])c([2H])c3c(oc4c([2H])c(-n5c6ccccc6c6cc(-c7ccc8c(c7)c7ccccc7n8-c7cccc(-c8ccccc8)c7)ccc65)c([2H])c([2H])c43)c2[2H])c([2H])c1[2H]]

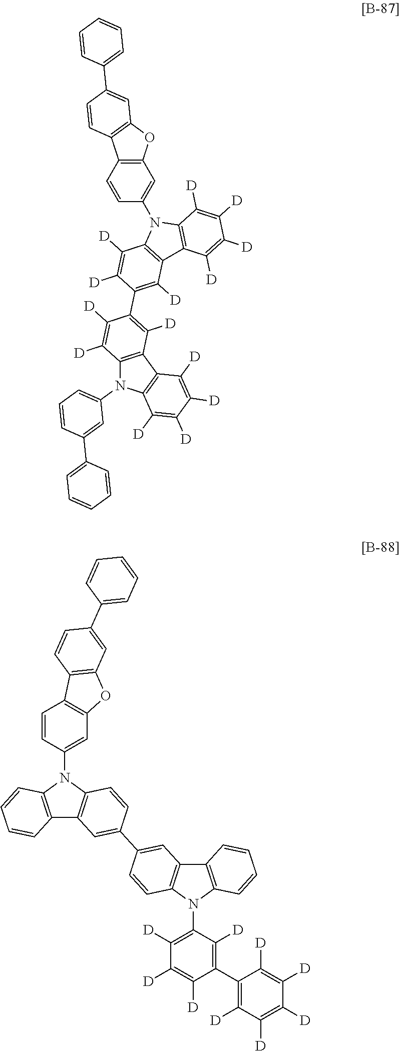 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3ccc4c(c3)oc3cc(-c5ccccc5)ccc34)c([2H])c([2H])c1n2-c1cccc(-c2ccccc2)c1 ; [2H]c1c([2H])c([2H])c(-c2c([2H])c([2H])c([2H])c(-n3c4ccccc4c4cc(-c5ccc6c(c5)c5ccccc5n6-c5ccc6c(c5)oc5cc(-c7ccccc7)ccc56)ccc43)c2[2H])c([2H])c1[2H]]

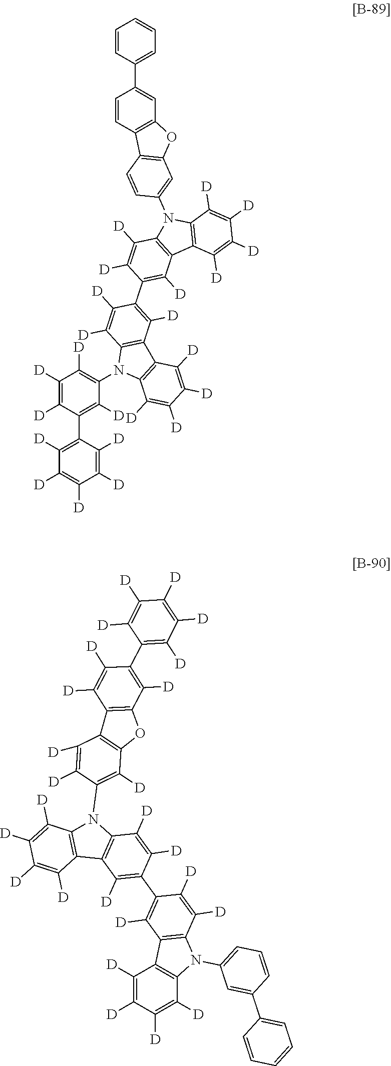 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5ccc6c(c5)oc5cc(-c7ccccc7)ccc56)c([2H])c([2H])c43)c2[2H])c([2H])c1[2H] ; [2H]c1c([2H])c([2H])c(-c2c([2H])c([2H])c3c(oc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7cccc(-c8ccccc8)c7)c([2H])c([2H])c65)c([2H])c([2H])c43)c2[2H])c([2H])c1[2H]]

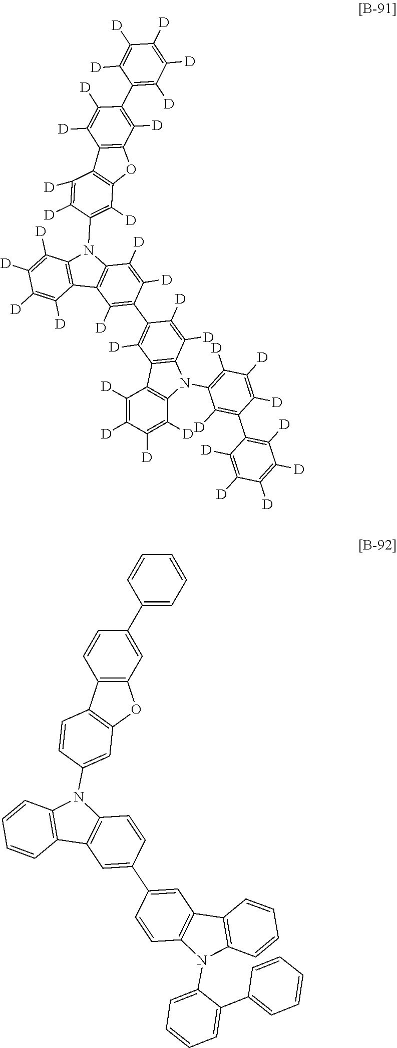 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c(oc7c([2H])c(-c8c([2H])c([2H])c([2H])c([2H])c8[2H])c([2H])c([2H])c76)c5[2H])c([2H])c([2H])c43)c2[2H])c([2H])c1[2H] ; c1ccc(-c2ccc3c(c2)oc2cc(-n4c5ccccc5c5cc(-c6ccc7c(c6)c6ccccc6n7-c6ccccc6-c6ccccc6)ccc54)ccc23)cc1]

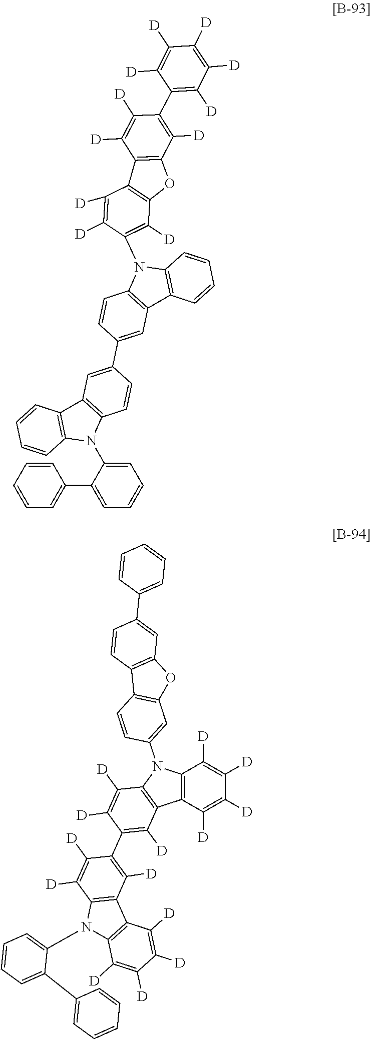 [SMILES: [2H]c1c([2H])c([2H])c(-c2c([2H])c([2H])c3c(oc4c([2H])c(-n5c6ccccc6c6cc(-c7ccc8c(c7)c7ccccc7n8-c7ccccc7-c7ccccc7)ccc65)c([2H])c([2H])c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3ccccc3-c3ccccc3)c([2H])c([2H])c1n2-c1ccc2c(c1)oc1cc(-c3ccccc3)ccc12]

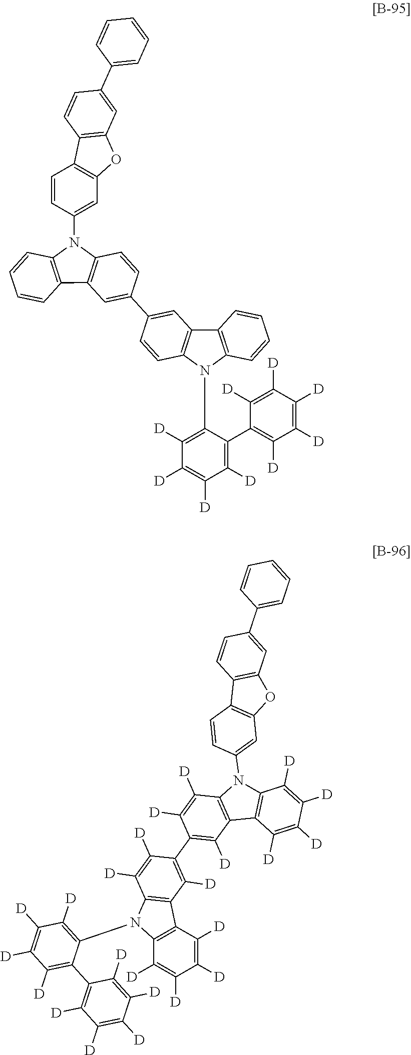 [SMILES: [2H]c1c([2H])c([2H])c(-c2c([2H])c([2H])c([2H])c([2H])c2-n2c3ccccc3c3cc(-c4ccc5c(c4)c4ccccc4n5-c4ccc5c(c4)oc4cc(-c6ccccc6)ccc45)ccc32)c([2H])c1[2H] ; [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c(c4)oc4cc(-c6ccccc6)ccc45)c([2H])c([2H])c32)c([2H])c1[2H]]

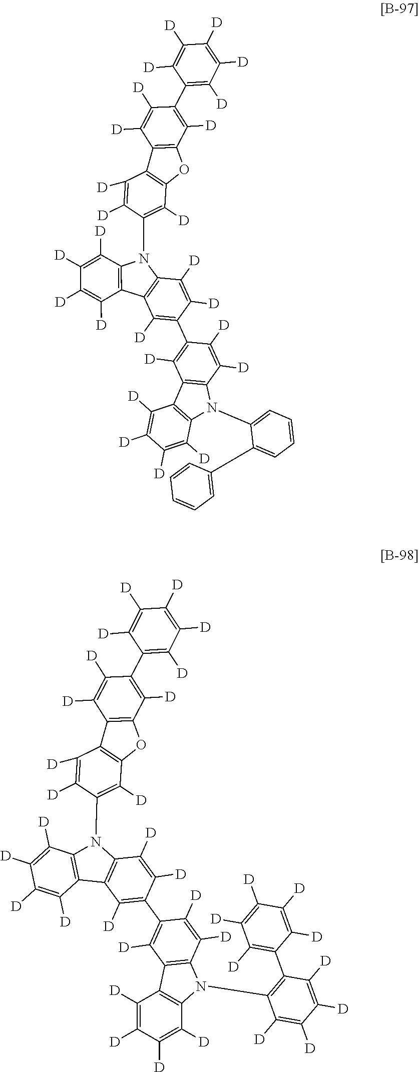

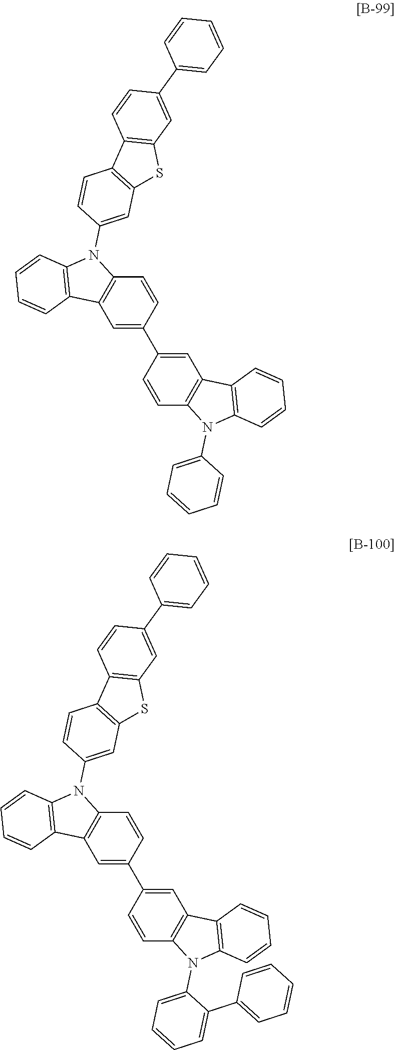 [SMILES: c1ccc(-c2ccc3c(c2)sc2cc(-n4c5ccccc5c5cc(-c6ccc7c(c6)c6ccccc6n7-c6ccccc6)ccc54)ccc23)cc1 ; c1ccc(-c2ccc3c(c2)sc2cc(-n4c5ccccc5c5cc(-c6ccc7c(c6)c6ccccc6n7-c6ccccc6-c6ccccc6)ccc54)ccc23)cc1]

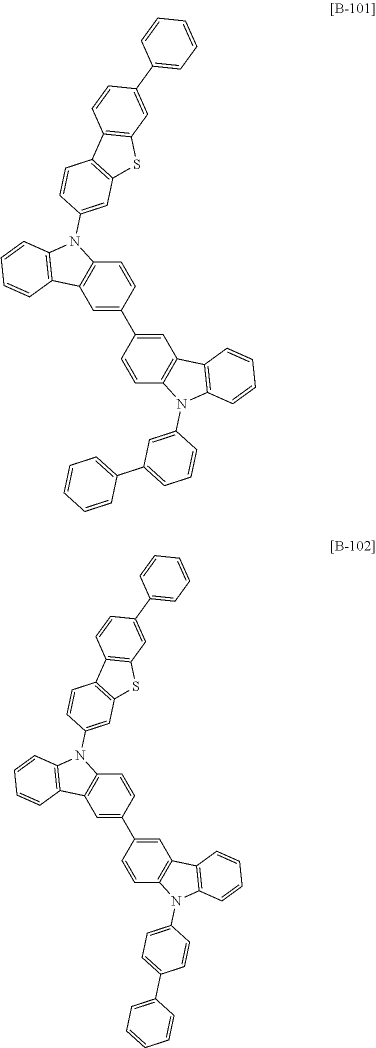 [SMILES: c1ccc(-c2cccc(-n3c4ccccc4c4cc(-c5ccc6c(c5)c5ccccc5n6-c5ccc6c(c5)sc5cc(-c7ccccc7)ccc56)ccc43)c2)cc1 ; c1ccc(-c2ccc(-n3c4ccccc4c4cc(-c5ccc6c(c5)c5ccccc5n6-c5ccc6c(c5)sc5cc(-c7ccccc7)ccc56)ccc43)cc2)cc1]

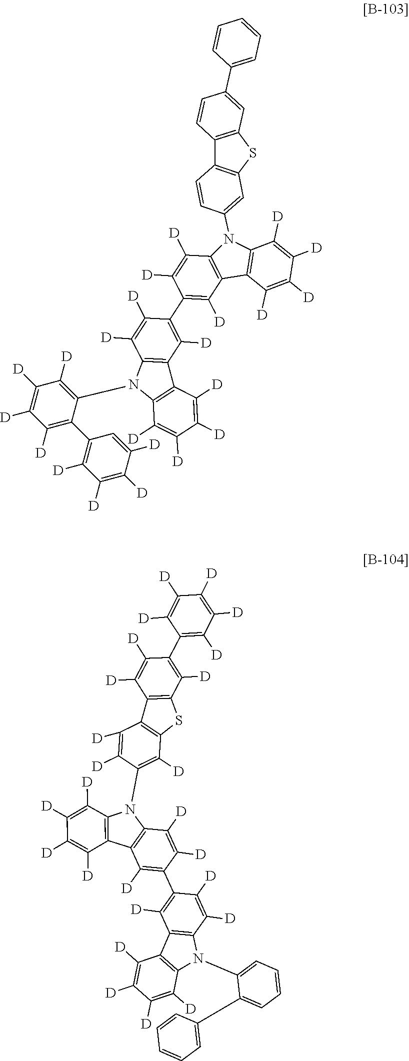 [SMILES: [2H]c1cc(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4ccc5c(c4)sc4cc(-c6ccccc6)ccc45)c([2H])c([2H])c32)c([2H])c([2H])c1[2H] ; [2H]c1c([2H])c([2H])c(-c2c([2H])c([2H])c3c(sc4c([2H])c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7-c7ccccc7)c([2H])c([2H])c65)c([2H])c([2H])c43)c2[2H])c([2H])c1[2H]]

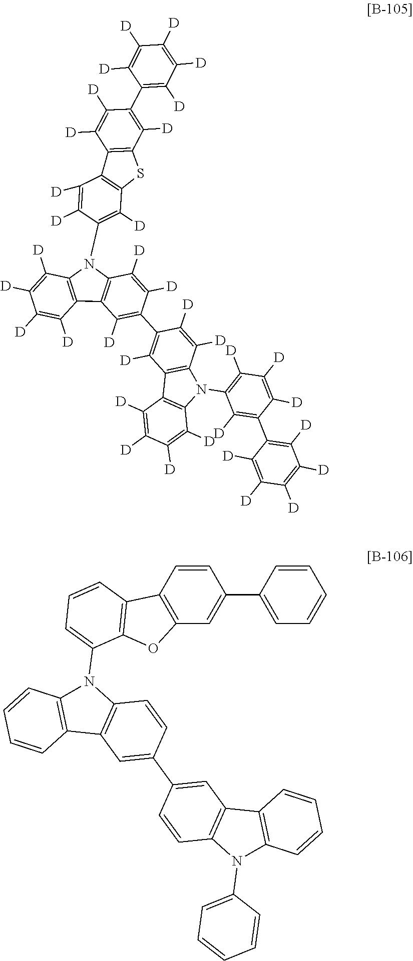 [SMILES: [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c6c(sc7c([2H])c(-c8c([2H])c([2H])c([2H])c([2H])c8[2H])c([2H])c([2H])c76)c5[2H])c([2H])c([2H])c43)c2[2H])c([2H])c1[2H] ; c1ccc(-c2ccc3c(c2)oc2c(-n4c5ccccc5c5cc(-c6ccc7c(c6)c6ccccc6n7-c6ccccc6)ccc54)cccc23)cc1]

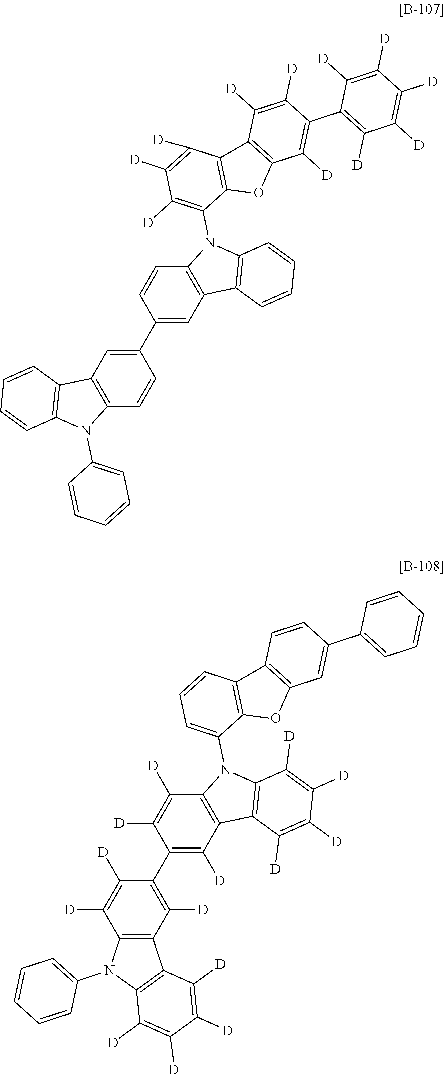 [SMILES: [2H]c1c([2H])c([2H])c(-c2c([2H])c([2H])c3c(oc4c(-n5c6ccccc6c6cc(-c7ccc8c(c7)c7ccccc7n8-c7ccccc7)ccc65)c([2H])c([2H])c([2H])c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3oc3cc(-c5ccccc5)ccc34)c([2H])c([2H])c1n2-c1ccccc1]

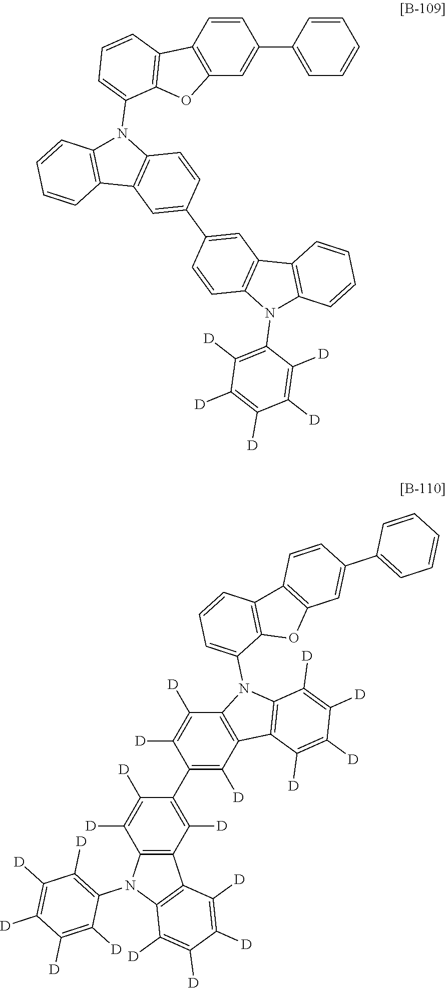 [SMILES: [2H]c1c([2H])c([2H])c(-n2c3ccccc3c3cc(-c4ccc5c(c4)c4ccccc4n5-c4cccc5c4oc4cc(-c6ccccc6)ccc45)ccc32)c([2H])c1[2H] ; [2H]c1c([2H])c([2H])c(-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5c4oc4cc(-c6ccccc6)ccc45)c([2H])c([2H])c32)c([2H])c1[2H]]

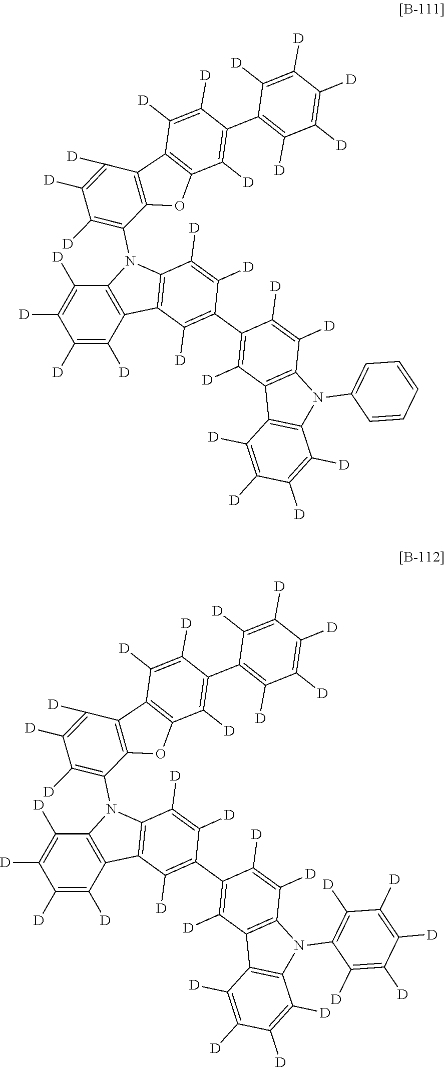 [SMILES: [2H]c1c([2H])c([2H])c(-c2c([2H])c([2H])c3c(oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7)c([2H])c([2H])c65)c([2H])c([2H])c([2H])c43)c2[2H])c([2H])c1[2H] ; [2H]c1c([2H])c([2H])c(-c2c([2H])c([2H])c3c(oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7c([2H])c([2H])c([2H])c([2H])c7[2H])c([2H])c([2H])c65)c([2H])c([2H])c([2H])c43)c2[2H])c([2H])c1[2H]]

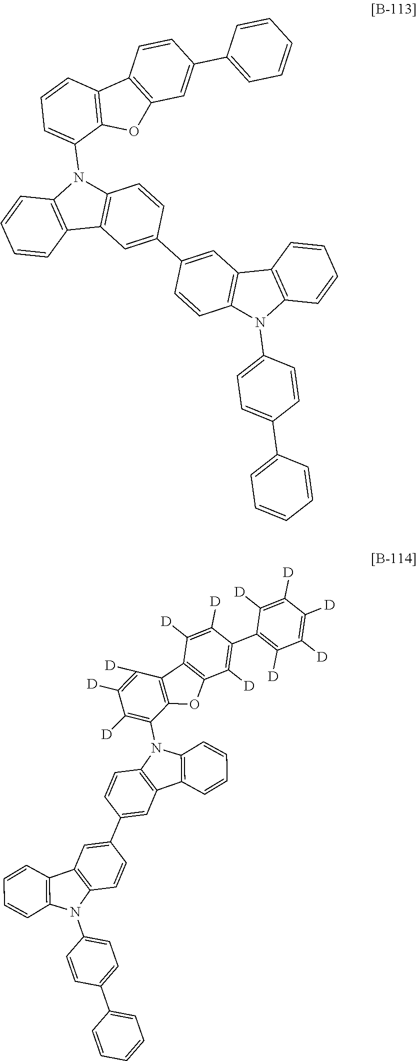 [SMILES: c1ccc(-c2ccc(-n3c4ccccc4c4cc(-c5ccc6c(c5)c5ccccc5n6-c5cccc6c5oc5cc(-c7ccccc7)ccc56)ccc43)cc2)cc1 ; [2H]c1c([2H])c([2H])c(-c2c([2H])c([2H])c3c(oc4c(-n5c6ccccc6c6cc(-c7ccc8c(c7)c7ccccc7n8-c7ccc(-c8ccccc8)cc7)ccc65)c([2H])c([2H])c([2H])c43)c2[2H])c([2H])c1[2H]]

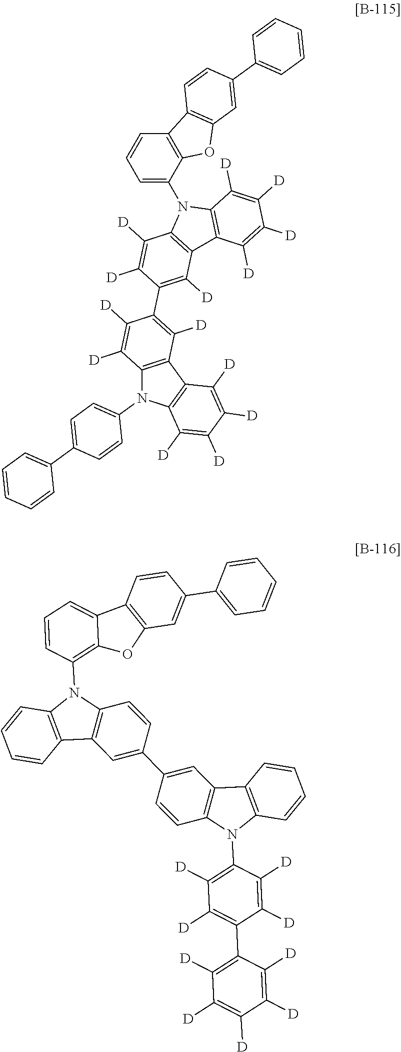 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3oc3cc(-c5ccccc5)ccc34)c([2H])c([2H])c1n2-c1ccc(-c2ccccc2)cc1 ; [2H]c1c([2H])c([2H])c(-c2c([2H])c([2H])c(-n3c4ccccc4c4cc(-c5ccc6c(c5)c5ccccc5n6-c5cccc6c5oc5cc(-c7ccccc7)ccc56)ccc43)c([2H])c2[2H])c([2H])c1[2H]]

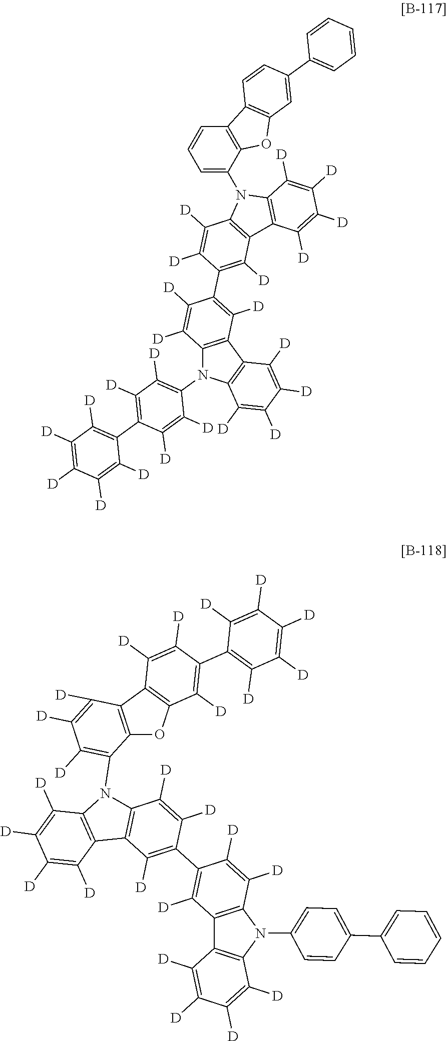 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c5oc5cc(-c7ccccc7)ccc56)c([2H])c([2H])c43)c([2H])c2[2H])c([2H])c1[2H] ; [2H]c1c([2H])c([2H])c(-c2c([2H])c([2H])c3c(oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccc(-c8ccccc8)cc7)c([2H])c([2H])c65)c([2H])c([2H])c([2H])c43)c2[2H])c([2H])c1[2H]]

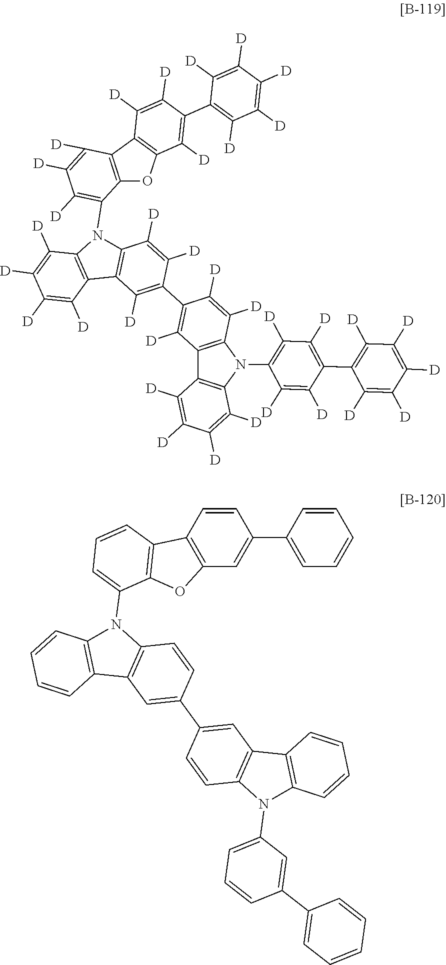 [SMILES: [2H]c1c([2H])c([2H])c(-c2c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5c([2H])c([2H])c([2H])c6c5oc5c([2H])c(-c7c([2H])c([2H])c([2H])c([2H])c7[2H])c([2H])c([2H])c56)c([2H])c([2H])c43)c([2H])c2[2H])c([2H])c1[2H] ; c1ccc(-c2cccc(-n3c4ccccc4c4cc(-c5ccc6c(c5)c5ccccc5n6-c5cccc6c5oc5cc(-c7ccccc7)ccc56)ccc43)c2)cc1]

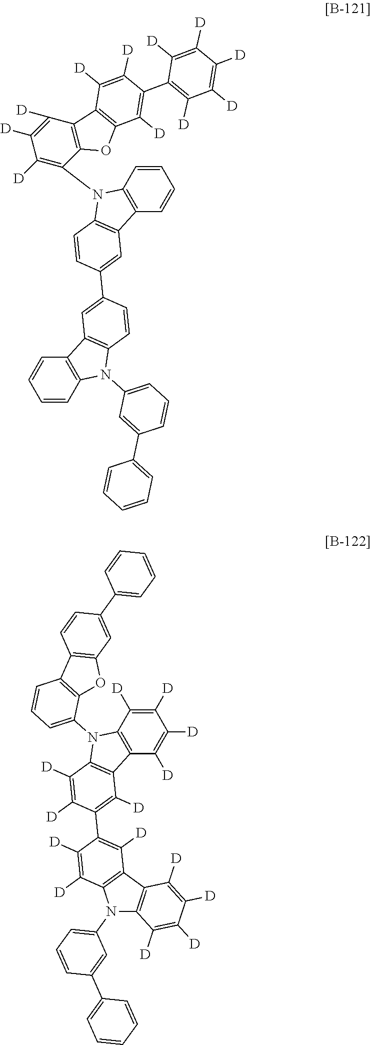 [SMILES: [2H]c1c([2H])c([2H])c(-c2c([2H])c([2H])c3c(oc4c(-n5c6ccccc6c6cc(-c7ccc8c(c7)c7ccccc7n8-c7cccc(-c8ccccc8)c7)ccc65)c([2H])c([2H])c([2H])c43)c2[2H])c([2H])c1[2H] ; [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3oc3cc(-c5ccccc5)ccc34)c([2H])c([2H])c1n2-c1cccc(-c2ccccc2)c1]

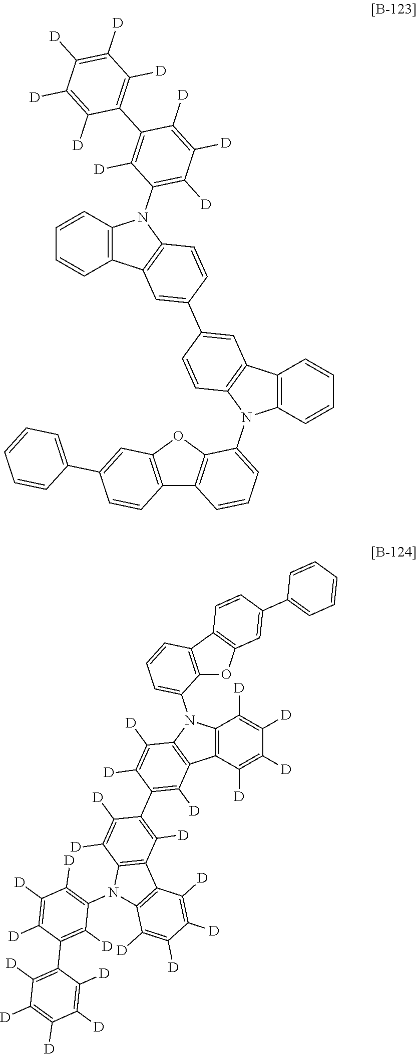 [SMILES: [2H]c1c([2H])c([2H])c(-c2c([2H])c([2H])c([2H])c(-n3c4ccccc4c4cc(-c5ccc6c(c5)c5ccccc5n6-c5cccc6c5oc5cc(-c7ccccc7)ccc56)ccc43)c2[2H])c([2H])c1[2H] ; [2H]c1c([2H])c([2H])c(-c2c([2H])c([2H])c([2H])c(-n3c4c([2H])c([2H])c([2H])c([2H])c4c4c([2H])c(-c5c([2H])c([2H])c6c(c5[2H])c5c([2H])c([2H])c([2H])c([2H])c5n6-c5cccc6c5oc5cc(-c7ccccc7)ccc56)c([2H])c([2H])c43)c2[2H])c([2H])c1[2H]]

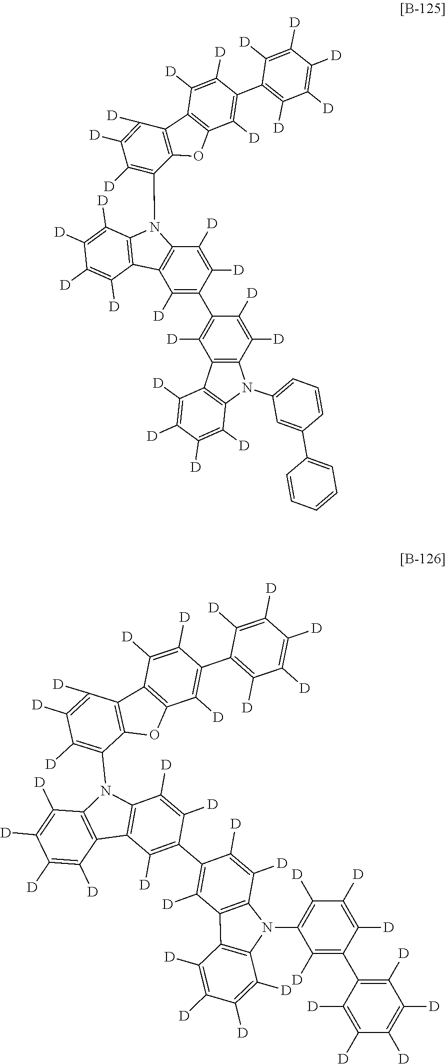

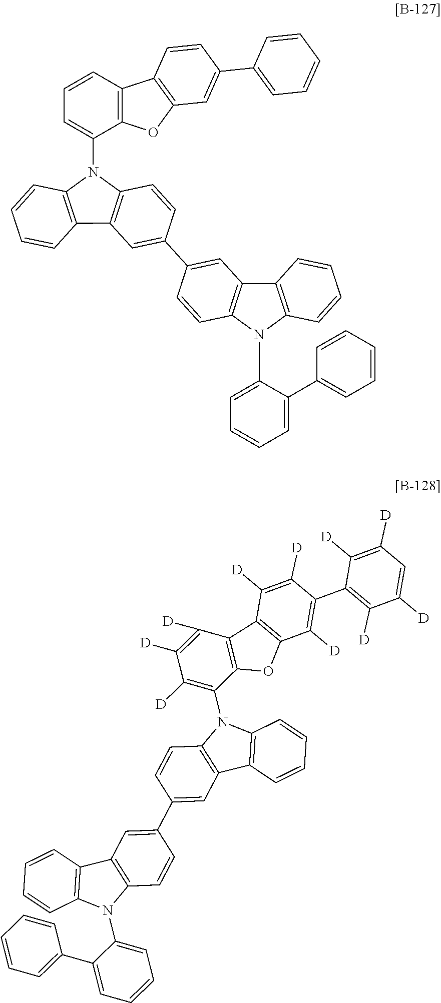 [SMILES: c1ccc(-c2ccc3c(c2)oc2c(-n4c5ccccc5c5cc(-c6ccc7c(c6)c6ccccc6n7-c6ccccc6-c6ccccc6)ccc54)cccc23)cc1 ; [2H]c1cc([2H])c([2H])c(-c2c([2H])c([2H])c3c(oc4c(-n5c6ccccc6c6cc(-c7ccc8c(c7)c7ccccc7n8-c7ccccc7-c7ccccc7)ccc65)c([2H])c([2H])c([2H])c43)c2[2H])c1[2H]]

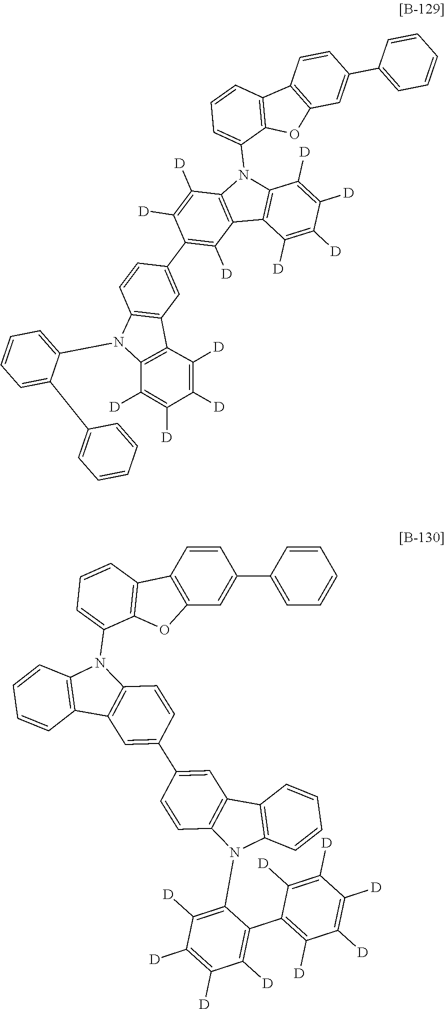 [SMILES: [2H]c1c([2H])c([2H])c2c(c1[2H])c1cc(-c3c([2H])c([2H])c4c(c3[2H])c3c([2H])c([2H])c([2H])c([2H])c3n4-c3cccc4c3oc3cc(-c5ccccc5)ccc34)ccc1n2-c1ccccc1-c1ccccc1 ; [2H]c1c([2H])c([2H])c(-c2c([2H])c([2H])c([2H])c([2H])c2-n2c3ccccc3c3cc(-c4ccc5c(c4)c4ccccc4n5-c4cccc5c4oc4cc(-c6ccccc6)ccc45)ccc32)c([2H])c1[2H]]

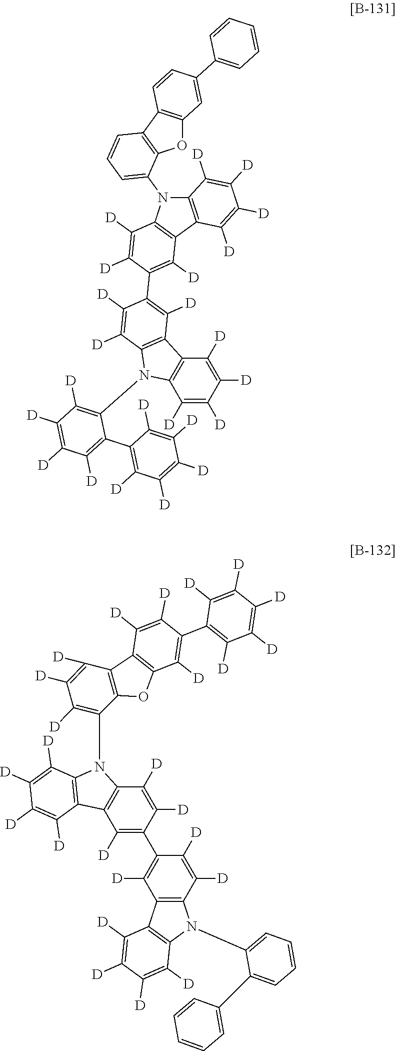 [SMILES: [2H]c1c([2H])c([2H])c(-c2c([2H])c([2H])c([2H])c([2H])c2-n2c3c([2H])c([2H])c([2H])c([2H])c3c3c([2H])c(-c4c([2H])c([2H])c5c(c4[2H])c4c([2H])c([2H])c([2H])c([2H])c4n5-c4cccc5c4oc4cc(-c6ccccc6)ccc45)c([2H])c([2H])c32)c([2H])c1[2H] ; [2H]c1c([2H])c([2H])c(-c2c([2H])c([2H])c3c(oc4c(-n5c6c([2H])c([2H])c([2H])c([2H])c6c6c([2H])c(-c7c([2H])c([2H])c8c(c7[2H])c7c([2H])c([2H])c([2H])c([2H])c7n8-c7ccccc7-c7ccccc7)c([2H])c([2H])c65)c([2H])c([2H])c([2H])c43)c2[2H])c([2H])c1[2H]]

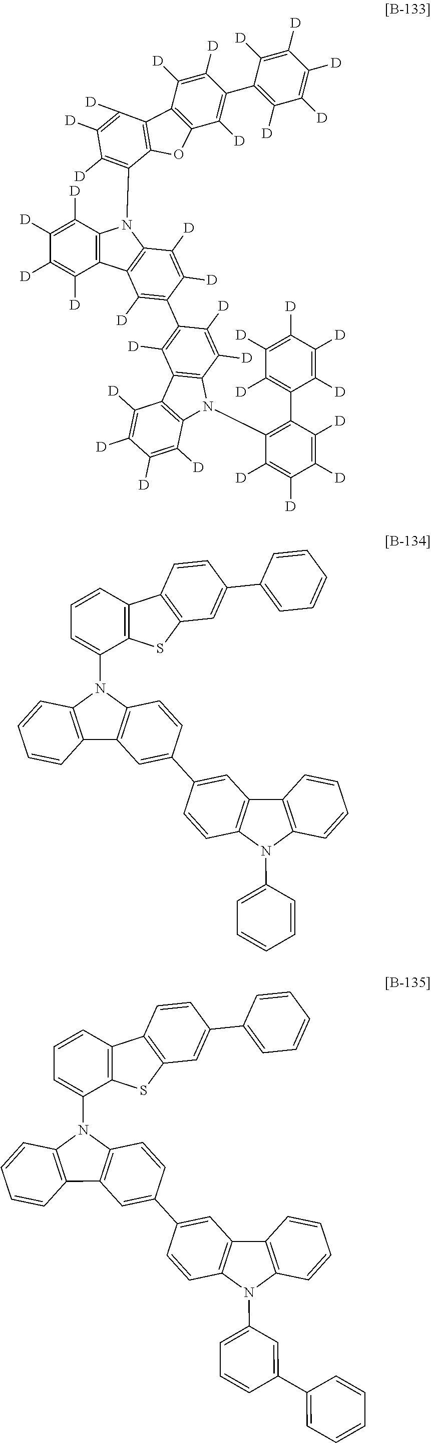

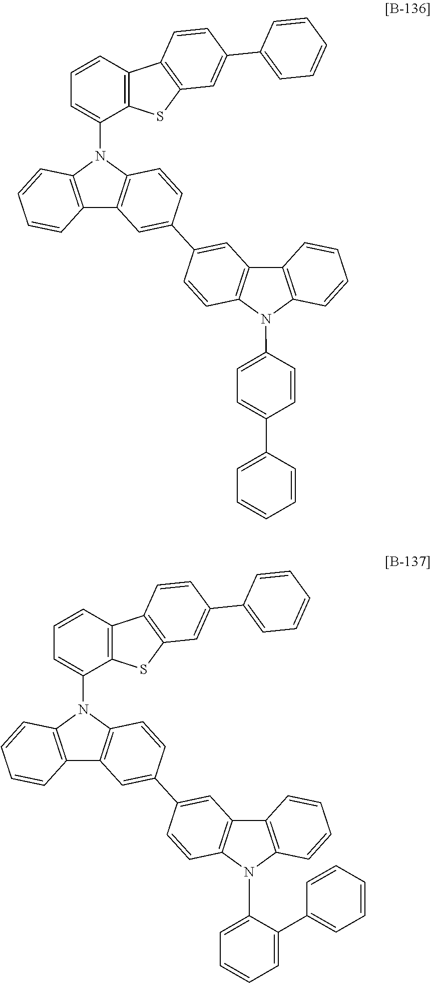 [SMILES: c1ccc(-c2ccc(-n3c4ccccc4c4cc(-c5ccc6c(c5)c5ccccc5n6-c5cccc6c5sc5cc(-c7ccccc7)ccc56)ccc43)cc2)cc1 ; c1ccc(-c2ccc3c(c2)sc2c(-n4c5ccccc5c5cc(-c6ccc7c(c6)c6ccccc6n7-c6ccccc6-c6ccccc6)ccc54)cccc23)cc1]

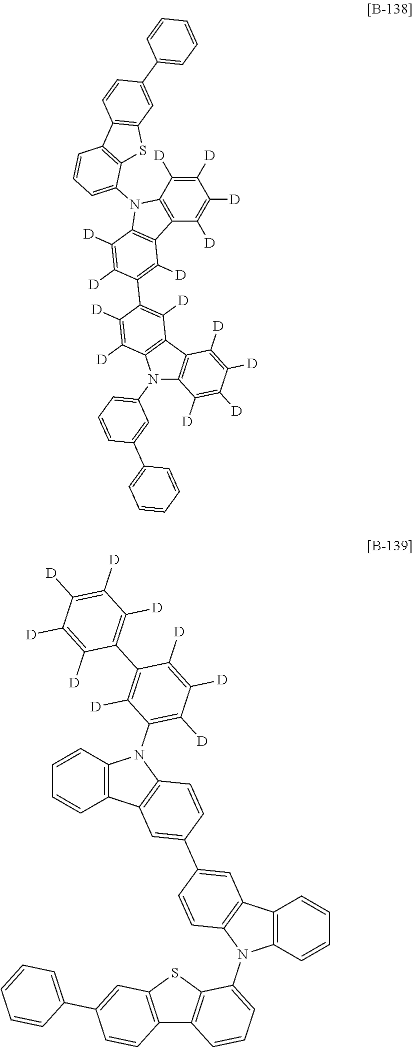 [SMILES: [2H]c1c([2H])c([2H])c2c(c1[2H])c1c([2H])c(-c3c([2H])c([2H])c4c(c3[2H])c3c([2H])c([2H])c([2H])c([2H])c3n4-c3cccc4c3sc3cc(-c5ccccc5)ccc34)c([2H])c([2H])c1n2-c1cccc(-c2ccccc2)c1 ; [2H]c1c([2H])c([2H])c(-c2c([2H])c([2H])c([2H])c(-n3c4ccccc4c4cc(-c5ccc6c(c5)c5ccccc5n6-c5cccc6c5sc5cc(-c7ccccc7)ccc56)ccc43)c2[2H])c([2H])c1[2H]]

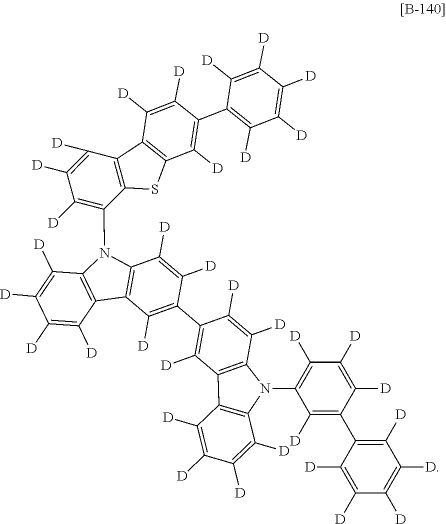

8. An organic optoelectronic device, comprising
an anode and a cathode facing each other, and
at least one organic layer between the anode and the cathode,
wherein the organic layer comprises the composition for the organic optoelectronic device as claimed in claim 1.

9. The organic optoelectronic device as claimed in claim 8, wherein
the organic layer comprises a light emitting layer, and
the light emitting layer comprises the composition for the organic optoelectronic device.

10. The organic optoelectronic device as claimed in claim 9, wherein
the composition for the organic optoelectronic device further comprises a phosphorescent dopant.

11. The organic optoelectronic device as claimed in claim 10, wherein
the composition for the organic optoelectronic device is a green light emitting composition.

12. A display device comprising the organic optoelectronic device as claimed in claim 8.
